SEQUENCE LISTING (557 sequences)

SEQ ID NO: 1 - DNA, 141 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 130 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 3 - DNA, 1923 bp, Artificial Sequence
  Description of Artificial Sequence: Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 4 - DNA, 1272 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 5 - DNA, 547 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 6 - DNA, 1179 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 8 bp, Artificial Sequence
  Description of Artificial Sequence: Synthetic oligonucleotide
gtttaaac

SEQ ID NO: 8 - DNA, 581 bp, Artificial Sequence
  Description of Artificial Sequence: Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 9 - DNA, 225 bp, Artificial Sequence
  Description of Artificial Sequence: Synthetic polynucleotide
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 10 - DNA, 213 bp, Artificial Sequence
  Description of Artificial Sequence: Synthetic polynucleotide
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 11 - DNA, 1260 bp, Adeno-associated virus - 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 12 - DNA, 1932 bp, Adeno-associated virus - 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 13 - DNA, 1876 bp, Artificial Sequence
  Description of Artificial Sequence: Synthetic polynucleotide
cgcagccaccatggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 1194 bp, Artificial Sequence
  Description of Artificial Sequence: Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 141 bp, Artificial Sequence
  Description of Artificial Sequence: Synthetic polynucleotide
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 16 - DNA, 556 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactga

SEQ ID NO: 17 - DNA, 80 bp, Artificial Sequence
  Description of Artificial Sequence: Synthetic oligonucleotide
gcgcgctcgctcgctcactgaggccgggcgaccaaaggtcgcccgacgcccgggcggcctcagtgagcgagcgagcgcgc

SEQ ID NO: 18 - DNA, 241 bp, Artificial Sequence
  Description of Artificial Sequence: Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 19 - DNA, 215 bp, Artificial Sequence
  Description of Artificial Sequence: Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 20 - DNA, 150 bp, Artificial Sequence
  Description of Artificial Sequence: Synthetic polynucleotide
ataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcggccatct

SEQ ID NO: 21 - DNA, 546 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 22 - DNA, 317 bp, Artificial Sequence
  Description of Artificial Sequence: Synthetic polynucleotide
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 23 - DNA, 576 bp, Artificial Sequence
  Description of Artificial Sequence: Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 24 - DNA, 1313 bp, Artificial Sequence
  Description of Artificial Sequence: Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaa

SEQ ID NO: 25 - protein, 19 aa, Artificial Sequence
  Description of Artificial Sequence: Synthetic peptide
MDWTWRILFLVAAATGAHS

SEQ ID NO: 26 - protein, 19 aa, Artificial Sequence
  Description of Artificial Sequence: Synthetic peptide
MLPSQLIGFLLLWVPASRG

SEQ ID NO: 27 - protein, 7 aa, Artificial Sequence
  Description of Artificial Sequence: Synthetic peptide
PKKKRKV

SEQ ID NO: 28-44 - skipped (17 entries)

SEQ ID NO: 45 - DNA, 6 bp, Artificial Sequence
  Description of Artificial Sequence: Synthetic oligonucleotide
ggttga

SEQ ID NO: 46 - DNA, 4 bp, Artificial Sequence
  Description of Artificial Sequence: Synthetic oligonucleotide
agtt

SEQ ID NO: 47 - DNA, 6 bp, Artificial Sequence
  Description of Artificial Sequence: Synthetic oligonucleotide
ggttgg

SEQ ID NO: 48 - DNA, 6 bp, Artificial Sequence
  Description of Artificial Sequence: Synthetic oligonucleotide
agttgg

SEQ ID NO: 49 - DNA, 6 bp, Artificial Sequence
  Description of Artificial Sequence: Synthetic oligonucleotide
agttga

SEQ ID NO: 50 - DNA, 6 bp, Artificial Sequence
  Description of Artificial Sequence: Synthetic oligonucleotide
rrttrr

SEQ ID NO: 51 - DNA, 141 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 52 - DNA, 130 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53 - DNA, 3123 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 54 - DNA, 3117 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 55-56 - skipped (2 entries)

SEQ ID NO: 57 - DNA, 3912 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcctggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 3906 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 59-62 - skipped (4 entries)

SEQ ID NO: 63 - DNA, 126 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcct

SEQ ID NO: 64 - DNA, 120 bp, Artificial Sequence
  Description of Artificial Sequence: Synthetic polynucleotide
aggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 65 - DNA, 80 bp, Artificial Sequence
  Description of Artificial Sequence: Synthetic oligonucleotide
gcgcgctcgctcgctcactgaggccgcccgggcgtcgggcgacctttggtcgcccggcctcagtgagcgagcgagcgcgc

SEQ ID NO: 66 - DNA, 141 bp, Artificial Sequence; identical to SEQ ID NO: 15
  Description of Artificial Sequence: Synthetic polynucleotide

SEQ ID NO: 67 - DNA, 1876 bp, Artificial Sequence
  Description of Artificial Sequence: Synthetic polynucleotide
cgcagccaccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 68 - DNA, 129 bp, Artificial Sequence
  Description of Artificial Sequence: Synthetic polynucleotide
atcatggagataattaaaatgataaccatctcgcaaataaataagtattttactgttttcgtaacagttttgtaataaaaaaacctataaatattccggattattcataccgtcccaccatcgggcgcg

SEQ ID NO: 69 - DNA, 1203 bp, Artificial Sequence
  Description of Artificial Sequence: Synthetic polynucleotide
gccgccaccatggagttggtgggctggctcgtggacaaaggcattacttcggaaaagcagtggattcaggaggatcaggcatcttacatctcattcaacgctgccagtaactcgaggtcccagatcaaggcagcgctggacaacgcgggaaagattatgagtctgaccaaaactgctccagactacctcgttggtcagcaaccggtggaagatatctccagcaacaggatctacaagattctggagctcaacggctacgaccctcaatacgctgcctcagtgttcttgggttgggccaccaagaaattcggcaagagaaacactatctggctgttcggccccgctaccactggaaagacaaacatcgcagaagcgattgctcacacggtgccattctacggctgcgtcaactggacaaacgagaacttcccgttcaacgactgtgtcgataagatggttatctggtgggaggaaggaaagatgacggccaaagtggtcgaaagcgccaaggcaattctgggtggctctaaagtgcgcgtcgaccagaagtgcaaatcttcagctcaaatcgatcctacccccgttattgtgacatcaaacacgaacatgtgtgccgtgatcgacggaaacagtacaacgttcgaacaccagcaacctctccaggatcgtatgttcaagttcgagctcacccgccgtttggaccatgatttcggcaaggtcactaaacaagaggttaaggacttcttccgctgggctaaagatcacgttgtggaggttgaacatgagttctacgtcaagaaaggaggtgctaagaaacgtccagccccgtcggacgcagatatctccgaacctaagagggtgagagagtcggtcgcacagccaagcacttctgacgcagaagcttccattaactacgcagataggtaccaaaacaagtgcagcagacacgtgggtatgaacttgatgctgttcccatgccgccagtgtgagcgtatgaaccaaaactctaacatctgtttcacacatggccagaaggactgcctcgaatgtttccctgtgtcagagagtcagcccgtctcagtcgttaagaaagcttaccaaaagttgtgctacatccaccatattatgggtaaagtccctgatgcctgtaccgcttgtgatctggtcaacgtggatttggacgactgtattttcgagcaataa

SEQ ID NO: 70 - DNA, 388 bp, Artificial Sequence
  Description of Artificial Sequence: Synthetic polynucleotide
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaag

SEQ ID NO: 71 - DNA, 1662 bp, Artificial Sequence
  Description of Artificial Sequence: Synthetic polynucleotide
gccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 72 - DNA, 581 bp, Artificial Sequence; identical to SEQ ID NO: 8
  Description of Artificial Sequence: Synthetic polynucleotide

SEQ ID NO: 73 - DNA, 225 bp, Artificial Sequence; identical to SEQ ID NO: 9
  Description of Artificial Sequence: Synthetic polynucleotide

SEQ ID NO: 74 - DNA, 1177 bp, Artificial Sequence
  Description of Artificial Sequence: Synthetic polynucleotide
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75-96 - skipped (22 entries)

SEQ ID NO: 97 - DNA, 80 bp, Artificial Sequence; identical to SEQ ID NO: 17
  Description of Artificial Sequence: Synthetic oligonucleotide

SEQ ID NO: 98 - DNA, 72 bp, Artificial Sequence
  Description of Artificial Sequence: Synthetic oligonucleotide
gcgcgctcgctcgctcactgaggccgggcgaccaaaggtcgcccgacggcctcagtgagcgagcgagcgcgc

SEQ ID NO: 99 - DNA, 122 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 100 - DNA, 130 bp, Artificial Sequence; identical to SEQ ID NO: 2
  Description of Artificial Sequence: Synthetic polynucleotide

SEQ ID NO: 101 - DNA, 70 bp, Artificial Sequence
  Description of Artificial Sequence: Synthetic oligonucleotide
gcgcgctcgctcgctcactgaggccgcccgggaaacccgggcgtgcgcctcagtgagcgagcgagcgcgc

SEQ ID NO: 102 - DNA, 70 bp, Artificial Sequence
  Description of Artificial Sequence: Synthetic oligonucleotide
gcgcgctcgctcgctcactgaggcgcacgcccgggtttcccgggcggcctcagtgagcgagcgagcgcgc

SEQ ID NO: 103 - DNA, 72 bp, Artificial Sequence
  Description of Artificial Sequence: Synthetic oligonucleotide
gcgcgctcgctcgctcactgaggccgtcgggcgacctttggtcgcccggcctcagtgagcgagcgagcgcgc

SEQ ID NO: 104 - DNA, 72 bp, Artificial Sequence; identical to SEQ ID NO: 98
  Description of Artificial Sequence: Synthetic oligonucleotide

SEQ ID NO: 105 - DNA, 72 bp, Artificial Sequence
  Description of Artificial Sequence: Synthetic oligonucleotide
gcgcgctcgctcgctcactgaggccgcccgggcaaagcccgggcgtcggcctcagtgagcgagcgagcgcgc

SEQ ID NO: 106 - DNA, 72 bp, Artificial Sequence
  Description of Artificial Sequence: Synthetic oligonucleotide
gcgcgctcgctcgctcactgaggccgacgcccgggctttgcccgggcggcctcagtgagcgagcgagcgcgc

SEQ ID NO: 107 - DNA, 83 bp, Artificial Sequence
  Description of Artificial Sequence: Synthetic oligonucleotide
gcgcgctcgctcgctcactgaggccgcccgggcaaagcccgggcgtcgggctttgcccggcctcagtgagcgagcgagcgcgc

SEQ ID NO: 108 - DNA, 83 bp, Artificial Sequence
  Description of Artificial Sequence: Synthetic oligonucleotide
gcgcgctcgctcgctcactgaggccgggcaaagcccgacgcccgggctttgcccgggcggcctcagtgagcgagcgagcgcgc

SEQ ID NO: 109 - DNA, 77 bp, Artificial Sequence
  Description of Artificial Sequence: Synthetic oligonucleotide
gcgcgctcgctcgctcactgaggccgaaacgtcgggcgacctttggtcgcccggcctcagtgagcgagcgagcgcgc

SEQ ID NO: 110 - DNA, 77 bp, Artificial Sequence
  Description of Artificial Sequence: Synthetic oligonucleotide
gcgcgctcgctcgctcactgaggccgggcgaccaaaggtcgcccgacgtttcggcctcagtgagcgagcgagcgcgc

SEQ ID NO: 111 - DNA, 51 bp, Artificial Sequence
  Description of Artificial Sequence: Synthetic oligonucleotide
gcgcgctcgctcgctcactgaggcaaagcctcagtgagcgagcgagcgcgc

SEQ ID NO: 112 - DNA, 51 bp, Artificial Sequence
  Description of Artificial Sequence: Synthetic oligonucleotide
gcgcgctcgctcgctcactgaggctttgcctcagtgagcgagcgagcgcgc

SEQ ID NO: 113 - DNA, 80 bp, Artificial Sequence; identical to SEQ ID NO: 65
  Description of Artificial Sequence: Synthetic oligonucleotide

SEQ ID NO: 114 - DNA, 80 bp, Artificial Sequence; identical to SEQ ID NO: 17
  Description of Artificial Sequence: Synthetic oligonucleotide

SEQ ID NO: 115 - DNA, 77 bp, Artificial Sequence
  Description of Artificial Sequence: Synthetic oligonucleotide
gcgcgctcgctcgctcactgaggcgcccgggcgtcgggcgacctttggtcgcccggcctcagtgagcgagcgagcgc

SEQ ID NO: 116 - DNA, 79 bp, Artificial Sequence
  Description of Artificial Sequence: Synthetic oligonucleotide
gcgcgctcgctcgctcactgaggccgggcgaccaaaggtcgcccgacgcccgggcgcctcagtgagcgagcgagcgcgc

SEQ ID NO: 117 - DNA, 89 bp, Artificial Sequence
  Description of Artificial Sequence: Synthetic oligonucleotide
gcgcgctcgctcgctcactgaggccgcccgggcaaagcccgggcgtcgggcgactttgtcgcccggcctcagtgagcgagcgagcgcgc

SEQ ID NO: 118 - DNA, 89 bp, Artificial Sequence
  Description of Artificial Sequence: Synthetic oligonucleotide
gcgcgctcgctcgctcactgaggccgggcgacaaagtcgcccgacgcccgggctttgcccgggcggcctcagtgagcgagcgagcgcgc

SEQ ID NO: 119 - DNA, 87 bp, Artificial Sequence
  Description of Artificial Sequence: Synthetic oligonucleotide
gcgcgctcgctcgctcactgaggccgcccgggcaaagcccgggcgtcgggcgattttcgcccggcctcagtgagcgagcgagcgcgc

SEQ ID NO: 120 - DNA, 87 bp, Artificial Sequence
  Description of Artificial Sequence: Synthetic oligonucleotide
gcgcgctcgctcgctcactgaggccgggcgaaaatcgcccgacgcccgggctttgcccgggcggcctcagtgagcgagcgagcgcgc

SEQ ID NO: 121 - DNA, 85 bp, Artificial Sequence
  Description of Artificial Sequence: Synthetic oligonucleotide
gcgcgctcgctcgctcactgaggccgcccgggcaaagcccgggcgtcgggcgtttcgcccggcctcagtgagcgagcgagcgcgc

SEQ ID NO: 122 - DNA, 85 bp, Artificial Sequence
  Description of Artificial Sequence: Synthetic oligonucleotide
gcgcgctcgctcgctcactgaggccgggcgaaacgcccgacgcccgggctttgcccgggcggcctcagtgagcgagcgagcgcgc

SEQ ID NO: 123 - DNA, 89 bp, Artificial Sequence
  Description of Artificial Sequence: Synthetic oligonucleotide
gcgcgctcgctcgctcactgaggccgcccgggaaacccgggcgtcgggcgacctttggtcgcccggcctcagtgagcgagcgagcgcgc

SEQ ID NO: 124 - DNA, 89 bp, Artificial Sequence
  Description of Artificial Sequence: Synthetic oligonucleotide
gcgcgctcgctcgctcactgaggccgggcgaccaaaggtcgcccgacgcccgggtttcccgggcggcctcagtgagcgagcgagcgcgc

SEQ ID NO: 125 - DNA, 87 bp, Artificial Sequence
  Description of Artificial Sequence: Synthetic oligonucleotide
gcgcgctcgctcgctcactgaggccgcccggaaaccgggcgtcgggcgacctttggtcgcccggcctcagtgagcgagcgagcgcgc

SEQ ID NO: 126 - DNA, 87 bp, Artificial Sequence
  Description of Artificial Sequence: Synthetic oligonucleotide
gcgcgctcgctcgctcactgaggccgggcgaccaaaggtcgcccgacgcccggtttccgggcggcctcagtgagcgagcgagcgcgc

SEQ ID NO: 127 - DNA, 85 bp, Artificial Sequence
  Description of Artificial Sequence: Synthetic oligonucleotide
gcgcgctcgctcgctcactgaggccgcccgaaacgggcgtcgggcgacctttggtcgcccggcctcagtgagcgagcgagcgcgc

SEQ ID NO: 128 - DNA, 85 bp, Artificial Sequence
  Description of Artificial Sequence: Synthetic oligonucleotide
gcgcgctcgctcgctcactgaggccgggcgaccaaaggtcgcccgacgcccgtttcgggcggcctcagtgagcgagcgagcgcgc

SEQ ID NO: 129 - DNA, 83 bp, Artificial Sequence
  Description of Artificial Sequence: Synthetic oligonucleotide
gcgcgctcgctcgctcactgaggccgcccaaagggcgtcgggcgacctttggtcgcccggcctcagtgagcgagcgagcgcgc

SEQ ID NO: 130 - DNA, 83 bp, Artificial Sequence
  Description of Artificial Sequence: Synthetic oligonucleotide
gcgcgctcgctcgctcactgaggccgggcgaccaaaggtcgcccgacgccctttgggcggcctcagtgagcgagcgagcgcgc

SEQ ID NO: 131 - DNA, 81 bp, Artificial Sequence
  Description of Artificial Sequence: Synthetic oligonucleotide
gcgcgctcgctcgctcactgaggccgccaaaggcgtcgggcgacctttggtcgcccggcctcagtgagcgagcgagcgcgc

SEQ ID NO: 132 - DNA, 81 bp, Artificial Sequence
  Description of Artificial Sequence: Synthetic oligonucleotide
gcgcgctcgctcgctcactgaggccgggcgaccaaaggtcgcccgacgcctttggcggcctcagtgagcgagcgagcgcgc

SEQ ID NO: 133 - DNA, 79 bp, Artificial Sequence
  Description of Artificial Sequence: Synthetic oligonucleotide
gcgcgctcgctcgctcactgaggccgcaaagcgtcgggcgacctttggtcgcccggcctcagtgagcgagcgagcgcgc

SEQ ID NO: 134 - DNA, 79 bp, Artificial Sequence
  Description of Artificial Sequence: Synthetic oligonucleotide
gcgcgctcgctcgctcactgaggccgggcgaccaaaggtcgcccgacgctttgcggcctcagtgagcgagcgagcgcgc

SEQ ID NO: 135 - DNA, 80 bp, Artificial Sequence
  Description of Artificial Sequence: Synthetic oligonucleotide
gcgcgctcgctcgctcactgaggccgggcgaccaaaggtcgcccgaagcccgggctgcctcagtgagcgagcgagcgcgc

SEQ ID NO: 136 - DNA, 80 bp, Artificial Sequence
  Description of Artificial Sequence: Synthetic oligonucleotide
gcgcgctcgctcgctcactgaggccgggcgaccaaaggtcgcccgacacccgggtggcctcagtgagcgagcgagcgcgc

SEQ ID NO: 137 - DNA, 80 bp, Artificial Sequence
  Description of Artificial Sequence: Synthetic oligonucleotide
gcgcgctcgctcgctcactgaggccgggcgaccaaaggtcgcccgacgaccggtcggcctcagtgagcgagcgagcgcgc

SEQ ID NO: 138 - DNA, 80 bp, Artificial Sequence
  Description of Artificial Sequence: Synthetic oligonucleotide
gcgcgctcgctcgctcactgaggccgggcgaccaaaggtcgcccgacgcacgtgcggcctcagtgagcgagcgagcgcgc

SEQ ID NO: 139 - DNA, 80 bp, Artificial Sequence
  Description of Artificial Sequence: Synthetic oligonucleotide
gcgcgctcgctcgctcactgaggccgggcgaccaaaggtcgcccgacgccatggcggcctcagtgagcgagcgagcgcgc

SEQ ID NO: 140 - DNA, 80 bp, Artificial Sequence
  Description of Artificial Sequence: Synthetic oligonucleotide
gcgcgctcgctcgctcactgaggccgggcgaccaaaggtcgcccgaagaccggtctgcctcagtgagcgagcgagcgcgc

SEQ ID NO: 141 - DNA, 80 bp, Artificial Sequence
  Description of Artificial Sequence: Synthetic oligonucleotide
gcgcgctcgctcgctcactgaggccgggcgaccaaaggtcgcccgacacacgtgtggcctcagtgagcgagcgagcgcgc

SEQ ID NO: 142 - DNA, 80 bp, Artificial Sequence
  Description of Artificial Sequence: Synthetic oligonucleotide
gcgcgctcgctcgctcactgaggccgggcgaccaaaggtcgcccgacgacatgtcggcctcagtgagcgagcgagcgcgc

SEQ ID NO: 143 - DNA, 80 bp, Artificial Sequence
  Description of Artificial Sequence: Synthetic oligonucleotide
gcgcgctcgctcgctcactgaggccgggcgaccaaaggtcgcccgaagcacgtgctgcctcagtgagcgagcgagcgcgc

SEQ ID NO: 144 - DNA, 80 bp, Artificial Sequence
  Description of Artificial Sequence: Synthetic oligonucleotide
gcgcgctcgctcgctcactgaggccgggcgaccaaaggtcgcccgaaacccgggttgcctcagtgagcgagcgagcgcgc

SEQ ID NO: 145 - DNA, 80 bp, Artificial Sequence
  Description of Artificial Sequence: Synthetic oligonucleotide
gcgcgctcgctcgctcactgaggccgggcgaccaaaggtcgcccgaagccatggctgcctcagtgagcgagcgagcgcgc

SEQ ID NO: 146 - DNA, 80 bp, Artificial Sequence
  Description of Artificial Sequence: Synthetic oligonucleotide
gcgcgctcgctcgctcactgaggccgggcgaccaaaggtcgcccgacaaccggttggcctcagtgagcgagcgagcgcgc

SEQ ID NO: 147 - DNA, 80 bp, Artificial Sequence
  Description of Artificial Sequence: Synthetic oligonucleotide
gcgcgctcgctcgctcactgaggccgggcgaccaaaggtcgcccgacaccatggtggcctcagtgagcgagcgagcgcgc

SEQ ID NO: 148 - DNA, 80 bp, Artificial Sequence
  Description of Artificial Sequence: Synthetic oligonucleotide
gcgcgctcgctcgctcactgaggccgggcgaccaaaggtcgcccgacgaacgttcggcctcagtgagcgagcgagcgcgc

SEQ ID NO: 149 - DNA, 80 bp, Artificial Sequence
  Description of Artificial Sequence: Synthetic oligonucleotide
gcgcgctcgctcgctcactgaggccgggcgaccaaaggtcgcccgacgcaattgcggcctcagtgagcgagcgagcgcgc

SEQ ID NO: 150 - DNA, 80 bp, Artificial Sequence
  Description of Artificial Sequence: Synthetic oligonucleotide
gcgcgctcgctcgctcactgaggccgggcgaccaaaggtcgcccgaaaaccggtttgcctcagtgagcgagcgagcgcgc

SEQ ID NO: 151 - DNA, 80 bp, Artificial Sequence
  Description of Artificial Sequence: Synthetic oligonucleotide
gcgcgctcgctcgctcactgaggccgggcgaccaaaggtcgcccgaagaacgttctgcctcagtgagcgagcgagcgcgc

SEQ ID NO: 152 - DNA, 80 bp, Artificial Sequence
  Description of Artificial Sequence: Synthetic oligonucleotide
gcgcgctcgctcgctcactgaggccgggcgaccaaaggtcgcccgaagcaattgctgcctcagtgagcgagcgagcgcgc

SEQ ID NO: 153 - DNA, 80 bp, Artificial Sequence
  Description of Artificial Sequence: Synthetic oligonucleotide
gcgcgctcgctcgctcactgaggccgggcgaccaaaggtcgcccgacaaacgtttggcctcagtgagcgagcgagcgcgc

SEQ ID NO: 154 - DNA, 80 bp, Artificial Sequence
  Description of Artificial Sequence: Synthetic oligonucleotide
gcgcgctcgctcgctcactgaggccgggcgaccaaaggtcgcccgacgaaatttcggcctcagtgagcgagcgagcgcgc

SEQ ID NO: 155 - DNA, 80 bp, Artificial Sequence; identical to SEQ ID NO: 152
  Description of Artificial Sequence: Synthetic oligonucleotide

SEQ ID NO: 156 - DNA, 80 bp, Artificial Sequence
  Description of Artificial Sequence: Synthetic oligonucleotide
gcgcgctcgctcgctcactgaggccgggcgaccaaaggtcgcccgaaaccatggttgcctcagtgagcgagcgagcgcgc

SEQ ID NO: 157 - DNA, 80 bp, Artificial Sequence
  Description of Artificial Sequence: Synthetic oligonucleotide
gcgcgctcgctcgctcactgaggccgggcgaccaaaggtcgcccgacaacatgttggcctcagtgagcgagcgagcgcgc

SEQ ID NO: 158 - DNA, 80 bp, Artificial Sequence
  Description of Artificial Sequence: Synthetic oligonucleotide
gcgcgctcgctcgctcactgaggccgggcgaccaaaggtcgcccgaagacatgtctgcctcagtgagcgagcgagcgcgc

SEQ ID NO: 159 - DNA, 80 bp, Artificial Sequence
  Description of Artificial Sequence: Synthetic oligonucleotide
gcgcgctcgctcgctcactgaggccgggcgaccaaaggtcgcccgacacaattgtggcctcagtgagcgagcgagcgcgc

SEQ ID NO: 160 - DNA, 80 bp, Artificial Sequence
  Description of Artificial Sequence: Synthetic oligonucleotide
gcgcgctcgctcgctcactgaggccgggcgaccaaaggtcgcccgaaaaacgttttgcctcagtgagcgagcgagcgcgc

SEQ ID NO: 161 - DNA, 80 bp, Artificial Sequence
  Description of Artificial Sequence: Synthetic oligonucleotide
gcgcgctcgctcgctcactgaggccgggcgaccaaaggtcgcccgaaaacatgtttgcctcagtgagcgagcgagcgcgc

SEQ ID NO: 162 - DNA, 80 bp, Artificial Sequence
  Description of Artificial Sequence: Synthetic oligonucleotide
gcgcgctcgctcgctcactgaggccgggcgaccaaaggtcgcccgaaacaattgttgcctcagtgagcgagcgagcgcgc

SEQ ID NO: 163 - DNA, 80 bp, Artificial Sequence
  Description of Artificial Sequence: Synthetic oligonucleotide
gcgcgctcgctcgctcactgaggccgggcgaccaaaggtcgcccgaagaaatttctgcctcagtgagcgagcgagcgcgc

SEQ ID NO: 164 - DNA, 80 bp, Artificial Sequence
  Description of Artificial Sequence: Synthetic oligonucleotide
gcgcgctcgctcgctcactgaggccgggcgaccaaaggtcgcccgacaaaattttggcctcagtgagcgagcgagcgcgc

SEQ ID NO: 165 - DNA, 80 bp, Artificial Sequence
  Description of Artificial Sequence: Synthetic oligonucleotide
gcgcgctcgctcgctcactgaggccgggcgaccaaaggtcgcccgaaaaaatttttgcctcagtgagcgagcgagcgcgc

SEQ ID NO: 166 - DNA, 80 bp, Artificial Sequence
  Description of Artificial Sequence: Synthetic oligonucleotide
gcgcgctcgctcgctcgctgaggccgggcgaccaaaggtcgcccgacgcccgggcggcctcagcgagcgagcgagcgcgc

SEQ ID NO: 167 - DNA, 80 bp, Artificial Sequence
  Description of Artificial Sequence: Synthetic oligonucleotide
gcgcgctcgctcgctcaatgaggccgggcgaccaaaggtcgcccgacgcccgggcggcctcattgagcgagcgagcgcgc

SEQ ID NO: 168 - DNA, 80 bp, Artificial Sequence
  Description of Artificial Sequence: Synthetic oligonucleotide
gcgcgctcgctcgctcaccgaggccgggcgaccaaaggtcgcccgacgcccgggcggcctcggtgagcgagcgagcgcgc

SEQ ID NO: 169 - DNA, 80 bp, Artificial Sequence
  Description of Artificial Sequence: Synthetic oligonucleotide
gcgcgctcgctcgctcactaaggccgggcgaccaaaggtcgcccgacgcccgggcggccttagtgagcgagcgagcgcgc

SEQ ID NO: 170 - DNA, 80 bp, Artificial Sequence
  Description of Artificial Sequence: Synthetic oligonucleotide
gcgcgctcgctcgctcactggggccgggcgaccaaaggtcgcccgacgcccgggcggccccagtgagcgagcgagcgcgc

SEQ ID NO: 171 - DNA, 80 bp, Artificial Sequence
  Description of Artificial Sequence: Synthetic oligonucleotide
gcgcgctcgctcgctcactgaagccgggcgaccaaaggtcgcccgacgcccgggcggcttcagtgagcgagcgagcgcgc

SEQ ID NO: 172 - DNA, 80 bp, Artificial Sequence
  Description of Artificial Sequence: Synthetic oligonucleotide
gcgcgctcgctcgctcactgagaccgggcgaccaaaggtcgcccgacgcccgggcggtctcagtgagcgagcgagcgcgc

SEQ ID NO: 173 - DNA, 80 bp, Artificial Sequence
  Description of Artificial Sequence: Synthetic oligonucleotide
gcgcgctcgctcgctcgatgaggccgggcgaccaaaggtcgcccgacgcccgggcggcctcatcgagcgagcgagcgcgc

SEQ ID NO: 174 - DNA, 80 bp, Artificial Sequence
  Description of Artificial Sequence: Synthetic oligonucleotide
gcgcgctcgctcgctcgcggaggccgggcgaccaaaggtcgcccgacgcccgggcggcctccgcgagcgagcgagcgcgc

SEQ ID NO: 175 - DNA, 80 bp, Artificial Sequence
  Description of Artificial Sequence: Synthetic oligonucleotide
gcgcgctcgctcgctcgctaaggccgggcgaccaaaggtcgcccgacgcccgggcggccttagtgagcgagcgagcgcgc

SEQ ID NO: 176 - DNA, 80 bp, Artificial Sequence
  Description of Artificial Sequence: Synthetic oligonucleotide
gcgcgctcgctcgctcgctggggccgggcgaccaaaggtcgcccgacgcccgggcggccccagcgagcgagcgagcgcgc

SEQ ID NO: 177 - DNA, 80 bp, Artificial Sequence
  Description of Artificial Sequence: Synthetic oligonucleotide
gcgcgctcgctcgctcgctgaagccgggcgaccaaaggtcgcccgacgcccgggcggcttcagcgagcgagcgagcgcgc

SEQ ID NO: 178 - DNA, 80 bp, Artificial Sequence
  Description of Artificial Sequence: Synthetic oligonucleotide
gcgcgctcgctcgctcgctgagaccgggcgaccaaaggtcgcccgacgcccgggcggtctcagcgagcgagcgagcgcgc

SEQ ID NO: 179 - DNA, 80 bp, Artificial Sequence
  Description of Artificial Sequence: Synthetic oligonucleotide
gcgcgctcgctcgctcgaggaggccgggcgaccaaaggtcgcccgacgcccgggcggcctcctcgagcgagcgagcgcgc

SEQ ID NO: 180 - DNA, 80 bp, Artificial Sequence
  Description of Artificial Sequence: Synthetic oligonucleotide
gcgcgctcgctcgctcgataaggccgggcgaccaaaggtcgcccgacgcccgggcggccttatcgagcgagcgagcgcgc

SEQ ID NO: 181 - DNA, 80 bp, Artificial Sequence
  Description of Artificial Sequence: Synthetic oligonucleotide
gcgcgctcgctcgctcgatggggccgggcgaccaaaggtcgcccgacgcccgggcggcctcatcgagcgagcgagcgcgc

SEQ ID NO: 182 - DNA, 80 bp, Artificial Sequence
  Description of Artificial Sequence: Synthetic oligonucleotide
gcgcgctcgctcgctcgatgaagccgggcgaccaaaggtcgcccgacgcccgggcggcttcatcgagcgagcgagcgcgc

SEQ ID NO: 183 - DNA, 80 bp, Artificial Sequence
  Description of Artificial Sequence: Synthetic oligonucleotide
gcgcgctcgctcgctcgatgagaccgggcgaccaaaggtcgcccgacgcccgggcggtctcatcgagcgagcgagcgcgc

SEQ ID NO: 184 - DNA, 80 bp, Artificial Sequence
  Description of Artificial Sequence: Synthetic oligonucleotide
gcgcgctcgctcgctcgagaaggccgggcgaccaaaggtcgcccgacgcccgggcggccttctcgagcgagcgagcgcgc

SEQ ID NO: 185 - DNA, 80 bp, Artificial Sequence
  Description of Artificial Sequence: Synthetic oligonucleotide
gcgcgctcgctcgctcgagggggccgggcgaccaaaggtcgcccgacgcccgggcggccccctcgagcgagcgagcgcgc

SEQ ID NO: 186 - DNA, 80 bp, Artificial Sequence
  Description of Artificial Sequence: Synthetic oligonucleotide
gcgcgctcgctcgctcgaggaagccgggcgaccaaaggtcgcccgacgcccgggcggcttcctcgagcgagcgagcgcgc

SEQ ID NO: 187 - DNA, 80 bp, Artificial Sequence; identical to SEQ ID NO: 185
  Description of Artificial Sequence: Synthetic oligonucleotide

SEQ ID NO: 188 - DNA, 80 bp, Artificial Sequence; identical to SEQ ID NO: 186
  Description of Artificial Sequence: Synthetic oligonucleotide

SEQ ID NO: 189 - DNA, 80 bp, Artificial Sequence
  Description of Artificial Sequence: Synthetic oligonucleotide
gcgcgctcgctcgctcgaggagaccgggcgaccaaaggtcgcccgacgcccgggcggtctcctcgagcgagcgagcgcgc

SEQ ID NO: 190 - DNA, 80 bp, Artificial Sequence
  Description of Artificial Sequence: Synthetic oligonucleotide
gcgcgctcgctcgctcgagagggccgggcgaccaaaggtcgcccgacgcccgggcggccctctcgagcgagcgagcgcgc

SEQ ID NO: 191-199 - skipped (9 entries)

SEQ ID NO: 200 - DNA, 80 bp, Artificial Sequence
  Description of Artificial Sequence: Synthetic oligonucleotide
gcgcgctcgctcgctcgagaaagccgggcgaccaaaggtcgcccgacgcccgggcggctttctcgagcgagcgagcgcgc

SEQ ID NO: 201 - DNA, 80 bp, Artificial Sequence
  Description of Artificial Sequence: Synthetic oligonucleotide
gcgcgctcgctcgctcgagaagaccgggcgaccaaaggtcgcccgacgcccgggcggccttctcgagcgagcgagcgcgc

SEQ ID NO: 202 - DNA, 80 bp, Artificial Sequence
  Description of Artificial Sequence: Synthetic oligonucleotide
gcgcgctcgctcgctcgagagagccgggcgaccaaaggtcgcccgacgcccgggcggctctctcgagcgagcgagcgcgc

SEQ ID NO: 203 - DNA, 80 bp, Artificial Sequence
  Description of Artificial Sequence: Synthetic oligonucleotide
gcgcgctcgctcgctcgagaggaccgggcgaccaaaggtcgcccgacgcccgggcggtcctctcgagcgagcgagcgcgc

SEQ ID NO: 204 - DNA, 80 bp, Artificial Sequence
  Description of Artificial Sequence: Synthetic oligonucleotide
gcgcgctcgctcgctcgagagaaccgggcgaccaaaggtcgcccgacgcccgggcggttctctcgagcgagcgagcgcgc

SEQ ID NO: 205 - DNA, 80 bp, Artificial Sequence
  Description of Artificial Sequence: Synthetic oligonucleotide
gcgcgctcgctcgctcaagagaaccgggcgaccaaaggtcgcccgacgcccgggcggttctcttgagcgagcgagcgcgc

SEQ ID NO: 206 - DNA, 80 bp, Artificial Sequence
  Description of Artificial Sequence: Synthetic oligonucleotide
gcgcgctcgctcgctcacgagaaccgggcgaccaaaggtcgcccgacgcccgggcggttctcgtgagcgagcgagcgcgc

SEQ ID NO: 207 - DNA, 80 bp, Artificial Sequence
  Description of Artificial Sequence: Synthetic oligonucleotide
gcgcgctcgctcgctcactagaaccgggcgaccaaaggtcgcccgacgcccgggcggttctagtgagcgagcgagcgcgc

SEQ ID NO: 208 - DNA, 80 bp, Artificial Sequence
  Description of Artificial Sequence: Synthetic oligonucleotide
gcgcgctcgctcgctcactggaaccgggcgaccaaaggtcgcccgacgcccgggcggttccagtgagcgagcgagcgcgc

SEQ ID NO: 209 - DNA, 80 bp, Artificial Sequence
  Description of Artificial Sequence: Synthetic oligonucleotide
gcgcgctcgctcgctcactgaaaccgggcgaccaaaggtcgcccgacgcccgggcggtttcagtgagcgagcgagcgcgc

SEQ ID NO: 210 - DNA, 80 bp, Artificial Sequence
  Description of Artificial Sequence: Synthetic oligonucleotide
gcgcgctcgctcgctcgcgggggccgggcgaccaaaggtcgcccgacgcccgggcggcccccgcgagcgagcgagcgcgc

SEQ ID NO: 211 - DNA, 80 bp, Artificial Sequence
  Description of Artificial Sequence: Synthetic oligonucleotide
gcgcgctcgctcgctcaataaaaccgggcgaccaaaggtcgcccgacgcccgggcggttttattgagcgagcgagcgcgc

SEQ ID NO: 212 - DNA, 80 bp, Artificial Sequence
  Description of Artificial Sequence: Synthetic oligonucleotide
gcgcgctcgctcgctcactgaggccaggcgaccaaaggtcgcctgacgcccgggcggcctcagtgagcgagcgagcgcgc

SEQ ID NO: 213 - DNA, 80 bp, Artificial Sequence
  Description of Artificial Sequence: Synthetic oligonucleotide
gcgcgctcgctcgctcactgaggccgagcgaccaaaggtcgctcgacgcccgggcggcctcagtgagcgagcgagcgcgc

SEQ ID NO: 214 - DNA, 80 bp, Artificial Sequence
  Description of Artificial Sequence: Synthetic oligonucleotide
gcgcgctcgctcgctcactgaggccggacgaccaaaggtcgtccgacgcccgggcggcctcagtgagcgagcgagcgcgc

SEQ ID NO: 215 - DNA, 80 bp, Artificial Sequence
  Description of Artificial Sequence: Synthetic oligonucleotide
gcgcgctcgctcgctcactgaggccgggagaccaaaggtctcccgacgcccgggcggcctcagtgagcgagcgagcgcgc

SEQ ID NO: 216 - DNA, 80 bp, Artificial Sequence
  Description of Artificial Sequence: Synthetic oligonucleotide
gcgcgctcgctcgctcactgaggccgggcaaccaaaggttgcccgacgcccgggcggcctcagtgagcgagcgagcgcgc

SEQ ID NO: 217 - DNA, 80 bp, Artificial Sequence
  Description of Artificial Sequence: Synthetic oligonucleotide
gcgcgctcgctcgctcactgaggccgggcggccaaaggccgcccgacgcccgggcggcctcagtgagcgagcgagcgcgc

SEQ ID NO: 218 - DNA, 80 bp, Artificial Sequence
  Description of Artificial Sequence: Synthetic oligonucleotide
gcgcgctcgctcgctcactgaggccgggcgaacaaagttcgcccgacgcccgggcggcctcagtgagcgagcgagcgcgc

SEQ ID NO: 219 - DNA, 80 bp, Artificial Sequence
  Description of Artificial Sequence: Synthetic oligonucleotide
gcgcgctcgctcgctcactgaggccgggcgacaaaatgtcgcccgacgcccgggcggcctcagtgagcgagcgagcgcgc

SEQ ID NO: 220 - DNA, 80 bp, Artificial Sequence
  Description of Artificial Sequence: Synthetic oligonucleotide
gcgcgctcgctcgctcactgaggccaagcgaccaaaggtcgcttgacgcccgggcggcctcagtgagcgagcgagcgcgc

SEQ ID NO: 221 - DNA, 80 bp, Artificial Sequence
  Description of Artificial Sequence: Synthetic oligonucleotide
gcgcgctcgctcgctcactgaggccaaacgaccaaaggtcgtttgacgcccgggcggcctcagtgagcgagcgagcgcgc

SEQ ID NO: 222 - DNA, 80 bp, Artificial Sequence
  Description of Artificial Sequence: Synthetic oligonucleotide
gcgcgctcgctcgctcactgaggccaaaagaccaaaggtcttttgacgcccgggcggcctcagtgagcgagcgagcgcgc

SEQ ID NO: 223 - DNA, 80 bp, Artificial Sequence
  Description of Artificial Sequence: Synthetic oligonucleotide
gcgcgctcgctcgctcactgaggccaaaaaaccaaaggttttttgacgcccgggcggcctcagtgagcgagcgagcgcgc

SEQ ID NO: 224 - DNA, 80 bp, Artificial Sequence
  Description of Artificial Sequence: Synthetic oligonucleotide
gcgcgctcgctcgctcactgaggccaaaaagccaaaggctttttgacgcccgggcggcctcagtgagcgagcgagcgcgc

SEQ ID NO: 225 - DNA, 80 bp, Artificial Sequence
  Description of Artificial Sequence: Synthetic oligonucleotide
gcgcgctcgctcgctcactgaggccaaaaagacaaagtctttttgacgcccgggcggcctcagtgagcgagcgagcgcgc

SEQ ID NO: 226 - DNA, 80 bp, Artificial Sequence
  Description of Artificial Sequence: Synthetic oligonucleotide
gcgcgctcgctcgctcactgaggccaaaaagaaaaattctttttgacgcccgggcggcctcagtgagcgagcgagcgcgc

SEQ ID NO: 227 - DNA, 80 bp, Artificial Sequence
  Description of Artificial Sequence: Synthetic oligonucleotide
gcgcgctcgctcgctcactgaggccaaaaaaaaaaattttttttgacgcccgggcggcctcagtgagcgagcgagcgcgc

SEQ ID NO: 228 - DNA, 80 bp, Artificial Sequence
  Description of Artificial Sequence: Synthetic oligonucleotide
gcgcgctcgctcgctcactgaggccgaaaaaaaaaatttttttcgacgcccgggcggcctcagtgagcgagcgagcgcgc

SEQ ID NO: 229 - DNA, 80 bp, Artificial Sequence
  Description of Artificial Sequence: Synthetic oligonucleotide
gcgcgctcgctcgctcactgaggccggaaagaaaaattctttccgacgcccgggcggcctcagtgagcgagcgagcgcgc

SEQ ID NO: 230 - DNA, 80 bp, Artificial Sequence
  Description of Artificial Sequence: Synthetic oligonucleotide
gcgcgctcgctcgctcactgaggccgggaagaaaaattcttcccgacgcccgggcggcctcagtgagcgagcgagcgcgc

SEQ ID NO: 231 - DNA, 80 bp, Artificial Sequence
  Description of Artificial Sequence: Synthetic oligonucleotide
gcgcgctcgctcgctcactgaggccgggcagaaaaattctgcccgacgcccgggcggcctcagtgagcgagcgagcgcgc

SEQ ID NO: 232 - DNA, 80 bp, Artificial Sequence
  Description of Artificial Sequence: Synthetic oligonucleotide
gcgcgctcgctcgctcactgaggccgggcggaaaaattccgcccgacgcccgggcggcctcagtgagcgagcgagcgcgc

SEQ ID NO: 233 - DNA, 80 bp, Artificial Sequence
  Description of Artificial Sequence: Synthetic oligonucleotide
gcgcgctcgctcgctcactgaggccgggcgaaaaaatttcgcccgacgcccgggcggcctcagtgagcgagcgagcgcgc

SEQ ID NO: 234 - DNA, 80 bp, Artificial Sequence
  Description of Artificial Sequence: Synthetic oligonucleotide
gcgcgctcgctcgctcactgaggccaggcgaccaaaggtcgcctgacacccgggtggcctcagtgagcgagcgagcgcgc

SEQ ID NO: 235 - DNA, 80 bp, Artificial Sequence
  Description of Artificial Sequence: Synthetic oligonucleotide
gcgcgctcgctcgctcactgaggccaggcgaccaaaggtcgcctgacgccatggcggcctcagtgagcgagcgagcgcgc

SEQ ID NO: 236 - DNA, 80 bp, Artificial Sequence
  Description of Artificial Sequence: Synthetic oligonucleotide
gcgcgctcgctcgctcactgaggccaggcgaccaaaggtcgcctgacgacatgtcggcctcagtgagcgagcgagcgcgc

SEQ ID NO: 237 - DNA, 80 bp, Artificial Sequence
  Description of Artificial Sequence: Synthetic oligonucleotide
gcgcgctcgctcgctcactgaggccaggcgaccaaaggtcgcctgacgaacgttcggcctcagtgagcgagcgagcgcgc

SEQ ID NO: 238 - DNA, 80 bp, Artificial Sequence
  Description of Artificial Sequence: Synthetic oligonucleotide
gcgcgctcgctcgctcactgaggccaggcgaccaaaggtcgcctgaagcaattgctgcctcagtgagcgagcgagcgcgc

SEQ ID NO: 239 - DNA, 80 bp, Artificial Sequence
  Description of Artificial Sequence: Synthetic oligonucleotide
gcgcgctcgctcgctcactgaggccgggcggccaaaggccgcccgacacccgggtggcctcagtgagcgagcgagcgcgc

SEQ ID NO: 240 - DNA, 80 bp, Artificial Sequence
  Description of Artificial Sequence: Synthetic oligonucleotide
gcgcgctcgctcgctcactgaggccgggcggccaaaggccgcccgacgccatggcggcctcagtgagcgagcgagcgcgc

SEQ ID NO: 241 - DNA, 80 bp, Artificial Sequence
  Description of Artificial Sequence: Synthetic oligonucleotide
gcgcgctcgctcgctcactgaggccgggcggccaaaggccgcccgacgacatgtcggcctcagtgagcgagcgagcgcgc

SEQ ID NO: 242 - DNA, 80 bp, Artificial Sequence
  Description of Artificial Sequence: Synthetic oligonucleotide
gcgcgctcgctcgctcactgaggccgggcggccaaaggccgcccgacgaacgttcggcctcagtgagcgagcgagcgcgc

SEQ ID NO: 243 - DNA, 80 bp, Artificial Sequence
  Description of Artificial Sequence: Synthetic oligonucleotide
gcgcgctcgctcgctcactgaggccgggcggccaaaggccgcccgaagcaattgctgcctcagtgagcgagcgagcgcgc

SEQ ID NO: 244 - DNA, 80 bp, Artificial Sequence
  Description of Artificial Sequence: Synthetic oligonucleotide
gcgcgctcgctcgctcactgaggccgggcgacaaaatgtcgcccgacacccgggtggcctcagtgagcgagcgagcgcgc

SEQ ID NO: 245 - DNA, 80 bp, Artificial Sequence
  Description of Artificial Sequence: Synthetic oligonucleotide
gcgcgctcgctcgctcactgaggccgggcgacaaaatgtcgcccgacgccatggcggcctcagtgagcgagcgagcgcgc

SEQ ID NO: 246 - DNA, 80 bp, Artificial Sequence
  Description of Artificial Sequence: Synthetic oligonucleotide
gcgcgctcgctcgctcactgaggccgggcgacaaaatgtcgcccgacgacatgtcggcctcagtgagcgagcgagcgcgc

SEQ ID NO: 247 - DNA, 80 bp, Artificial Sequence
  Description of Artificial Sequence: Synthetic oligonucleotide
gcgcgctcgctcgctcactgaggccgggcgacaaaatgtcgcccgacgaacgttcggcctcagtgagcgagcgagcgcgc

SEQ ID NO: 248 - DNA, 80 bp, Artificial Sequence
  Description of Artificial Sequence: Synthetic oligonucleotide
gcgcgctcgctcgctcactgaggccgggcgacaaaatgtcgcccgaagcaattgctgcctcagtgagcgagcgagcgcgc

SEQ ID NO: 249 - DNA, 80 bp, Artificial Sequence
  Description of Artificial Sequence: Synthetic oligonucleotide
gcgcgctcgctcgctcactgaggccaaaagaccaaaggtcttttgacacccgggtggcctcagtgagcgagcgagcgcgc

SEQ ID NO: 250 - DNA, 80 bp, Artificial Sequence
  Description of Artificial Sequence: Synthetic oligonucleotide
gcgcgctcgctcgctcactgaggccaaaagaccaaaggtcttttgacgccatggcggcctcagtgagcgagcgagcgcgc

SEQ ID NO: 251 - DNA, 80 bp, Artificial Sequence
  Description of Artificial Sequence: Synthetic oligonucleotide
gcgcgctcgctcgctcactgaggccaaaagaccaaaggtcttttgacgacatgtcggcctcagtgagcgagcgagcgcgc

SEQ ID NO: 252 - DNA, 80 bp, Artificial Sequence
  Description of Artificial Sequence: Synthetic oligonucleotide
gcgcgctcgctcgctcactgaggccaaaagaccaaaggtcttttgacgaacgttcggcctcagtgagcgagcgagcgcgc

SEQ ID NO: 253 - DNA, 80 bp, Artificial Sequence
  Description of Artificial Sequence: Synthetic oligonucleotide
gcgcgctcgctcgctcactgaggccaaaagaccaaaggtcttttgaagcaattgctgcctcagtgagcgagcgagcgcgc

SEQ ID NO: 254 - DNA, 80 bp, Artificial Sequence
  Description of Artificial Sequence: Synthetic oligonucleotide
gcgcgctcgctcgctcactgaggccaaaaaaaaaaattttttttgacacccgggtggcctcagtgagcgagcgagcgcgc

SEQ ID NO: 255 - DNA, 80 bp, Artificial Sequence
  Description of Artificial Sequence: Synthetic oligonucleotide
gcgcgctcgctcgctcactgaggccaaaaaaaaaaattttttttgacgccatggcggcctcagtgagcgagcgagcgcgc

SEQ ID NO: 256 - DNA, 80 bp, Artificial Sequence
  Description of Artificial Sequence: Synthetic oligonucleotide
gcgcgctcgctcgctcactgaggccaaaaaaaaaaattttttttgacgacatgtcggcctcagtgagcgagcgagcgcgc

SEQ ID NO: 257 - DNA, 80 bp, Artificial Sequence
  Description of Artificial Sequence: Synthetic oligonucleotide
gcgcgctcgctcgctcactgaggccaaaaaaaaaaattttttttgacgaacgttcggcctcagtgagcgagcgagcgcgc

SEQ ID NO: 258 - DNA, 80 bp, Artificial Sequence
  Description of Artificial Sequence: Synthetic oligonucleotide
gcgcgctcgctcgctcactgaggccaaaaaaaaaaattttttttgaagcaattgctgcctcagtgagcgagcgagcgcgc

SEQ ID NO: 259 - DNA, 80 bp, Artificial Sequence
  Description of Artificial Sequence: Synthetic oligonucleotide
gcgcgctcgctcgctcactgaggccgggcagaaaaattctgcccgacacccgggtggcctcagtgagcgagcgagcgcgc

SEQ ID NO: 260 - DNA, 80 bp, Artificial Sequence
  Description of Artificial Sequence: Synthetic oligonucleotide
gcgcgctcgctcgctcactgaggccgggcagaaaaattctgcccgacgccatggcggcctcagtgagcgagcgagcgcgc

SEQ ID NO: 261 - DNA, 80 bp, Artificial Sequence
  Description of Artificial Sequence: Synthetic oligonucleotide
gcgcgctcgctcgctcactgaggccgggcagaaaaattctgcccgacgacatgtcggcctcagtgagcgagcgagcgcgc

SEQ ID NO: 262 - DNA, 80 bp, Artificial Sequence
  Description of Artificial Sequence: Synthetic oligonucleotide
gcgcgctcgctcgctcactgaggccgggcagaaaaattctgcccgacgaacgttcggcctcagtgagcgagcgagcgcgc

SEQ ID NO: 263 - DNA, 80 bp, Artificial Sequence
  Description of Artificial Sequence: Synthetic oligonucleotide
gcgcgctcgctcgctcactgaggccgggcagaaaaattctgcccgaagcaattgctgcctcagtgagcgagcgagcgcgc

SEQ ID NO: 264 - DNA, 80 bp, Artificial Sequence
  Description of Artificial Sequence: Synthetic oligonucleotide
gcgcgctcgctcgctcactaaggccaggcgaccaaaggtcgcctgacgcccgggcggccttagtgagcgagcgagcgcgc

SEQ ID NO: 265 - DNA, 80 bp, Artificial Sequence
  Description of Artificial Sequence: Synthetic oligonucleotide
gcgcgctcgctcgctcactaaggccgggcggccaaaggccgcccgacgcccgggcggccttagtgagcgagcgagcgcgc

SEQ ID NO: 266 - DNA, 80 bp, Artificial Sequence
  Description of Artificial Sequence: Synthetic oligonucleotide
gcgcgctcgctcgctcactaaggccgggcgacaaaatgtcgcccgacgcccgggcggccttagtgagcgagcgagcgcgc

SEQ ID NO: 267 - DNA, 80 bp, Artificial Sequence
  Description of Artificial Sequence: Synthetic oligonucleotide
gcgcgctcgctcgctcactaaggccaaaagaccaaaggtcttttgacgcccgggcggccttagtgagcgagcgagcgcgc

SEQ ID NO: 268 - DNA, 80 bp, Artificial Sequence
  Description of Artificial Sequence: Synthetic oligonucleotide
gcgcgctcgctcgctcactaaggccaaaaaaaaaaattttttttgacgcccgggcggccttagtgagcgagcgagcgcgc

SEQ ID NO: 269 - DNA, 80 bp, Artificial Sequence
  Description of Artificial Sequence: Synthetic oligonucleotide
gcgcgctcgctcgctcactaaggccgggcagaaaaattctgcccgacgcccgggcggccttagtgagcgagcgagcgcgc

SEQ ID NO: 270 - DNA, 80 bp, Artificial Sequence
  Description of Artificial Sequence: Synthetic oligonucleotide
gcgcgctcgctcgctcgatggggccaggcgaccaaaggtcgcctgacgcccgggcggcctcatcgagcgagcgagcgcgc

SEQ ID NO: 271 - DNA, 80 bp, Artificial Sequence
  Description of Artificial Sequence: Synthetic oligonucleotide
gcgcgctcgctcgctcgatggggccgggcggccaaaggccgcccgacgcccgggcggcctcatcgagcgagcgagcgcgc

SEQ ID NO: 272 - DNA, 80 bp, Artificial Sequence
  Description of Artificial Sequence: Synthetic oligonucleotide
gcgcgctcgctcgctcgatggggccgggcgacaaaatgtcgcccgacgcccgggcggcctcatcgagcgagcgagcgcgc

SEQ ID NO: 273 - DNA, 80 bp, Artificial Sequence
  Description of Artificial Sequence: Synthetic oligonucleotide
gcgcgctcgctcgctcgatggggccaaaagaccaaaggtcttttgacgcccgggcggcctcatcgagcgagcgagcgcgc

SEQ ID NO: 274 - DNA, 80 bp, Artificial Sequence
  Description of Artificial Sequence: Synthetic oligonucleotide
gcgcgctcgctcgctcgatggggccaaaaaaaaaaattttttttgacgcccgggcggcctcatcgagcgagcgagcgcgc

SEQ ID NO: 275 - DNA, 80 bp, Artificial Sequence
  Description of Artificial Sequence: Synthetic oligonucleotide
gcgcgctcgctcgctcgatggggccgggcagaaaaattctgcccgacgcccgggcggcctcatcgagcgagcgagcgcgc

SEQ ID NO: 276 - DNA, 80 bp, Artificial Sequence
  Description of Artificial Sequence: Synthetic oligonucleotide
gcgcgctcgctcgctcgagagaaccaggcgaccaaaggtcgcctgacgcccgggcggttctctcgagcgagcgagcgcgc

SEQ ID NO: 277 - DNA, 80 bp, Artificial Sequence
  Description of Artificial Sequence: Synthetic oligonucleotide
gcgcgctcgctcgctcgagagaaccgggcggccaaaggccgcccgacgcccgggcggttctctcgagcgagcgagcgcgc

SEQ ID NO: 278 - DNA, 80 bp, Artificial Sequence
  Description of Artificial Sequence: Synthetic oligonucleotide
gcgcgctcgctcgctcgagagaaccgggcgacaaaatgtcgcccgacgcccgggcggttctctcgagcgagcgagcgcgc

SEQ ID NO: 279 - DNA, 80 bp, Artificial Sequence
  Description of Artificial Sequence: Synthetic oligonucleotide
gcgcgctcgctcgctcgagagaaccaaaagaccaaaggtcttttgacgcccgggcggttctctcgagcgagcgagcgcgc

SEQ ID NO: 280 - DNA, 80 bp, Artificial Sequence
  Description of Artificial Sequence: Synthetic oligonucleotide
gcgcgctcgctcgctcgagagaaccaaaaaaaaaaattttttttgacgcccgggcggttctctcgagcgagcgagcgcgc

SEQ ID NO: 281 - DNA, 80 bp, Artificial Sequence
  Description of Artificial Sequence: Synthetic oligonucleotide
gcgcgctcgctcgctcgagagaaccgggcagaaaaattctgcccgacgcccgggcggttctctcgagcgagcgagcgcgc

SEQ ID NO: 282 - DNA, 80 bp, Artificial Sequence
  Description of Artificial Sequence: Synthetic oligonucleotide
gcgcgctcgctcgctcgcgggggccaggcgaccaaaggtcgcctgacgcccgggcggcccccgcgagcgagcgagcgcgc

SEQ ID NO: 283 - DNA, 80 bp, Artificial Sequence
  Description of Artificial Sequence: Synthetic oligonucleotide
gcgcgctcgctcgctcgcgggggccgggcggccaaaggccgcccgacgcccgggcggcccccgcgagcgagcgagcgcgc

SEQ ID NO: 284 - DNA, 80 bp, Artificial Sequence
  Description of Artificial Sequence: Synthetic oligonucleotide
gcgcgctcgctcgctcgcgggggccgggcgacaaaatgtcgcccgacgcccgggcggcccccgcgagcgagcgagcgcgc

SEQ ID NO: 285 - DNA, 80 bp, Artificial Sequence
  Description of Artificial Sequence: Synthetic oligonucleotide
gcgcgctcgctcgctcgcgggggccaaaagaccaaaggtcttttgacgcccgggcggcccccgcgagcgagcgagcgcgc

SEQ ID NO: 286 - DNA, 80 bp, Artificial Sequence
  Description of Artificial Sequence: Synthetic oligonucleotide
gcgcgctcgctcgctcgcgggggccaaaaaaaaaaattttttttgacgcccgggcggcccccgcgagcgagcgagcgcgc

SEQ ID NO: 287 - DNA, 80 bp, Artificial Sequence
  Description of Artificial Sequence: Synthetic oligonucleotide
gcgcgctcgctcgctcgcgggggccgggcagaaaaattctgcccgacgcccgggcggcccccgcgagcgagcgagcgcgc

SEQ ID NO: 288 - DNA, 80 bp, Artificial Sequence
  Description of Artificial Sequence: Synthetic oligonucleotide
gcgcgctcgctcgctcaataaaaccaggcgaccaaaggtcgcctgacgcccgggcggttttattgagcgagcgagcgcgc

SEQ ID NO: 289 - DNA, 80 bp, Artificial Sequence
  Description of Artificial Sequence: Synthetic oligonucleotide
gcgcgctcgctcgctcaataaaaccgggcggccaaaggccgcccgacgcccgggcggttttattgagcgagcgagcgcgc

SEQ ID NO: 290 - DNA, 80 bp, Artificial Sequence
  Description of Artificial Sequence: Synthetic oligonucleotide
gcgcgctcgctcgctcaataaaaccgggcgacaaaatgtcgcccgacgcccgggcggttttattgagcgagcgagcgcgc

SEQ ID NO: 291 - DNA, 80 bp, Artificial Sequence
  Description of Artificial Sequence: Synthetic oligonucleotide
gcgcgctcgctcgctcaataaaaccaaaagaccaaaggtcttttgacgcccgggcggttttattgagcgagcgagcgcgc

SEQ ID NO: 292 - DNA, 80 bp, Artificial Sequence
  Description of Artificial Sequence: Synthetic oligonucleotide
gcgcgctcgctcgctcaataaaaccaaaaaaaaaaattttttttgacgcccgggcggttttattgagcgagcgagcgcgc

SEQ ID NO: 293 - DNA, 80 bp, Artificial Sequence
  Description of Artificial Sequence: Synthetic oligonucleotide
gcgcgctcgctcgctcaataaaaccgggcagaaaaattctgcccgacgcccgggcggttttattgagcgagcgagcgcgc

SEQ ID NO: 294 - DNA, 80 bp, Artificial Sequence
  Description of Artificial Sequence: Synthetic oligonucleotide
gcgcgctcgctcgctcactaaggccgggcgaccaaaggtcgcccgacacccgggtggccttagtgagcgagcgagcgcgc

SEQ ID NO: 295 - DNA, 80 bp, Artificial Sequence
  Description of Artificial Sequence: Synthetic oligonucleotide
gcgcgctcgctcgctcactaaggccgggcgaccaaaggtcgcccgacgccatggcggccttagtgagcgagcgagcgcgc

SEQ ID NO: 296 - DNA, 80 bp, Artificial Sequence
  Description of Artificial Sequence: Synthetic oligonucleotide
gcgcgctcgctcgctcactaaggccgggcgaccaaaggtcgcccgacgacatgtcggccttagtgagcgagcgagcgcgc

SEQ ID NO: 297 - DNA, 80 bp, Artificial Sequence
  Description of Artificial Sequence: Synthetic oligonucleotide
gcgcgctcgctcgctcactaaggccgggcgaccaaaggtcgcccgacgaacgttcggccttagtgagcgagcgagcgcgc

SEQ ID NO: 298 - DNA, 80 bp, Artificial Sequence
  Description of Artificial Sequence: Synthetic oligonucleotide
gcgcgctcgctcgctcactaaggccgggcgaccaaaggtcgcccgaagcaattgctgccttagtgagcgagcgagcgcgc

SEQ ID NO: 299 - DNA, 80 bp, Artificial Sequence
  Description of Artificial Sequence: Synthetic oligonucleotide
gcgcgctcgctcgctcgatggggccgggcgaccaaaggtcgcccgacacccgggtggcctcatcgagcgagcgagcgcgc

SEQ ID NO: 300 - DNA, 80 bp, Artificial Sequence
  Description of Artificial Sequence: Synthetic oligonucleotide
gcgcgctcgctcgctcgatggggccgggcgaccaaaggtcgcccgacgccatggcggcctcatcgagcgagcgagcgcgc

SEQ ID NO: 301 - DNA, 80 bp, Artificial Sequence
  Description of Artificial Sequence: Synthetic oligonucleotide
gcgcgctcgctcgctcgatggggccgggcgaccaaaggtcgcccgacgacatgtcggcctcatcgagcgagcgagcgcgc

SEQ ID NO: 302 - DNA, 80 bp, Artificial Sequence
  Description of Artificial Sequence: Synthetic oligonucleotide
gcgcgctcgctcgctcgatggggccgggcgaccaaaggtcgcccgacgaacgttcggcctcatcgagcgagcgagcgcgc

SEQ ID NO: 303 - DNA, 80 bp, Artificial Sequence
  Description of Artificial Sequence: Synthetic oligonucleotide
gcgcgctcgctcgctcgatggggccgggcgaccaaaggtcgcccgaagcaattgctgcctcatcgagcgagcgagcgcgc

SEQ ID NO: 304 - DNA, 80 bp, Artificial Sequence
  Description of Artificial Sequence: Synthetic oligonucleotide
gcgcgctcgctcgctcgagagaaccgggcgaccaaaggtcgcccgacacccgggtggttctctcgagcgagcgagcgcgc

SEQ ID NO: 305 - DNA, 80 bp, Artificial Sequence
  Description of Artificial Sequence: Synthetic oligonucleotide
gcgcgctcgctcgctcgagagaaccgggcgaccaaaggtcgcccgacgccatggcggttctctcgagcgagcgagcgcgc

SEQ ID NO: 306 - DNA, 80 bp, Artificial Sequence
  Description of Artificial Sequence: Synthetic oligonucleotide
gcgcgctcgctcgctcgagagaaccgggcgaccaaaggtcgcccgacgacatgtcggttctctcgagcgagcgagcgcgc

SEQ ID NO: 307 - DNA, 80 bp, Artificial Sequence
  Description of Artificial Sequence: Synthetic oligonucleotide
gcgcgctcgctcgctcgagagaaccgggcgaccaaaggtcgcccgacgaacgttcggttctctcgagcgagcgagcgcgc

SEQ ID NO: 308 - DNA, 80 bp, Artificial Sequence
  Description of Artificial Sequence: Synthetic oligonucleotide
gcgcgctcgctcgctcgagagaaccgggcgaccaaaggtcgcccgaagcaattgctgttctctcgagcgagcgagcgcgc

SEQ ID NO: 309 - DNA, 80 bp, Artificial Sequence
  Description of Artificial Sequence: Synthetic oligonucleotide
gcgcgctcgctcgctcgcgggggccgggcgaccaaaggtcgcccgacacccgggtggcccccgcgagcgagcgagcgcgc

SEQ ID NO: 310 - DNA, 80 bp, Artificial Sequence
  Description of Artificial Sequence: Synthetic oligonucleotide
gcgcgctcgctcgctcgcgggggccgggcgaccaaaggtcgcccgacgccatggcggcccccgcgagcgagcgagcgcgc

SEQ ID NO: 311 - DNA, 80 bp, Artificial Sequence
  Description of Artificial Sequence: Synthetic oligonucleotide
gcgcgctcgctcgctcgcgggggccgggcgaccaaaggtcgcccgacgacatgtcggcccccgcgagcgagcgagcgcgc

SEQ ID NO: 312 - DNA, 80 bp, Artificial Sequence
  Description of Artificial Sequence: Synthetic oligonucleotide
gcgcgctcgctcgctcgcgggggccgggcgaccaaaggtcgcccgacgaacgttcggcccccgcgagcgagcgagcgcgc

SEQ ID NO: 313 - DNA, 80 bp, Artificial Sequence
  Description of Artificial Sequence: Synthetic oligonucleotide
gcgcgctcgctcgctcgcgggggccgggcgaccaaaggtcgcccgaagcaattgctgcccccgcgagcgagcgagcgcgc

SEQ ID NO: 314 - DNA, 80 bp, Artificial Sequence
  Description of Artificial Sequence: Synthetic oligonucleotide
gcgcgctcgctcgctcaataaaaccgggcgaccaaaggtcgcccgacacccgggtggttttattgagcgagcgagcgcgc

SEQ ID NO: 315 - DNA, 80 bp, Artificial Sequence
  Description of Artificial Sequence: Synthetic oligonucleotide
gcgcgctcgctcgctcaataaaaccgggcgaccaaaggtcgcccgacgccatggcggttttattgagcgagcgagcgcgc

SEQ ID NO: 316 - DNA, 80 bp, Artificial Sequence
  Description of Artificial Sequence: Synthetic oligonucleotide
gcgcgctcgctcgctcaataaaaccgggcgaccaaaggtcgcccgacgacatgtcggttttattgagcgagcgagcgcgc

SEQ ID NO: 317 - DNA, 80 bp, Artificial Sequence
  Description of Artificial Sequence: Synthetic oligonucleotide
gcgcgctcgctcgctcaataaaaccgggcgaccaaaggtcgcccgacgaacgttcggttttattgagcgagcgagcgcgc

SEQ ID NO: 318 - DNA, 80 bp, Artificial Sequence
  Description of Artificial Sequence: Synthetic oligonucleotide
gcgcgctcgctcgctcaataaaaccgggcgaccaaaggtcgcccgaagcaattgctgttttattgagcgagcgagcgcgc

SEQ ID NO: 319 - DNA, 80 bp, Artificial Sequence
  Description of Artificial Sequence: Synthetic oligonucleotide
gcgcgctcgctcgctcactaaggccaggcgaccaaaggtcgcctgacacccgggtggccttagtgagcgagcgagcgcgc

SEQ ID NO: 320 - DNA, 80 bp, Artificial Sequence
  Description of Artificial Sequence: Synthetic oligonucleotide
gcgcgctcgctcgctcactaaggccaggcgaccaaaggtcgcctgacgccatggcggccttagtgagcgagcgagcgcgc

SEQ ID NO: 321 - DNA, 80 bp, Artificial Sequence
  Description of Artificial Sequence: Synthetic oligonucleotide
gcgcgctcgctcgctcactaaggccaggcgaccaaaggtcgcctgacgacatgtcggccttagtgagcgagcgagcgcgc

SEQ ID NO: 322 - DNA, 80 bp, Artificial Sequence
  Description of Artificial Sequence: Synthetic oligonucleotide
gcgcgctcgctcgctcactaaggccaggcgaccaaaggtcgcctgacgaacgttcggccttagtgagcgagcgagcgcgc

SEQ ID NO: 323 - DNA, 80 bp, Artificial Sequence
  Description of Artificial Sequence: Synthetic oligonucleotide
gcgcgctcgctcgctcactaaggccaggcgaccaaaggtcgcctgaagcaattgctgccttagtgagcgagcgagcgcgc

SEQ ID NO: 324 - DNA, 80 bp, Artificial Sequence
  Description of Artificial Sequence: Synthetic oligonucleotide
gcgcgctcgctcgctcactaaggccgggcggccaaaggccgcccgacacccgggtggccttagtgagcgagcgagcgcgc

SEQ ID NO: 325 - DNA, 80 bp, Artificial Sequence
  Description of Artificial Sequence: Synthetic oligonucleotide
gcgcgctcgctcgctcactaaggccgggcggccaaaggccgcccgacgccatggcggccttagtgagcgagcgagcgcgc

SEQ ID NO: 326 - DNA, 80 bp, Artificial Sequence
  Description of Artificial Sequence: Synthetic oligonucleotide
gcgcgctcgctcgctcactaaggccgggcggccaaaggccgcccgacgacatgtcggccttagtgagcgagcgagcgcgc

SEQ ID NO: 327 - DNA, 80 bp, Artificial Sequence
  Description of Artificial Sequence: Synthetic oligonucleotide
gcgcgctcgctcgctcactaaggccgggcggccaaaggccgcccgacgaacgttcggccttagtgagcgagcgagcgcgc

SEQ ID NO: 328 - DNA, 80 bp, Artificial Sequence
  Description of Artificial Sequence: Synthetic oligonucleotide
gcgcgctcgctcgctcactaaggccgggcggccaaaggccgcccgaagcaattgctgccttagtgagcgagcgagcgcgc

SEQ ID NO: 329 - DNA, 80 bp, Artificial Sequence
  Description of Artificial Sequence: Synthetic oligonucleotide
gcgcgctcgctcgctcactaaggccgggcgacaaaatgtcgcccgacacccgggtggccttagtgagcgagcgagcgcgc

SEQ ID NO: 330 - DNA, 80 bp, Artificial Sequence
  Description of Artificial Sequence: Synthetic oligonucleotide
gcgcgctcgctcgctcactaaggccgggcgacaaaatgtcgcccgacgccatggcggccttagtgagcgagcgagcgcgc

SEQ ID NO: 331 - DNA, 80 bp, Artificial Sequence
  Description of Artificial Sequence: Synthetic oligonucleotide
gcgcgctcgctcgctcactaaggccgggcgacaaaatgtcgcccgacgacatgtcggccttagtgagcgagcgagcgcgc

SEQ ID NO: 332 - DNA, 80 bp, Artificial Sequence
  Description of Artificial Sequence: Synthetic oligonucleotide
gcgcgctcgctcgctcactaaggccgggcgacaaaatgtcgcccgacgaacgttcggccttagtgagcgagcgagcgcgc

SEQ ID NO: 333 - DNA, 80 bp, Artificial Sequence
  Description of Artificial Sequence: Synthetic oligonucleotide
gcgcgctcgctcgctcactaaggccgggcgacaaaatgtcgcccgaagcaattgctgccttagtgagcgagcgagcgcgc

SEQ ID NO: 334 - DNA, 80 bp, Artificial Sequence
  Description of Artificial Sequence: Synthetic oligonucleotide
gcgcgctcgctcgctcactaaggccaaaagaccaaaggtcttttgacacccgggtggccttagtgagcgagcgagcgcgc

SEQ ID NO: 335 - DNA, 80 bp, Artificial Sequence
  Description of Artificial Sequence: Synthetic oligonucleotide
gcgcgctcgctcgctcactaaggccaaaagaccaaaggtcttttgacgccatggcggccttagtgagcgagcgagcgcgc

SEQ ID NO: 336 - DNA, 80 bp, Artificial Sequence
  Description of Artificial Sequence: Synthetic oligonucleotide
gcgcgctcgctcgctcactaaggccaaaagaccaaaggtcttttgacgacatgtcggccttagtgagcgagcgagcgcgc

SEQ ID NO: 337 - DNA, 80 bp, Artificial Sequence
  Description of Artificial Sequence: Synthetic oligonucleotide
gcgcgctcgctcgctcactaaggccaaaagaccaaaggtcttttgacgaacgttcggccttagtgagcgagcgagcgcgc

SEQ ID NO: 338 - DNA, 80 bp, Artificial Sequence
  Description of Artificial Sequence: Synthetic oligonucleotide
gcgcgctcgctcgctcactaaggccaaaagaccaaaggtcttttgaagcaattgctgccttagtgagcgagcgagcgcgc

SEQ ID NO: 339 - DNA, 80 bp, Artificial Sequence
  Description of Artificial Sequence: Synthetic oligonucleotide
gcgcgctcgctcgctcactaaggccaaaaaaaaaaattttttttgacacccgggtggccttagtgagcgagcgagcgcgc

SEQ ID NO: 340 - DNA, 80 bp, Artificial Sequence
  Description of Artificial Sequence: Synthetic oligonucleotide
gcgcgctcgctcgctcactaaggccaaaaaaaaaaattttttttgacgccatggcggccttagtgagcgagcgagcgcgc

SEQ ID NO: 341 - DNA, 80 bp, Artificial Sequence
  Description of Artificial Sequence: Synthetic oligonucleotide
gcgcgctcgctcgctcactaaggccaaaaaaaaaaattttttttgacgacatgtcggccttagtgagcgagcgagcgcgc

SEQ ID NO: 342 - DNA, 80 bp, Artificial Sequence
  Description of Artificial Sequence: Synthetic oligonucleotide
gcgcgctcgctcgctcactaaggccaaaaaaaaaaattttttttgacgaacgttcggccttagtgagcgagcgagcgcgc

SEQ ID NO: 343 - DNA, 80 bp, Artificial Sequence
  Description of Artificial Sequence: Synthetic oligonucleotide
gcgcgctcgctcgctcactaaggccaaaaaaaaaaattttttttgaagcaattgctgccttagtgagcgagcgagcgcgc

SEQ ID NO: 344 - DNA, 80 bp, Artificial Sequence
  Description of Artificial Sequence: Synthetic oligonucleotide
gcgcgctcgctcgctcactaaggccgggcagaaaaattctgcccgacacccgggtggccttagtgagcgagcgagcgcgc

SEQ ID NO: 345 - DNA, 80 bp, Artificial Sequence
  Description of Artificial Sequence: Synthetic oligonucleotide
gcgcgctcgctcgctcactaaggccgggcagaaaaattctgcccgacgccatggcggccttagtgagcgagcgagcgcgc

SEQ ID NO: 346 - DNA, 80 bp, Artificial Sequence
  Description of Artificial Sequence: Synthetic oligonucleotide
gcgcgctcgctcgctcactaaggccgggcagaaaaattctgcccgacgacatgtcggccttagtgagcgagcgagcgcgc

SEQ ID NO: 347 - DNA, 80 bp, Artificial Sequence
  Description of Artificial Sequence: Synthetic oligonucleotide
gcgcgctcgctcgctcactaaggccgggcagaaaaattctgcccgacgaacgttcggccttagtgagcgagcgagcgcgc

SEQ ID NO: 348 - DNA, 80 bp, Artificial Sequence
  Description of Artificial Sequence: Synthetic oligonucleotide
gcgcgctcgctcgctcactaaggccgggcagaaaaattctgcccgaagcaattgctgccttagtgagcgagcgagcgcgc

SEQ ID NO: 349 - DNA, 80 bp, Artificial Sequence
  Description of Artificial Sequence: Synthetic oligonucleotide
gcgcgctcgctcgctcgatggggccaggcgaccaaaggtcgcctgacacccgggtggcctcatcgagcgagcgagcgcgc

SEQ ID NO: 350 - DNA, 80 bp, Artificial Sequence
  Description of Artificial Sequence: Synthetic oligonucleotide
gcgcgctcgctcgctcgatggggccaggcgaccaaaggtcgcctgacgccatggcggcctcatcgagcgagcgagcgcgc

SEQ ID NO: 351 - DNA, 80 bp, Artificial Sequence
  Description of Artificial Sequence: Synthetic oligonucleotide
gcgcgctcgctcgctcgatggggccaggcgaccaaaggtcgcctgacgacatgtcggcctcatcgagcgagcgagcgcgc

SEQ ID NO: 352 - DNA, 80 bp, Artificial Sequence
  Description of Artificial Sequence: Synthetic oligonucleotide
gcgcgctcgctcgctcgatggggccaggcgaccaaaggtcgcctgacgaacgttcggcctcatcgagcgagcgagcgcgc

SEQ ID NO: 353 - DNA, 80 bp, Artificial Sequence
  Description of Artificial Sequence: Synthetic oligonucleotide
gcgcgctcgctcgctcgatggggccaggcgaccaaaggtcgcctgaagcaattgctgcctcatcgagcgagcgagcgcgc

SEQ ID NO: 354 - DNA, 80 bp, Artificial Sequence
  Description of Artificial Sequence: Synthetic oligonucleotide
gcgcgctcgctcgctcgatggggccgggcggccaaaggccgcccgacacccgggtggcctcatcgagcgagcgagcgcgc

SEQ ID NO: 355 - DNA, 80 bp, Artificial Sequence
  Description of Artificial Sequence: Synthetic oligonucleotide
gcgcgctcgctcgctcgatggggccgggcggccaaaggccgcccgacgccatggcggcctcatcgagcgagcgagcgcgc

SEQ ID NO: 356 - DNA, 80 bp, Artificial Sequence
  Description of Artificial Sequence: Synthetic oligonucleotide
gcgcgctcgctcgctcgatggggccgggcggccaaaggccgcccgacgacatgtcggcctcatcgagcgagcgagcgcgc

SEQ ID NO: 357 - DNA, 80 bp, Artificial Sequence
  Description of Artificial Sequence: Synthetic oligonucleotide
gcgcgctcgctcgctcgatggggccgggcggccaaaggccgcccgacgaacgttcggcctcatcgagcgagcgagcgcgc

SEQ ID NO: 358 - DNA, 80 bp, Artificial Sequence
  Description of Artificial Sequence: Synthetic oligonucleotide
gcgcgctcgctcgctcgatggggccgggcggccaaaggccgcccgaagcaattgctgcctcatcgagcgagcgagcgcgc

SEQ ID NO: 359 - DNA, 80 bp, Artificial Sequence
  Description of Artificial Sequence: Synthetic oligonucleotide
gcgcgctcgctcgctcgatggggccgggcgacaaaatgtcgcccgacacccgggtggcctcatcgagcgagcgagcgcgc

SEQ ID NO: 360 - DNA, 80 bp, Artificial Sequence
  Description of Artificial Sequence: Synthetic oligonucleotide
gcgcgctcgctcgctcgatggggccgggcgacaaaatgtcgcccgacgccatggcggcctcatcgagcgagcgagcgcgc

SEQ ID NO: 361 - DNA, 80 bp, Artificial Sequence
  Description of Artificial Sequence: Synthetic oligonucleotide
gcgcgctcgctcgctcgatggggccgggcgacaaaatgtcgcccgacgacatgtcggcctcatcgagcgagcgagcgcgc

SEQ ID NO: 362 - DNA, 80 bp, Artificial Sequence
  Description of Artificial Sequence: Synthetic oligonucleotide
gcgcgctcgctcgctcgatggggccgggcgacaaaatgtcgcccgacgaacgttcggcctcatcgagcgagcgagcgcgc

SEQ ID NO: 363 - DNA, 80 bp, Artificial Sequence
  Description of Artificial Sequence: Synthetic oligonucleotide
gcgcgctcgctcgctcgatggggccgggcgacaaaatgtcgcccgaagcaattgctgcctcatcgagcgagcgagcgcgc

SEQ ID NO: 364 - DNA, 80 bp, Artificial Sequence
  Description of Artificial Sequence: Synthetic oligonucleotide
gcgcgctcgctcgctcgatggggccaaaagaccaaaggtcttttgacacccgggtggcctcatcgagcgagcgagcgcgc

SEQ ID NO: 365 - DNA, 80 bp, Artificial Sequence
  Description of Artificial Sequence: Synthetic oligonucleotide
gcgcgctcgctcgctcgatggggccaaaagaccaaaggtcttttgacgccatggcggcctcatcgagcgagcgagcgcgc

SEQ ID NO: 366 - DNA, 80 bp, Artificial Sequence
  Description of Artificial Sequence: Synthetic oligonucleotide
gcgcgctcgctcgctcgatggggccaaaagaccaaaggtcttttgacgacatgtcggcctcatcgagcgagcgagcgcgc

SEQ ID NO: 367 - DNA, 80 bp, Artificial Sequence
  Description of Artificial Sequence: Synthetic oligonucleotide
gcgcgctcgctcgctcgatggggccaaaagaccaaaggtcttttgacgaacgttcggcctcatcgagcgagcgagcgcgc

SEQ ID NO: 368 - DNA, 80 bp, Artificial Sequence
  Description of Artificial Sequence: Synthetic oligonucleotide
gcgcgctcgctcgctcgatggggccaaaagaccaaaggtcttttgaagcaattgctgcctcatcgagcgagcgagcgcgc

SEQ ID NO: 369 - DNA, 80 bp, Artificial Sequence
  Description of Artificial Sequence: Synthetic oligonucleotide
gcgcgctcgctcgctcgatggggccaaaaaaaaaaattttttttgacacccgggtggcctcatcgagcgagcgagcgcgc

SEQ ID NO: 370 - DNA, 80 bp, Artificial Sequence
  Description of Artificial Sequence: Synthetic oligonucleotide
gcgcgctcgctcgctcgatggggccaaaaaaaaaaattttttttgacgccatggcggcctcatcgagcgagcgagcgcgc

SEQ ID NO: 371 - DNA, 80 bp, Artificial Sequence
  Description of Artificial Sequence: Synthetic oligonucleotide
gcgcgctcgctcgctcgatggggccaaaaaaaaaaattttttttgacgacatgtcggcctcatcgagcgagcgagcgcgc

SEQ ID NO: 372 - DNA, 80 bp, Artificial Sequence
  Description of Artificial Sequence: Synthetic oligonucleotide
gcgcgctcgctcgctcgatggggccaaaaaaaaaaattttttttgacgaacgttcggcctcatcgagcgagcgagcgcgc

SEQ ID NO: 373 - DNA, 80 bp, Artificial Sequence
  Description of Artificial Sequence: Synthetic oligonucleotide
gcgcgctcgctcgctcgatggggccaaaaaaaaaaattttttttgaagcaattgctgcctcatcgagcgagcgagcgcgc

SEQ ID NO: 374 - DNA, 80 bp, Artificial Sequence
  Description of Artificial Sequence: Synthetic oligonucleotide
gcgcgctcgctcgctcgatggggccgggcagaaaaattctgcccgacacccgggtggcctcatcgagcgagcgagcgcgc

SEQ ID NO: 375 - DNA, 80 bp, Artificial Sequence
  Description of Artificial Sequence: Synthetic oligonucleotide
gcgcgctcgctcgctcgatggggccgggcagaaaaattctgcccgacgccatggcggcctcatcgagcgagcgagcgcgc

SEQ ID NO: 376 - DNA, 80 bp, Artificial Sequence
  Description of Artificial Sequence: Synthetic oligonucleotide
gcgcgctcgctcgctcgatggggccgggcagaaaaattctgcccgacgacatgtcggcctcatcgagcgagcgagcgcgc

SEQ ID NO: 377 - DNA, 80 bp, Artificial Sequence
  Description of Artificial Sequence: Synthetic oligonucleotide
gcgcgctcgctcgctcgatggggccgggcagaaaaattctgcccgacgaacgttcggcctcatcgagcgagcgagcgcgc

SEQ ID NO: 378 - DNA, 80 bp, Artificial Sequence
  Description of Artificial Sequence: Synthetic oligonucleotide
gcgcgctcgctcgctcgatggggccgggcagaaaaattctgcccgaagcaattgctgcctcatcgagcgagcgagcgcgc

SEQ ID NO: 379 - DNA, 80 bp, Artificial Sequence
  Description of Artificial Sequence: Synthetic oligonucleotide
gcgcgctcgctcgctcgagagaaccaggcgaccaaaggtcgcctgacacccgggtggttctctcgagcgagcgagcgcgc

SEQ ID NO: 380 - DNA, 80 bp, Artificial Sequence
  Description of Artificial Sequence: Synthetic oligonucleotide
gcgcgctcgctcgctcgagagaaccaggcgaccaaaggtcgcctgacgccatggcggttctctcgagcgagcgagcgcgc

SEQ ID NO: 381 - DNA, 80 bp, Artificial Sequence
  Description of Artificial Sequence: Synthetic oligonucleotide
gcgcgctcgctcgctcgagagaaccaggcgaccaaaggtcgcctgacgacatgtcggttctctcgagcgagcgagcgcgc

SEQ ID NO: 382 - DNA, 80 bp, Artificial Sequence
  Description of Artificial Sequence: Synthetic oligonucleotide
gcgcgctcgctcgctcgagagaaccaggcgaccaaaggtcgcctgacgaacgttcggttctctcgagcgagcgagcgcgc

SEQ ID NO: 383 - DNA, 80 bp, Artificial Sequence
  Description of Artificial Sequence: Synthetic oligonucleotide
gcgcgctcgctcgctcgagagaaccaggcgaccaaaggtcgcctgaagcaattgctgttctctcgagcgagcgagcgcgc

SEQ ID NO: 384 - DNA, 80 bp, Artificial Sequence
  Description of Artificial Sequence: Synthetic oligonucleotide
gcgcgctcgctcgctcgagagaaccgggcggccaaaggccgcccgacacccgggtggttctctcgagcgagcgagcgcgc

SEQ ID NO: 385 - DNA, 80 bp, Artificial Sequence
  Description of Artificial Sequence: Synthetic oligonucleotide
gcgcgctcgctcgctcgagagaaccgggcggccaaaggccgcccgacgccatggcggttctctcgagcgagcgagcgcgc

SEQ ID NO: 386 - DNA, 80 bp, Artificial Sequence
  Description of Artificial Sequence: Synthetic oligonucleotide
gcgcgctcgctcgctcgagagaaccgggcggccaaaggccgcccgacgacatgtcggttctctcgagcgagcgagcgcgc

SEQ ID NO: 387 - DNA, 80 bp, Artificial Sequence
  Description of Artificial Sequence: Synthetic oligonucleotide
gcgcgctcgctcgctcgagagaaccgggcggccaaaggccgcccgacgaacgttcggttctctcgagcgagcgagcgcgc

SEQ ID NO: 388 - DNA, 80 bp, Artificial Sequence
  Description of Artificial Sequence: Synthetic oligonucleotide
gcgcgctcgctcgctcgagagaaccgggcggccaaaggccgcccgaagcaattgctgttctctcgagcgagcgagcgcgc

SEQ ID NO: 389 - DNA, 80 bp, Artificial Sequence
  Description of Artificial Sequence: Synthetic oligonucleotide
gcgcgctcgctcgctcgagagaaccgggcgacaaaatgtcgcccgacacccgggtggttctctcgagcgagcgagcgcgc

SEQ ID NO: 390 - DNA, 80 bp, Artificial Sequence
  Description of Artificial Sequence: Synthetic oligonucleotide
gcgcgctcgctcgctcgagagaaccgggcgacaaaatgtcgcccgacgccatggcggttctctcgagcgagcgagcgcgc

SEQ ID NO: 391 - DNA, 80 bp, Artificial Sequence
  Description of Artificial Sequence: Synthetic oligonucleotide
gcgcgctcgctcgctcgagagaaccgggcgacaaaatgtcgcccgacgacatgtcggttctctcgagcgagcgagcgcgc

SEQ ID NO: 392 - DNA, 80 bp, Artificial Sequence
  Description of Artificial Sequence: Synthetic oligonucleotide
gcgcgctcgctcgctcgagagaaccgggcgacaaaatgtcgcccgacgaacgttcggttctctcgagcgagcgagcgcgc

SEQ ID NO: 393 - DNA, 80 bp, Artificial Sequence
  Description of Artificial Sequence: Synthetic oligonucleotide
gcgcgctcgctcgctcgagagaaccgggcgacaaaatgtcgcccgaagcaattgctgttctctcgagcgagcgagcgcgc

SEQ ID NO: 394 - DNA, 80 bp, Artificial Sequence
  Description of Artificial Sequence: Synthetic oligonucleotide
gcgcgctcgctcgctcgagagaaccaaaagaccaaaggtcttttgacacccgggtggttctctcgagcgagcgagcgcgc

SEQ ID NO: 395 - DNA, 80 bp, Artificial Sequence
  Description of Artificial Sequence: Synthetic oligonucleotide
gcgcgctcgctcgctcgagagaaccaaaagaccaaaggtcttttgacgccatggcggttctctcgagcgagcgagcgcgc

SEQ ID NO: 396 - DNA, 80 bp, Artificial Sequence
  Description of Artificial Sequence: Synthetic oligonucleotide
gcgcgctcgctcgctcgagagaaccaaaagaccaaaggtcttttgacgacatgtcggttctctcgagcgagcgagcgcgc

SEQ ID NO: 397 - DNA, 80 bp, Artificial Sequence
  Description of Artificial Sequence: Synthetic oligonucleotide
gcgcgctcgctcgctcgagagaaccaaaagaccaaaggtcttttgacgaacgttcggttctctcgagcgagcgagcgcgc

SEQ ID NO: 398 - DNA, 80 bp, Artificial Sequence
  Description of Artificial Sequence: Synthetic oligonucleotide
gcgcgctcgctcgctcgagagaaccaaaagaccaaaggtcttttgaagcaattgctgttctctcgagcgagcgagcgcgc

SEQ ID NO: 399 - DNA, 80 bp, Artificial Sequence
  Description of Artificial Sequence: Synthetic oligonucleotide
gcgcgctcgctcgctcgagagaaccaaaaaaaaaaattttttttgacacccgggtggttctctcgagcgagcgagcgcgc

SEQ ID NO: 400 - DNA, 80 bp, Artificial Sequence
  Description of Artificial Sequence: Synthetic oligonucleotide
gcgcgctcgctcgctcgagagaaccaaaaaaaaaaattttttttgacgccatggcggttctctcgagcgagcgagcgcgc

SEQ ID NO: 401 - DNA, 80 bp, Artificial Sequence
  Description of Artificial Sequence: Synthetic oligonucleotide
gcgcgctcgctcgctcgagagaaccaaaaaaaaaaattttttttgacgacatgtcggttctctcgagcgagcgagcgcgc

SEQ ID NO: 402 - DNA, 80 bp, Artificial Sequence
  Description of Artificial Sequence: Synthetic oligonucleotide
gcgcgctcgctcgctcgagagaaccaaaaaaaaaaattttttttgacgaacgttcggttctctcgagcgagcgagcgcgc

SEQ ID NO: 403 - DNA, 80 bp, Artificial Sequence
  Description of Artificial Sequence: Synthetic oligonucleotide
gcgcgctcgctcgctcgagagaaccaaaaaaaaaaattttttttgaagcaattgctgttctctcgagcgagcgagcgcgc

SEQ ID NO: 404 - DNA, 80 bp, Artificial Sequence
  Description of Artificial Sequence: Synthetic oligonucleotide
gcgcgctcgctcgctcgagagaaccgggcagaaaaattctgcccgacacccgggtggttctctcgagcgagcgagcgcgc

SEQ ID NO: 405 - DNA, 80 bp, Artificial Sequence
  Description of Artificial Sequence: Synthetic oligonucleotide
gcgcgctcgctcgctcgagagaaccgggcagaaaaattctgcccgacgccatggcggttctctcgagcgagcgagcgcgc

SEQ ID NO: 406 - DNA, 80 bp, Artificial Sequence
  Description of Artificial Sequence: Synthetic oligonucleotide
gcgcgctcgctcgctcgagagaaccgggcagaaaaattctgcccgacgacatgtcggttctctcgagcgagcgagcgcgc

SEQ ID NO: 407 - DNA, 80 bp, Artificial Sequence
  Description of Artificial Sequence: Synthetic oligonucleotide
gcgcgctcgctcgctcgagagaaccgggcagaaaaattctgcccgacgaacgttcggttctctcgagcgagcgagcgcgc

SEQ ID NO: 408 - DNA, 80 bp, Artificial Sequence
  Description of Artificial Sequence: Synthetic oligonucleotide
gcgcgctcgctcgctcgagagaaccgggcagaaaaattctgcccgaagcaattgctgttctctcgagcgagcgagcgcgc

SEQ ID NO: 409 - DNA, 80 bp, Artificial Sequence
  Description of Artificial Sequence: Synthetic oligonucleotide
gcgcgctcgctcgctcgcgggggccaggcgaccaaaggtcgcctgacacccgggtggcccccgcgagcgagcgagcgcgc

SEQ ID NO: 410 - DNA, 80 bp, Artificial Sequence
  Description of Artificial Sequence: Synthetic oligonucleotide
gcgcgctcgctcgctcgcgggggccaggcgaccaaaggtcgcctgacgccatggcggcccccgcgagcgagcgagcgcgc

SEQ ID NO: 411 - DNA, 80 bp, Artificial Sequence
  Description of Artificial Sequence: Synthetic oligonucleotide
gcgcgctcgctcgctcgcgggggccaggcgaccaaaggtcgcctgacgacatgtcggcccccgcgagcgagcgagcgcgc

SEQ ID NO: 412 - DNA, 80 bp, Artificial Sequence
  Description of Artificial Sequence: Synthetic oligonucleotide
gcgcgctcgctcgctcgcgggggccaggcgaccaaaggtcgcctgacgaacgttcggcccccgcgagcgagcgagcgcgc

SEQ ID NO: 413 - DNA, 80 bp, Artificial Sequence
  Description of Artificial Sequence: Synthetic oligonucleotide
gcgcgctcgctcgctcgcgggggccaggcgaccaaaggtcgcctgaagcaattgctgcccccgcgagcgagcgagcgcgc

SEQ ID NO: 414 - DNA, 80 bp, Artificial Sequence
  Description of Artificial Sequence: Synthetic oligonucleotide
gcgcgctcgctcgctcgcgggggccgggcggccaaaggccgcccgacacccgggtggcccccgcgagcgagcgagcgcgc

SEQ ID NO: 415 - DNA, 80 bp, Artificial Sequence
  Description of Artificial Sequence: Synthetic oligonucleotide
gcgcgctcgctcgctcgcgggggccgggcggccaaaggccgcccgacgccatggcggcccccgcgagcgagcgagcgcgc

SEQ ID NO: 416 - DNA, 80 bp, Artificial Sequence
  Description of Artificial Sequence: Synthetic oligonucleotide
gcgcgctcgctcgctcgcgggggccgggcggccaaaggccgcccgacgacatgtcggcccccgcgagcgagcgagcgcgc

SEQ ID NO: 417 - DNA, 80 bp, Artificial Sequence
  Description of Artificial Sequence: Synthetic oligonucleotide
gcgcgctcgctcgctcgcgggggccgggcggccaaaggccgcccgacgaacgttcggcccccgcgagcgagcgagcgcgc

SEQ ID NO: 418 - DNA, 80 bp, Artificial Sequence
  Description of Artificial Sequence: Synthetic oligonucleotide
gcgcgctcgctcgctcgcgggggccgggcggccaaaggccgcccgaagcaattgctgcccccgcgagcgagcgagcgcgc

SEQ ID NO: 419 - DNA, 80 bp, Artificial Sequence
  Description of Artificial Sequence: Synthetic oligonucleotide
gcgcgctcgctcgctcgcgggggccgggcgacaaaatgtcgcccgacacccgggtggcccccgcgagcgagcgagcgcgc

SEQ ID NO: 420 - DNA, 80 bp, Artificial Sequence
  Description of Artificial Sequence: Synthetic oligonucleotide
gcgcgctcgctcgctcgcgggggccgggcgacaaaatgtcgcccgacgccatggcggcccccgcgagcgagcgagcgcgc

SEQ ID NO: 421 - DNA, 80 bp, Artificial Sequence
  Description of Artificial Sequence: Synthetic oligonucleotide
gcgcgctcgctcgctcgcgggggccgggcgacaaaatgtcgcccgacgacatgtcggcccccgcgagcgagcgagcgcgc

SEQ ID NO: 422 - DNA, 80 bp, Artificial Sequence
  Description of Artificial Sequence: Synthetic oligonucleotide
gcgcgctcgctcgctcgcgggggccgggcgacaaaatgtcgcccgacgaacgttcggcccccgcgagcgagcgagcgcgc

SEQ ID NO: 423 - DNA, 80 bp, Artificial Sequence
  Description of Artificial Sequence: Synthetic oligonucleotide
gcgcgctcgctcgctcgcgggggccgggcgacaaaatgtcgcccgaagcaattgctgcccccgcgagcgagcgagcgcgc

SEQ ID NO: 424 - DNA, 80 bp, Artificial Sequence
  Description of Artificial Sequence: Synthetic oligonucleotide
gcgcgctcgctcgctcgcgggggccaaaagaccaaaggtcttttgacacccgggtggcccccgcgagcgagcgagcgcgc

SEQ ID NO: 425 - DNA, 80 bp, Artificial Sequence
  Description of Artificial Sequence: Synthetic oligonucleotide
gcgcgctcgctcgctcgcgggggccaaaagaccaaaggtcttttgacgccatggcggcccccgcgagcgagcgagcgcgc

SEQ ID NO: 426 - DNA, 80 bp, Artificial Sequence
  Description of Artificial Sequence: Synthetic oligonucleotide
gcgcgctcgctcgctcgcgggggccaaaagaccaaaggtcttttgacgacatgtcggcccccgcgagcgagcgagcgcgc

SEQ ID NO: 427 - DNA, 80 bp, Artificial Sequence
  Description of Artificial Sequence: Synthetic oligonucleotide
gcgcgctcgctcgctcgcgggggccaaaagaccaaaggtcttttgacgaacgttcggcccccgcgagcgagcgagcgcgc

SEQ ID NO: 428 - DNA, 80 bp, Artificial Sequence
  Description of Artificial Sequence: Synthetic oligonucleotide
gcgcgctcgctcgctcgcgggggccaaaagaccaaaggtcttttgaagcaattgctgcccccgcgagcgagcgagcgcgc

SEQ ID NO: 429 - DNA, 80 bp, Artificial Sequence
  Description of Artificial Sequence: Synthetic oligonucleotide
gcgcgctcgctcgctcgcgggggccaaaaaaaaaaattttttttgacacccgggtggcccccgcgagcgagcgagcgcgc

SEQ ID NO: 430 - DNA, 80 bp, Artificial Sequence
  Description of Artificial Sequence: Synthetic oligonucleotide
gcgcgctcgctcgctcgcgggggccaaaaaaaaaaattttttttgacgccatggcggcccccgcgagcgagcgagcgcgc

SEQ ID NO: 431 - DNA, 80 bp, Artificial Sequence
  Description of Artificial Sequence: Synthetic oligonucleotide
gcgcgctcgctcgctcgcgggggccaaaaaaaaaaattttttttgacgacatgtcggcccccgcgagcgagcgagcgcgc

SEQ ID NO: 432 - DNA, 80 bp, Artificial Sequence
  Description of Artificial Sequence: Synthetic oligonucleotide
gcgcgctcgctcgctcgcgggggccaaaaaaaaaaattttttttgacgaacgttcggcccccgcgagcgagcgagcgcgc

SEQ ID NO: 433 - DNA, 80 bp, Artificial Sequence
  Description of Artificial Sequence: Synthetic oligonucleotide
gcgcgctcgctcgctcgcgggggccaaaaaaaaaaattttttttgaagcaattgctgcccccgcgagcgagcgagcgcgc

SEQ ID NO: 434 - DNA, 80 bp, Artificial Sequence
  Description of Artificial Sequence: Synthetic oligonucleotide
gcgcgctcgctcgctcgcgggggccgggcagaaaaattctgcccgacacccgggtggcccccgcgagcgagcgagcgcgc

SEQ ID NO: 435 - DNA, 80 bp, Artificial Sequence
  Description of Artificial Sequence: Synthetic oligonucleotide
gcgcgctcgctcgctcgcgggggccgggcagaaaaattctgcccgacgccatggcggcccccgcgagcgagcgagcgcgc

SEQ ID NO: 436 - DNA, 80 bp, Artificial Sequence
  Description of Artificial Sequence: Synthetic oligonucleotide
gcgcgctcgctcgctcgcgggggccgggcagaaaaattctgcccgacgacatgtcggcccccgcgagcgagcgagcgcgc

SEQ ID NO: 437 - DNA, 80 bp, Artificial Sequence
  Description of Artificial Sequence: Synthetic oligonucleotide
gcgcgctcgctcgctcgcgggggccgggcagaaaaattctgcccgacgaacgttcggcccccgcgagcgagcgagcgcgc

SEQ ID NO: 438 - DNA, 80 bp, Artificial Sequence
  Description of Artificial Sequence: Synthetic oligonucleotide
gcgcgctcgctcgctcgcgggggccgggcagaaaaattctgcccgaagcaattgctgcccccgcgagcgagcgagcgcgc

SEQ ID NO: 439 - DNA, 80 bp, Artificial Sequence
  Description of Artificial Sequence: Synthetic oligonucleotide
gcgcgctcgctcgctcaataaaaccaggcgaccaaaggtcgcctgacacccgggtggttttattgagcgagcgagcgcgc

SEQ ID NO: 440 - DNA, 80 bp, Artificial Sequence
  Description of Artificial Sequence: Synthetic oligonucleotide
gcgcgctcgctcgctcaataaaaccaggcgaccaaaggtcgcctgacgccatggcggttttattgagcgagcgagcgcgc

SEQ ID NO: 441 - DNA, 80 bp, Artificial Sequence
  Description of Artificial Sequence: Synthetic oligonucleotide
gcgcgctcgctcgctcaataaaaccaggcgaccaaaggtcgcctgacgacatgtcggttttattgagcgagcgagcgcgc

SEQ ID NO: 442 - DNA, 80 bp, Artificial Sequence
  Description of Artificial Sequence: Synthetic oligonucleotide
gcgcgctcgctcgctcaataaaaccaggcgaccaaaggtcgcctgacgaacgttcggttttattgagcgagcgagcgcgc

SEQ ID NO: 443 - DNA, 80 bp, Artificial Sequence
  Description of Artificial Sequence: Synthetic oligonucleotide
gcgcgctcgctcgctcaataaaaccaggcgaccaaaggtcgcctgaagcaattgctgttttattgagcgagcgagcgcgc

SEQ ID NO: 444 - DNA, 80 bp, Artificial Sequence
  Description of Artificial Sequence: Synthetic oligonucleotide
gcgcgctcgctcgctcaataaaaccgggcggccaaaggccgcccgacacccgggtggttttattgagcgagcgagcgcgc

SEQ ID NO: 445 - DNA, 80 bp, Artificial Sequence
  Description of Artificial Sequence: Synthetic oligonucleotide
gcgcgctcgctcgctcaataaaaccgggcggccaaaggccgcccgacgccatggcggttttattgagcgagcgagcgcgc

SEQ ID NO: 446 - DNA, 80 bp, Artificial Sequence
  Description of Artificial Sequence: Synthetic oligonucleotide
gcgcgctcgctcgctcaataaaaccgggcggccaaaggccgcccgacgacatgtcggttttattgagcgagcgagcgcgc

SEQ ID NO: 447 - DNA, 80 bp, Artificial Sequence
  Description of Artificial Sequence: Synthetic oligonucleotide
gcgcgctcgctcgctcaataaaaccgggcggccaaaggccgcccgacgaacgttcggttttattgagcgagcgagcgcgc

SEQ ID NO: 448 - DNA, 80 bp, Artificial Sequence
  Description of Artificial Sequence: Synthetic oligonucleotide
gcgcgctcgctcgctcaataaaaccgggcggccaaaggccgcccgaagcaattgctgttttattgagcgagcgagcgcgc

SEQ ID NO: 449 - DNA, 80 bp, Artificial Sequence
  Description of Artificial Sequence: Synthetic oligonucleotide
gcgcgctcgctcgctcaataaaaccgggcgacaaaatgtcgcccgacacccgggtggttttattgagcgagcgagcgcgc

SEQ ID NO: 450 - DNA, 80 bp, Artificial Sequence
  Description of Artificial Sequence: Synthetic oligonucleotide
gcgcgctcgctcgctcaataaaaccgggcgacaaaatgtcgcccgacgccatggcggttttattgagcgagcgagcgcgc

SEQ ID NO: 451 - DNA, 80 bp, Artificial Sequence
  Description of Artificial Sequence: Synthetic oligonucleotide
gcgcgctcgctcgctcaataaaaccgggcgacaaaatgtcgcccgacgacatgtcggttttattgagcgagcgagcgcgc

SEQ ID NO: 452 - DNA, 80 bp, Artificial Sequence
  Description of Artificial Sequence: Synthetic oligonucleotide
gcgcgctcgctcgctcaataaaaccgggcgacaaaatgtcgcccgacgaacgttcggttttattgagcgagcgagcgcgc

SEQ ID NO: 453 - DNA, 80 bp, Artificial Sequence
  Description of Artificial Sequence: Synthetic oligonucleotide
gcgcgctcgctcgctcaataaaaccgggcgacaaaatgtcgcccgaagcaattgctgttttattgagcgagcgagcgcgc

SEQ ID NO: 454 - DNA, 80 bp, Artificial Sequence
  Description of Artificial Sequence: Synthetic oligonucleotide
gcgcgctcgctcgctcaataaaaccaaaagaccaaaggtcttttgacacccgggtggttttattgagcgagcgagcgcgc

SEQ ID NO: 455 - DNA, 80 bp, Artificial Sequence
  Description of Artificial Sequence: Synthetic oligonucleotide
gcgcgctcgctcgctcaataaaaccaaaagaccaaaggtcttttgacgccatggcggttttattgagcgagcgagcgcgc

SEQ ID NO: 456 - DNA, 80 bp, Artificial Sequence
  Description of Artificial Sequence: Synthetic oligonucleotide
gcgcgctcgctcgctcaataaaaccaaaagaccaaaggtcttttgacgacatgtcggttttattgagcgagcgagcgcgc

SEQ ID NO: 457 - DNA, 80 bp, Artificial Sequence
  Description of Artificial Sequence: Synthetic oligonucleotide
gcgcgctcgctcgctcaataaaaccaaaagaccaaaggtcttttgacgaacgttcggttttattgagcgagcgagcgcgc

SEQ ID NO: 458 - DNA, 80 bp, Artificial Sequence
  Description of Artificial Sequence: Synthetic oligonucleotide
gcgcgctcgctcgctcaataaaaccaaaagaccaaaggtcttttgaagcaattgctgttttattgagcgagcgagcgcgc

SEQ ID NO: 459 - DNA, 80 bp, Artificial Sequence
  Description of Artificial Sequence: Synthetic oligonucleotide
gcgcgctcgctcgctcaataaaaccaaaaaaaaaaattttttttgacacccgggtggttttattgagcgagcgagcgcgc

SEQ ID NO: 460 - DNA, 80 bp, Artificial Sequence
  Description of Artificial Sequence: Synthetic oligonucleotide
gcgcgctcgctcgctcaataaaaccaaaaaaaaaaattttttttgacgccatggcggttttattgagcgagcgagcgcgc

SEQ ID NO: 461 - DNA, 80 bp, Artificial Sequence
  Description of Artificial Sequence: Synthetic oligonucleotide
gcgcgctcgctcgctcaataaaaccaaaaaaaaaaattttttttgacgacatgtcggttttattgagcgagcgagcgcgc

SEQ ID NO: 462 - DNA, 80 bp, Artificial Sequence
  Description of Artificial Sequence: Synthetic oligonucleotide
gcgcgctcgctcgctcaataaaaccaaaaaaaaaaattttttttgacgaacgttcggttttattgagcgagcgagcgcgc

SEQ ID NO: 463 - DNA, 80 bp, Artificial Sequence
  Description of Artificial Sequence: Synthetic oligonucleotide
gcgcgctcgctcgctcaataaaaccaaaaaaaaaaattttttttgaagcaattgctgttttattgagcgagcgagcgcgc

SEQ ID NO: 464 - DNA, 80 bp, Artificial Sequence
  Description of Artificial Sequence: Synthetic oligonucleotide
gcgcgctcgctcgctcaataaaaccgggcagaaaaattctgcccgacacccgggtggttttattgagcgagcgagcgcgc

SEQ ID NO: 465 - DNA, 80 bp, Artificial Sequence
  Description of Artificial Sequence: Synthetic oligonucleotide
gcgcgctcgctcgctcaataaaaccgggcagaaaaattctgcccgacgccatggcggttttattgagcgagcgagcgcgc

SEQ ID NO: 466 - DNA, 80 bp, Artificial Sequence
  Description of Artificial Sequence: Synthetic oligonucleotide
gcgcgctcgctcgctcaataaaaccgggcagaaaaattctgcccgacgacatgtcggttttattgagcgagcgagcgcgc

SEQ ID NO: 467 - DNA, 80 bp, Artificial Sequence
  Description of Artificial Sequence: Synthetic oligonucleotide
gcgcgctcgctcgctcaataaaaccgggcagaaaaattctgcccgacgaacgttcggttttattgagcgagcgagcgcgc

SEQ ID NO: 468 - DNA, 80 bp, Artificial Sequence
  Description of Artificial Sequence: Synthetic oligonucleotide
gcgcgctcgctcgctcaataaaaccgggcagaaaaattctgcccgaagcaattgctgttttattgagcgagcgagcgcgc

SEQ ID NO: 469 - DNA, 120 bp, Artificial Sequence
  Description of Artificial Sequence: Synthetic polynucleotide
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 470 - DNA, 122 bp, Artificial Sequence
  Description of Artificial Sequence: Synthetic polynucleotide
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 471 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 472 - DNA, 101 bp, Artificial Sequence
  Description of Artificial Sequence: Synthetic polynucleotide
aggaacccctagtgatggagttggccactccctctctgcgcgctcgctcgctcactgaggctttgcctcagtgagcgagcgagcgcgcagctgcctgcagg

SEQ ID NO: 473 - DNA, 139 bp, Artificial Sequence
  Description of Artificial Sequence: Synthetic polynucleotide
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 474 - DNA, 137 bp, Artificial Sequence
  Description of Artificial Sequence: Synthetic polynucleotide
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 475 - DNA, 135 bp, Artificial Sequence
  Description of Artificial Sequence: Synthetic polynucleotide
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 476 - DNA, 133 bp, Artificial Sequence
  Description of Artificial Sequence: Synthetic polynucleotide
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 477 - DNA, 139 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 478 - DNA, 137 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 479 - DNA, 135 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 480 - DNA, 133 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 481 - DNA, 131 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 482 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 483 - DNA, 127 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 484 - DNA, 120 bp, Artificial Sequence
  Description of Artificial Sequence: Synthetic polynucleotide
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 485 - DNA, 122 bp, Artificial Sequence
  Description of Artificial Sequence: Synthetic polynucleotide
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 486 - DNA, 122 bp, Artificial Sequence
  Description of Artificial Sequence: Synthetic polynucleotide
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 487 - DNA, 129 bp, Artificial Sequence
  Description of Artificial Sequence: Synthetic polynucleotide
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 488 - DNA, 101 bp, Artificial Sequence
  Description of Artificial Sequence: Synthetic polynucleotide
cctgcaggcagctgcgcgctcgctcgctcactgaggcaaagcctcagtgagcgagcgagcgcgcagagagggagtggccaactccatcactaggggttcct

SEQ ID NO: 489 - DNA, 139 bp, Artificial Sequence
  Description of Artificial Sequence: Synthetic polynucleotide
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 490 - DNA, 137 bp, Artificial Sequence
  Description of Artificial Sequence: Synthetic polynucleotide
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 491 - DNA, 135 bp, Artificial Sequence
  Description of Artificial Sequence: Synthetic polynucleotide
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 492 - DNA, 133 bp, Artificial Sequence
  Description of Artificial Sequence: Synthetic polynucleotide
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 493 - DNA, 139 bp, Artificial Sequence
  Description of Artificial Sequence: Synthetic polynucleotide
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 494 - DNA, 137 bp, Artificial Sequence
  Description of Artificial Sequence: Synthetic polynucleotide
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 495 - DNA, 135 bp, Artificial Sequence
  Description of Artificial Sequence: Synthetic polynucleotide
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 496 - DNA, 133 bp, Artificial Sequence
  Description of Artificial Sequence: Synthetic polynucleotide
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 497 - DNA, 131 bp, Artificial Sequence
  Description of Artificial Sequence: Synthetic polynucleotide
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 498 - DNA, 129 bp, Artificial Sequence
  Description of Artificial Sequence: Synthetic polynucleotide
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 499 - DNA, 127 bp, Artificial Sequence
  Description of Artificial Sequence: Synthetic polynucleotide
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 500 - DNA, 43 bp, Artificial Sequence
  Description of Artificial Sequence: Synthetic oligonucleotide
gcccgctggtttccagcgggctgcgggcccgaaacgggcccgc

SEQ ID NO: 501 - DNA, 28 bp, Artificial Sequence
  Description of Artificial Sequence: Synthetic oligonucleotide
cgggcccgtgcgggcccaaagggcccgc

SEQ ID NO: 502 - DNA, 28 bp, Artificial Sequence
  Description of Artificial Sequence: Synthetic oligonucleotide
gcccgggcacgcccgggtttcccgggcg

SEQ ID NO: 503 - DNA, 22 bp, Artificial Sequence
  Description of Artificial Sequence: Synthetic oligonucleotide
cgtgcgggcccaaagggcccgc

SEQ ID NO: 504 - DNA, 21 bp, Artificial Sequence
  Description of Artificial Sequence: Synthetic oligonucleotide
cgggcgaccaaaggtcgcccg

SEQ ID NO: 505 - DNA, 20 bp, Artificial Sequence
  Description of Artificial Sequence: Synthetic oligonucleotide
cgcccgggctttgcccgggc

SEQ ID NO: 506 - DNA, 42 bp, Artificial Sequence
  Description of Artificial Sequence: Synthetic oligonucleotide
cgggcgaccaaaggtcgcccgacgcccgggctttgcccgggc

SEQ ID NO: 507 - DNA, 21 bp, Artificial Sequence
  Description of Artificial Sequence: Synthetic oligonucleotide
cgggcgaccaaaggtcgcccg

SEQ ID NO: 508 - DNA, 20 bp, Artificial Sequence
  Description of Artificial Sequence: Synthetic oligonucleotide
cgcccgggctttgcccgggc

SEQ ID NO: 509 - DNA, 34 bp, Artificial Sequence
  Description of Artificial Sequence: Synthetic oligonucleotide
cgggcgaccaaaggtcgcccgacgcccgggcggc

SEQ ID NO: 510 - DNA, 21 bp, Artificial Sequence
  Description of Artificial Sequence: Synthetic oligonucleotide
cgggcgaccaaaggtcgcccg

SEQ ID NO: 511 - DNA, 20 bp, Artificial Sequence
  Description of Artificial Sequence: Synthetic oligonucleotide
cgcccgggctttgcccgggc

SEQ ID NO: 512 - DNA, 30 bp, Artificial Sequence
  Description of Artificial Sequence: Synthetic oligonucleotide
cggggcccgacgcccgggctttgcccgggc

SEQ ID NO: 513 - DNA, 21 bp, Artificial Sequence
  Description of Artificial Sequence: Synthetic oligonucleotide
cgggcgaccaaaggtcgcccg

SEQ ID NO: 514 - DNA, 20 bp, Artificial Sequence
  Description of Artificial Sequence: Synthetic oligonucleotide
cgcccgggctttgcccgggc

SEQ ID NO: 515 - DNA, 29 bp, Artificial Sequence
  Description of Artificial Sequence: Synthetic oligonucleotide
cgggcccgacgcccgggctttgcccgggc

SEQ ID NO: 516 - DNA, 21 bp, Artificial Sequence
  Description of Artificial Sequence: Synthetic oligonucleotide
cgggcgaccaaaggtcgcccg

SEQ ID NO: 517 - DNA, 20 bp, Artificial Sequence
  Description of Artificial Sequence: Synthetic oligonucleotide
cgcccgggctttgcccgggc

SEQ ID NO: 518 - DNA, 20 bp, Artificial Sequence
  Description of Artificial Sequence: Synthetic oligonucleotide
gcccgggcaaagcccgggcg

SEQ ID NO: 519 - DNA, 21 bp, Artificial Sequence
  Description of Artificial Sequence: Synthetic oligonucleotide
cgggcgacctttggtcgcccg

SEQ ID NO: 520 - DNA, 42 bp, Artificial Sequence
  Description of Artificial Sequence: Synthetic oligonucleotide
gcccgggcaaagcccgggcgtcgggcgacctttggtcgcccg

SEQ ID NO: 521 - DNA, 20 bp, Artificial Sequence
  Description of Artificial Sequence: Synthetic oligonucleotide
gcccgggcaaagcccgggcg

SEQ ID NO: 522 - DNA, 31 bp, Artificial Sequence
  Description of Artificial Sequence: Synthetic oligonucleotide
gcccgggcgtcgggcgacctttggtcgcccg

SEQ ID NO: 523 - DNA, 20 bp, Artificial Sequence
  Description of Artificial Sequence: Synthetic oligonucleotide
gcccgggcaaagcccgggcg

SEQ ID NO: 524 - DNA, 21 bp, Artificial Sequence
  Description of Artificial Sequence: Synthetic oligonucleotide
cgggcgacctttggtcgcccg

SEQ ID NO: 525 - DNA, 34 bp, Artificial Sequence
  Description of Artificial Sequence: Synthetic oligonucleotide
gccgcccgggcgacgggcgacctttggtcgcccg

SEQ ID NO: 526 - DNA, 20 bp, Artificial Sequence
  Description of Artificial Sequence: Synthetic oligonucleotide
gcccgggcaaagcccgggcg

SEQ ID NO: 527 - DNA, 21 bp, Artificial Sequence
  Description of Artificial Sequence: Synthetic oligonucleotide
cgggcgacctttggtcgcccg

SEQ ID NO: 528 - DNA, 31 bp, Artificial Sequence; identical to SEQ ID NO: 522
  Description of Artificial Sequence: Synthetic oligonucleotide

SEQ ID NO: 529 - DNA, 21 bp, Artificial Sequence
  Description of Artificial Sequence: Synthetic oligonucleotide
cgggcgacctttggtcgcccg

SEQ ID NO: 530 - DNA, 1653 bp, Artificial Sequence
  Description of Artificial Sequence: Synthetic polynucleotide
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 531 - DNA, 16 bp, Artificial Sequence
  Description of Artificial Sequence: Synthetic oligonucleotide
gcgcgctcgctcgctc

SEQ ID NO: 532 - DNA, 8 bp, Artificial Sequence
  Description of Artificial Sequence: Synthetic oligonucleotide
actgaggc

SEQ ID NO: 533 - DNA, 22 bp, Artificial Sequence
  Description of Artificial Sequence: Synthetic oligonucleotide
cgggcgaccaaaggtcgcccga

SEQ ID NO: 534 - DNA, 10 bp, Artificial Sequence
  Description of Artificial Sequence: Synthetic oligonucleotide
cgcccgggcg

SEQ ID NO: 535 - DNA, 8 bp, Artificial Sequence
  Description of Artificial Sequence: Synthetic oligonucleotide
gcctcagt

SEQ ID NO: 536 - DNA, 16 bp, Artificial Sequence
  Description of Artificial Sequence: Synthetic oligonucleotide
gagcgagcgagcgcgc

SEQ ID NO: 537 - DNA, 20 bp, Artificial Sequence
  Description of Artificial Sequence: Synthetic oligonucleotide
gcccgggcaaagcccgggcg

SEQ ID NO: 538 - DNA, 165 bp, Artificial Sequence
  Description of Artificial Sequence: Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 539 - DNA, 140 bp, Artificial Sequence
  Description of Artificial Sequence: Synthetic polynucleotide
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 540 - DNA, 91 bp, Artificial Sequence
  Description of Artificial Sequence: Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 541 - DNA, 91 bp, Artificial Sequence
  Description of Artificial Sequence: Synthetic oligonucleotide
gcgcgctcgctcgctcactgaggccgggcgaccaaaggtcgcccgacgcccgggctttgcccgggcggcctcagtgagcgagcgagcgcgc

SEQ ID NO: 542 - DNA, 8 bp, Artificial Sequence
  Description of Artificial Sequence: Synthetic oligonucleotide
ttaattaa

SEQ ID NO: 543 - DNA, 80 bp, Artificial Sequence; identical to SEQ ID NO: 65
  Description of Artificial Sequence: Synthetic oligonucleotide

SEQ ID NO: 544 - DNA, 52 bp, Artificial Sequence
  Description of Artificial Sequence: Synthetic oligonucleotide
gcgcgctcgctcgctcactgaggcctttgcctcagtgagcgagcgagcgcgc

SEQ ID NO: 545 - DNA, 79 bp, Artificial Sequence
  Description of Artificial Sequence: Synthetic oligonucleotide
gcgcgctcgctcgctcactgaggcgcccgggcgtcgggcgacctttggtcgcccggcctcagtgagcgagcgagcgcgc

SEQ ID NO: 546 - DNA, 81 bp, Artificial Sequence
  Description of Artificial Sequence: Synthetic oligonucleotide
ctgcgcgctcgctcgctcactgaggccgggcgaccaaaggtcgcccgacgtttcggcctcagtgagcgagcgagcgcgcag

SEQ ID NO: 547 - DNA, 81 bp, Artificial Sequence
  Description of Artificial Sequence: Synthetic oligonucleotide
ctgcgcgctcgctcgctcactgaggccgaaacgtcgggcgacctttggtcgcccggcctcagtgagcgagcgagcgcgcag

SEQ ID NO: 548 - DNA, 42 bp, Artificial Sequence; identical to SEQ ID NO: 506
  Description of Artificial Sequence: Synthetic oligonucleotide

SEQ ID NO: 549 - DNA, 42 bp, Artificial Sequence; identical to SEQ ID NO: 520
  Description of Artificial Sequence: Synthetic oligonucleotide

SEQ ID NO: 550 - DNA, 144 bp, Artificial Sequence
  Description of Artificial Sequence: Synthetic polynucleotide
aggaaccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 551 - DNA, 43 bp, Artificial Sequence; identical to SEQ ID NO: 500
  Description of Artificial Sequence: Synthetic oligonucleotide

SEQ ID NO: 552 - DNA, 28 bp, Artificial Sequence
  Description of Artificial Sequence: Synthetic oligonucleotide
cgggcccgtgcgggcccaaagggcccgc

SEQ ID NO: 553 - DNA, 28 bp, Artificial Sequence
  Description of Artificial Sequence: Synthetic oligonucleotide
gcccgggcacgcccgggtttcccgggcg

SEQ ID NO: 554 - DNA, 22 bp, Artificial Sequence
  Description of Artificial Sequence: Synthetic oligonucleotide
cgtgcgggcccaaagggcccgc

SEQ ID NO: 555 - DNA, 43 bp, Artificial Sequence
  Description of Artificial Sequence: Synthetic oligonucleotide
gcgggccggaaacgggcccgctgcccgctggtttccagcgggc

SEQ ID NO: 556 - DNA, 28 bp, Artificial Sequence
  Description of Artificial Sequence: Synthetic oligonucleotide
cgcccgggaaacccgggcgtgcccgggc

SEQ ID NO: 557 - DNA, 28 bp, Artificial Sequence
  Description of Artificial Sequence: Synthetic oligonucleotide
gggccgcccgggaaacccgggcgtgccc

(19) United States
(12) Patent Application Publication
(10) Pub. No.: US 2020/0283794 A1
(43) Pub. Date: Sep. 10, 2020

(54) MODIFIED CLOSED-ENDED DNA (CEDNA)

(71) Applicant: GENERATION BIO CO., Cambridge, MA (US)

(72) Inventors: Robert Michael Kotin, Cambridge, MA (US); Ozan Alkan, Cambridge, MA (US); Annaliese Jones, Cambridge, MA (US); Douglas Anthony Kerr, Cambridge, MA (US); Ara Karl Malakian, Cambridge, MA (US); Matthew John Simmons, Cambridge, MA (US); Teresa L. Wright, Cambridge, MA (US)

(21) Appl. No.: 16/644,568

(22) PCT Filed: Sep. 7, 2018
(86) PCT No.: PCT/US2018/049996

§ 371 Date: Mar. 5, 2020

Related U.S. Application Data
(60) Provisional application No. 62/556,319, filed on Sep. 8, 2017.
(60) Provisional application No. 62/556,324, filed on Sep. 8, 2017.
(60) Provisional application No. 62/556,329, filed on Sep. 8, 2017.
(60) Provisional application No. 62/556,281, filed on Sep. 8, 2017.
(60) Provisional application No. 62/556,335, filed on Sep. 8, 2017.
(60) Provisional application No. 62/556,331, filed on Sep. 8, 2017.

(51) Int. Cl. C12N 15/85; C12N 15/11; C12N 15/64; A61K 48/00

(52) U.S. Cl. CPC C12N 15/85; C12N 15/11; C12N 15/64; C12N 2310/531; C12N 2800/00; C12N 2750/14121; A61K 48/005

(57) ABSTRACT

CeDNA vectors having linear and continuous structure can be produced in high yields and used for effective transfer and expression of a transgene. ceDNA vectors comprise an expression cassette and two different ITR sequences derived from AAV genomes in a specified order. Some ceDNA vectors provided herein further comprise cis-regulatory elements and provide high gene expression efficiencies. Further provided herein are methods and cell lines for reliable and efficient production of the linear, continuous and capsid-free DNA vectors.

### CROSS REFERENCE TO RELATED APPLICATIONS

This application claims benefit under 35 U.S.C. § 119(e) of U.S. Provisional Application Nos. 62/556,319; 62/556,324; 62/556,329; 62/556,331; 62/556,281 and 62/556,335, each of which were filed on Sep. 8, 2017, the contents of each are incorporated herein by reference in their entireties.

### SEQUENCE LISTIING

The instant application contains a Sequence Listing which has been submitted electronically in ASCII format and is hereby incorporated by reference in its entirety. Said ASCII copy, created on Sep. 7, 2018, is named 080170-090580WOPT_SL.txt and is 205,991 bytes in size.

### TECHNICAL FIELD

The present invention relates to the field of gene therapy, including the delivery of exogenous DNA sequences to a target cell, tissue, organ or organism.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g. underexpression or overexpression, that can result in a disorder, disease, malignancy, etc. For example, a disease or disorder caused by a defective gene might be treated, prevented or ameliorated by delivery of a corrective genetic material to a patient resulting in the therapeutic expression of the genetic material within the patient. The basis of gene therapy is to supply a transcription cassette with an active gene product (sometimes referred to as a transgene), e.g., that can result in a positive gain-of-function effect, a negative loss-of-function effect, or another outcome, such as, e.g., an oncolytic effect. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors. Among the many virus-derived vectors available (e.g, recombinant retrovirus, recombinant lentivirus, recombinant adenovirus, and the like), recombinant adeno-associated virus (rAAV) is gaining popularity as a versatile vector in gene therapy.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically-stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129).

Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes. AAV vectors can also be produced and formulated at high titer and delivered via intra-arterial, intra-venous, or intra-peritoneal injections allowing vector distribution and gene transfer to significant muscle regions through a single injection in rodents (Goyenvalle et al., 2004; Fougerousse et al., 2007; Koppanati et al., 2010; Wang et al., 2009) and dogs. In a clinical study to treat spinal muscular dystrophy type 1, AAV vectors were delivered systemically with the intention of targeting the brain resulting in apparent clinical improvements.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010). As a result, use of AAV vectors has been limited to less than 150,000 Da protein coding capacity. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression. While attempts have been made to circumvent this issue by constructing double-stranded DNA vectors, this strategy further limits the size of the transgene expression cassette that can be integrated into the AAV vector (McCarty, 2008; Varenika et al., 2009; Foust et al., 2009).

Additionally, conventional AAV virions with capsids are produced by introducing a plasmid or plasmids containing the AAV genome, rep genes, and cap genes (Grimm et al., 1998). Upon introduction of these helper plasmids in trans, the AAV genome is “rescued” (i.e., released and subsequently amplified) from the host genome, and is further encapsidated (viral capsids) to produce biologically active AAV vectors. However, such encapsidated AAV virus vectors were found to inefficiently transduce certain cell and tissue types. The capsids also induce an immune response.

Accordingly, use of adeno-associated virus (AAV) vectors for gene therapy is limited due to the single administration to patients (owing to the patient immune response), the limited range of transgene genetic material suitable for delivery in AAV vectors due to minimal viral packaging capacity (about 4.5 kb) of the associated AAV capsid, as well as the slow AAV-mediated gene expression. The applications for rAAV clinical gene therapies are further encumbered by patient-to-patient variability not predicted by dose response in syngeneic mouse models or in other model species.

Recombinant capsid-free AAV vectors can be obtained as an isolated linear nucleic acid molecule comprising an expressible transgene and promoter regions flanked by two wild-type AAV inverted terminal repeat sequences (ITRs) including the Rep binding and terminal resolution sites. These recombinant AAV vectors are devoid of AAV capsid protein encoding sequences, and can be single-stranded, double-stranded or duplex with one or both ends covalently linked through the two wild-type ITR palindrome sequences (e.g., WO2012/123430, U.S. Pat. No. 9,598,703). They avoid many of the problems of AAV-mediated gene therapy in that the transgene capacity is much higher, transgene expression onset is rapid, and the patient immune system does recognize the DNA molecules as a virus to be cleared. However, constant expression of a transgene may not be desirable in all instances, and AAV canonical wild type ITRs may not be optimized for ceDNA function. Therefore, there remains an important unmet need for controllable recombinant DNA vectors with improved production and/or expression properties.

### BRIEF DESCRIPTION OF THE INVENTION

The invention described herein is a non-viral capsid-free DNA vector with covalently-closed ends (referred to herein as a “closed-ended DNA vector” or a “ceDNA vector”). The ceDNA vectors described herein are capsid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence that are different, or asymmetrical with respect to each other.

The technology described herein relates to a ceDNA vector containing at least one modified AAV inverted terminal repeat sequence (ITR) and an expressible transgene. The ceDNA vectors disclosed herein can be produced in eukaryotic cells, thus devoid of prokaryotic DNA modifications and bacterial endotoxin contamination in insect cells.

In one aspect, non-viral capsid-free DNA vectors with covalently-closed ends are preferably linear duplex molecules, and are obtainable from a vector polynucleotide that encodes a heterologous nucleic acid operatively positioned between two different inverted terminal repeat sequences (ITRs) (e.g. AAV ITRs), wherein at least one of the ITRs comprises a terminal resolution site and a replication protein binding site (RPS) (sometimes referred to as a replicative protein binding site), e.g. a Rep binding site, and one of the ITRs comprises a deletion, insertion, or substitution with respect to the other ITR. That is, one of the ITRs is asymmetrical relative to the other ITR. In one embodiment, at least one of the ITRs is an AAV ITR, e.g. a wild type AAV ITR or modified AAV ITR. In one embodiment, at least one of the ITRs is a modified ITR relative to the other ITR—that is, the ceDNA comprises ITRs that are asymmetric relative to each other. In one embodiment, at least one of the ITRs is a non-functional ITR.

In some embodiments, the ceDNA vector comprises: (1) an expression cassette comprising a cis-regulatory element, a promoter and at least one transgene; or (2) a promoter operably linked to at least one transgene, and (3) two self-complementary sequences, e.g., ITRs, flanking said expression cassette, wherein the ceDNA vector is not associated with a capsid protein. In some embodiments, the ceDNA vector comprises two self-complementary sequences found in an AAV genome, where at least one comprises an operative Rep-binding element (RBE) (also sometimes referred to herein as “RBS”) and a terminal resolution site (trs) of AAV or a functional variant of the RBE, and one or more cis-regulatory elements operatively linked to a transgene. In some embodiments, the ceDNA vector comprises additional components to regulate expression of the transgene, for example, regulatory switches, which are described herein in the section entitled “Regulatory Switches” for controlling and regulating the expression of the transgene, and can include a regulatory switch, e.g., a kill switch to enable controlled cell death of a cell comprising a ceDNA vector.

In some embodiments, the two self-complementary sequences can be ITR sequences from any known parvovirus, for example a dependovirus such as AAV (e.g., AAV1-AAV12). Any AAV serotype can be used, including but not limited to a modifed AAV2 ITR sequence, that retains a Rep-binding site (RBS) such as 5′-GCGCGCTCGCTCGCTC-3′ (SEQ ID NO: 531) and a terminal resolution site (trs) in addition to a variable palindromic sequence allowing for hairpin secondary structure formation. In some embodiments, the ITR is a synthetic ITR sequence that retains a functional Rep-binding site (RBS) such as 5′-GCGCGCTCGCTCGCTC-3′ (SEQ ID NO: 531) and a terminal resolution site (TRS) in addition to a variable palindromic sequence allowing for hairpin secondary structure formation. In some examples, a modified ITR sequence retains the sequence of the RBS, trs and the structure and position of a Rep binding element forming the terminal loop portion of one of the ITR hairpin secondary structure from the corresponding sequence of the wild-type AAV2 ITR.

Exemplary ITR sequences for use in the ceDNA vectors are disclosed in any one or more of Tables 2-10A and 10B, or SEQ ID NO: 2, 52, 101-499 and 545-547 or the partial ITR sequences shown in FIG. 26A-26B. In some embodiments, the ceDNA vectors do not have an ITR that comprises any sequence selected from SEQ ID NOs: 500-529.

In some embodiments, a ceDNA vector can comprise an ITR with a modification in the ITR corresponding to any of the modifications in ITR sequences or ITR partial sequences shown in any one or more of Tables 2, 3, 4, 5, 6, 7, 8, 9, 10A and 10B herein.

As an exemplary example, the present disclosure provides a closed-ended DNA vector comprising a promoter operably linked to a transgene, where the ceDNA is devoid of capsid proteins and is: (a) produced from a ceDNA-plasmid (e.g., see Examples 1-2 and/or FIGS. 1A-B) that encodes a mutated right side AAV2 ITR having the same number of intramolecularly duplexed base pairs as SEQ ID NO:2 or a mutated left side AAV2 ITR having the same number of intramolecularly duplexed base pairs as SEQ ID NO:51 in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1. Examples of such modified ITR sequences are provided in Tables 2, 3, 4, 5, 6, 7, 8, 9, 10A and 10B herein.

The technology described herein further relates to a ceDNA vector that can deliver and encode one or more transgenes in a target cell, for example, where the ceDNA vector comprises a multicistronic sequence, or where the transgene and its native genomic context (e.g., transgene, introns and endogenous untranslated regions) are together incorporated into the ceDNA vector. The transgenes can be protein encoding transcripts, non-coding transcripts, or both. The ceDNA vector can comprise multiple coding sequences, and a non-canonical translation initiation site or more than one promoter to express protein encoding transcripts, non-coding transcripts, or both. The transgene can comprise a sequence encoding more than one proteins, or can be a sequence of a non-coding transcript. The expression cassette can comprise, e.g., more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. The ceDNA vectors do not have the size limitations of encapsidated AAV vectors, thus enable delivery of a large-size expression cassette to provide efficient expression of transgenes. In some embodiments, the ceDNA vector is devoid of prokaryote-specific methylation.

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene. For example, the additional regulatory component can be a regulator switch as disclosed herein, including but not limited to a kill switch, which can kill the ceDNA infected cell, if necessary, and other inducible and/or repressible elements.

The technology described herein further provides novel methods of delivering and efficiently and selectively expressing one or more transgenes using the ceDNA vectors. A ceDNA vector has the capacity to be taken up into host cells, as well as to be transported into the nucleus in the absence of the AAV capsid. In addition, the ceDNA vectors described herein lack a capsid and thus avoid the immune response that can arise in response to capsid-containing vectors.

Aspects of the invention relate to methods to produce the ceDNA vectors described herein. Other embodiments relate to a ceDNA vector produced by the method provided herein. In one embodiment, the capsid free non-viral DNA vector (ceDNA vector) is obtained from a plasmid (referred to herein as a “ceDNA-plasmid”) comprising a polynucleotide expression construct template comprising in this order: a first 5′ inverted terminal repeat (e.g. AAV ITR); an expression cassette; and a 3′ ITR (e.g. AAV ITR), where at least one of the 5′ and 3′ ITR is a modified ITR, or where when both the 5′ and 3′ ITRs are modified, they have different modifications from one another and are not the same sequence.

The ceDNA vector disclosed herein is obtainable by a number of means that would be known to the ordinarily skilled artisan after reading this disclosure. For example, a polynucleotide expression construct template used for generating the ceDNA vectors of the present invention can be a ceDNA-plasmid (e.g. see Table 12 or FIG. 10B), a ceDNA-bacmid, and/or a ceDNA-baculovirus. In one embodiment, the ceDNA-plasmid comprises a restriction cloning site (e.g. SEQ ID NO: 7) operably positioned between the ITRs where an expression cassette comprising e.g., a promoter operatively linked to a transgene, e.g., a reporter gene and/or a therapeutic gene) can be inserted. In some embodiments, ceDNA vectors are produced from a polynucleotide template (e.g., ceDNA-plasmid, ceDNA-bacmid, ceDNA-baculovirus) containing an ITR modified as compared to the corresponding flanking AAV3 ITR or wild-type AAV2 ITR sequence, where the modification is any one or more of deletion, insertion, and/or substitution.

In a permissive host cell, in the presence of e.g., Rep, the polynucleotide template having at least one modified ITR replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector. Rep proteins and Rep binding sites of the various AAV serotypes are well known to those of ordinary skill in the art. One of ordinary skill understands to choose a Rep protein from a serotype that binds to and replicates the nucleic acid sequence based upon at least one functional ITR. For example, if the replication competent ITR is from AAV serotype 2, the corresponding Rep would be from an AAV serotype that works with that serotype such as AAV2 ITR with AAV2 or AAV4 Rep but not AAV5 Rep, which does not. Upon replication, the covalently-closed ended ceDNA vector continues to accumulate in permissive cells and ceDNA vector is preferably sufficiently stable over time in the presence of Rep protein under standard replication conditions, e.g. to accumulate in an amount that is at least 1 pg/cell, preferably at least 2 pg/cell, preferably at least 3 pg/cell, more preferably at least 4 pg/cell, even more preferably at least 5 pg/cell.

Accordingly, one aspect of the invention relates to a process comprising the steps of: a) incubating a population of host cells (e.g. insect cells) harboring the polynucleotide expression construct template (e.g., a ceDNA-plasmid, a ceDNA-bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no virion-enforced size limitation.

The presence of the ceDNA vector isolated from the host cells can be confirmed by digesting DNA isolated from the host cell with a restriction enzyme having a single recognition site on the ceDNA vector and analyzing the digested DNA material on denaturing and non-denaturing gels to confirm the presence of characteristic bands of linear and continuous DNA as compared to linear and non-continuous DNA.

In some embodiments, the present application may be defined in any of the following paragraphs:
1. A non-viral capsid-free DNA vector with covalently-closed ends (ceDNA vector), wherein the ceDNA vector comprises at least one heterologous nucleotide sequence operably positioned between asymmetric inverted terminal repeat sequences (asymmetric ITRs), wherein at least one of the asymmetric ITRs comprises a functional terminal resolution site and a Rep binding site.
2. The ceDNA vector of paragraph 1, wherein the ceDNA vector when digested with a restriction enzyme having a single recognition site on the ceDNA vector and analyzed by both native and denaturing gel electrophoresis displays characteristic bands of linear and continuous DNA as compared to linear and non-continuous DNA controls.
3. The ceDNA vector of paragraphs 1 or 2, wherein one or more of the asymmetric ITR sequences are from a virus selected from a parvovirus, a dependovirus, and an adeno-associated virus (AAV).
4. The ceDNA vector of paragraph 3, wherein the asymmetric ITRs are from different viral serotypes.
5. The ceDNA vector of paragraph 4, wherein the one or more asymmetric ITRs are from an AAV serotype selected from AAV1, AAV2, AAV3, AAV4, AAV5, AAV6, AAV7, AAV8, AAV9, AAV10, AAV11, and AAV12.
6. The ceDNA vector of any one of paragraphs 1-3, wherein one or more of the asymmetric ITR sequences are synthetic.
7. The ceDNA vector of any one of paragraphs 1-3 and 6, wherein one or more of the ITRs is not a wild type ITR.
8. The ceDNA vector of any one of paragraphs 1-7, wherein one or more both of the asymmetric ITRs is modified by a deletion, insertion, and/or substitution in at least one of the ITR regions selected from A, A′, B, B′, C, C′, D, and D′. In some embodiments, one or both of the asymmetric ITRs can have a deletion, insertion, and/or substitution in any combination of B, B′, C or C′ regions as described in Table 1. In some embodiments, one or both of the asymmetric ITRs can have a deletion, insertion, and/or substitution in the B region, and/or B′ region, and/or C region, and/or C′ region. In some embodiments, one or both of the asymmetric ITRs can have one or more deletions, insertions, and/or substitutions in the A region and/or A′ region, and/or B region, and/or B′ region, and/or C region, and/or C′ region, and/or D region and/or D′ region. For example, in some embodiments, a modified ITR can have the removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-6). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm. In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned. In some embodiments, a modified ITR lacks a B-B′ arm. In some embodiments, a modified ITR lacks the C-C′ arm.
9. The ceDNA vector of paragraph 8, wherein the deletion, insertion, and/or substitution results in the deletion of all or part of a stem-loop structure normally formed by the A, A′, B, B′ C, or C′ regions. For example, in some embodiments, 1, 2, 3, 4, 5, 6, 7, 8, 9 or more complementary base pairs are removed from each of the C portion and the C′ portion of the C-C′ arm such that the C-C′ arm is truncated, and/or 1, 2, 3, 4, 5, 6, 7, 8, 9 or more complementary base pairs are removed from each of the B portion and the B′ portion of the B-B′ arm, such that the C-C′ arm and/or B′-B arm is truncated. In alternative embodiments, 1, 2, 3, 4, 5, 6, 7, 8, 9 or more base pairs are removed from the C portion of the C-C′ arm such that only C′ portion of the arm remains. In alternative embodiments, 1, 2, 3, 4, 5, 6, 7, 8, 9 or more base pairs are removed from the C′ portion of the C-C′ arm such that only C portion of the arm remains. In some embodiments, 1, 2, 3, 4, 5, 6, 7, 8, 9 or more base pairs are removed from the B portion of the B-B′ arm such that only B′ portion of the arm remains. In alternative embodiments, 1, 2, 3, 4, 5, 6, 7, 8, 9 or more base pairs are removed from the B′ portion of the B-B′ arm such that only B portion of the arm remains. In some embodiments, any deletion in the C region, C′ region, B region or B′ region still preserves the terminal loop of the stem-loop. In some embodiments, the terminal loop is at least three amino acids. In some embodiments, the terminal loop of a C-C′ arm, and/or B-B′ arm has at least three sequential TTTs or three sequential AAAs.
10. The ceDNA vector of paragraph 8 or paragraph 9, wherein one or both of the asymmetric ITRs is modified by a deletion, insertion, and/or substitution that results in the deletion of all or part of a stem-loop structure normally formed by the B and B′ regions.
11. The ceDNA vector of any one of paragraphs 8-10, wherein one or both of the asymmetric ITRs is modified by a deletion, insertion, and/or substitution that results in the deletion of all or part of a stem-loop structure normally formed by the C and C′ regions.
12. The ceDNA vector of paragraph 10 or paragraph 11, wherein one or both of the asymmetric ITRs is modified by a deletion, insertion, and/or substitution that results in the deletion of part of a stem-loop structure normally formed by the B and B′ regions and/or part of a stem-loop structure normally formed by the C and C′ regions.
13. The ceDNA vector of any one of paragraphs 1-12, wherein one or both of the asymmetric ITRs comprises a single stem-loop structure in the region that normally comprises a first stem-loop structure formed by the B and B′ regions and a second stem-loop structure formed by the C and C′ regions.
14. The ceDNA vector of paragraph 13, wherein one or both of the asymmetric ITRs comprises a single stem and two loops in the region that normally comprises a first stem-loop structure formed by the B and B′ regions and a second stem-loop structure formed by the C and C′ regions.
15. The ceDNA vector of paragraph 13 or paragraph 14, wherein one or both of the asymmetric ITRs comprises a single stem and a single loop in the region that normally comprises a first stem-loop structure formed by the B and B′ regions and a second stem-loop structure formed by the C and C′ regions.
16. The ceDNA vector of any one of paragraphs 1-15, wherein at least one asymmetric ITR is a modified AAV2 ITR comprising a nucleotide sequence selected from: the ITRs in FIG. 26A or 26B, SEQ ID NOS: 101-499 or 545-547, an ITR having at least 95% sequence identity to an ITR in FIG. 26A or 26B, and an ITR having at least 95% sequence identity to SEQ ID NOS: 101-499 and 545-547.
17. The ceDNA vector of any one of paragraphs 1-16, wherein at least one asymmetric ITR is a modified AAV2 ITR comprising a nucleotide sequence of SEQ ID NOS. 2, 52, 63, or 64, or a nucleotide sequence having at least 95% sequence identity to SEQ ID NOS. 2, 52, 63, or 64.
18. The ceDNA vector of any one of paragraphs 1-16, wherein the 5′ ITR is a wild type AAV ITR and the 3′ ITR comprises a sequence selected from SEQ ID NO: 2, 64, 102, 104, 106, 108, 110, 112, 114, 116, 118, 120, 122, 124, 126, 128, 130, 132, 134, 469-483 and 546, and ITR sequences shown in FIG. 26A, and sequences having at least 95% sequence identity to any of the foregoing sequences.
19. The ceDNA vector of any one of paragraphs 1-16, wherein the 3′ ITR is a wild type AAV ITR and the 5′ ITR comprises a sequence selected from SEQ ID NO: 52, 63, 101, 103, 105, 107, 109, 111, 113, 115, 117, 119, 121, 123, 125, 127, 129, 131, 133, 484-499, 545 and 547, and ITR sequences shown in FIG. 26B, and sequences having at least 95% sequence identity to any of the foregoing sequences.
20. The ceDNA vector of any one of paragraphs 1-16, wherein the 5′ ITR comprises a sequence selected from SEQ ID NO: 52, 63, 101, 103, 105, 107, 109, 111, 113, 115, 117, 119, 121, 123, 125, 127, 129, 131, 133, 484-499, 545 and 547, and ITR sequences shown in FIG. 26B, and sequences having at least 95% sequence identity to any of the foregoing sequences; and the 3′ ITR comprises a sequence selected from SEQ ID NO: 2, 64, 102, 104, 106, 108, 110, 112, 114, 116, 118, 120, 122, 124, 126, 128, 130, 132, 134, 469-483 and 546, and ITR sequences shown in FIG. 26A, and sequences having at least 95% sequence identity to any of the foregoing sequences.
21. The ceDNA vector of paragraph 1, comprising at least two asymmetric ITRs selected from: (a) SEQ ID NO: 1 and SEQ ID NO:52; and (b) SEQ ID NO: 2 and SEQ ID NO: 51.
22. The ceDNA vector of paragraph 1, comprising a pair of asymmetric ITRs selected from: (a) SEQ ID NO:1 and SEQ ID NO:52; and (b) SEQ ID NO:2 and SEQ ID NO:51.
23. The ceDNA vector of any one of paragraphs 1-20, wherein one or both asymmetric ITRs comprises a sequence other than SEQ ID NO: 2, 52, 63 64, 113, 114, and 557.
24. The ceDNA vector of any one of paragraphs 1-24, wherein all or part of the heterologous nucleotide sequence is under the control of at least one regulatory switch.
25. The ceDNA vector of paragraph 24, wherein at least one regulatory switch is selected from any or a combination of regulatory switches listed in Table 11, or in the section entiled “Regulatory switches” herein. By way of example only, a regulatory switch serves to fine tune expression of the heterologous nucleotide sequence, for example, a transgene, and can serve, in some embodiments as a biocontainment function of the ceDNA vector. In some embodiments, a regulatory switch is an “ON/OFF” switch. Without wishing to be limited to theory, an “ON/OFF” switch is designed to start or stop (i.e., shut down) expression of the heterologous nucleotide sequence or transgene expressed from the ceDNA vector in a controllable and regulatable fashion. In some embodiments, the regulatory switch is a “kill switch” that can instruct the cell comprising the ceDNA vector to undergo cell programmed death once the switch is activated. In some embodiments, the regulatory switch is selected from any of: a binary switch (e.g., inducible promoters, co-factors or exogenous agents de-repress transcription), a small molecule regulatory switch (e.g., drug-induced or pro-drugs activate or stop transcription), a “passcode” regulatory switch (e.g., a combination of conditions need to be present for transgene expression and/or repression to occur), a nucleic-acid based regulatory switch (e.g., a nucleic-acid based mechanism to control expression and/or repression), a post-translation and/or post-transcriptional regulatory switch (e.g., transgenes expressed with sihnal response elements (SRE) or destabilizing domains (DD) preventing functional transgenes until post-translation modification has occurred) or a kill switch (e.g., a switch to induce programmed cell death as a means permanently remove an introduced ceDNA vector from the subject's system).
26. The ceDNA vector of any one of paragraphs 1-25, wherein the vector is in a nanocarrier.
27. The ceDNA vector of paragraph 26, wherein the nanocarrier comprises a lipid nanoparticle (LNP).
28. A non-viral capsid-free DNA vector with covalently-closed ends (ceDNA vector) of any one of paragraphs 1-25, the ceDNA vector being obtained from a process comprising the steps of: (a) incubating a population of insect cells harboring a ceDNA expression construct in the presence of at least one Rep protein, wherein the ceDNA expression construct encodes the ceDNA vector, under conditions effective and for a time sufficient to induce production of the ceDNA vector within the insect cells; and (b) isolating the ceDNA vector from the insect cells.
29. The ceDNA vector of paragraph 28, wherein the ceDNA expression construct is selected from a ceDNA plasmid, a ceDNA bacmid, and a ceDNA baculovirus.
30. The ceDNA vector of paragraph 28 or paragraph 29, wherein the insect cell expresses at least one Rep protein.
31. The ceDNA vector of paragraph 30, wherein at least one Rep protein is from a virus selected from a parvovirus, a dependovirus, and an adeno-associated virus (AAV).
32. The ceDNA vector of paragraph 31, wherein at least one Rep protein is from an AAV serotype selected from AAV1, AAV2, AAV3, AAV4, AAV5, AAV6, AAV7, AAV8, AAV9, AAV10, AAV11, and AAV12.
33. A ceDNA expression construct that encodes the ceDNA vector of any one of paragraphs 1-25.
34. The ceDNA expression construct of paragraph 33, which is a ceDNA plasmid, ceDNA bacmid, or ceDNA baculovirus.
35. A host cell comprising the ceDNA expression construct of paragraph 33 or paragraph 34.
36. The host cell of paragraph 35, which expresses at least one Rep protein.
37. The host cell of paragraph 36, wherein at least one Rep protein is from a virus selected from a parvovirus, a dependovirus, and an adeno-associated virus (AAV).
38. The host cell of paragraph 37, wherein at least one Rep protein is from an AAV serotype selected from AAV1, AAV2, AAV3, AAV4, AAV5, AAV6, AAV7, AAV8, AAV9, AAV10, AAV11, and AAV12.
39. The host cell of any one of paragraphs 35 to 38, which is an insect cell.
40. The host cell of paragraph 39, which is an Sf9 cell.
41. A method of producing a ceDNA vector, comprising: (a) incubating the host cell of any one of paragraphs 35-40 under conditions effective and for time sufficient to induce production of the ceDNA vector; and (b) isolating the ceDNA from the host cells.
42. A method for treating, preventing, ameliorating, monitoring, or diagnosing a disease or disorder in a subject, the method comprising: administering to a subject in need thereof, a composition comprising the ceDNA vector of any one of paragraphs 1-25, wherein the at least one heterologous nucleotide sequence is selected to treat, prevent, ameliorate, diagnose, or monitor the disease or disorder.
43. The method of paragraph 42, wherein the at least one heterologous nucleotide sequence, when transcribed or translated corrects for an abnormal amount of an endogenous protein in the subject.
44. The method of paragraph 42, wherein the at least one heterologous nucleotide sequence, when transcribed or translated corrects for an abnormal function or activity of an endogenous protein or pathway in the subject.
45. The method of any one of paragraphs 42-44, wherein the at least one heterologous nucleotide sequence encodes or comprises an nucleotide molecule selected from an RNAi, an siRNA, an miRNA, an lncRNA, and an antisense oligo- or polynucleotide.
46. The method of any one of paragraphs 42-44, wherein the at least one heterologous nucleotide sequence encodes a protein.
47. The method of paragraph 42, wherein the at least one heterologous nucleotide sequence encodes a marker protein (e.g., a reporter protein).
48. The method of any one of paragraphs 42-46, wherein the at least one heterologous nucleotide sequence encodes an agonist or an antagonist of an endogenous protein or pathway associated with the disease or disorder.
49. The method of any one of paragraphs 42-46, wherein the at least one heterologous nucleotide sequence encodes an antibody.
50. The method of any one of paragraphs 42-49, wherein the disease or disorder is selected from: a metabolic disease or disorder, a CNS disease or disorder, an ocular disease or disorder, a blood disease or disorder, a liver disease or disorder, an immune disease or disorder, an infectious disease, a muscular disease or disorder, cancer, and a disease or disorder based on an abnormal level and/or function of a gene product.
51. The method of paragraph 50, wherein the metabolic disease or disorder is selected from diabetes, a lysosomal storage disorder, a mucopolysaccharide disorder, a urea cycle disease or disorder, and a glycogen storage disease or disorder.
52. The method of paragraph 51, wherein the lysosomal storage disorder is selected from Gaucher's disease, Pompe disease, metachromatic leukodystrophy (MLD), phenylketonuria (PKU), and Fabry disease.
53. The method of paragraph 51, wherein the urea cycle disease or disorder is ornithine transcarbamylase (OTC) deficiency.
54. The method of paragraph 51, wherein the mucopolysaccharide disorder is selected from Sly syndrome, Hurler Syndrome, Scheie Syndrome, Hurler-Scheie Syndrome, Hunter's Syndrome, Sanfilippo Syndrome, Morquio Syndrome, and Maroteaux-Lamy Syndrome.
55. The method of paragraph 50, wherein the CNS disease or disorder is selected from Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders, schizophrenia, drug dependency, neuroses, psychosis, dementia, paranoia, attention deficit disorder, sleep disorders, pain disorders, eating or weight disorders, and cancers and tumors of the CNS.
56. The method of paragraph 50, wherein the ocular disease or disorder is selected from ophthalmic disorders involving the retina, posterior tract, and/or optic nerve.
57. The method of paragraph 55, wherein the ophthalmic disorder involving the retina, posterior tract, and/or optic nerve are selected from diabetic retinopathy, macular degeneration including age-related macular degeneration, geographic atrophy and vascular or “wet” macular degeneration, glaucoma, uveitis, retinitis pigmentosa, Stargardt, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.
58. The method of paragraph 50, wherein the blood disease or disorder is selected from hemophilia A, hemophilia B, thalassemia, anemia, and blood cancers.
59. The method of paragraph 50, wherein the liver disease or disorder is selected from progressive familial intrahepatic cholestasis (PFIC) and liver cancer and tumors.
60. The method of paragraph 42, where the disease or disorder is cystic fibrosis.
61. The method of paragraphs 42-60, wherein the ceDNA vector is administered in combination with a pharmaceutically acceptable carrier.
62. A method for delivering a therapeutic protein to a subject, the method comprising administering to a subject a composition comprising the ceDNA vector of any of paragraphs 1-25, wherein at least one heterologous nucleotide sequence encodes a therapeutic protein.
63. The method of paragraph 62, wherein the therapeutic protein is a therapeutic antibody.
64. The method of paragraph 62, wherein the therapeutic protein is selected from an enzyme, erythropoietin, angiostatin, endostatin, superoxide dismutase, globin, leptin, catalase, tyrosine hydroxylase, a cytokine, cystic fibrosis transmembrane conductance regulator (CFTR), a peptide growth factor, and a hormone.
65. A kit comprising a ceDNA vector of any of paragraphs 1-25, and a nanocarrier, packaged in a container with a packet insert.
66. A kit for producing a ceDNA vector, the kit comprising an expression construct comprising at least one restriction site for insertion of at least one heterologous nucleotide sequence, or regulatory switch, or both, the at least one restriction site operatively positioned between asymmetric inverted terminal repeat sequences (asymmetric ITRs), wherein at least one of the asymmetric ITRs comprises a functional terminal resolution site and a Rep binding site.
67. The kit of paragraph 66, which is suitable for producing the ceDNA vector of any one of paragraphs 1-25.
68. The kit of paragraph 66 or paragraph 67, further comprising a population of insect cells which is devoid of viral capsid coding sequences, that in the presence of Rep protein can induce production of the ceDNA vector.
69. The kit of any one of paragraphs 66-68, further comprising a vector comprising a polynucleotide sequence that encodes at least one Rep protein, wherein the vector is suitable for expressing the at least one Rep protein in an insect cell.

In some embodiments, one aspect of the technology described herein relates to a non-viral capsid-free DNA vector with covalently-closed ends (ceDNA vector), wherein the ceDNA vector comprises at least one heterologous nucleotide sequence, operably positioned between asymmetric inverted terminal repeat sequences (asymmetric ITRs), wherein at least one of the asymmetric ITRs comprises a functional terminal resolution site and a Rep binding site, and optionally the heterologous nucleic acid sequence encodes a transgene, and wherein the vector is not in a viral capsid.

These and other aspects of the invention are described in further detail below.

### DESCRIPTION OF DRAWINGS

FIG. 1A illustrates an exemplary structure of a ceDNA vector. In this embodiment, the exemplary ceDNA vector comprises an expression cassette containing CAG promoter, WPRE, and BGHpA. An open reading frame (ORF) encoding a luciferase transgene is inserted into the cloning site (R3/R4) between the CAG promoter and WPRE. The expression cassette is flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1B illustrates an exemplary structure of a ceDNA vector with an expression cassette containing CAG promoter, WPRE, and BGHpA. An open reading frame (ORF) encoding Luciferase transgene is inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector with an expression cassette containing an enhancer/promoter, an open reading frame (ORF) for insertion of a transgene, a post transcriptional element (WPRE), and a polyA signal. An open reading frame (ORF) allows insertion of a transgene into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifiations).

FIG. 2A provides the T-shaped stem-loop structure of a wild-type left ITR of AAV2 (SEQ ID NO: 538) with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR (SEQ ID NO: 539), including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR (SEQ ID NO: 540). FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left) (SEQ ID NO: 113). FIG. 3C shows the primary structure (left) and the secondary structure (right) of the RBE-containing portion of the A-A′ loop, and the B-B′ and C-C′ arms of wild type right AAV2 ITR (SEQ ID NO: 541). FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right) (SEQ ID NO: 114). Any combination of left and right ITR (e.g., AAV2 ITRs or other viral serotype or synthetic ITRs) can be used, provided the left ITR is asymmetric or different from the right ITR. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of ceDNA in the process described in the schematic in FIG. 4B. FIG. 4B is a schematic of an exemplary method of ceDNA production and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions).

FIG. 5 is an exemplary picture of a denaturing gel running examples of ceDNA vectors with (+) or without (−) digestion with endonucleases (EcoRI for ceDNA construct 1 and 2; BamH1 for ceDNA construct 3 and 4; Spel for ceDNA construct 5 and 6; and Xhol for ceDNA construct 7 and 8). Sizes of bands highlighted with an asterisk were determined and provided on the bottom of the picture.

FIG. 6A shows results from an in vitro protein expression assay measuring Luciferase activity (y-axis, RQ (Luc)) in HEK293 cells 48 hours after transfection of 400 ng (black), 200 ng (gray), or 100 ng (white) of the constructs identified on the x-axis (construct-1, construct-3, construct-5, construct-7 (Table 12). FIG. 6B shows Luciferase activity (y-axis, RQ (Luc)) measured in HEK293 cells 48 hours after transfection of 400 ng (black), 200 ng (gray), or 100 ng (white) of the constructs identified on the x-axis (construct-2, construct-4, construct-6, construct-8) (Table 12). Luciferase activities measured in HEK293 cells treated with Fugene without any plasmids (“Fugene”), or in untreated HEK293 cells (“Untreated”) are also provided.

FIG. 7A shows viability of HEK293 cells (y-axis) 48 hours after transfection of 400 ng (black), 200 ng (gray), or 100 ng (white) of the constructs identified on the x-axis (construct-1, construct-3, construct-5, construct-7). FIG. 7B shows viability of HEK293 cells (y-axis) 48 hours after transfection of 400 ng (black), 200 ng (gray), or 100 ng (white) of the constructs identified on the x-axis (construct-2, construct-4, construct-6, construct-8).

FIG. 8A is an exemplary Rep-bacmid in the pFBDLSR plasmid comprising the nucleic acid sequences for Rep proteins Rep52 and Rep78. This exemplary Rep-bacmid comprises: IE1 promoter fragment (SEQ ID NO:66); Rep78 nucleotide sequence, including Kozak sequence (SEQ ID NO:67), polyhedron promoter sequence for Rep52 (SEQ ID NO:68) and Rep58 nucleotide sequence, starting with Kozak sequence gccgccacc) (SEQ ID NO:69). FIG. 8B is a schematic of an exemplary ceDNA-plasmid-1, with the wt-L ITR, CAG promoter, luciferase transgene, WPRE and polyadenylation sequence, and mod-R ITR.

FIG. 9A shows the predicted lowest energy structure of the RBE containing portion of the A-A′ arm and the C-C′ arm of an exemplary modified left ITR (“ITR-2 (Left)” SEQ ID NO: 101) and FIG. 9B shows the predicted lowest energy structure of the RBE-containing portion of the A-A′ arm and the C-C′ arm of an exemplary a modified right ITR (“ITR-2 (Right)” SEQ ID NO: 102). They are predicted to form a structure with a single arm (C-C′) and a single unpaired loop. Their Gibbs free energies of unfolding are predicted to be −72.6 kcal/mol.

FIG. 10A shows the predicted lowest energy structure of the RBE containing portion of the A-A′ arm and the B-B′ arm of an exemplary modified left ITR (“ITR-3 (Left)” SEQ ID NO: 103) and FIG. 10B shows the predicted lowest energy structure of the RBE containing portion of the A-A′ arm and the B-B′ arm of an exemplary modified right ITR (“ITR-3 (Right)” SEQ ID NO: 104). They are predicted to form a structure with a single arm (B-B′) and a single unpaired loop. Their Gibbs free energies of unfolding are predicted to be −74.8 kcal/mol.

FIG. 11A shows the predicted lowest energy structure of the RBE containing portion of the A-A′ arm and the C-C′ arm of an exemplary modified left ITR (“ITR-4 (Left)” SEQ ID NO: 105) and FIG. 11B shows the predicted lowest energy structure of the RBE-containing portion of the A-A′ arm and the C-C′ arm of an exemplary modified right ITR (“ITR-4 (Right)” SEQ ID NO: 106). They are predicted to form a structure with a single arm (C-C′) and a single unpaired loop. Their Gibbs free energies of unfolding are predicted to be −76.9 kcal/mol.

FIG. 12A shows the predicted lowest energy structure of the RBE containing portion of the A-A′ arm and the C-C′ and B-B′ portions of an exemplary modified left ITR, showing complementary base pairing of the C-B′ and C′-B portions (“ITR-10 (Left)” SEQ ID NO: 107) and FIG. 12B shows the predicted lowest energy structure of the RBE containing portion of the A-A′ arm and the B-B′ and C-C′ portions of an exemplary modified right ITR, showing complementary base pairing of the B-C′ and B′-C portions (“ITR-10 (Right)” SEQ ID NO: 108). They are predicted to form a structure with a single arm (a portion of C′-B and C-B′ or a portion of B′-C and B-C′) and a single unpaired loop. Their Gibbs free energies of unfolding are predicted to be −83.7 kcal/mol.

FIG. 13A shows the predicted lowest energy structure of the RBE containing portion of the A-A′ arm and the C-C′ and B-B′ portions of an exemplary modified left ITR (“ITR-17 (Left)” SEQ ID NO: 109) and FIG. 13B shows the predicted lowest energy structure of the RBE containing portion of the A-A′ arm and the C-C′ and B-B′ portions of an exemplary modified right ITR (“ITR-17 (Right)” SEQ ID NO: 110). Both ITR-17 (left) and ITR-17 (right) are predicted to form a structure with a single arm (B-B′) and a single unpaired loop. Their Gibbs free energies of unfolding are predicted to be −73.3 kcal/mol.

FIG. 14A shows the predicted lowest energy structure of the RBE containing portion of the A-A′ arm of an exemplary modified ITR (“ITR-6 (Left)” SEQ ID NO: 111) and FIG. 14B shows the predicted lowest energy structure of the RBE containing portion of the A-A′ arm of an exemplary modified ITR (“ITR-6 (Right)” SEQ ID NO: 112). Both ITR-6 (left) and ITR-6 (right) are predicted to form a structure with a single arm. Their Gibbs free energies of unfolding are predicted to be −54.4 kcal/mol.

FIG. 15A shows the predicted lowest energy structure of the RBE containing portion of the A-A′ arm and the C arm and B-B′ arm of an exemplary a modified left ITR (“ITR-1 (Left)” SEQ ID NO: 113) and FIG. 15B shows the predicted lowest energy structure of the RBE-containing portion of the A-A′ arm and the C arm and B-B′ arm of an exemplary modified right ITR (“ITR-1 (Right)” SEQ ID NO: 114). Both ITR-1 (left) and ITR-1 (right) are predicted to form a structure with two arms, one of which is truncated. Their Gibbs free energies of unfolding are predicted to be −74.7 kcal/mol.

FIG. 16A shows the predicted lowest energy structure of the RBE-containing portion of the A-A′ arm and the C′ arm and B-B′ arm of an exemplary modified left ITR (“ITR-5 (Left)” SEQ ID NO: 545) and FIG. 16B shows the predicted lowest energy structure of the RBE containing portion of the A-A′ arm and the B-B′ arm and C′ arm of an exemplary modified right ITR (“ITR-5 (Right)” SEQ ID NO: 116). Both ITR-5 (left) and ITR-5 (right) are predicted to form a structure with two arms, one of which is (e.g., the C′ arm) truncated. Their Gibbs free energies of unfolding are predicted to be −73.4 kcal/mol.

FIG. 17A shows the predicted lowest energy structure of the RBE-containing portion of the A-A′ arm and the C-C′ arm and B-B′ arm of an exemplary modified left ITR (“ITR-7 (Left)” SEQ ID NO: 117) and FIG. 17B shows the predicted lowest energy structure of the RBE-containing portion of the A-A′ arm and the B-B′ arm and C-C′ arm of an exemplary modified right ITR (“ITR-7 (Right)” SEQ ID NO: 118). Both ITR-17 (left) and ITR-17 (right) are predicted to form a structure with two arms, one of which (e.g., B-B′ arm) is truncated. Their Gibbs free energies of unfolding are predicted to be −89.6 kcal/mol.

FIG. 18A shows the predicted lowest energy structure of the RBE-containing portion of the A-A′ arm and the C-C′ arm and B-B′ arm of an exemplary modified left ITR (“ITR-8 (Left)” SEQ ID NO: 119) and FIG. 18B shows the predicted lowest energy structure of the RBE-containing portion of the A-A′ arm and the B-B′ arm and C-C′ arm of an exemplary modified right ITR (“ITR-8 (Right)” SEQ ID NO: 120). Both ITR-8 (left) and ITR-8 (right) are predicted to form a structure with two arms, one of which is truncated. Their Gibbs free energies of unfolding are predicted to be −86.9 kcal/mol.

FIG. 19A shows the predicted lowest energy structure of the RBE-containing portion of the A-A′ arm and the C-C′ arm and B-B′ arm of an exemplary modified left ITR (“ITR-9 (Left)” SEQ ID NO: 121) and FIG. 19B shows the predicted lowest energy structure of the RBE-containing portion of the A-A′ arm and the B-B′ arm and C-C′ arm of an exemplary modified right ITR (“ITR-9 (Right)” SEQ ID NO: 122). Both ITR-9 (left) and ITR-9 (right) are predicted to form a structure with two arms, one of which is truncated. Their Gibbs free energies of unfolding are predicted to be −85.0 kcal/mol.

FIG. 20A shows the predicted lowest energy structure of the RBE-containing portion of the A-A′ arm and the C-C′ arm and B-B′ arm of an exemplary modified left ITR (“ITR-11 (Left)” SEQ ID NO: 123) and FIG. 20B shows the predicted lowest energy structure of the RBE-containing portion of the A-A′ arm and the B-B′ arm and C-C′ arm of an exemplary modified right ITR (“ITR-11 (Right)” SEQ ID NO: 124). Both ITR-11 (left) and ITR-11 (right) are predicted to form a structure with two arms, one of which is truncated. Their Gibbs free energies of unfolding are predicted to be −89.5 kcal/mol.

FIG. 21A shows the predicted lowest energy structure of the RBE-containing portion of the A-A′ arm and the C-C′ arm and B-B′ arm of an exemplary modified left ITR (“ITR-12 (Left)” SEQ ID NO: 125) and FIG. 21B shows the predicted lowest energy structure of the RBE-containing portion of the A-A′ arm and the B-B′ arm and C-C′ arm of an exemplary modified right ITR (“ITR-12 (Right)” SEQ ID NO: 126). Both ITR-12 (left) and ITR-12 (right) They are predicted to form a structure with two arms, one of which is truncated. Their Gibbs free energies of unfolding are predicted to be −86.2 kcal/mol.

FIG. 22A shows the predicted lowest energy structure of the RBE-containing portion of the A-A′ arm and the C-C′ arm and B-B′ arm of an exemplary modified left ITR (“ITR-13 (Left)” SEQ ID NO: 127) and FIG. 22B shows the predicted lowest energy structure of the RBE-containing portion of the A-A′ arm and the B-B′ arm and C-C′ arm of an exemplary a modified right ITR (“ITR-13 (Right)” SEQ ID NO: 128). Both ITR-13 (left) and ITR-13 (right) are predicted to form a structure with two arms, one of which (e.g., C-C′ arm) is truncated. Their Gibbs free energies of unfolding are predicted to be −82.9 kcal/mol.

FIG. 23A shows the predicted lowest energy structure of the RBE-containing portion of the A-A′ arm and the C-C′ arm and B-B′ arm of an exemplary modified left ITR (“ITR-14 (Left)” SEQ ID NO: 129) and FIG. 23B shows the predicted lowest energy structure of the RBE-containing portion of the A-A′ arm and the B-B′ arm and C-C′ arm of an exemplary modified right ITR (“ITR-14 (Right)” SEQ ID NO: 130). Both ITR-14 (left) and ITR-14 (right) are predicted to form a structure with two arms, one of which (e.g., C-C′ arm) is truncated. Their Gibbs free energies of unfolding are predicted to be −80.5 kcal/mol.

FIG. 24A shows the predicted lowest energy structure of the RBE-containing portion of the A-A′ arm and the C-C′ arm and B-C′ arm of an exemplary modified left ITR (“ITR-15 (Left)” SEQ ID NO: 131) and FIG. 24B shows the predicted lowest energy structure of the RBE-containing portion of the A-A′ arm and the B-B′ arm and C-C′ arm of an exemplary modified right ITR (“ITR-15 (Right)” SEQ ID NO: 132). Both ITR-15 (left) and ITR-15 (right) are predicted to form a structure with two arms, one of which (e.g., the C-C′ arm) is truncated. Their Gibbs free energies of unfolding are predicted to be −77.2 kcal/mol.

FIG. 25A shows the predicted lowest energy structure of the RBE-containing portion of the A-A′ arm and the C-C′ arm and B-C′ arm of an exemplary modified left ITR (“ITR-16 (Left) SEQ ID NO: 133) and FIG. 25B shows the predicted lowest energy structure of the RBE-containing portion of the A-A′ arm and the B-B′ arm and C-C′ arm of an exemplary a modified right ITR (“ITR-16 (Right)” SEQ ID NO: 134). Both ITR-16 (left) and ITR-16 (right) are predicted to form a structure with two arms, one of which (e.g., C-C′ arm) is truncated. Their Gibbs free energies of unfolding are predicted to be −73.9 kcal/mol.

FIG. 26A shows predicted structures of the RBE-containing portion of the A-A′ arm and modified B-B′ arm and/or modified C-C′ arm of exemplary modified right ITRs listed in Table 10A. FIG. 26B shows predicted structures of the RBE-containing portion of the A-A′ arm and modified C-C′ arm and/or modified B-B′ arm of exemplary modified left ITRs listed in Table 10B. The structures shown are the predicted lowest free energy structure. Color code: red=>99% probability; orange=99%-95% probability; beige=95-90% probability; dark green 90%-80%; bright green=80%-70%; light blue=70%-60%; dark blue 60%-50% and pink=<50%.

FIG. 27 shows luciferase activity of Sf9 GlycoBac insect cells transfected with selected asymmetric ITR mutant variants from Table 10A and 10B. The ceDNA vector had a luciferase gene flanked by a wt ITR and a modified asymmetric ITR selected from Table 10A or 10B. “ITR-50 R no rep” is the known rescuable mutant without co-infection of Rep containing baculovirus. “Mock” conditions are transfection reagents only, without donor DNA.

FIG. 28 shows a native agarose gel (1% agarose, 1×TAE buffer) of representative crude ceDNA extracts from Sf9 insect cell cultures transfected with ceDNA-plasmids comprising a Left wt-ITR with the other ITR selected from various mutant Right ITRs disclosed in Table 10A. 2 ug of total extract was loaded per lane. From left to right: Lane 1) 1 kb plus ladder, Lane 2) ITR-18 Right, Lane 3) ITR-49 Right Lane 4) ITR-19 Right, Lane 5) ITR-20 Right, Lane 6) ITR-21 Right, Lane 7) ITR-22 Right, Lane 8) ITR-23 Right, Lane 9) ITR-24 Right, Lane 10) ITR-25 Right, Lane 11) ITR-26 Right, Lane 12) ITR-27 Right, Lane 13) ITR-28 Right, Lane 14) ITR-50 Right, lane 15) 1 kb plus ladder.

FIG. 29 shows a denaturing gel (0.8% alkaline agarose) of representative constructs from ITR mutant library. The ceDNA vector is produced from plasmids constucts comprising a Left wt-ITR with the other ITR selected from various mutant Right ITRs disclosed in Table 10A. From left to right, Lane 1) 1 kb Plus DNA Ladder, Lane 2) ITR-18 Right un-cut, Lane 3) ITR-18 Right restriction digest, Lane 4) ITR-19 Right un-cut, Lane 5) ITR-19 Right restriction digest, Lane 6) ITR-21 Right un-cut, Lane 7) ITR-21 Right restriction digest, Lane 8) ITR-25 Right un-cut, Lane 9) ITR-25 Right restriction digest. Extracts were treated with EcoRI restriction endonuclease. Each mutant ceDNA is expected to have a single EcoRI recognition site, producing two characteristic fragments, 2,000 bp and 3,000 bp, which will run at 4,000 and 6,000 bp, respectively, under denaturing conditions. Untreated ceDNA extracts are 5,000 bp and expected to migrate at 11,000 bp under denaturing conditions.

FIG. 30 shows luciferase activity in vitro in HEK293 cells of ITR mutants ITR-18 Right, ITR-19 Right, ITR-21 Right and ITR-25 Right, and ITR-49, where the left ITR in the ceDNA vector is WT ITR. “Mock” conditions are transfection reagents only, without donor DNA, and untreated is the negative control.

### DETAILED DESCRIPTION OF THE INVENTION

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein. Transgenes of interest include, but are not limited to, nucleic acids encoding polypeptides, preferably therapeutic (e.g., for medical, diagnostic, or veterinary uses) or immunogenic polypeptides (e.g., for vaccines). In some embodiments, nucleic acids of interest include nucleic acids that are transcribed into therapeutic RNA. Transgenes included for use in the ceDNA vectors of the invention include, but are not limited to, those that express or encode one or more polypeptides, peptides, ribozymes, aptamers, peptide nucleic acids, siRNAs, RNAis, miRNAs, lncRNAs, antisense oligo- or polynucleotides, antibodies, antigen binding fragments, or any combination thereof.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species.

As used herein, the term “asymmetric ITRs” refers to a pair of ITRs within a single ceDNA genome or ceDNA vector that are not inverse complements across their full length. The difference in sequence between the two ITRs may be due to nucleotide addition, deletion, truncation, or point mutation. In one embodiment, one ITR of the pair may be a wild-type AAV sequence and the other a non-wild-type or synthetic sequence. In another embodiment, neither ITR of the pair is a wild-type AAV sequence and the two ITRs differ in sequence from one another. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 531), an RBS sequence identified in AAV2. Any known RBS sequence may be used in the embodiments of the invention, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 531). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 45), the hexanucleotide sequence identified in AAV2. Any known TRS sequence may be used in the embodiments of the invention, including other known AAV TRS sequences and other naturally known or synthetic TRS sequences such as AGTT (SEQ ID NO: 46), GGTTGG (SEQ ID NO: 47), AGTTGG (SEQ ID NO: 48), AGTTGA (SEQ ID NO: 49), and other motifs such as RRTTRR (SEQ ID NO: 50).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the terms “closed-ended DNA vector”, “ceDNA vector” and “ceDNA” are used interchangeably and refer to a non-virus capsid-free DNA vector with at least one covalently-closed end (i.e., an intramolecular duplex). In some embodiments, the ceDNA comprises two covalently-closed ends.

As defined herein, “reporters” refer to proteins that can be used to provide deteactable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself, or that of another promoter used in another modular component of the synthetic biological circuits described herein. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herien.

The term “enhancer” as used herein refers a cis-acting regulatory sequence (e.g., 50-1,500 base pairs) that bind one or more proteins (e.g., activator proteins, or transcription factor) to increase transcriptional activation of a nucleic acid sequence Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal. Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment.

As used herein, the term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the same antigen to which the intact antibody binds. In one embodiment, the antibody or antibody fragment comprises an immunoglobulin chain or antibody fragment and at least one immunoglobulin variable domain sequence. Examples of antibodies or fragments thereof include, but are not limited to, an Fv, an scFv, a Fab fragment, a Fab′, a F(ab′)2, a Fab′-SH, a single domain antibody (dAb), a heavy chain, a light chain, a heavy and light chain, a full antibody (e.g., includes each of the Fc, Fab, heavy chains, light chains, variable regions etc.), a bispecific antibody, a diabody, a linear antibody, a single chain antibody, an intrabody, a monoclonal antibody, a chimeric antibody, a multispecific antibody, or a multimeric antibody. An antibody or fragment thereof can be of any class, including but not limited to IgA, IgD, IgE, IgG, and IgM, and of any subclass thereof including but not limited to IgG1, IgG2, IgG3, IgG4, IgA1 and IgA2. In addition, an antibody can be derived from any mammal, for example, primates, humans, rats, mice, horses, goats etc. In one embodiment, the antibody is human or humanized. In some embodiments, the antibody is a modified antibody. In some embodiments, the components of an antibody can be expressed separately such that the antibody self-assembles following expression of the protein components. In some embodiments, the antibody is “humanized” to reduce immunogenic reactions in a human. In some embodiments, the antibody has a desired function, for example, interaction and inhibition of a desired protein for the purpose of treating a disease or a symptom of a disease. In one embodiment, the antibody or antibody fragment comprises a framework region or an F_{c} region.

As used herein, the term “antigen-binding domain” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, the term “full length antibody” refers to an immunoglobulin (Ig) molecule (e.g., an IgG antibody), for example, that is naturally occurring, and formed by normal immunoglobulin gene fragment recombinatorial processes.

As used herein, the term “functional antibody fragment” refers to a fragment that binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. ceDNA Vector

Provided herein are novel non-viral, capsid-free ceDNA molecules with covalently-closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I or exonuclease III), e.g. for over an hour at 37° C.

The ceDNA vectors disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR and the second ITR are asymmetric with respect to each other—that is, they are different from one another. As an exemplary embodiment, the first ITR can be a wild-type ITR and the second ITR can be a mutated or modified ITR. In some embodiments, the first ITR can be a mutated or modified ITR and the second ITR a wild-type ITR. In another embodiment, the first ITR and the second ITR are both modified but are different sequences, or have different modifications, or are not identical modified ITRs. Stated differently, the ITRs are asymmetric in that any changes in one ITR are not reflected in the other ITR; or alternatively, where the ITRs are different with respect to each other. Exemplary ITRs in the ceDNA vector and for use to generate a ceDNA-plasmid are discussed below in the section entitled “ITRs”.

The wild-type or mutated or otherwise modified ITR sequences provided herein represent DNA sequences included in the expression construct (e.g., ceDNA-plasmid, ce-DNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In some embodiments, a ceDNA vector described herein comprising the expression cassette with a transgene, which can be, for example, a regulatory sequence, a sequence encoding a nucleic acid (e.g., such as a miR or an antisense sequence), or a sequence encoding a polypeptide (e.g., such as a transgene). In one embodiment, the transgene may be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other.

In one embodiment in each of these aspects, an expression cassette is located between two ITRs comprised in the following order with one or more of: a promoter operably linked to a transgene, a posttranscriptional regulatory element, and a polyadenylation and termination signal. In one embodiment, the promoter is regulatable—inducible or repressible. The promoter can be any sequence that facilitates the transcription of the transgene. In one embodiment the promoter is a CAG promoter (e.g. SEQ ID NO: 03), or variation thereof. The posttranscriptional regulatory element is a sequence that modulates expression of the transgene, as a non-limiting example, any sequence that creates a tertiary structure that enhances expression of the transgene.

In one embodiment, the posttranscriptional regulatory element comprises WPRE (e.g. SEQ ID NO: 08). In one embodiment, the polyadenylation and termination signal comprises BGHpolyA (e.g. SEQ ID NO: 09). Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene or nucleic acid in the range of 500 to 50,000 nucleotides in length. In some embodiments, the expression cassette can comprise a transgene or nucleic acid in the range of 500 to 75,000 nucleotides in length. In some embodiments, the expression cassette can comprise a transgene or nucleic acid is in the range of 500 to 10,000 nucleotides in length. In some embodiments, the expression cassette can comprise a transgene or nucleic acid is in the range of 1000 to 10,000 nucleotides in length. In some embodiments, the expression cassette can comprise a transgene or nucleic acid is in the range of 500 to 5,000 nucleotides in length. The ceDNA vectors do not have the size limitations of encapsidated AAV vectors, thus enable delivery of a large-size expression cassette to provide efficient expression of transgenes. In some embodiments, the ceDNA vector is devoid of prokaryote-specific methylation.

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, a regulatory switches, which are described herein in the section entitled “Regulatory Switches” for controlling and regulating the expression of the transgene, and can include if desired, a regulatory switch which is a kill switch to enable controlled cell death of a cell comprising a ceDNA vector.

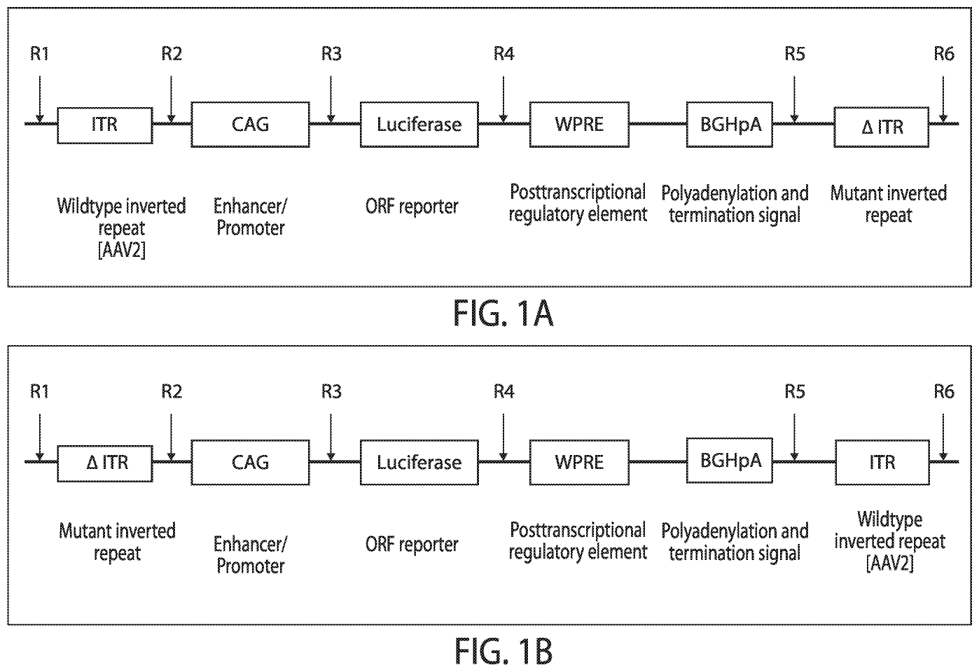

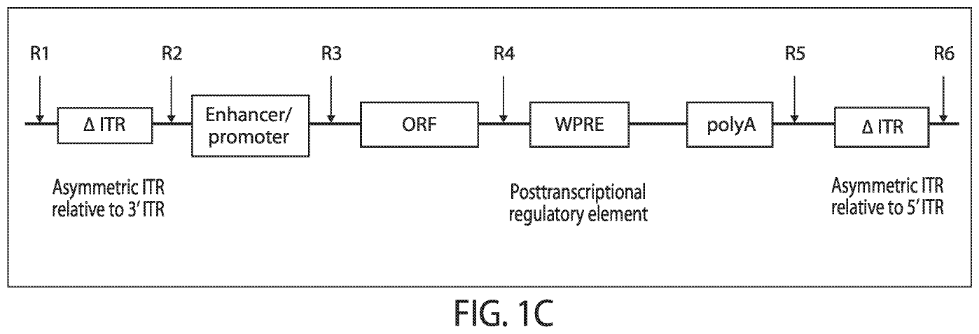

FIG. 1A-1C show schematics of nonlimiting, exemplary ceDNA vectors, or the corresponding sequence of ceDNA plasmids. ceDNA vectors are capsid-free and can be obtained from a plasmid encoding in this order: a first ITR, expressible transgene cassette and a second ITR, where at least one of the first and/or second ITR sequence is mutated with respect to the corresponding wild type AAV2 ITR sequence. The expressible transgene cassette preferably includes one or more of, in this order: an enhancer/promoter, an ORF reporter (transgene), a post-transcription regulatory element (e.g., WPRE), and a polyadenylation and termination signal (e.g., BGH polyA).

The expression cassette can comprise any transgene of interest. Transgenes of interest include but are not limited to, nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the transgenes in the expression cassette encodes one or more polypeptides, peptides, ribozymes, peptide nucleic acids, siRNAs, RNAis, antisense oligonucleotides, antisense polynucleotides, antibodies, antigen binding fragments, or any combination thereof. In some embodiments, the transgene is a therapeutic gene, or a marker protein. In some embodiments, the transgene is an agonist or antagonist. In some embodiments, the antagonist is a mimetic or antibody, or antibody fragment, or antigen-binding fragment thereof, e.g., a neutralizing antibody or antibody fragment and the like. In some embodiments, the transgene encodes an antibody, including a full-length antibody or antibody fragment, as defined herein. In some embodiments, the antibody is an antigen-binding domain or an immunoglobulin variable domain sequence, as that is defined herein.

In particular, the transgene can encode one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary transgenes are described herein in the section entitled “Method of Treatment”.

There are many structural features of ceDNA vectors that differ from plasmid-based expression vectors. ceDNA vectors may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, of the eukaryotic origin (i.e., they are produced in eukaryotic cells), and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-stranded DNA.

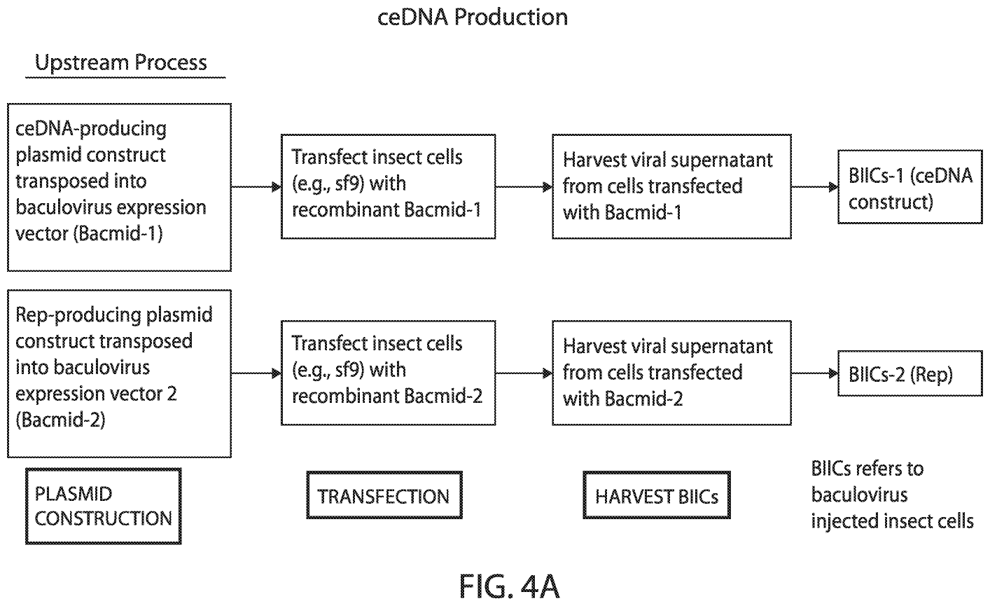

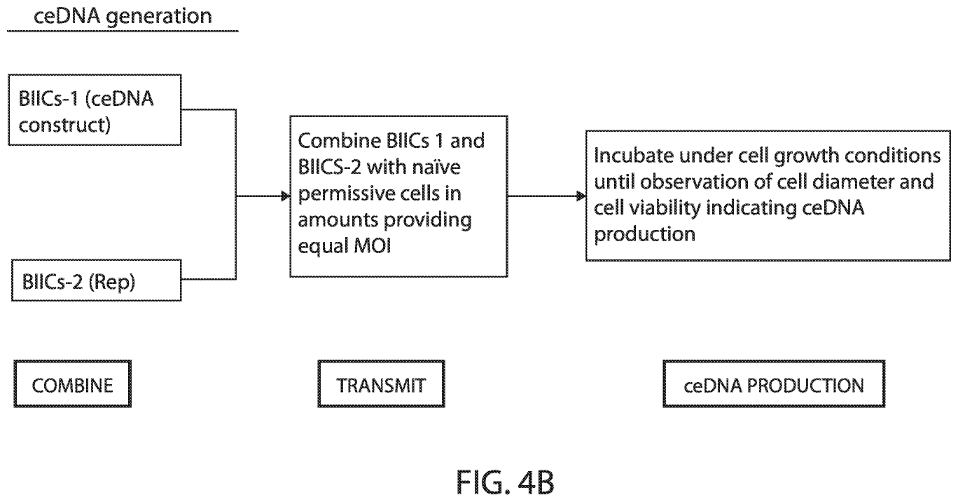

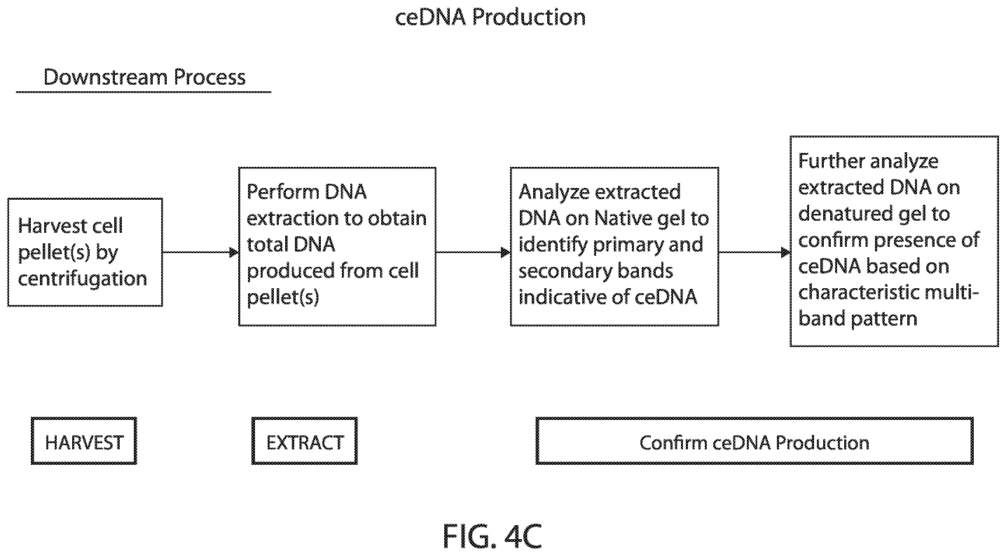

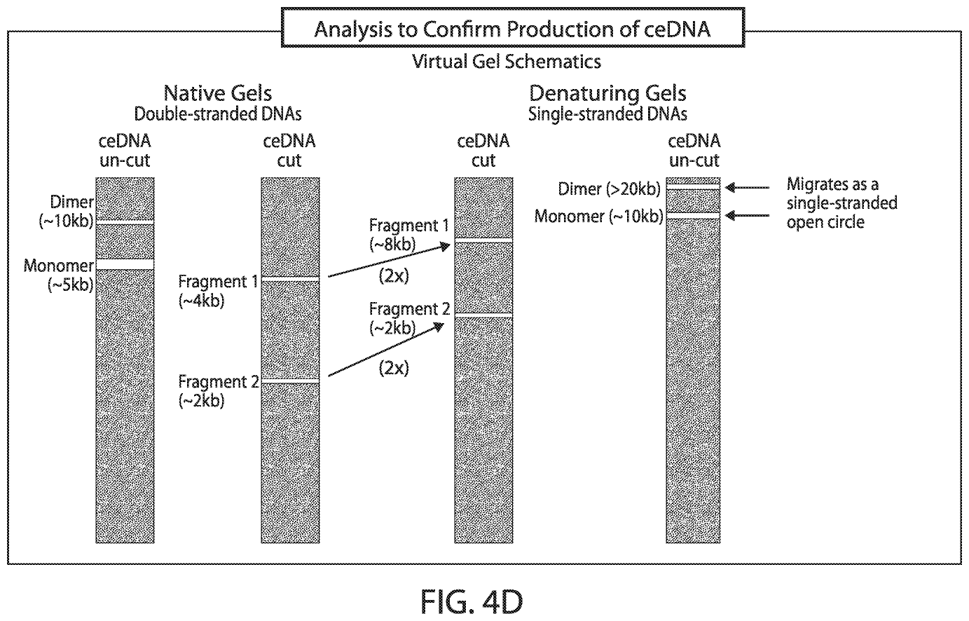

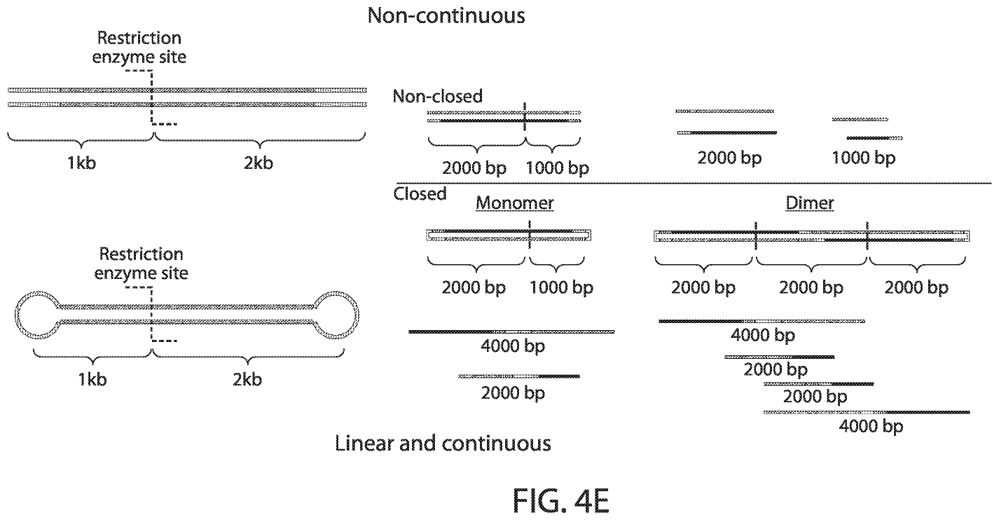

ceDNA vectors produced by the methods provided herein preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

Several advantages of a ceDNA vector described herein over plasmid-based expression vectors include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: 531) for AAV2) and a terminal resolution site (TRS; 5′-AGTTGG-3′ (SEQ ID NO: 48) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### III. ITRs

As disclosed herein, ceDNA vectors contain a heterologous gene positioned between two inverted terminal repeat (ITR) sequences, that differ with respect to each other (i.e. are asymmetric ITRs). In some embodiments, at least one of the ITRs is modified by deletion, insertion, and/or substitution as compared to a wild-type ITR sequence (e.g. AAV ITR); and at least one of the ITRs comprises a functional Rep binding site (RBS; e.g. 5′-GCGCGCTCGCTCGCTC-3′ for AAV2, SEQ ID NO: 531) and a functional terminal resolution site (TRS; e.g. 5′-AGTT-3′, SEQ ID NO: 46.) In one embodiment, at least one of the ITRs is a non-functional ITR. In one embodiment, the different ITRs are not each wild type ITRs from different serotypes.

While the ITRs exemplified in the specification and Examples herein are AAV2 ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvoviris (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148).

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

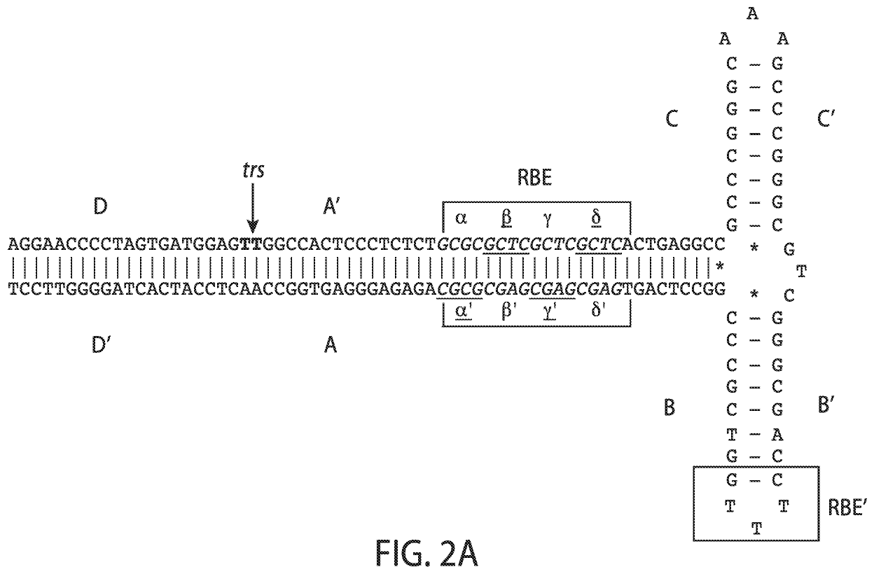

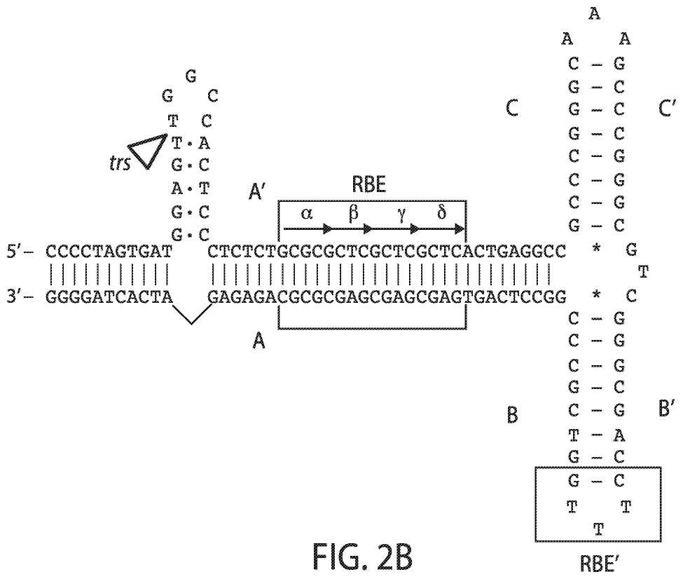

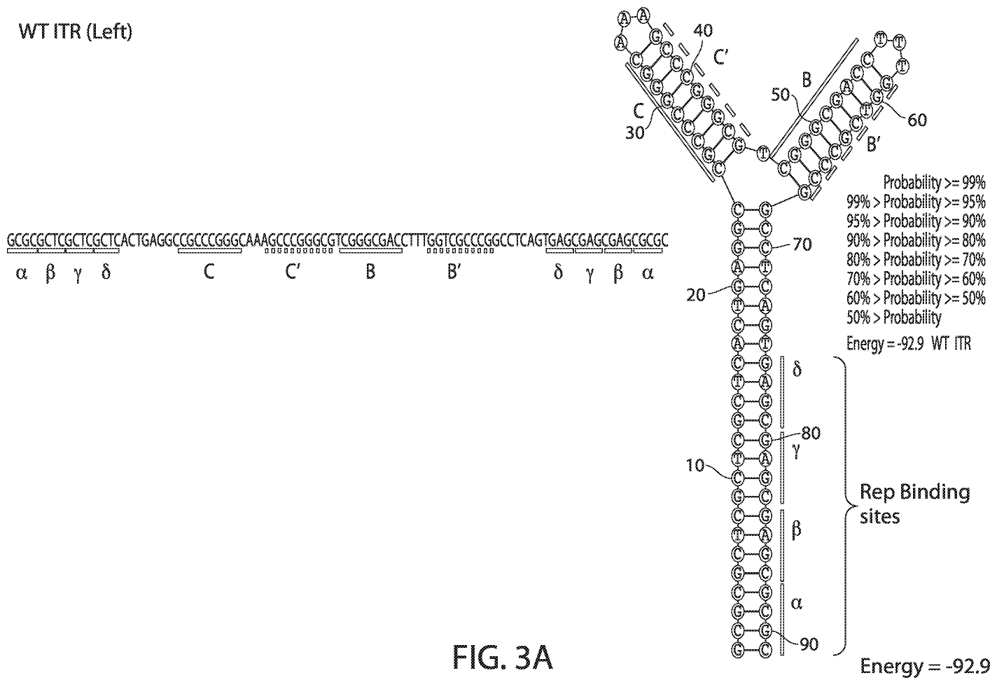

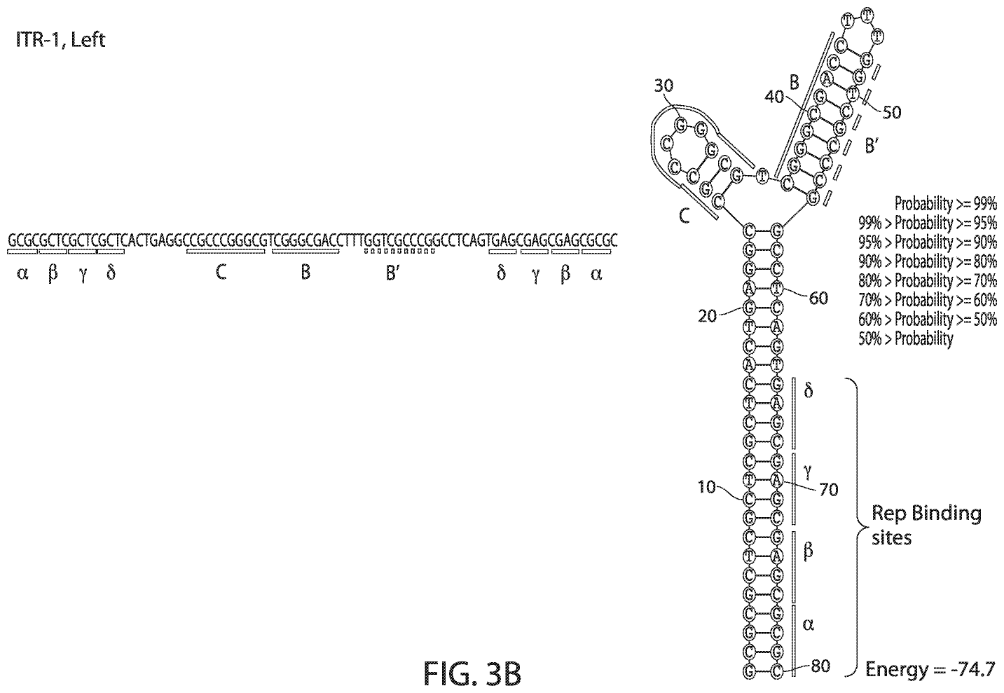

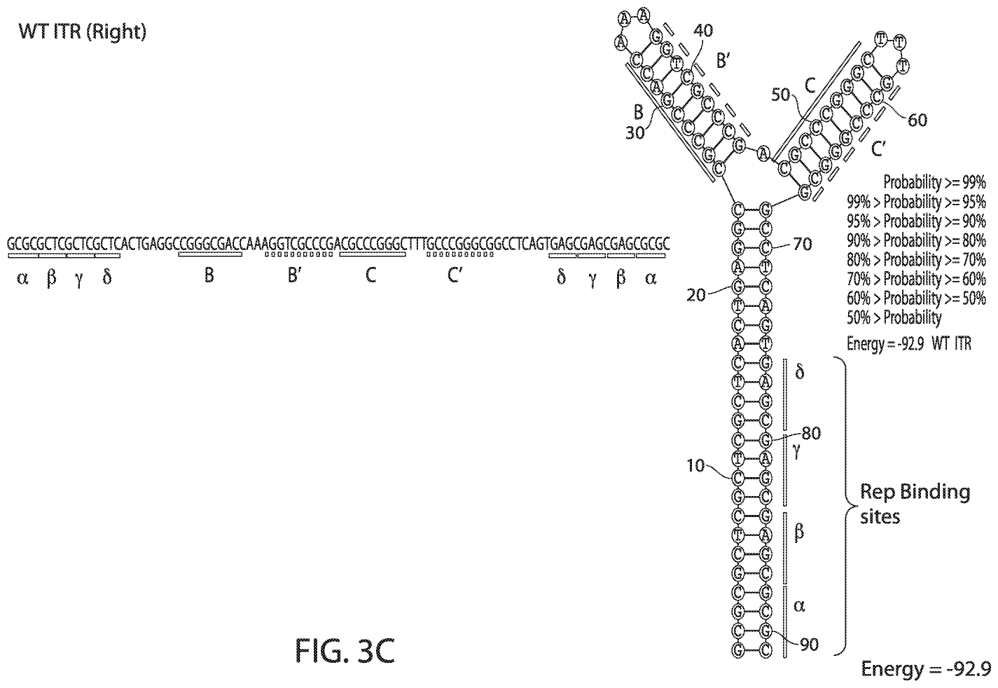

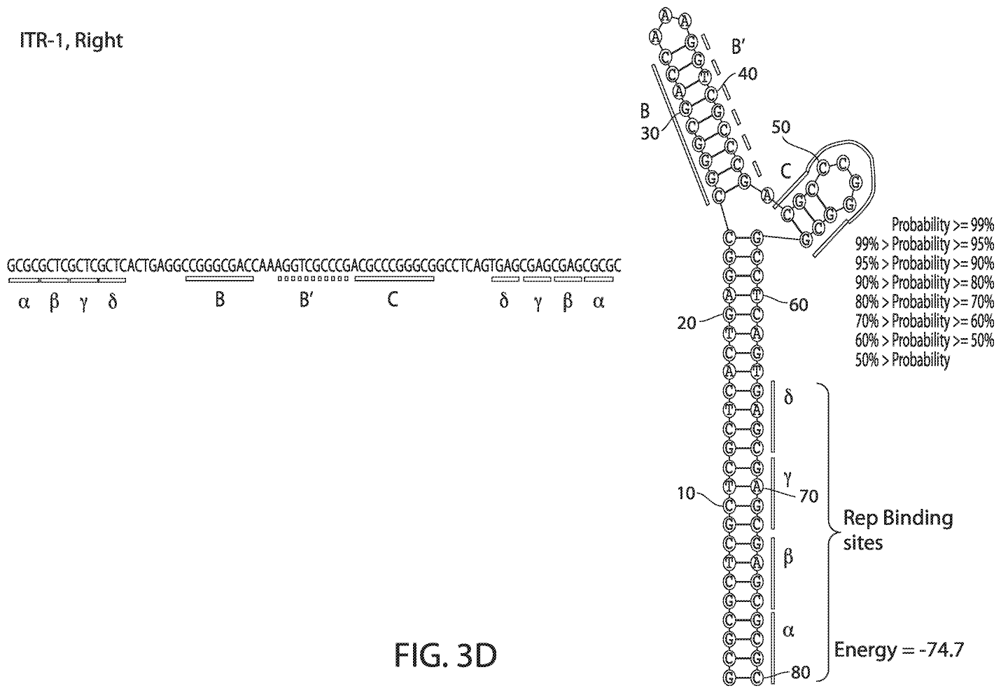

An ordinarily skilled artisan is aware that ITR sequences have a common structure of a double-stranded Holliday junction, which typically is a T-shaped or Y-shaped hairpin structure (see e.g., FIG. 2A and FIG. 3A), where each ITR is formed by two palindromic arms or loops (B-B′ and C-C′) embedded in a larger palindromic arm (A-A′), and a single stranded D sequence, (where the order of these palindromic sequences defines the flip or flop orientation of the ITR), one can readily determine corresponding modified ITR sequences from any AAV serotype for use in a ceDNA vector or ceDNA-plasmid based on the exemplary AAV2 ITR sequences provided herein. See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14.

Specific alterations and mutations in the ITRs are described in detail herein, but in the context of ITRs, “altered” or “mutated” indicates that nucleotides have been inserted, deleted, and/or substituted relative to the wild-type, reference, or original ITR sequence, and can be altered relative to the other flanking ITR in a ceDNA vector having two flanking ITRs. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, an ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wildtype Rep or a Rep of a specific serotype, or in some instances will not be recognized by a wild-type Rep and be recognized only by a mutated Rep.

ITR sequences have a common structure of a double-stranded Holliday junction, which typically is a T-shaped or Y-shaped hairpin structure (see, e.g., FIG. 2A and FIG. 3A), where each ITR is formed by two palindromic arms or loops (B-B′ and C-C′) embedded in a larger palindromic arm (A-A′), and a single stranded D sequence, (where the order of these palindromic sequences defines the ‘flip’ or ‘flop’ orientation of the ITR). One of ordinary skill in the art can readily determine ITR sequences or modified ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

Accordingly, while the AAV2 ITRs are used as exemplary ITRs in the ceDNA vectors disclosed herein, a ceDNA vector disclosed herein may be prepared with or based on ITRs of any known AAV serotype, including, for example, AAV serotype 1 (AAV1), AAV serotype 2 (AAV2), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12). The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The invention further provides populations and pluralities of ceDNA vectors comprising ITRs from a combination of different AAV serotypes—that is, one ITR can be from one AAV serotype and the other ITR can be from a different serotype. Without wishing to be bound by theory, in one embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR. For example, it is selected from the group consisting of: SEQ ID NO:2 and SEQ ID NO:52. In one embodiment of each of these aspects, the vector polynucleotide comprises a pair of ITRs, selected from the group consisting of: SEQ ID NO:1 and SEQ ID NO:52; and SEQ ID NO:2 and SEQ ID NO:51. In one embodiment of each of these aspects, the vector polynucleotide or the non-viral, capsid-free DNA vectors with covalently-closed ends comprises a pair of different ITRs selected from the group consisting of: SEQ ID NO:101 and SEQ ID NO:102; SEQ ID NO:103, and SEQ ID NO:104, SEQ ID NO:105, and SEQ ID NO:106; SEQ ID NO:107, and SEQ ID NO:108; SEQ ID NO:109, and SEQ ID NO:110; SEQ ID NO:111, and SEQ ID NO:112; SEQ ID NO:113 and SEQ ID NO:114; and SEQ ID NO:115 and SEQ ID NO:116. In some embodiments, a modified ITR is selected from any of the ITRs, or partial ITR sequences of SEQ ID NOS: 2, 52, 63, 64, 101-499 or 545-547.

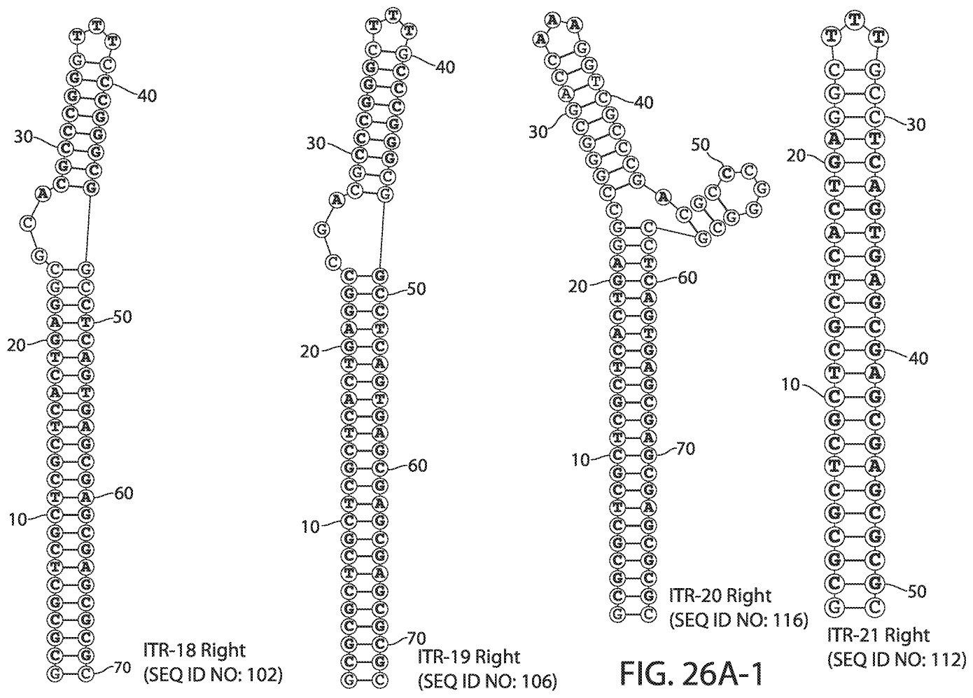

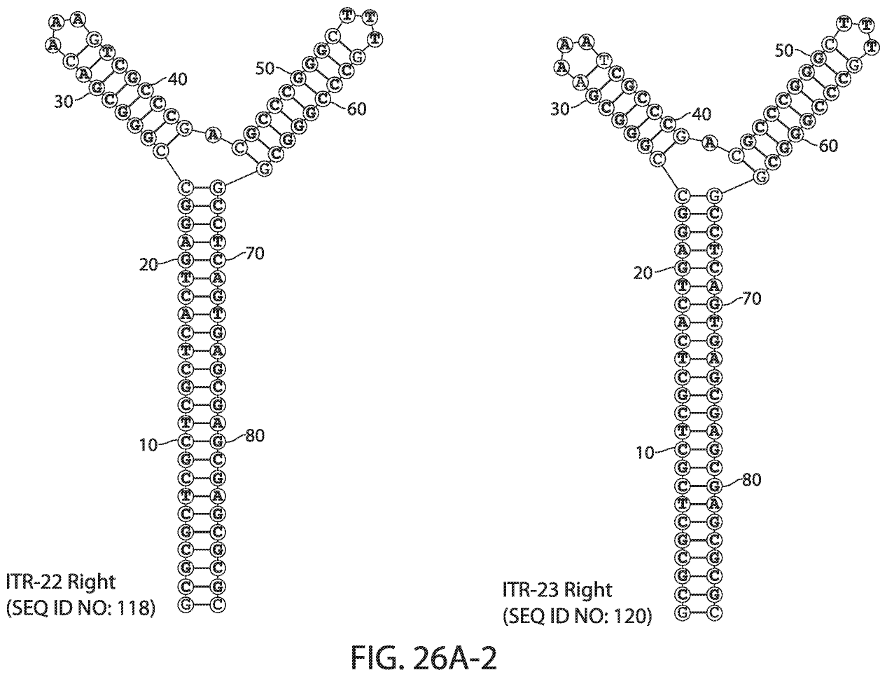

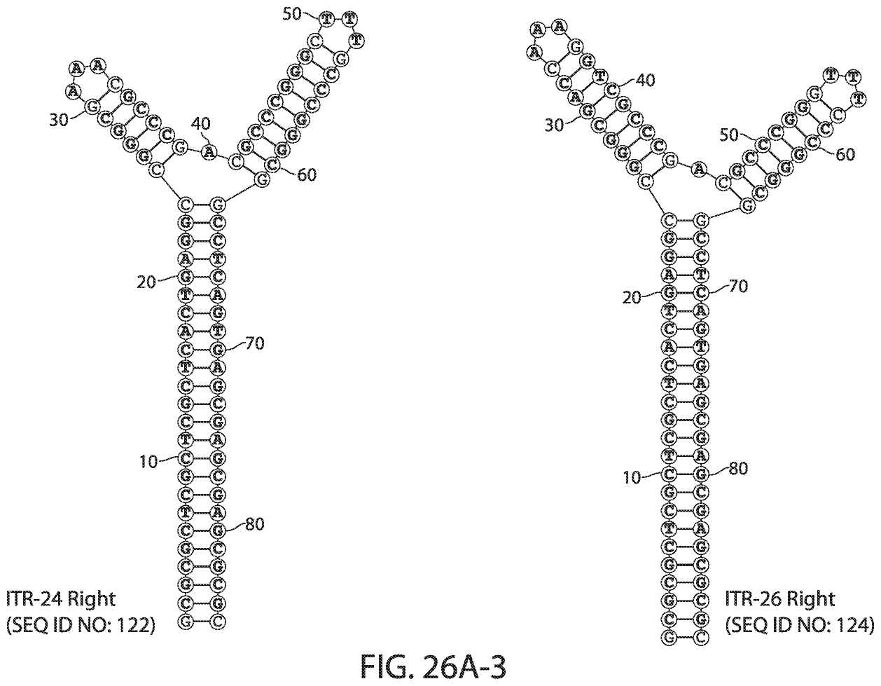

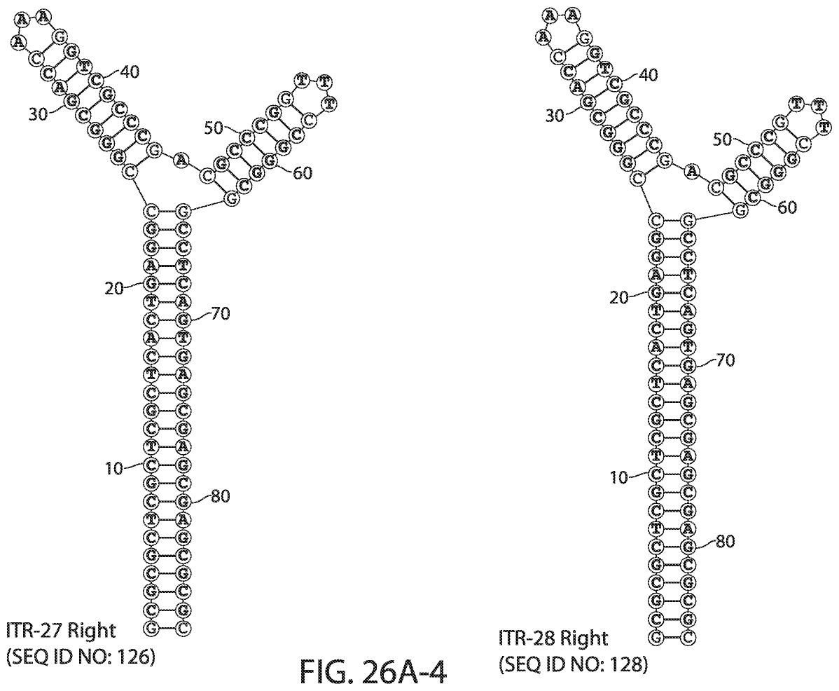

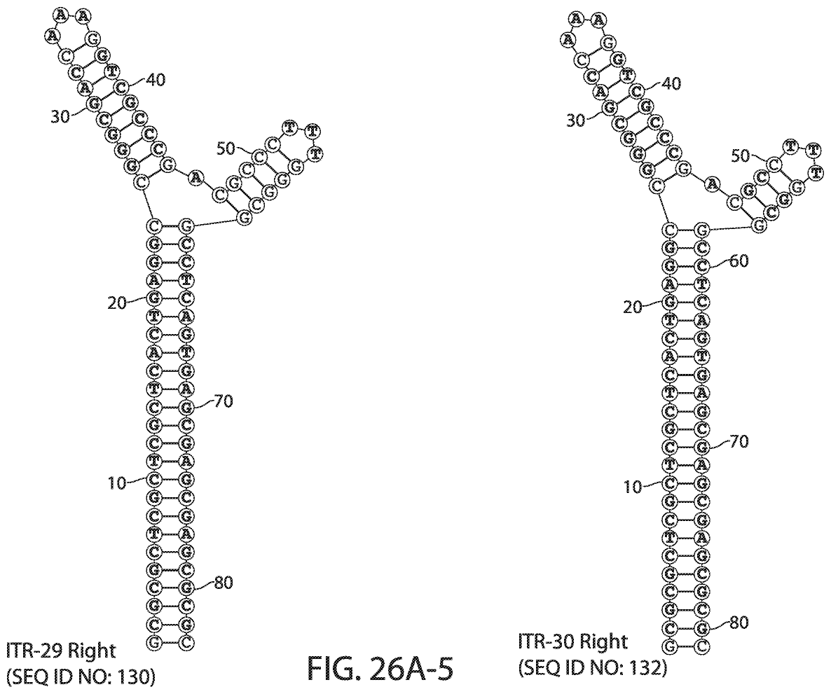

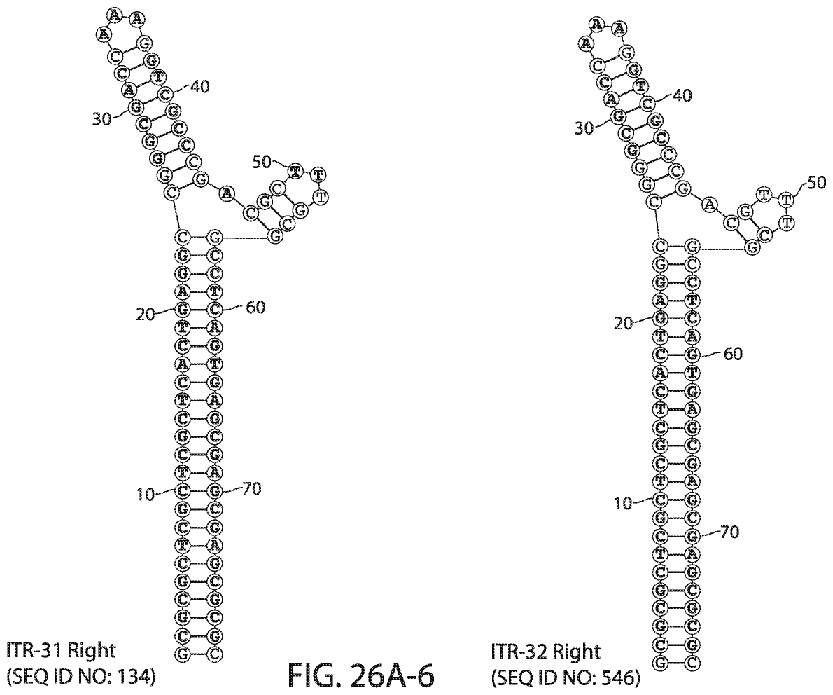

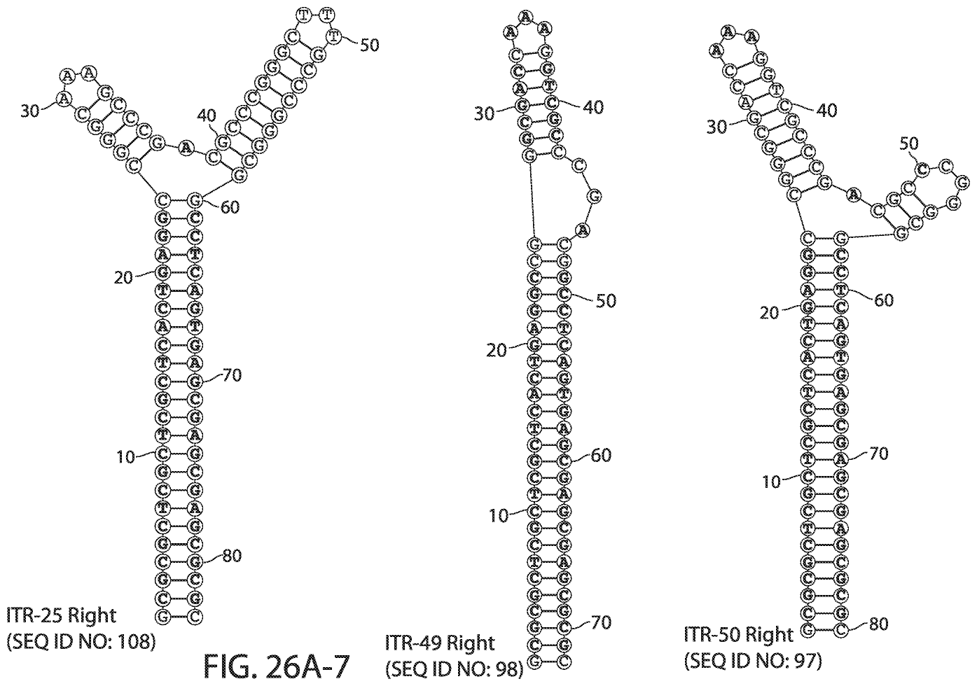

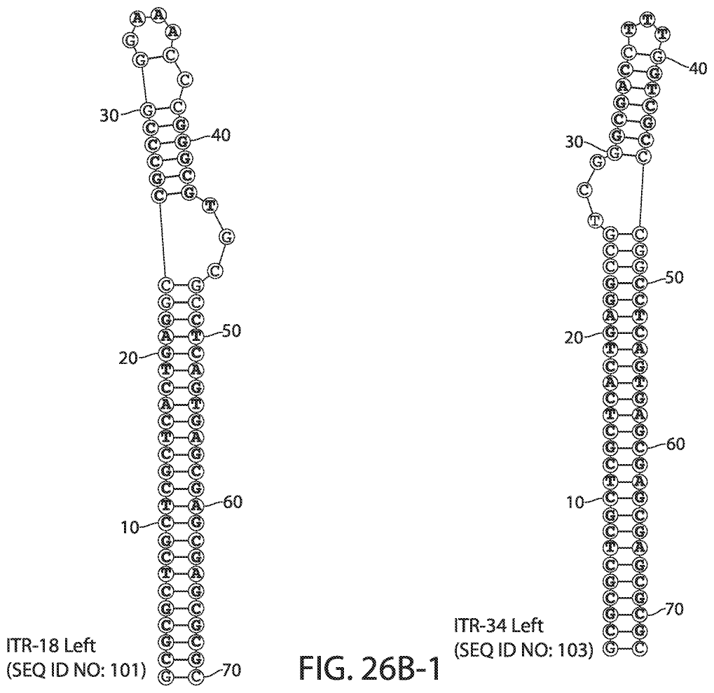

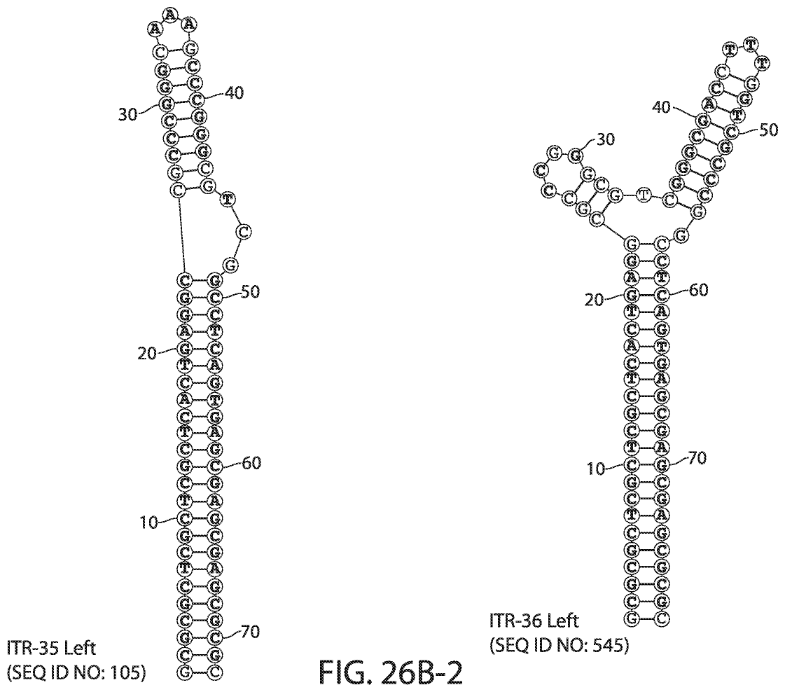

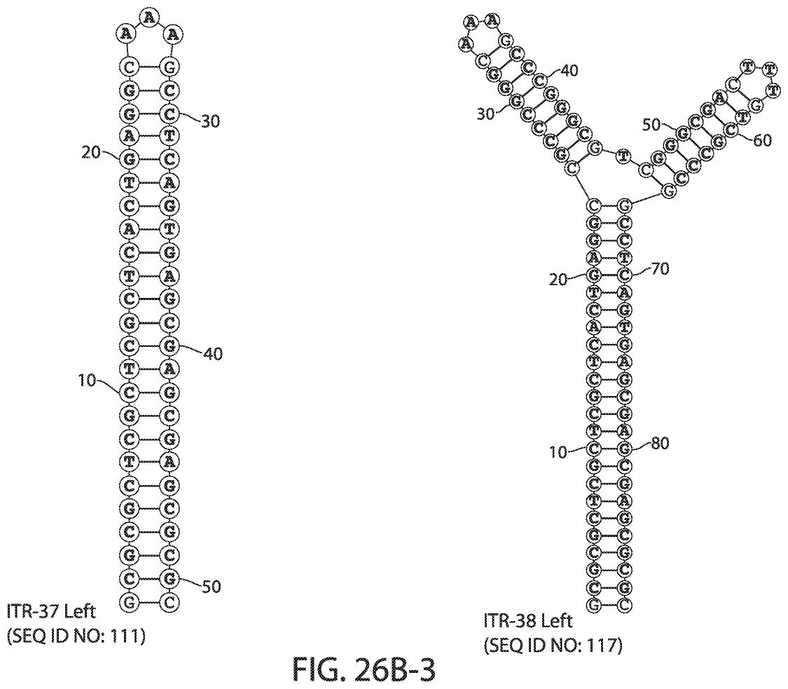

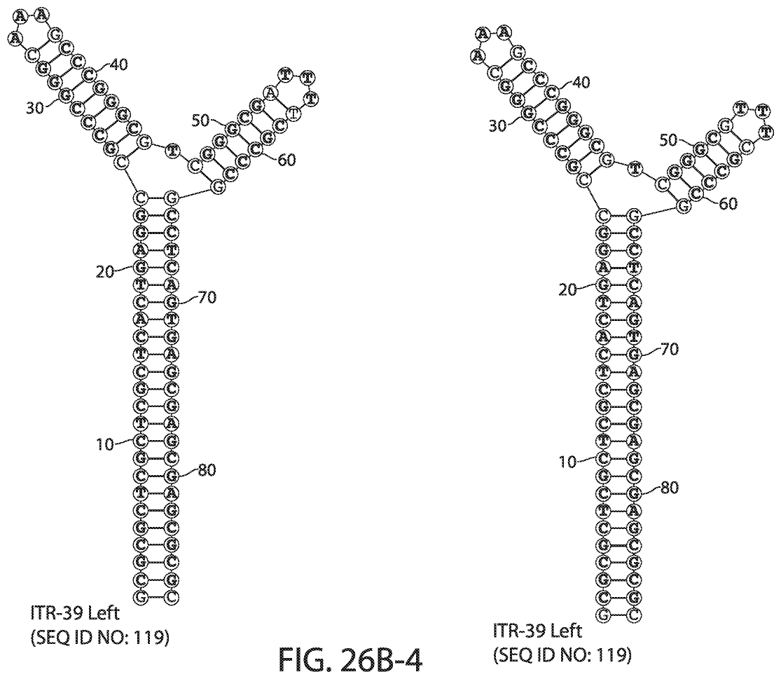

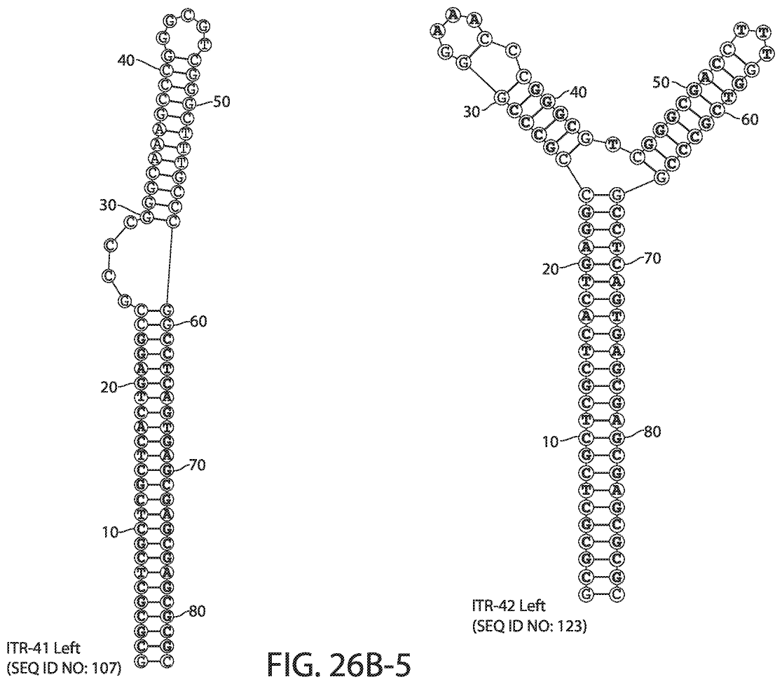

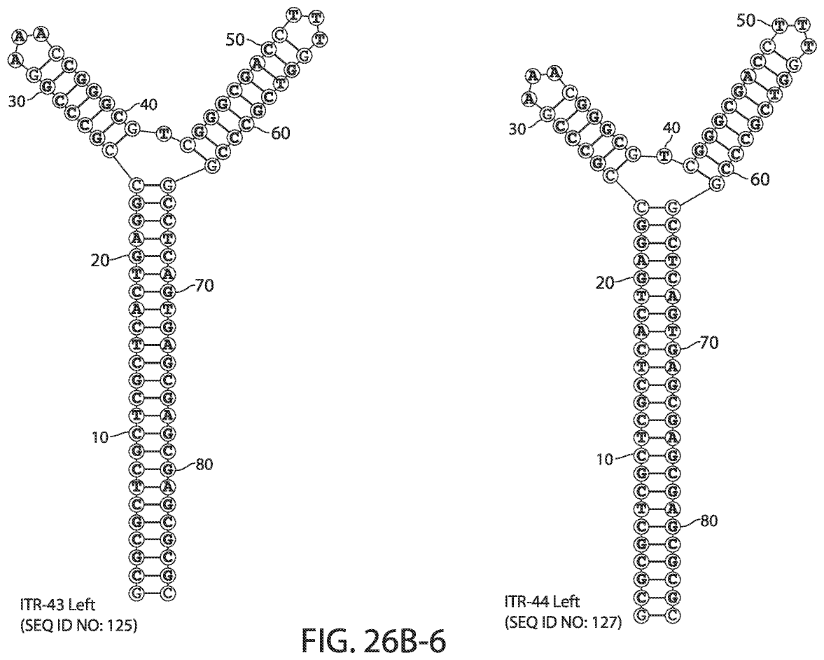

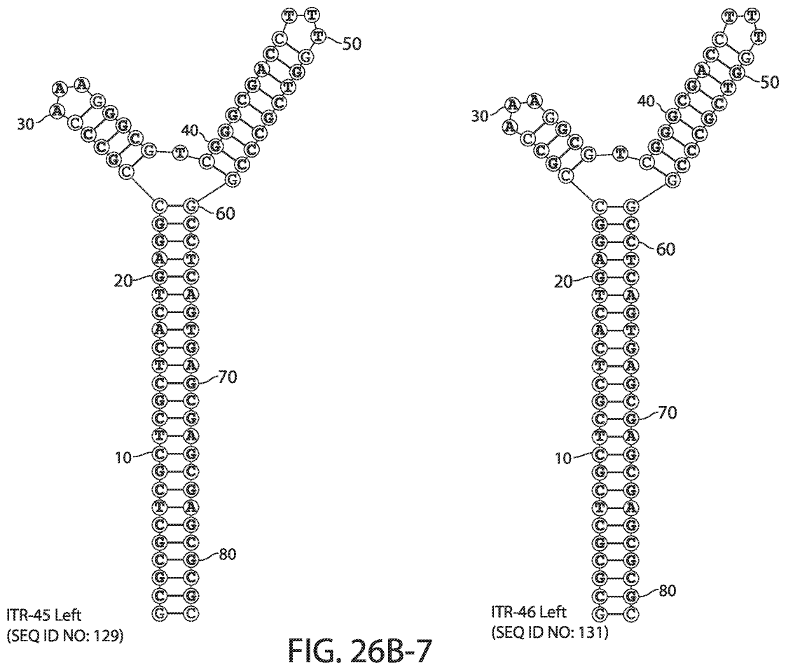

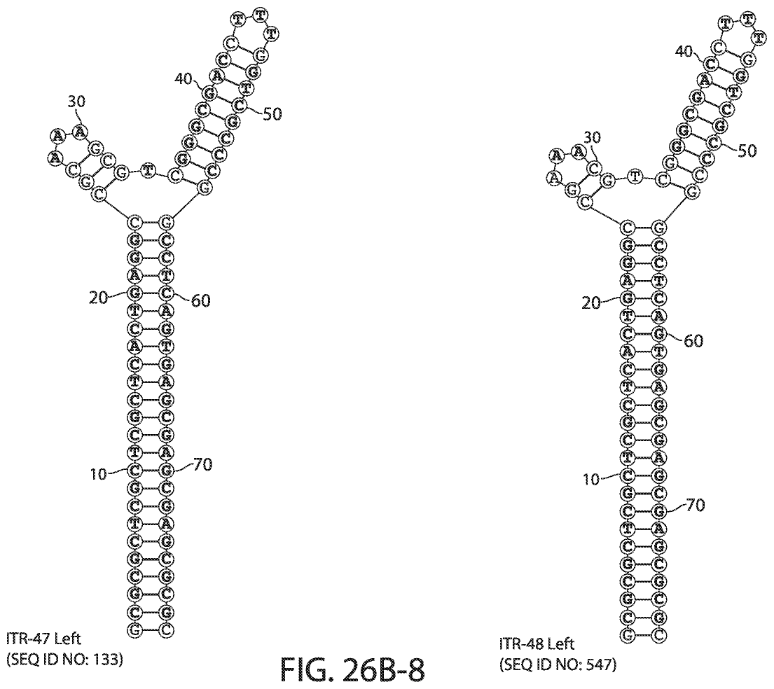

In some embodiments, a ceDNA vector can comprise an ITR with a modification in the ITR corresponding to any of the modifications in ITR sequences or ITR partial sequences shown in any one or more of Tables 2, 3, 4, 5, 6, 7, 8, 9, 10A and 10B herein, or the sequences shown in FIG. 26A or 26B.

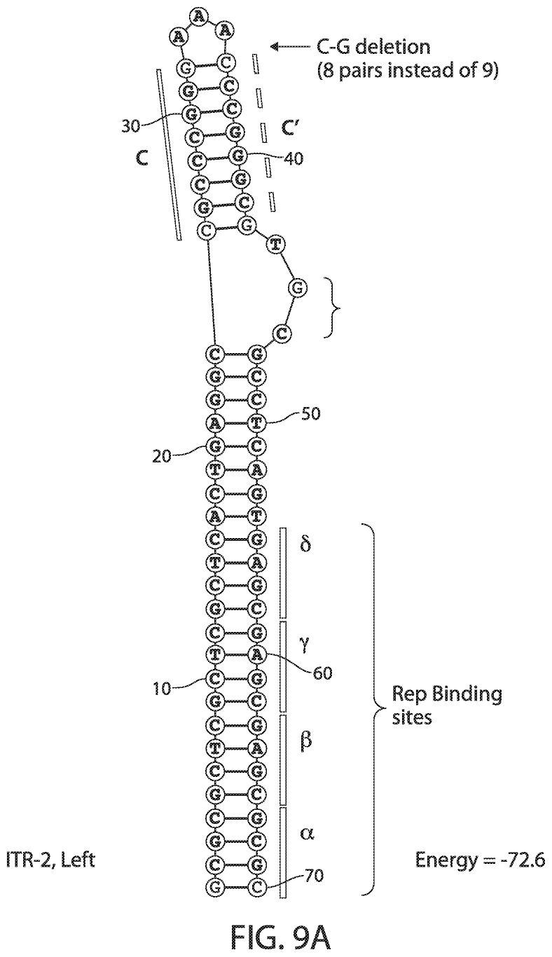

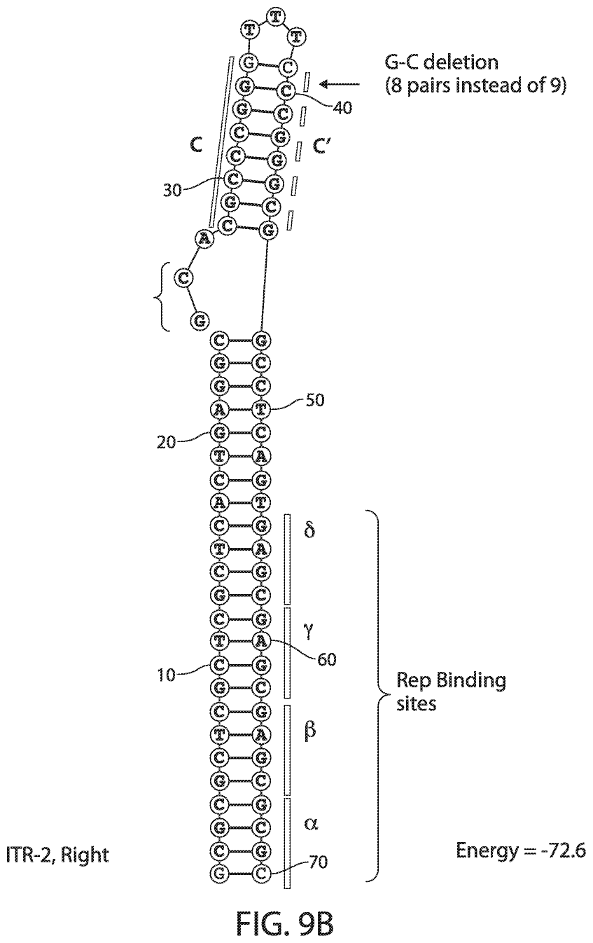

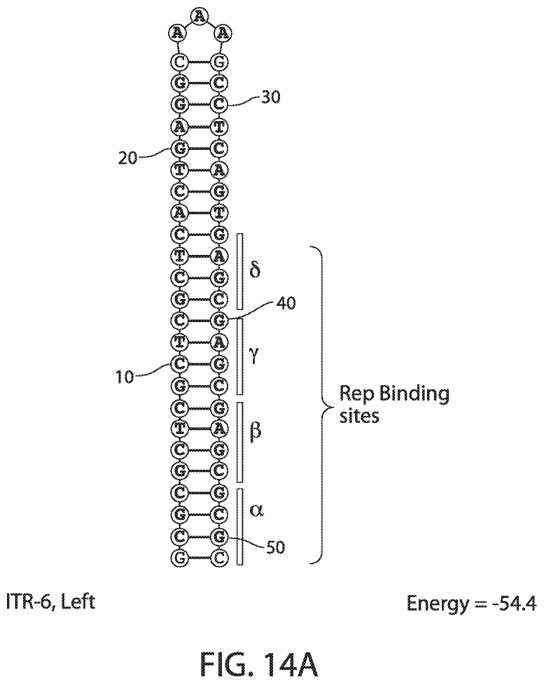

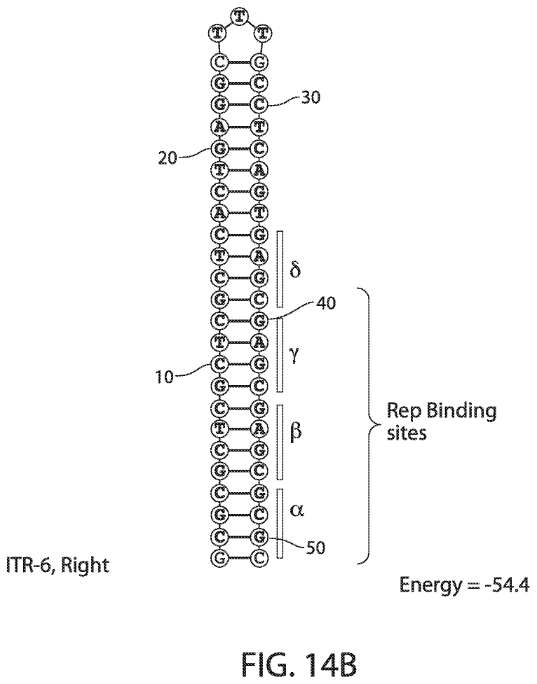

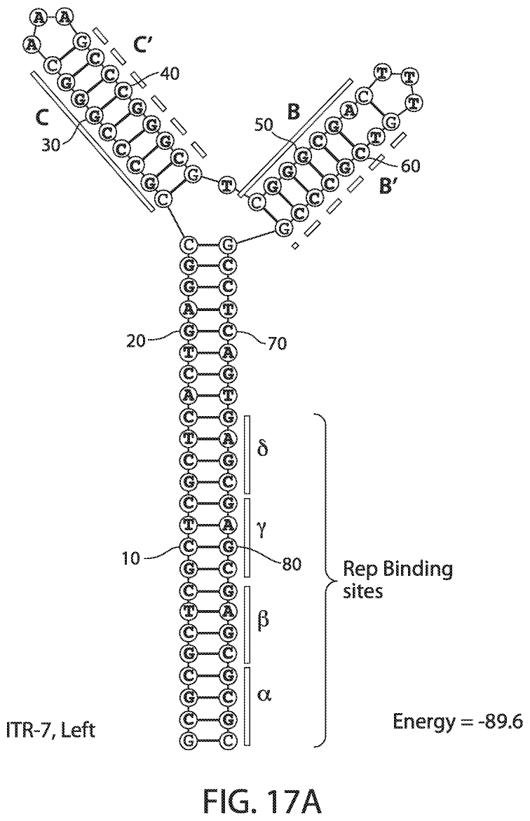

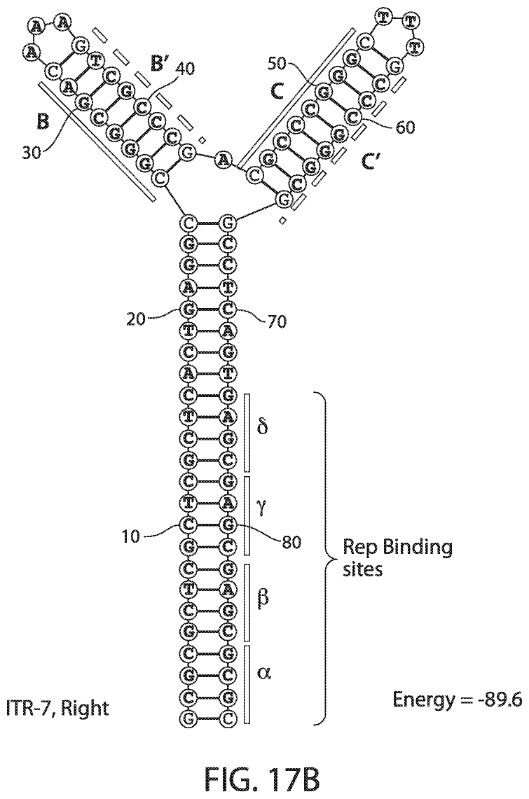

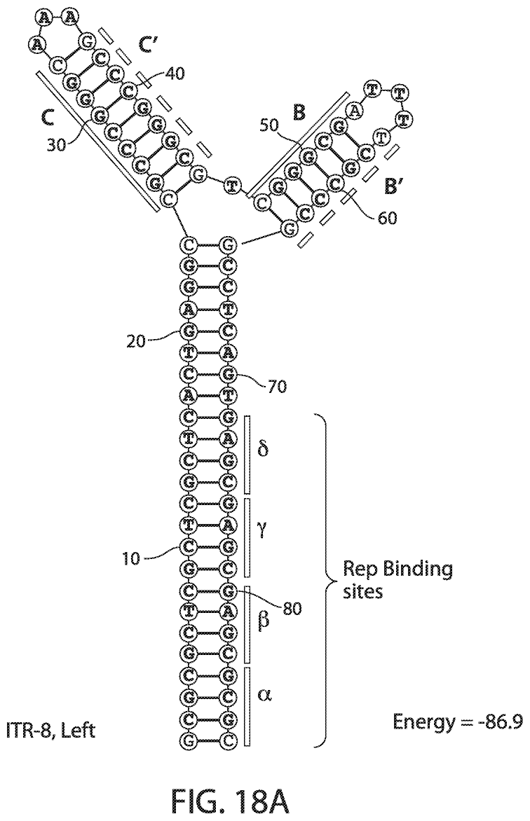

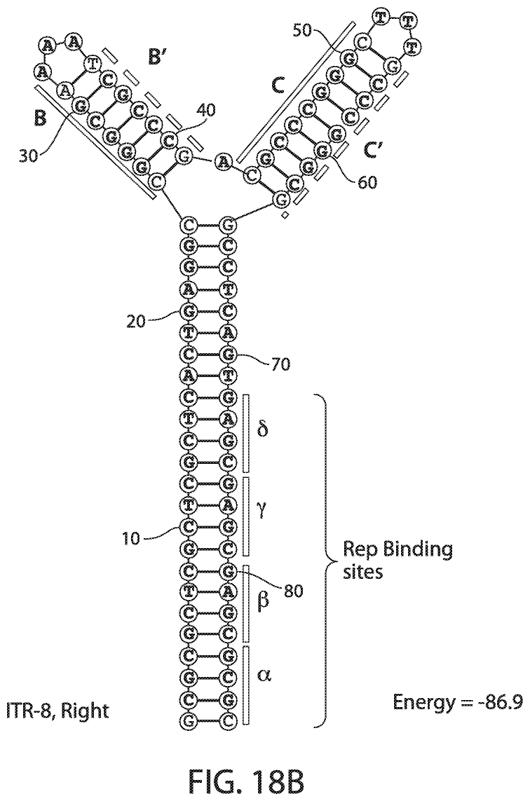

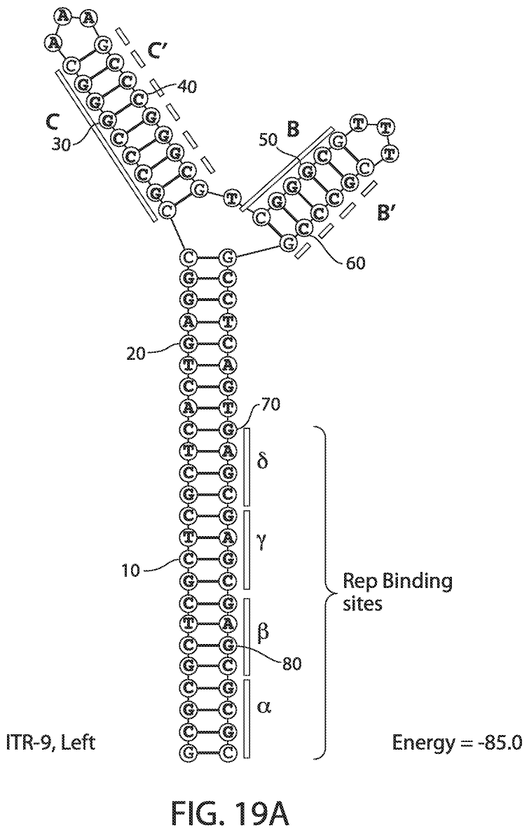

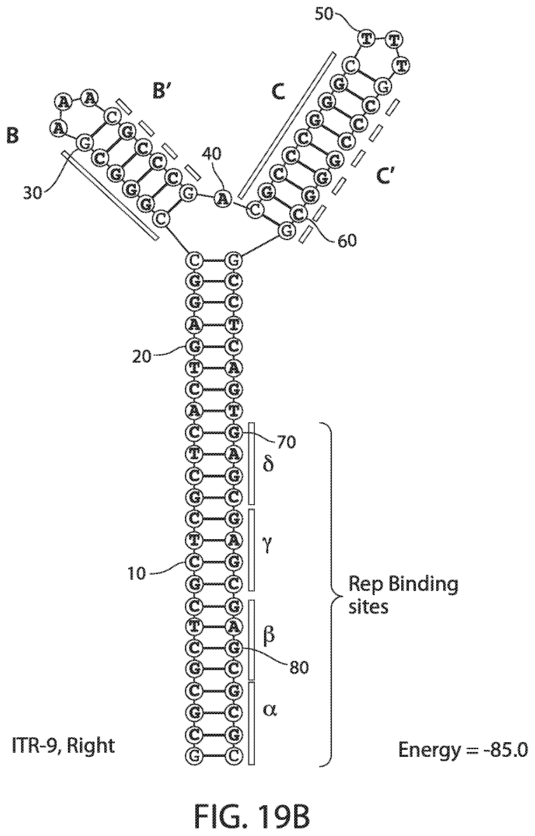

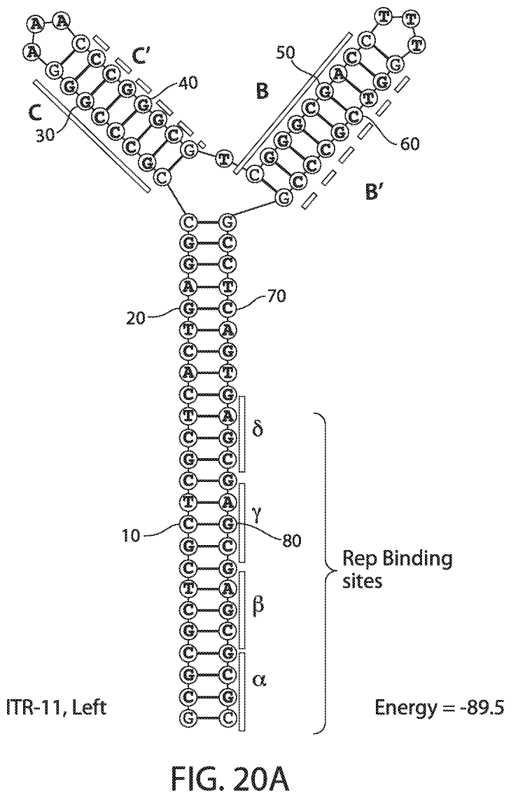

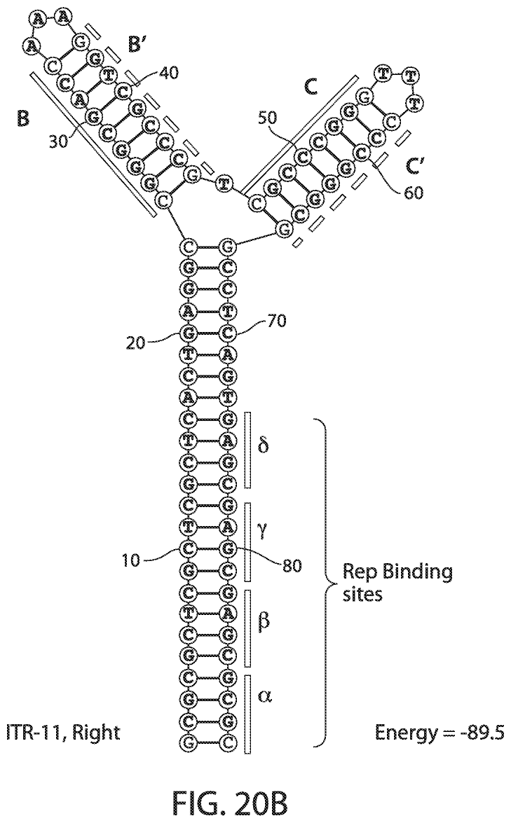

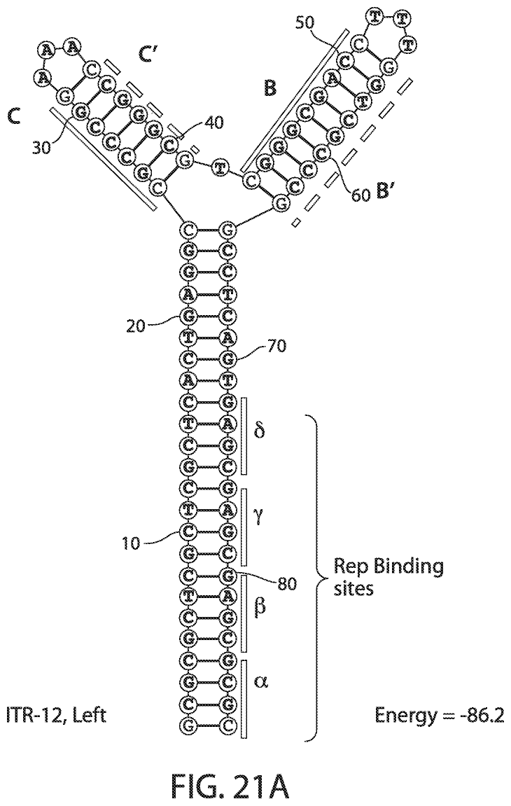

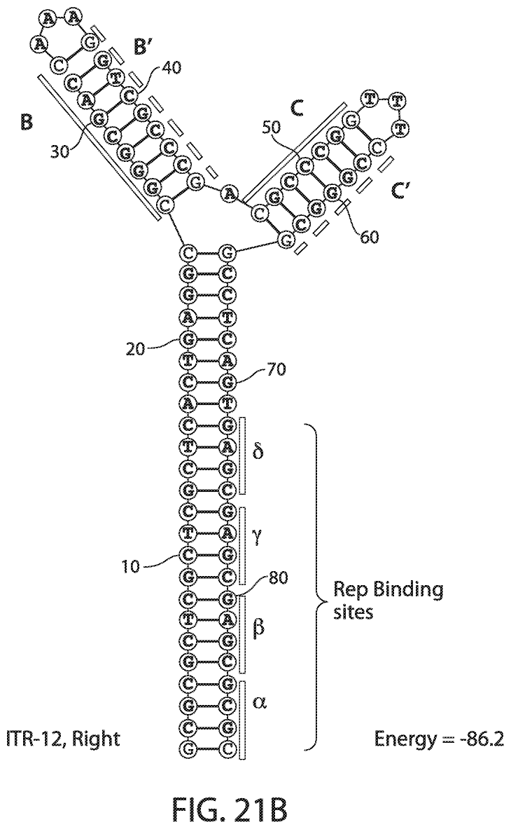

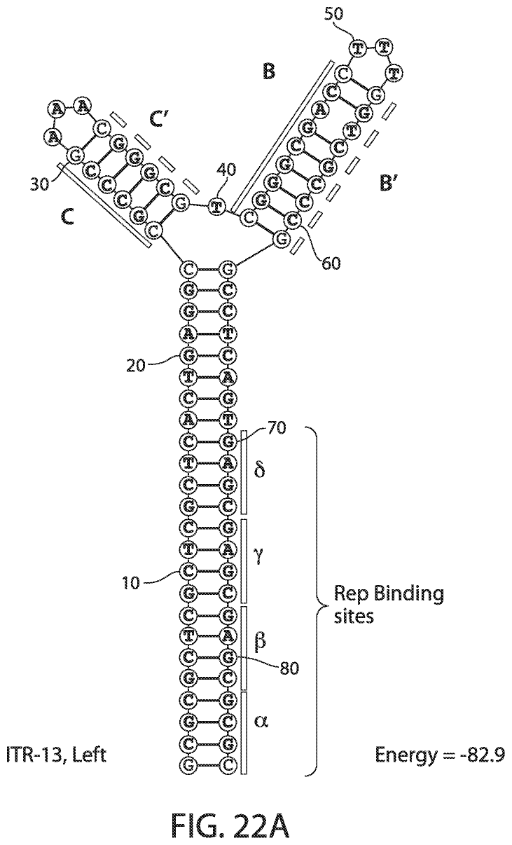

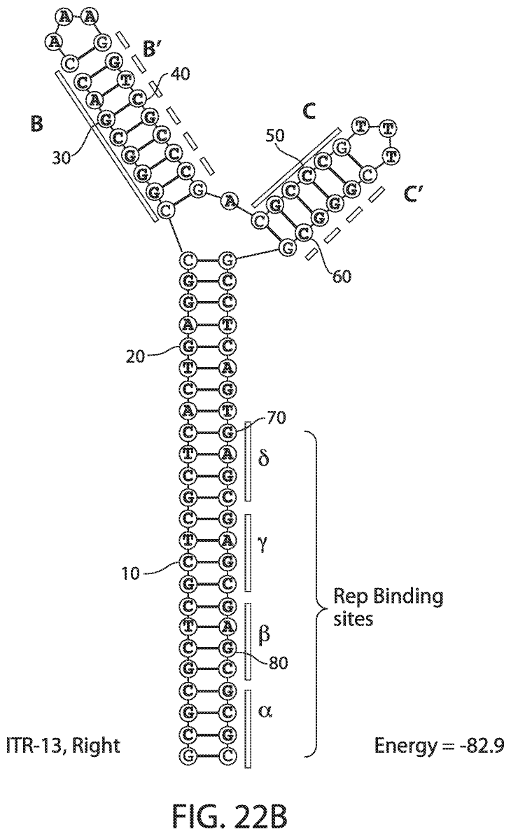

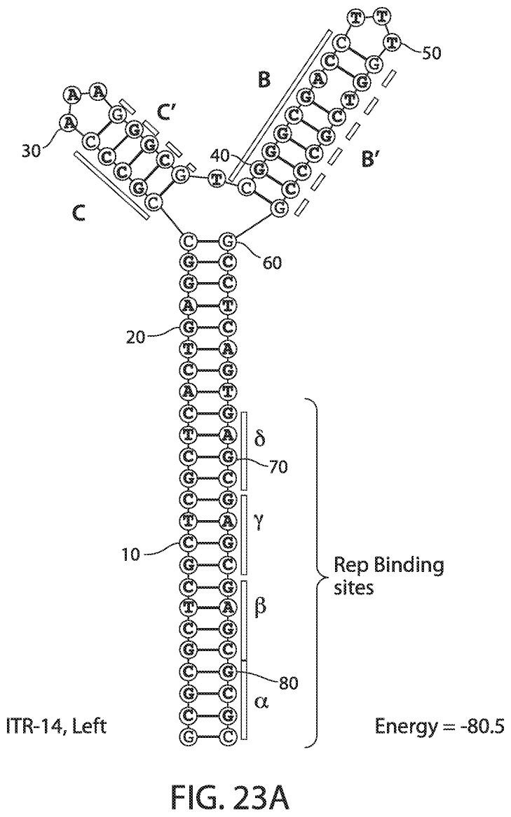

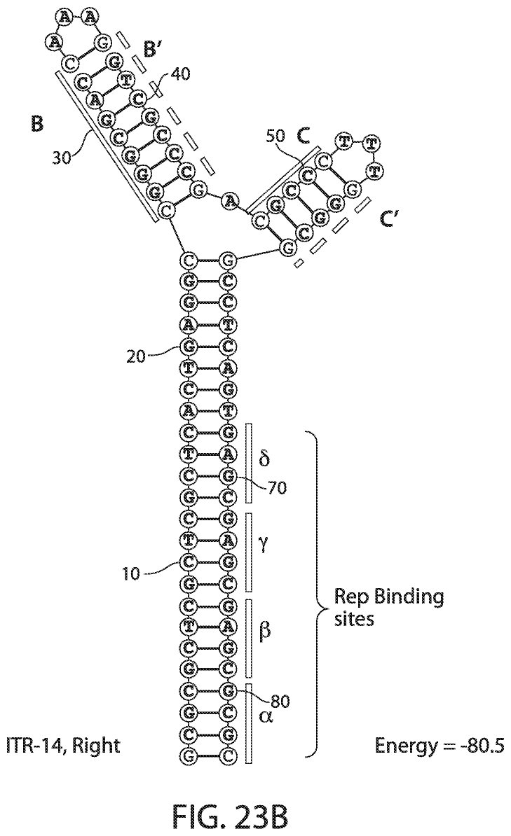

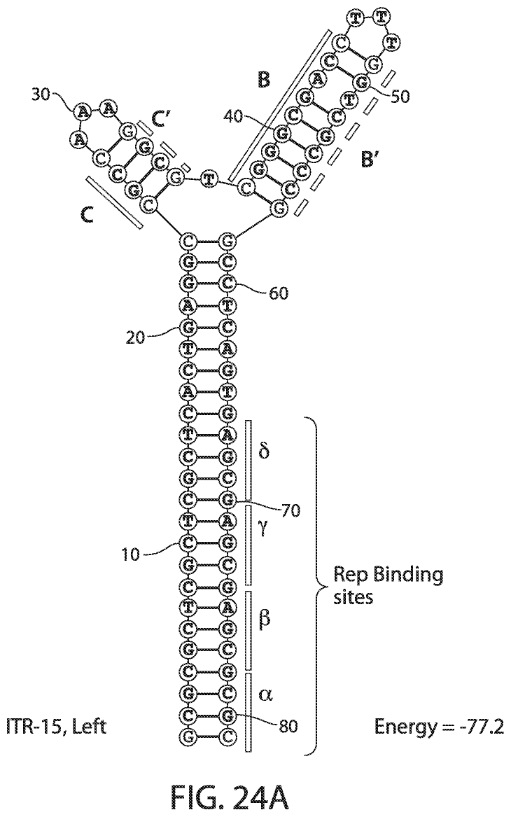

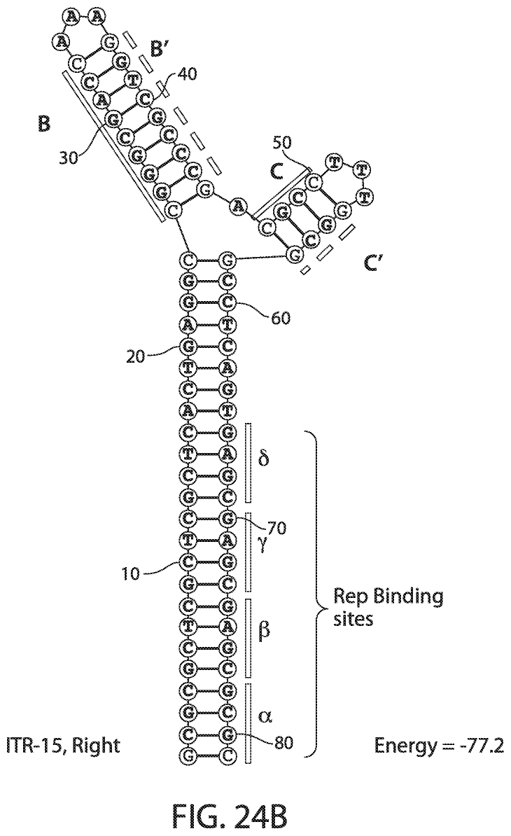

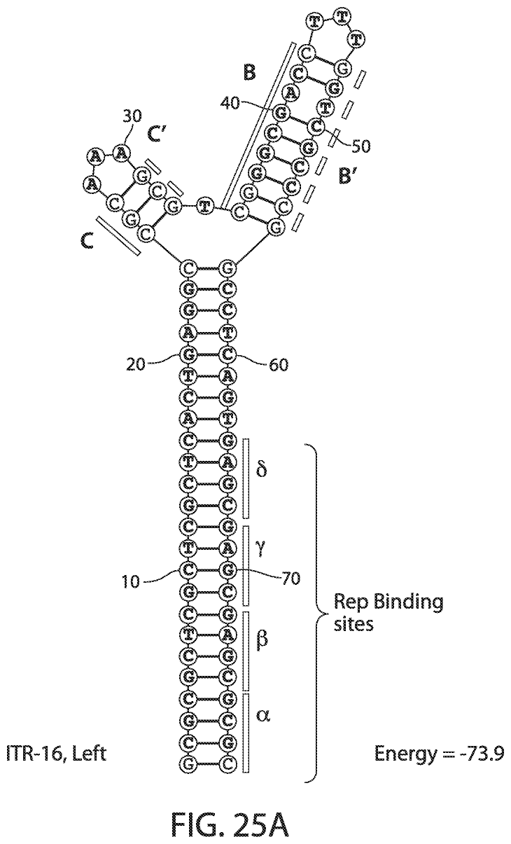

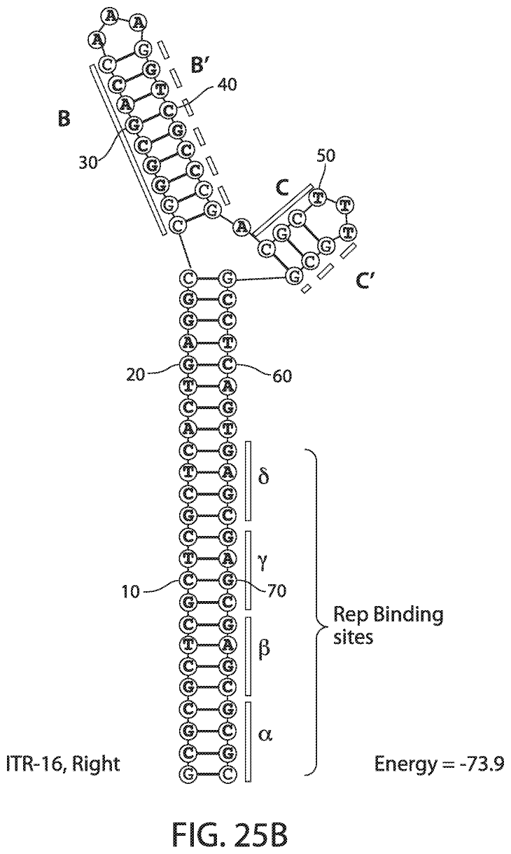

In some embodiments, ceDNA can form an intramolecular duplex secondary structure. The secondary structure of the first ITR and the asymmetric second ITR are exemplified in the context of wild-type ITRs (see, e.g., FIGS. 2A, 3A, 3C) and modified ITR structures (see e.g., FIG. 2B and FIGS. 3B, 3D). Secondary structures are inferred or predicted based on the ITR sequences of the plasmid used to produce the ceDNA vector. Exemplary secondary structures of the modified ITRs in which part of the stem-loop structure is deleted are shown in FIGS. 9A-25B and FIGS. 26A-26B, and also shown in Tables 10A and 10B. Exemplary secondary structures of the modified ITRs comprising a single stem and two loops are shown in FIGS. 9A-13B. Exemplary secondary structure of a modified ITR with a single stem and single loop is shown in FIG. 14. In some embodiments, the secondary structure can be inferred as shown herein using thermodynamic methods based on nearest neighbor rules that predict the stability of a structure as quantified by folding free energy change. For example, the structure can be predicted by finding the lowest free energy structure. In some embodiments, an algorithm disclosed in Reuter, J. S., & Mathews, D. H. (2010) RNAstructure: software for RNA secondary structure prediction and analysis. BMC Bioinformatics. 11,129 and implemented in the RNAstructure software (available at world wide web address: “rna.urmc.rochester.edu/RNAstructureWeb/index.html”) can be used for prediction of the ITR structure. The algorithm can also include both free energy change parameters at 37° C. and enthalpy change parameters derived from experimental literature to allow prediction of conformation stability at an arbitrary temperature. Using the RNA structure software, some of the modifed ITR structures can be predicted as modified T-shaped stem-loop structures with estimated Gibbs free energy (ΔG) of unfolding under physiological conditions shown in FIGS. 3A-3D. Using the RNAstructure software, the three types of modified ITRs are predicted to have a Gibbs free energy of unfolding higher than a wild-type ITR of AAV2 (−92.9 kcal/mol) and are as follows: (a) The modified ITRs with a single-arm/single-unpaired-loop structure provided herein are predicted to have a Gibbs free energy of unfolding that ranges between −85 and −70 kcal/mol. (b) The modified ITRs with a single-hairpin structure provided herein are predicted to have a Gibbs free energy of unfolding that ranges between −70 and −40 kcal/mol. (c) The modified ITRs with a two-arm structure provided herein are predicted to have a Gibbs free energy of unfolding that ranges between −90 and −70 kcal/mol. Without wishing to be bound by a theory, the structures with higher Gibbs free energy are easier to be unfold for replication by Rep 68 or Rep 78 replication proteins. Thus, modified ITRs having higher Gibbs free energy of unfolding—e.g., a single-arm/single-unpaired-loop structure, a single-hairpin structure, a truncated structure—tend to be replicated more efficiently than wild-type ITRs.

In one embodiment, the left ITR of the ceDNA vector is modified or mutated with respect to a wild type (wt) AAV ITR structure, and the right ITR is a wild type AAV ITR. In one embodiment, the right ITR of the ceDNA vector is modified with respect to a wild type AAV ITR structure, and the left ITR is a wild type AAV ITR. In such an embodiment, a modification of the ITR (e.g., the left or right ITR) can be generated by a deletion, an insertion, or substitution of one or more nucleotides from the wild type ITR derived from the AAV genome.

The ITRs used herein can be resolvable and non-resolvable, and selected for use in the ceDNA vectors are preferably AAV sequences, with serotypes 1, 2, 3, 4, 5, 6, 7, 8 and 9 being preferred. Resolvable AAV ITRs do not require a wild-type ITR sequence (e.g., the endogenous or wild-type AAV ITR sequence may be altered by insertion, deletion, truncation and/or missense mutations), as long as the terminal repeat mediates the desired functions, e.g., replication, virus packaging, integration, and/or provirus rescue, and the like. Typically, but not necessarily, the ITRs are from the same AAV serotype, e.g., both ITR sequences of the ceDNA vector are from AAV2. The ITRs may be synthetic sequences that function as AAV inverted terminal repeats, such as the “double-D sequence” as described in U.S. Pat. No. 5,478,745 to Samulski et al. While not necessary, the ITRs can be from the same parvovirus, e.g., both ITR sequences are from AAV2.

In one embodiment, ceDNA can include an ITR structure that is mutated with respect to one of the wild type ITRs disclosed herein, but where the mutant or modified ITR still retains an operable Rep binding site (RBE or RBE′) and terminal resolution site (trs). In one embodiment, the mutant ceDNA ITR includes a functional replication protein site (RPS-1) and a replication competent protein that binds the RPS-1 site is used in production.

In one embodiment, at least one of the ITRs is a defective ITR with respect to Rep binding and/or Rep nicking. In one embodiment, the defect is at least 30% relative to a wild type reduction ITR, in other embodiments it is at least 35% . . . , 50% . . . , 65% . . . , 75% . . . , 85% . . . , 90% . . . , 95% . . . , 98% . . . , or completely lacking in function or any point in-between. The host cells do not express viral capsid proteins and the polynucleotide vector template is devoid of any viral capsid coding sequences. In one embodiment, the polynucleotide vector templates and host cells that are devoid of AAV capsid genes and the resultant protein also do not encode or express capsid genes of other viruses. In addition, in a particular embodiment, the nucleic acid molecule is also devoid of AAV Rep protein coding sequences

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complentary RBE sequence), and a terminal resolution sire (trs).

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 1 indicates exemplary modifications of at least one nucleotide (e.g., a deletion, insertion and/or substitution) in regions of modified ITRs, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. In some embodiments, a truncated C-C′ arm and/or a truncated B-B′ arm has three sequential T nucleotides (i.e., TTT) in the terminal loop.

TABLE 1
Exemplary combinations of modifications
of at least one nucleotide (e.g., a deletion,
insertion and/or substitution) to
different B-B’ and C-C’ regions
or arms of ITRs (X indicates
a nucleotide modification, e.g.,
addition, deletion or substitution of
at least one nucleotide in the region).

| B region | B’ region | C region | C’ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

In some embodiments, a modified ITR for use herein can comprise any one of the combinations of modifications shown in Table 1, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotide (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop In some embodiments, a modified ITR for use herein can comprise any one of the combinations of modifications shown in Table 1, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 1, and also a modification of at least one nucleotide (e.g., a deletion, insertion and/or substitution) in the A region. In some embodiments, a modified ITR for use herein can comprise any one of the combinations of modifications shown in Table 1, and also a modification of at least one nucleotide (e.g., a deletion, insertion and/or substitution) in the A′ region. In some embodiments, a modified ITR for use herein can comprise any one of the combinations of modifications shown in Table 1, and also a modification of at least one nucleotide (e.g., a deletion, insertion and/or substitution) in the A and/or A′ region. In some embodiments, a modified ITR for use herein can comprise any one of the combinations of modifications shown in Table 1, and also a modification of at least one nucleotide (e.g., a deletion, insertion and/or substitution) in the D region.

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified herein (e.g., SEQ ID NOS: 2, 52, 63, 64, 101-499, or 545-547). In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 469-499 or 545-547, or the RBE-containing section of the A-A′ arm and C-C′ and B-B′ arms of SEQ ID NO: 101-134 or 545-547.

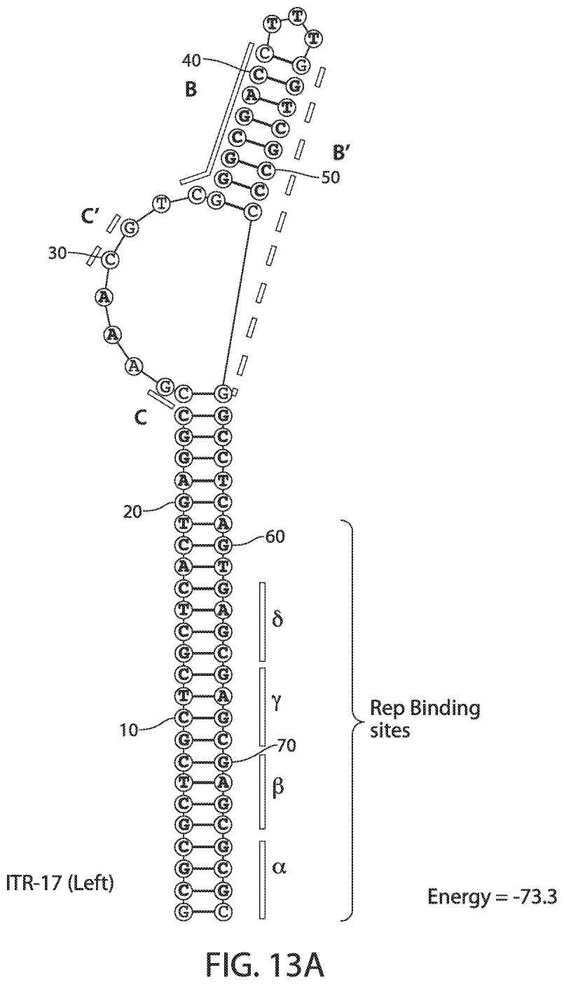

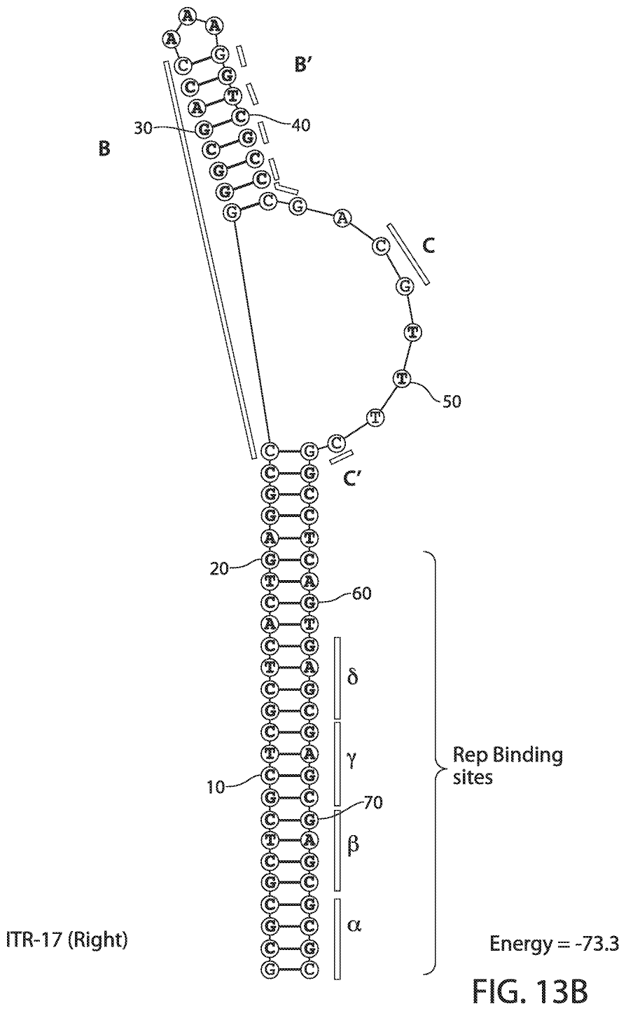

In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-6). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm. In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 13A-13B show an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. Note in this example, as the modified ITR comprises at least one base pair deletion from each of the B region and B′ regions, arm B-B′ is also truncated relative to WT ITR.

In some embodiments, 1, 2, 3, 4, 5, 6, 7, 8, 9 or more complementary base pairs are removed from each of the C portion and the C′ portion of the C-C′ arm such that the C-C′ arm is truncated. That is, if a base is removed in the C portion of the C-C′ arm, the complementary base pair in the C′ portion is removed, thereby truncating the C-C′ arm. In such embodiments, 2, 4, 6, 8 or more base pairs are removed from the C-C′ arm such that the C-C′ arm is truncated. In alternative embodiments, 1, 2, 3, 4, 5, 6, 7, 8, 9 or more base pairs are removed from the C portion of the C-C′ arm such that only C′ portion of the arm remains. In alternative embodiments, 1, 2, 3, 4, 5, 6, 7, 8, 9 or more base pairs are removed from the C′ portion of the C-C′ arm such that only C portion of the arm remains.

In some embodiments, 1, 2, 3, 4, 5, 6, 7, 8, 9 or more complementary base pairs are removed from each of the B portion and the B′ portion of the B-B′ arm such that the B-B′ arm is truncated. That is, if a base is removed in the B portion of the B-B′ arm, the complementary base pair in the B′ portion is removed, thereby truncating the B-B′ arm. In such embodiments, 2, 4, 6, 8 or more base pairs are removed from the B-B′ arm such that the B-B′ arm is truncated. In alternative embodiments, 1, 2, 3, 4, 5, 6, 7, 8, 9 or more base pairs are removed from the B portion of the B-B′ arm such that only B′ portion of the arm remains. In alternative embodiments, 1, 2, 3, 4, 5, 6, 7, 8, 9 or more base pairs are removed from the B′ portion of the B-B′ arm such that only B portion of the arm remains.

In some embodiments, a modified ITR can have between 1 and 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR forms two opposing, lengthwise-asymmetric stem-loops, e.g., C-C′ loop is a different length to the B-B′ loop. In some embodiments, one of the opposing, lengthwise-asymmetric stem-loops of a modified ITR has a C-C′ and/or B-B′ stem portion in the range of 8 to 10 base pairs in length and a loop portion (e.g., between C-C′ or between B-B′) having 2 to 5 unpaired deoxyribonucleotides. In some embodiments, a one lengthwise-asymmetric stem-loop of a modified ITR has a C-C′ and/or B-B′ stem portion of less than 8, or less than 7, 6, 5, 4, 3, 2, 1 base pairs in length and a loop portion (e.g., between C-C′ or between B-B′) having between 0-5 nucleotides. In some embodiments, a modified ITR with a lengthwise-asymmetric stem-loop has a C-C′ and/or B-B′ stem portion less than 3 base pairs in length.

In some embodiments, a modified ITR does not contain any nucleotide deletions in the RBE-containing portion of the A or A′ regions, so as not to interfere with DNA replication (e.g. binding to a RBE by Rep protein, or nicking at a terminal resolution site). In some embodiments, a modified ITR encompassed for use herein has one or more deletions in the B, B′, C, and/or C region as described herein. Several non-limiting examples of modified ITRS are shown in FIGS. 9A-26B.

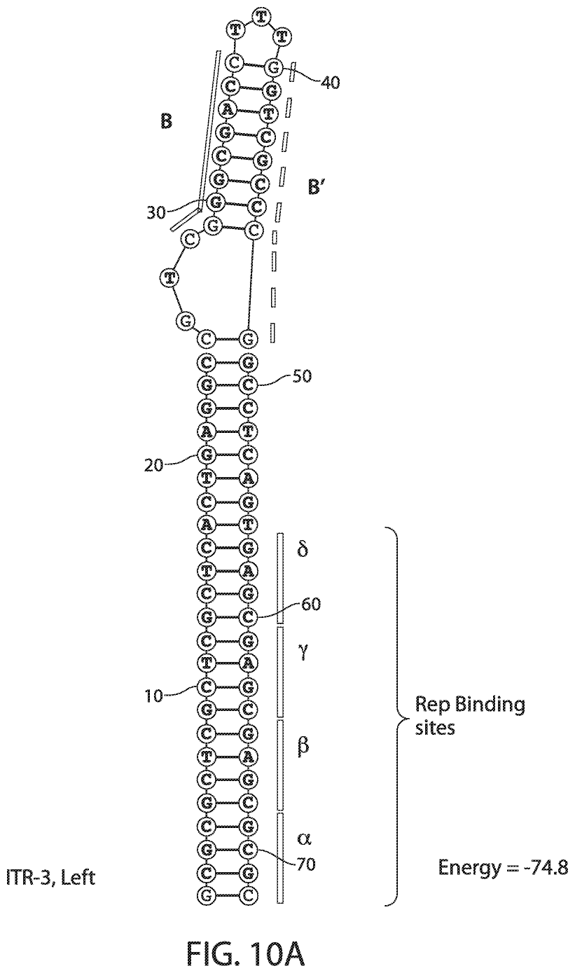

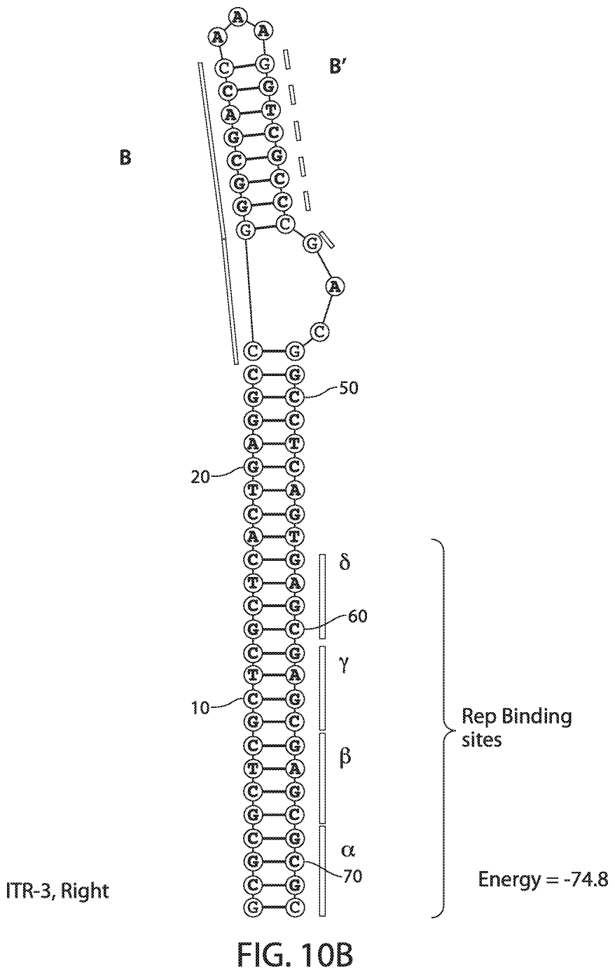

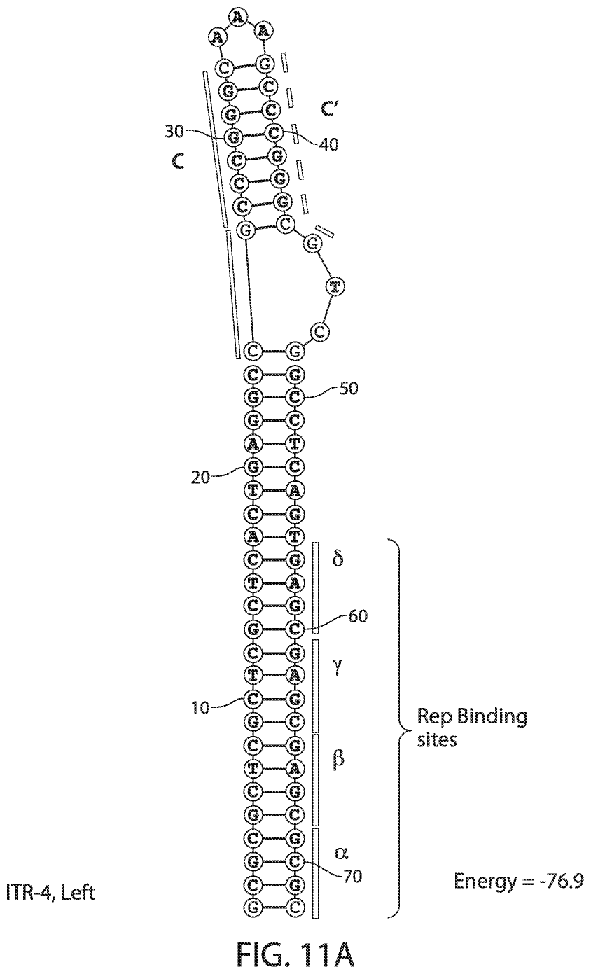

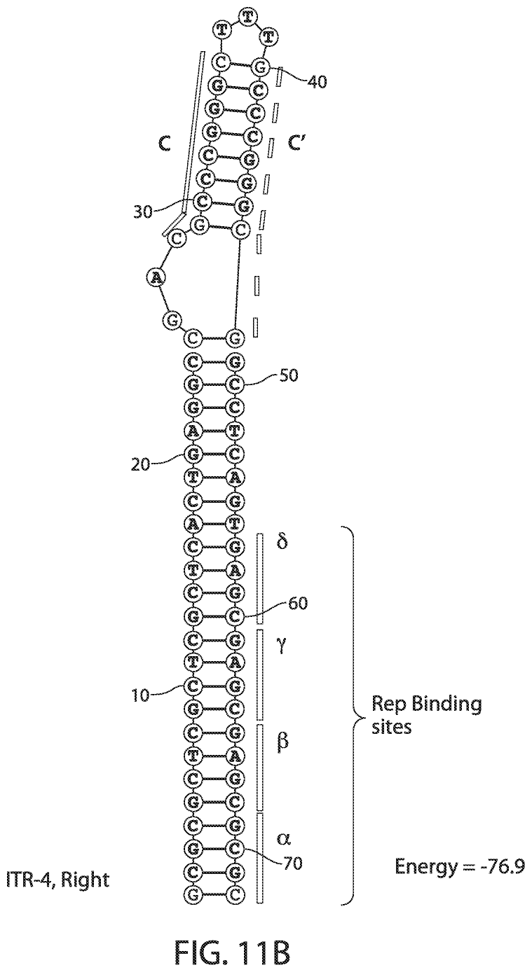

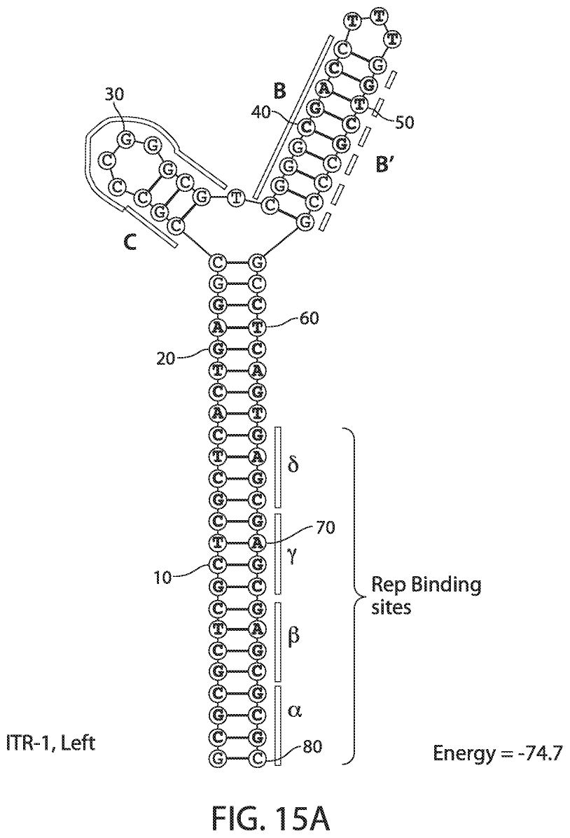

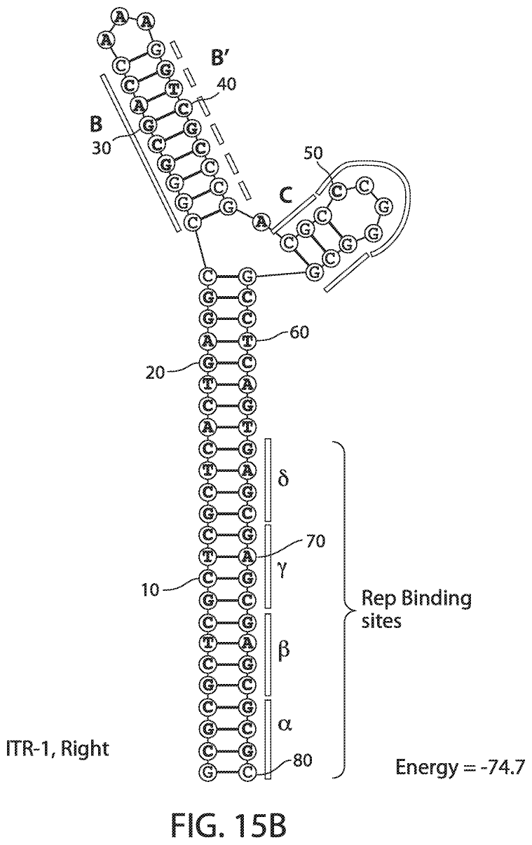

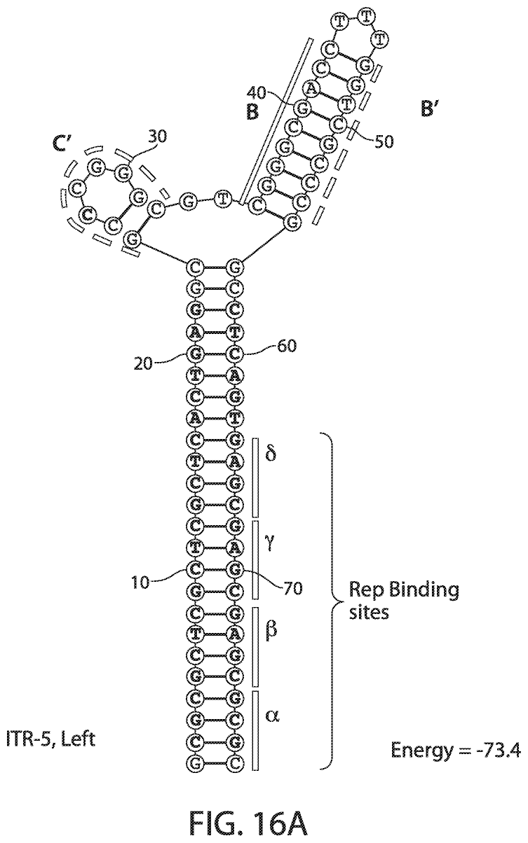

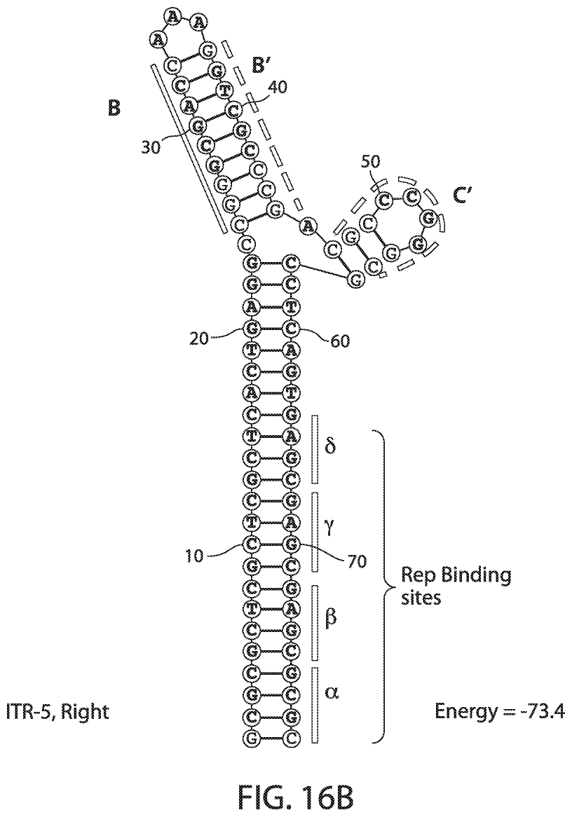

In some embodiments, a modified ITR can comprise a deletion of the B-B′ arm, so that the C-C′ arm remains, for example, see exemplary ITR-2 (left) and ITR-2 (right) shown in FIG. 9A-9B and ITR-4 (left) and ITR-4 (right) (FIGS. 11A-11B). In some embodiments, a modified ITR can comprise a deletion of the C-C′ arm such that the B-B′ arm remains, for example, see exemplary ITR-3 (left) and ITR-3 (right) shown in FIG. 10A-10B. In some embodiments, a modified ITR can comprise a deletion of the B-B′ arm and C-C′ arm such that a single stem-loop remains, for example, see exemplary ITR-6 (left) and ITR-6 (right) shown in FIG. 14A-14B, and ITR-21 and ITR-37. In some embodiments, a modified ITR can comprise a deletion of the C′ region such that a truncated C-loop and B-B′ arm remains, for example, see exemplary ITR-1 (left) and ITR-1 (right) shown in FIG. 15A-15B. Similarily, in some embodiments, a modified ITR can comprise a deletion of the C region such that a truncated C′-loop and B-B′ arm remains, for example, see exemplary ITR-5 (left) and ITR-5 (right) shown in FIG. 16A-16B.

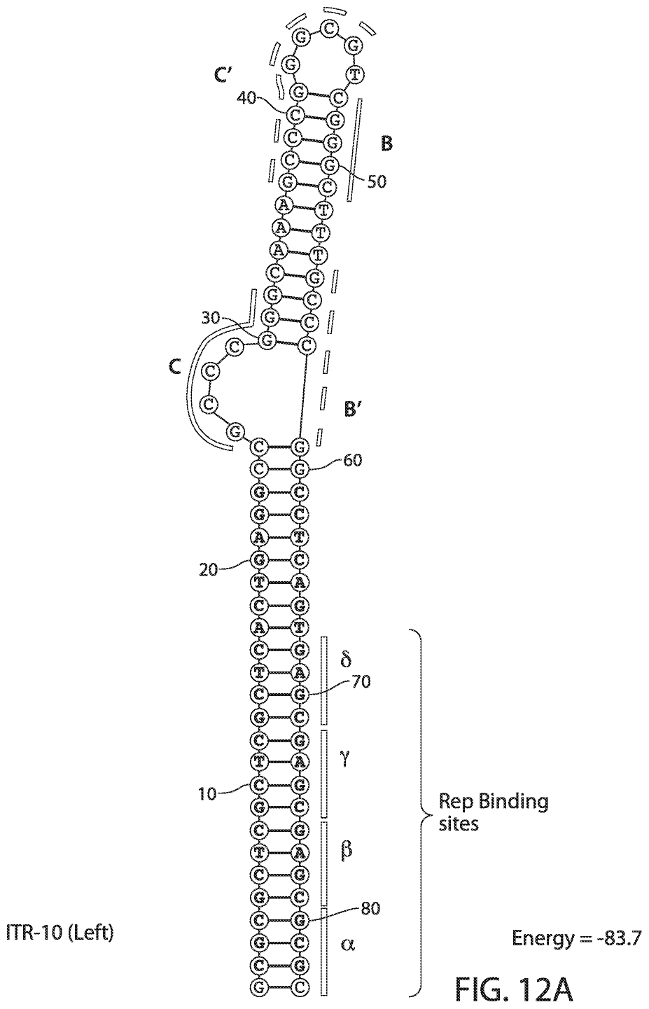

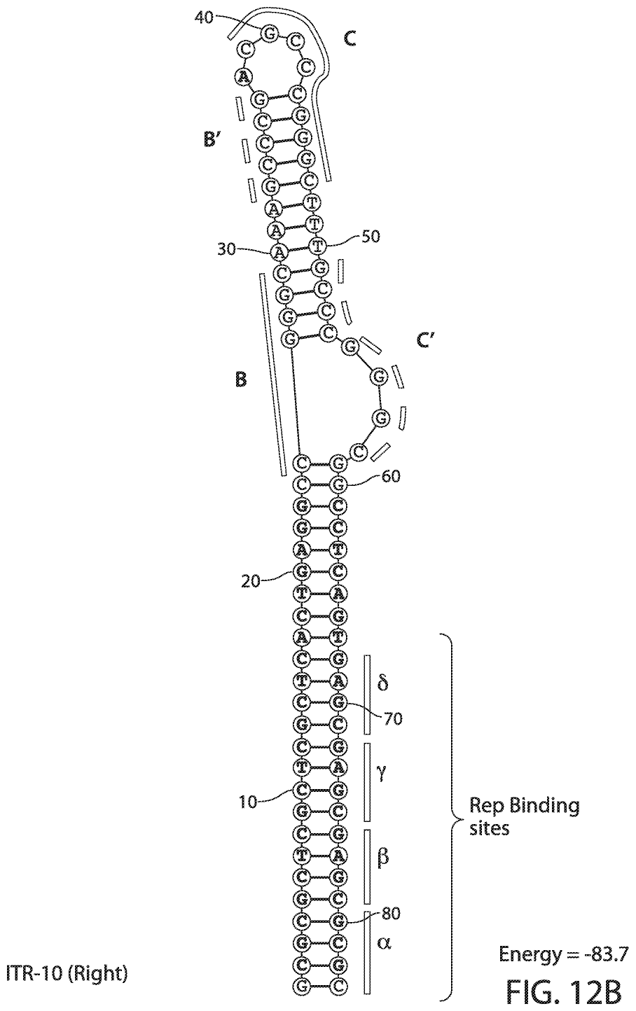

In some embodiments, a modified ITR can comprise a deletion of base pairs in any one or more of: the C portion, the C′ portion, the B portion or the B′ portion, such that complementary base pairing occurs between the C-B′ portions and the C′-B portions to produce a single arm, for example, see ITR-10 (right) and ITR-10 (left) (FIG. 12A-12B).

In some embodiments, in addition to a modification in one or more nucleotides in the C, C′, B and/or B′ regions, a modified ITR for use herein can comprise a modification (e.g., deletion, substitution or addition) of at least 1, 2, 3, 4, 5, 6 nucleotides in any one or more of the regions selected from: between A′ and C, between C and C′, between C′ and B, between B and B′ and between B′ and A. For example, the nucleotide between B′ and C in a modified right ITR can be substituted from a nA to a G, C or A or deleted or one or more nucleotides added; a nucleotide between C′ and B in a modified left ITR can be changed from a T to a G, C or A, or deleted or one or more nucleotides added.

In certain embodiments of the present invention, the ceDNA vector does not have a modified ITR consisting of the nucleotide sequence selected from any of: SEQ ID NOs: 550-557. In certain embodiments of the present invention, the ceDNA vector does not have a modified ITR comprising the nucleotide sequence selected from any of: SEQ ID NOs: 550-557.

In some embodiments, the ceDNA vector comprises a regulatory switch as disclosed herein and a modified ITR selected having the nucleotide sequence selected from any of the group consisting of: SEQ ID NO: 550-557.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector. In some embodiments, the ceDNA vector contains one or more functional ITR polynucleotide sequences that comprise a Rep-binding site (RBS; 5′-GCGCGCTCGCTCGCTC-3′ (SEQ ID NO: 531) for AAV2) and a terminal resolution site (TRS; 5′-AGTT (SEQ ID NO: 46)). In some embodiments, at least one ITR (wt or modified ITR) is functional. In alternative embodiments, where a ceDNA vector comprises two modified ITRs that are different or asymmetrical to each other, at least one modified ITR is functional and at least one modified ITR is non-functional.

In some embodiments, a ceDNA vector does not have a modified ITR selected from any sequence consisting of, or consisting essentially of: SEQ ID NOs:500-529, as provided herein. In some embodiments, a ceDNA vector does not have an ITR that is selected from any sequence selected from SEQ ID NOs: 500-529.

In some embodiments, the modified ITR (e.g., the left or right ITR) of the ceDNA vector described herein has modifications within the loop arm, the truncated arm, or the spacer. Exemplary sequences of ITRs having modifications within the loop arm, the truncated arm, or the spacer are listed in Table 2.

In some embodiments, the modified ITR (e.g., the left or right ITR) of the ceDNA vector described herein has modifications within the loop arm and the truncated arm. Exemplary sequences of ITRs having modifications within the loop arm and the truncated arm are listed in Table 3.

In some embodiments, the modified ITR (e.g., the left or right ITR) of the ceDNA vector described herein has modifications within the loop arm and the spacer. Exemplary sequences of ITRs having modifications within the loop arm and the spacer are listed in Table 4.

In some embodiments, the modified ITR (e.g., the left or right ITR) of the ceDNA vector described herein has modifications within the truncated arm and the spacer. Exemplary sequences of ITRs having modifications within the truncated arm and the spacer are listed in Table 5.

In some embodiments, the modified ITR (e.g., the left or right ITR) of the ceDNA vector described herein has modifications within the loop arm, the truncated arm, and the spacer. Exemplary sequences of ITRs having modifications within the loop arm, the truncated arm, and the spacer are listed in Table 6.

In some embodiments, the ITR (e.g., the left or right ITR) is modified such that it comprises the lowest energy of unfolding (“low energy structure”). A low energy will have reduced Gibbs free energy as compared to a wild type ITR. Exemplary sequences of ITRs that are modified to low (i.e., reduced) energy of unfolding are presented herein in Table 7-9.

In some embodiments, the modified ITR is selected from any or a combination of those shown in Table 2-9, 10A or 10B.

TABLE 2
ITR Sequences with Modifications in Loop Arm,
Truncated Arm or Spacer. These include the RBS sequence
GCGCGCTCGCTCGCTC (SEQ ID NO: 531) at the 5′ end and the
complementary RBE′ sequence GAGCGAGCGAGCGCGC
(SEQ ID NO: 536) on the most 3′ end.

| SEQ | Modified | | | No. |
|---|---|---|---|---|
| ID | Region | Sequence | ΔG | Strut. |
| 135 | Truncated | GCGCGCTCGCTCGCTCACTGAGGCCGGGCGACCAAAGGTCGCC | -73.6 | 1 |
| | Arm | CGAAGCCCGGGCTGCCTCAGTGAGCGAGCGAGCGCGC | | |
| 136 | | GCGCGCTCGCTCGCTCACTGAGGCCGGGCGACCAAAGGTCGCC | -73.7 | 3 |
| | | CGACACCCGGGTGGCCTCAGTGAGCGAGCGAGCGCGC | | |
| 137 | | GCGCGCTCGCTCGCTCACTGAGGCCGGGCGACCAAAGGTCGCC | -74.2 | 1 |
| | | CGACGACCGGTCGGCCTCAGTGAGCGAGCGAGCGCGC | | |
| 138 | | GCGCGCTCGCTCGCTCACTGAGGCCGGGCGACCAAAGGTCGCC | -75.7 | 2 |
| | | CGACGCACGTGCGGCCTCAGTGAGCGAGCGAGCGCGC | | |
| 139 | | GCGCGCTCGCTCGCTCACTGAGGCCGGGCGACCAAAGGTCGCC | -75.2 | 1 |
| | | CGACGCCATGGCGGCCTCAGTGAGCGAGCGAGCGCGC | | |
| 140 | | GCGCGCTCGCTCGCTCACTGAGGCCGGGCGACCAAAGGTCGCC | -72.1 | 1 |
| | | CGAAGACCGGTCTGCCTCAGTGAGCGAGCGAGCGCGC | | |
| 141 | | GCGCGCTCGCTCGCTCACTGAGGCCGGGCGACCAAAGGTCGCC | -74.2 | 1 |
| | | CGACACACGTGTGGCCTCAGTGAGCGAGCGAGCGCGC | | |
| 142 | | GCGCGCTCGCTCGCTCACTGAGGCCGGGCGACCAAAGGTCGCC | -73.3 | 2 |
| | | CGACGACATGTCGGCCTCAGTGAGCGAGCGAGCGCGC | | |
| 143 | | GCGCGCTCGCTCGCTCACTGAGGCCGGGCGACCAAAGGTCGCC | -74.1 | 1 |
| | | CGAAGCACGTGCTGCCTCAGTGAGCGAGCGAGCGCGC | | |
| 144 | | GCGCGCTCGCTCGCTCACTGAGGCCGGGCGACCAAAGGTCGCC | -72.1 | 1 |
| | | CGAAACCCGGGTTGCCTCAGTGAGCGAGCGAGCGCGC | | |
| 145 | | GCGCGCTCGCTCGCTCACTGAGGCCGGGCGACCAAAGGTCGCC | -73.6 | 1 |
| | | CGAAGCCATGGCTGCCTCAGTGAGCGAGCGAGCGCGC | | |
| 146 | | GCGCGCTCGCTCGCTCACTGAGGCCGGGCGACCAAAGGTCGCC | -73.0 | 1 |
| | | CGACAACCGGTTGGCCTCAGTGAGCGAGCGAGCGCGC | | |
| 147 | | GCGCGCTCGCTCGCTCACTGAGGCCGGGCGACCAAAGGTCGCC | -73.7 | 1 |
| | | CGACACCATGGTGGCCTCAGTGAGCGAGCGAGCGCGC | | |
| 148 | | GCGCGCTCGCTCGCTCACTGAGGCCGGGCGACCAAAGGTCGCC | -73.7 | 1 |
| | | CGACGAACGTTCGGCCTCAGTGAGCGAGCGAGCGCGC | | |
| 149 | | GCGCGCTCGCTCGCTCACTGAGGCCGGGCGACCAAAGGTCGCC | -75.7 | 1 |
| | | CGACGCAATTGCGGCCTCAGTGAGCGAGCGAGCGCGC | | |
| 150 | | GCGCGCTCGCTCGCTCACTGAGGCCGGGCGACCAAAGGTCGCC | -72.1 | 1 |
| | | CGAAAACCGGTTTGCCTCAGTGAGCGAGCGAGCGCGC | | |
| 151 | | GCGCGCTCGCTCGCTCACTGAGGCCGGGCGACCAAAGGTCGCC | -72.1 | 1 |
| | | CGAAGAACGTTCTGCCTCAGTGAGCGAGCGAGCGCGC | | |
| 152 | | GCGCGCTCGCTCGCTCACTGAGGCCGGGCGACCAAAGGTCGCC | -74.1 | 1 |
| | | CGAAGCAATTGCTGCCTCAGTGAGCGAGCGAGCGCGC | | |
| 153 | | GCGCGCTCGCTCGCTCACTGAGGCCGGGCGACCAAAGGTCGCC | -73.0 | 1 |
| | | CGACAAACGTTTGGCCTCAGTGAGCGAGCGAGCGCGC | | |
| 154 | | GCGCGCTCGCTCGCTCACTGAGGCCGGGCGACCAAAGGTCGCC | -73.7 | 2 |
| | | CGACGAAATTTCGGCCTCAGTGAGCGAGCGAGCGCGC | | |
| 155 | | GCGCGCTCGCTCGCTCACTGAGGCCGGGCGACCAAAGGTCGCC | -74.1 | 1 |
| | | CGAAGCAATTGCTGCCTCAGTGAGCGAGCGAGCGCGC | | |
| 156 | | GCGCGCTCGCTCGCTCACTGAGGCCGGGCGACCAAAGGTCGCC | -72.1 | 1 |
| | | CGAAACCATGGTTGCCTCAGTGAGCGAGCGAGCGCGC | | |
| 157 | | GCGCGCTCGCTCGCTCACTGAGGCCGGGCGACCAAAGGTCGCC | -73.0 | 1 |
| | | CGACAACATGTTGGCCTCAGTGAGCGAGCGAGCGCGC | | |
| 158 | | GCGCGCTCGCTCGCTCACTGAGGCCGGGCGACCAAAGGTCGCC | -72.1 | 2 |
| | | CGAAGACATGTCTGCCTCAGTGAGCGAGCGAGCGCGC | | |
| 159 | | GCGCGCTCGCTCGCTCACTGAGGCCGGGCGACCAAAGGTCGCC | -74.2 | 1 |
| | | CGACACAATTGTGGCCTCAGTGAGCGAGCGAGCGCGC | | |
| 160 | | GCGCGCTCGCTCGCTCACTGAGGCCGGGCGACCAAAGGTCGCC | -72.1 | 1 |
| | | CGAAAAACGTTTTGCCTCAGTGAGCGAGCGAGCGCGC | | |
| 161 | | GCGCGCTCGCTCGCTCACTGAGGCCGGGCGACCAAAGGTCGCC | -72.1 | 2 |
| | | CGAAAACATGTTTGCCTCAGTGAGCGAGCGAGCGCGC | | |
| 162 | | GCGCGCTCGCTCGCTCACTGAGGCCGGGCGACCAAAGGTCGCC | -72.1 | 1 |
| | | CGAAACAATTGTTGCCTCAGTGAGCGAGCGAGCGCGC | | |
| 163 | | GCGCGCTCGCTCGCTCACTGAGGCCGGGCGACCAAAGGTCGCC | -72.1 | 1 |
| | | CGAAGAAATTTCTGCCTCAGTGAGCGAGCGAGCGCGC | | |
| 164 | | GCGCGCTCGCTCGCTCACTGAGGCCGGGCGACCAAAGGTCGCC | -73.0 | 1 |
| | | CGACAAAATTTTGGCCTCAGTGAGCGAGCGAGCGCGC | | |
| 165 | | GCGCGCTCGCTCGCTCACTGAGGCCGGGCGACCAAAGGTCGCC | -72.1 | 1 |
| | | CGAAAAAATTTTTGCCTCAGTGAGCGAGCGAGCGCGC | | |
| 166 | Spacer | GCGCGCTCGCTCGCTCGCTGAGGCCGGGCGACCAAAGGTCGCC | -76.7 | 1 |
| | | CGACGCCCGGGCGGCCTCAGCGAGCGAGCGAGCGCGC | | |
| 167 | | GCGCGCTCGCTCGCTCAATGAGGCCGGGCGACCAAAGGTCGCC | -72.9 | 1 |
| | | CGACGCCCGGGCGGCCTCATTGAGCGAGCGAGCGCGC | | |
| 168 | | GCGCGCTCGCTCGCTCACCGAGGCCGGGCGACCAAAGGTCGCC | -76.7 | 1 |
| | | CGACGCCCGGGCGGCCTCGGTGAGCGAGCGAGCGCGC | | |
| 169 | | GCGCGCTCGCTCGCTCACTAAGGCCGGGCGACCAAAGGTCGCC | -72.9 | 1 |
| | | CGACGCCCGGGCGGCCTTAGTGAGCGAGCGAGCGCGC | | |
| 170 | | GCGCGCTCGCTCGCTCACTGGGGCCGGGCGACCAAAGGTCGCC | -77.3 | 2 |
| | | CGACGCCCGGGCGGCCCCAGTGAGCGAGCGAGCGCGC | | |
| 171 | | GCGCGCTCGCTCGCTCACTGAAGCCGGGCGACCAAAGGTCGCC | -72.8 | 1 |
| | | CGACGCCCGGGCGGCTTCAGTGAGCGAGCGAGCGCGC | | |
| 172 | | GCGCGCTCGCTCGCTCACTGAGACCGGGCGACCAAAGGTCGCC | -73.1 | 1 |
| | | CGACGCCCGGGCGGTCTCAGTGAGCGAGCGAGCGCGC | | |
| 173 | | GCGCGCTCGCTCGCTCGATGAGGCCGGGCGACCAAAGGTCGCC | -74.7 | 1 |
| | | CGACGCCCGGGCGGCCTCATCGAGCGAGCGAGCGCGC | | |
| 174 | | GCGCGCTCGCTCGCTCGCGGAGGCCGGGCGACCAAAGGTCGCC | -78.2 | 2 |
| | | CGACGCCCGGGCGGCCTCCGCGAGCGAGCGAGCGCGC | | |
| 175 | | GCGCGCTCGCTCGCTCGCTAAGGCCGGGCGACCAAAGGTCGCC | -72.5 | 1 |
| | | CGACGCCCGGGCGGCCTTAGTGAGCGAGCGAGCGCGC | | |
| 176 | | GCGCGCTCGCTCGCTCGCTGGGGCCGGGCGACCAAAGGTCGCC | -78.8 | 2 |
| | | CGACGCCCGGGCGGCCCCAGCGAGCGAGCGAGCGCGC | | |
| 177 | | GCGCGCTCGCTCGCTCGCTGAAGCCGGGCGACCAAAGGTCGCC | -74.3 | 1 |
| | | CGACGCCCGGGCGGCTTCAGCGAGCGAGCGAGCGCGC | | |
| 178 | | GCGCGCTCGCTCGCTCGCTGAGACCGGGCGACCAAAGGTCGCC | -74.6 | 1 |
| | | CGACGCCCGGGCGGTCTCAGCGAGCGAGCGAGCGCGC | | |
| 179 | | GCGCGCTCGCTCGCTCGAGGAGGCCGGGCGACCAAAGGTCGCC | -76.9 | 1 |
| | | CGACGCCCGGGCGGCCTCCTCGAGCGAGCGAGCGCGC | | |
| 180 | | GCGCGCTCGCTCGCTCGATAAGGCCGGGCGACCAAAGGTCGCC | -72.4 | 1 |
| | | CGACGCCCGGGCGGCCTTATCGAGCGAGCGAGCGCGC | | |
| 181 | | GCGCGCTCGCTCGCTCGATGGGGCCGGGCGACCAAAGGTCGCC | -73.8 | 2 |
| | | CGACGCCCGGGCGGCCTCATCGAGCGAGCGAGCGCGC | | |
| 182 | | GCGCGCTCGCTCGCTCGATGAAGCCGGGCGACCAAAGGTCGCC | -72.3 | 1 |
| | | CGACGCCCGGGCGGCTTCATCGAGCGAGCGAGCGCGC | | |
| 183 | | GCGCGCTCGCTCGCTCGATGAGACCGGGCGACCAAAGGTCGCC | -72.6 | 1 |
| | | CGACGCCCGGGCGGTCTCATCGAGCGAGCGAGCGCGC | | |
| 184 | | GCGCGCTCGCTCGCTCGAGAAGGCCGGGCGACCAAAGGTCGCC | -74.5 | 1 |
| | | CGACGCCCGGGCGGCCTTCTCGAGCGAGCGAGCGCGC | | |
| 185 | | GCGCGCTCGCTCGCTCGAGGGGGCCGGGCGACCAAAGGTCGCC | -79 | 2 |
| | | CGACGCCCGGGCGGCCCCCTCGAGCGAGCGAGCGCGC | | |
| 186 | | GCGCGCTCGCTCGCTCGAGGAAGCCGGGCGACCAAAGGTCGCC | -74.5 | 1 |
| | | CGACGCCCGGGCGGCTTCCTCGAGCGAGCGAGCGCGC | | |
| 189 | | GCGCGCTCGCTCGCTCGAGGAGACCGGGCGACCAAAGGTCGCC | -74.8 | 1 |
| | | CGACGCCCGGGCGGTCTCCTCGAGCGAGCGAGCGCGC | | |
| 187 | | GCGCGCTCGCTCGCTCGAGGGGGCCGGGCGACCAAAGGTCGCC | -79 | 2 |
| | | CGACGCCCGGGCGGCCCCCTCGAGCGAGCGAGCGCGC | | |
| 188 | | GCGCGCTCGCTCGCTCGAGGAAGCCGGGCGACCAAAGGTCGCC | -74.5 | 1 |
| | | CGACGCCCGGGCGGCTTCCTCGAGCGAGCGAGCGCGC | | |
| 189 | | GCGCGCTCGCTCGCTCGAGGAGACCGGGCGACCAAAGGTCGCC | -74.8 | 1 |
| | | CGACGCCCGGGCGGTCTCCTCGAGCGAGCGAGCGCGC | | |
| 190 | | GCGCGCTCGCTCGCTCGAGAGGGCCGGGCGACCAAAGGTCGCC | -76.9 | 2 |
| | | CGACGCCCGGGCGGCCCTCTCGAGCGAGCGAGCGCGC | | |
| 200 | | GCGCGCTCGCTCGCTCGAGAAAGCCGGGCGACCAAAGGTCGCC | -72.1 | 1 |
| | | CGACGCCCGGGCGGCTTTCTCGAGCGAGCGAGCGCGC | | |
| 201 | | GCGCGCTCGCTCGCTCGAGAAGACCGGGCGACCAAAGGTCGCC | -69.1 | 2 |
| | | CGACGCCCGGGCGGCCTTCTCGAGCGAGCGAGCGCGC | | |
| 202 | | GCGCGCTCGCTCGCTCGAGAGAGCCGGGCGACCAAAGGTCGCC | -74.8 | 1 |
| | | CGACGCCCGGGCGGCTCTCTCGAGCGAGCGAGCGCGC | | |
| 203 | | GCGCGCTCGCTCGCTCGAGAGGACCGGGCGACCAAAGGTCGCC | -74.8 | 1 |
| | | CGACGCCCGGGCGGTCCTCTCGAGCGAGCGAGCGCGC | | |
| 204 | | GCGCGCTCGCTCGCTCGAGAGAACCGGGCGACCAAAGGTCGCC | -72.4 | 1 |
| | | CGACGCCCGGGCGGTTCTCTCGAGCGAGCGAGCGCGC | | |
| 205 | | GCGCGCTCGCTCGCTCAAGAGAACCGGGCGACCAAAGGTCGCC | -70.6 | 1 |
| | | CGACGCCCGGGCGGTTCTCTTGAGCGAGCGAGCGCGC | | |
| 206 | | GCGCGCTCGCTCGCTCACGAGAACCGGGCGACCAAAGGTCGCC | -72.2 | 1 |
| | | CGACGCCCGGGCGGTTCTCGTGAGCGAGCGAGCGCGC | | |
| 207 | | GCGCGCTCGCTCGCTCACTAGAACCGGGCGACCAAAGGTCGCC | -70.8 | 1 |
| | | CGACGCCCGGGCGGTTCTAGTGAGCGAGCGAGCGCGC | | |
| 208 | | GCGCGCTCGCTCGCTCACTGGAACCGGGCGACCAAAGGTCGCC | -72.8 | 1 |
| | | CGACGCCCGGGCGGTTCCAGTGAGCGAGCGAGCGCGC | | |
| 209 | | GCGCGCTCGCTCGCTCACTGAAACCGGGCGACCAAAGGTCGCC | -70.4 | 1 |
| | | CGACGCCCGGGCGGTTTCAGTGAGCGAGCGAGCGCGC | | |
| 210 | | GCGCGCTCGCTCGCTCGCGGGGGCCGGGCGACCAAAGGTCGCC | -80.3 | 2 |
| | | CGACGCCCGGGCGGCCCCCGCGAGCGAGCGAGCGCGC | | |
| 211 | | GCGCGCTCGCTCGCTCAATAAAACCGGGCGACCAAAGGTCGCC | -65.8 | 1 |
| | | CGACGCCCGGGCGGTTTTATTGAGCGAGCGAGCGCGC | | |
| 212 | Loop Arm | GCGCGCTCGCTCGCTCACTGAGGCCAGGCGACCAAAGGTCGCC | -73.7 | 1 |
| | | TGACGCCCGGGCGGCCTCAGTGAGCGAGCGAGCGCGC | | |
| 213 | | GCGCGCTCGCTCGCTCACTGAGGCCGAGCGACCAAAGGTCGCT | -73.1 | 1 |
| | | CGACGCCCGGGCGGCCTCAGTGAGCGAGCGAGCGCGC | | |
| 214 | | GCGCGCTCGCTCGCTCACTGAGGCCGGACGACCAAAGGTCGTC | -73.1 | 2 |
| | | CGACGCCCGGGCGGCCTCAGTGAGCGAGCGAGCGCGC | | |
| 215 | | GCGCGCTCGCTCGCTCACTGAGGCCGGGAGACCAAAGGTCTCC | -73.9 | 1 |
| | | CGACGCCCGGGCGGCCTCAGTGAGCGAGCGAGCGCGC | | |
| 216 | | GCGCGCTCGCTCGCTCACTGAGGCCGGGCAACCAAAGGTTGCC | -73.4 | 1 |
| | | CGACGCCCGGGCGGCCTCAGTGAGCGAGCGAGCGCGC | | |
| 217 | | GCGCGCTCGCTCGCTCACTGAGGCCGGGCGGCCAAAGGCCGCC | -77.3 | 2 |
| | | CGACGCCCGGGCGGCCTCAGTGAGCGAGCGAGCGCGC | | |
| 218 | | GCGCGCTCGCTCGCTCACTGAGGCCGGGCGAACAAAGTTCGCC | -72.8 | 2 |
| | | CGACGCCCGGGCGGCCTCAGTGAGCGAGCGAGCGCGC | | |
| 219 | | GCGCGCTCGCTCGCTCACTGAGGCCGGGCGACAAAATGTCGCC | -73.5 | 1 |
| | | CGACGCCCGGGCGGCCTCAGTGAGCGAGCGAGCGCGC | | |
| 220 | | GCGCGCTCGCTCGCTCACTGAGGCCAAGCGACCAAAGGTCGCT | -71.3 | 1 |
| | | TGACGCCCGGGCGGCCTCAGTGAGCGAGCGAGCGCGC | | |
| 221 | | GCGCGCTCGCTCGCTCACTGAGGCCAAACGACCAAAGGTCGTT | -68.9 | 1 |
| | | TGACGCCCGGGCGGCCTCAGTGAGCGAGCGAGCGCGC | | |
| 222 | | GCGCGCTCGCTCGCTCACTGAGGCCAAAAGACCAAAGGTCTTT | -67.3 | 2 |
| | | TGACGCCCGGGCGGCCTCAGTGAGCGAGCGAGCGCGC | | |
| 223 | | GCGCGCTCGCTCGCTCACTGAGGCCAAAAAACCAAAGGTTTTT | -64.6 | 2 |
| | | TGACGCCCGGGCGGCCTCAGTGAGCGAGCGAGCGCGC | | |
| 224 | | GCGCGCTCGCTCGCTCACTGAGGCCAAAAAGCCAAAGGCTTTT | -67 | 2 |
| | | TGACGCCCGGGCGGCCTCAGTGAGCGAGCGAGCGCGC | | |
| 225 | | GCGCGCTCGCTCGCTCACTGAGGCCAAAAAGACAAAGTCTTTT | -64.9 | 1 |
| | | TGACGCCCGGGCGGCCTCAGTGAGCGAGCGAGCGCGC | | |
| 226 | | GCGCGCTCGCTCGCTCACTGAGGCCAAAAAGAAAAATTCTTTT | -63.1 | 1 |
| | | TGACGCCCGGGCGGCCTCAGTGAGCGAGCGAGCGCGC | | |
| 227 | | GCGCGCTCGCTCGCTCACTGAGGCCAAAAAAAAAAATTTTTTT | -60.4 | 1 |
| | | TGACGCCCGGGCGGCCTCAGTGAGCGAGCGAGCGCGC | | |
| 228 | | GCGCGCTCGCTCGCTCACTGAGGCCGAAAAAAAAAATTTTTTT | -62.2 | 1 |
| | | CGACGCCCGGGCGGCCTCAGTGAGCGAGCGAGCGCGC | | |
| 229 | | GCGCGCTCGCTCGCTCACTGAGGCCGGAAAGAAAAATTCTTTC | -67.3 | 1 |
| | | CGACGCCCGGGCGGCCTCAGTGAGCGAGCGAGCGCGC | | |
| 230 | | GCGCGCTCGCTCGCTCACTGAGGCCGGGAAGAAAAATTCTTCC | -69.7 | 2 |
| | | CGACGCCCGGGCGGCCTCAGTGAGCGAGCGAGCGCGC | | |
| 231 | | GCGCGCTCGCTCGCTCACTGAGGCCGGGCAGAAAAATTCTGCC | -71.9 | 1 |
| | | CGACGCCCGGGCGGCCTCAGTGAGCGAGCGAGCGCGC | | |
| 232 | | GCGCGCTCGCTCGCTCACTGAGGCCGGGCGGAAAAATTCCGCC | -73.4 | 2 |
| | | CGACGCCCGGGCGGCCTCAGTGAGCGAGCGAGCGCGC | | |
| 233 | | GCGCGCTCGCTCGCTCACTGAGGCCGGGCGAAAAAATTTCGCC | -71.0 | 2 |
| | | CGACGCCCGGGCGGCCTCAGTGAGCGAGCGAGCGCGC | | |

TABLE 3
modified ITR Sequences with
Modifications in Loop Arm and Truncated Arm

| SEQ | Modified | | | No. |
|---|---|---|---|---|
| ID | Region | Sequence | ΔG | Strut. |
| 234 | Loop | GCGCGCTCGCTCGCTCACTGAGGCCAGGCGACCAAAGGTCGCCTGA | -72.2 | 2 |
| | Arm & | CACCCGGGTGGCCTCAGTGAGCGAGCGAGCGCGC | | |
| 235 | Truncated | GCGCGCTCGCTCGCTCACTGAGGCCAGGCGACCAAAGGTCGCCTGA | -73.7 | 1 |
| | Arm | CGCCATGGCGGCCTCAGTGAGCGAGCGAGCGCGC | | |
| 236 | | GCGCGCTCGCTCGCTCACTGAGGCCAGGCGACCAAAGGTCGCCTGA | -71.8 | 1 |
| | | CGACATGTCGGCCTCAGTGAGCGAGCGAGCGCGC | | |
| 237 | | GCGCGCTCGCTCGCTCACTGAGGCCAGGCGACCAAAGGTCGCCTGA | -72.2 | 1 |
| | | CGAACGTTCGGCCTCAGTGAGCGAGCGAGCGCGC | | |
| 238 | | GCGCGCTCGCTCGCTCACTGAGGCCAGGCGACCAAAGGTCGCCTGA | -72.6 | 1 |
| | | AGCAATTGCTGCCTCAGTGAGCGAGCGAGCGCGC | | |
| 239 | | GCGCGCTCGCTCGCTCACTGAGGCCGGGCGGCCAAAGGCCGCCCGA | -75.8 | 2 |
| | | CACCCGGGTGGCCTCAGTGAGCGAGCGAGCGCGC | | |
| 240 | | GCGCGCTCGCTCGCTCACTGAGGCCGGGCGGCCAAAGGCCGCCCGA | -77.3 | 1 |
| | | CGCCATGGCGGCCTCAGTGAGCGAGCGAGCGCGC | | |
| 241 | | GCGCGCTCGCTCGCTCACTGAGGCCGGGCGGCCAAAGGCCGCCCGA | -75.4 | 1 |
| | | CGACATGTCGGCCTCAGTGAGCGAGCGAGCGCGC | | |
| 242 | | GCGCGCTCGCTCGCTCACTGAGGCCGGGCGGCCAAAGGCCGCCCGA | -75.8 | 1 |
| | | CGAACGTTCGGCCTCAGTGAGCGAGCGAGCGCGC | | |
| 243 | | GCGCGCTCGCTCGCTCACTGAGGCCGGGCGGCCAAAGGCCGCCCGA | -76.2 | 1 |
| | | AGCAATTGCTGCCTCAGTGAGCGAGCGAGCGCGC | | |
| 244 | | GCGCGCTCGCTCGCTCACTGAGGCCGGGCGACAAAATGTCGCCCGA | -72 | 1 |
| | | CACCCGGGTGGCCTCAGTGAGCGAGCGAGCGCGC | | |
| 245 | | GCGCGCTCGCTCGCTCACTGAGGCCGGGCGACAAAATGTCGCCCGA | -73.5 | 1 |
| | | CGCCATGGCGGCCTCAGTGAGCGAGCGAGCGCGC | | |
| 246 | | GCGCGCTCGCTCGCTCACTGAGGCCGGGCGACAAAATGTCGCCCGA | -71.6 | 2 |
| | | CGACATGTCGGCCTCAGTGAGCGAGCGAGCGCGC | | |
| 247 | | GCGCGCTCGCTCGCTCACTGAGGCCGGGCGACAAAATGTCGCCCGA | -72 | 2 |
| | | CGAACGTTCGGCCTCAGTGAGCGAGCGAGCGCGC | | |
| 248 | | GCGCGCTCGCTCGCTCACTGAGGCCGGGCGACAAAATGTCGCCCGA | -72.4 | 1 |
| | | AGCAATTGCTGCCTCAGTGAGCGAGCGAGCGCGC | | |
| 249 | | GCGCGCTCGCTCGCTCACTGAGGCCAAAAGACCAAAGGTCTTTTGA | -65.8 | 3 |
| | | CACCCGGGTGGCCTCAGTGAGCGAGCGAGCGCGC | | |
| 250 | | GCGCGCTCGCTCGCTCACTGAGGCCAAAAGACCAAAGGTCTTTTGA | -67.3 | 2 |
| | | CGCCATGGCGGCCTCAGTGAGCGAGCGAGCGCGC | | |
| 251 | | GCGCGCTCGCTCGCTCACTGAGGCCAAAAGACCAAAGGTCTTTTGA | -65.4 | 2 |
| | | CGACATGTCGGCCTCAGTGAGCGAGCGAGCGCGC | | |
| 252 | | GCGCGCTCGCTCGCTCACTGAGGCCAAAAGACCAAAGGTCTTTTGA | -65.8 | 2 |
| | | CGAACGTTCGGCCTCAGTGAGCGAGCGAGCGCGC | | |
| 253 | | GCGCGCTCGCTCGCTCACTGAGGCCAAAAGACCAAAGGTCTTTTGA | -66.2 | 1 |
| | | AGCAATTGCTGCCTCAGTGAGCGAGCGAGCGCGC | | |
| 254 | | GCGCGCTCGCTCGCTCACTGAGGCCAAAAAAAAAAATTTTTTTTGAC | -59.6 | 2 |
| | | ACCCGGGTGGCCTCAGTGAGCGAGCGAGCGCGC | | |
| 255 | | GCGCGCTCGCTCGCTCACTGAGGCCAAAAAAAAAAATTTTTTTTGAC | -60.4 | 1 |
| | | GCCATGGCGGCCTCAGTGAGCGAGCGAGCGCGC | | |
| 256 | | GCGCGCTCGCTCGCTCACTGAGGCCAAAAAAAAAAATTTTTTTTGAC | -59.8 | 1 |
| | | GACATGTCGGCCTCAGTGAGCGAGCGAGCGCGC | | |
| 257 | | GCGCGCTCGCTCGCTCACTGAGGCCAAAAAAAAAAATTTTTTTTGAC | -58.9 | 2 |
| | | GAACGTTCGGCCTCAGTGAGCGAGCGAGCGCGC | | |
| 258 | | GCGCGCTCGCTCGCTCACTGAGGCCAAAAAAAAAAATTTTTTTTGAA | -59.3 | 2 |
| | | GCAATTGCTGCCTCAGTGAGCGAGCGAGCGCGC | | |
| 259 | | GCGCGCTCGCTCGCTCACTGAGGCCGGGCAGAAAAATTCTGCCCGA | -70.4 | 1 |
| | | CACCCGGGTGGCCTCAGTGAGCGAGCGAGCGCGC | | |
| 260 | | GCGCGCTCGCTCGCTCACTGAGGCCGGGCAGAAAAATTCTGCCCGA | -71.9 | 1 |
| | | CGCCATGGCGGCCTCAGTGAGCGAGCGAGCGCGC | | |
| 261 | | GCGCGCTCGCTCGCTCACTGAGGCCGGGCAGAAAAATTCTGCCCGA | -70 | 1 |
| | | CGACATGTCGGCCTCAGTGAGCGAGCGAGCGCGC | | |
| 262 | | GCGCGCTCGCTCGCTCACTGAGGCCGGGCAGAAAAATTCTGCCCGA | -70.4 | 1 |
| | | CGAACGTTCGGCCTCAGTGAGCGAGCGAGCGCGC | | |
| 263 | | GCGCGCTCGCTCGCTCACTGAGGCCGGGCAGAAAAATTCTGCCCGA | -70.8 | 1 |
| | | AGCAATTGCTGCCTCAGTGAGCGAGCGAGCGCGC | | |

TABLE 4
ITR Sequences with Modifications in Loop Arm and Spacer

| SEQ | Modified | | | No. |
|---|---|---|---|---|
| ID | Region | Sequence | ΔG | Strut. |
| 264 | Loop Arm | GCGCGCTCGCTCGCTCACTAAGGCCAGGCGACCAAAGGTCGCCTGAC | −71.4 | 1 |
| | & Spacer | GCCCGGGCGGCCTTAGTGAGCGAGCGAGCGCGC | | |
| 265 | | GCGCGCTCGCTCGCTCACTAAGGCCGGGCGGCCAAAGGCCGCCCGAC | −75 | 2 |
| | | GCCCGGGCGGCCTTAGTGAGCGAGCGAGCGCGC | | |
| 266 | | GCGCGCTCGCTCGCTCACTAAGGCCGGGCGACAAAATGTCGCCCGAC | −71.2 | 1 |
| | | GCCCGGGCGGCCTTAGTGAGCGAGCGAGCGCGC | | |
| 267 | | GCGCGCTCGCTCGCTCACTAAGGCCAAAAGACCAAAGGTCTTTTGAC | −65 | 2 |
| | | GCCCGGGCGGCCTTAGTGAGCGAGCGAGCGCGC | | |
| 268 | | GCGCGCTCGCTCGCTCACTAAGGCCAAAAAAAAAAATTTTTTTTGAC | −58.1 | 1 |
| | | GCCCGGGCGGCCTTAGTGAGCGAGCGAGCGCGC | | |
| 269 | | GCGCGCTCGCTCGCTCACTAAGGCCGGGCAGAAAAATTCTGCCCGAC | −69.6 | 1 |
| | | GCCCGGGCGGCCTTAGTGAGCGAGCGAGCGCGC | | |
| 270 | | GCGCGCTCGCTCGCTCGATGGGGCCAGGCGACCAAAGGTCGCCTGAC | −72.3 | 2 |
| | | GCCCGGGCGGCCTCATCGAGCGAGCGAGCGCGC | | |
| 271 | | GCGCGCTCGCTCGCTCGATGGGGCCGGGCGGCCAAAGGCCGCCCGAC | −75.9 | 3 |
| | | GCCCGGGCGGCCTCATCGAGCGAGCGAGCGCGC | | |
| 272 | | GCGCGCTCGCTCGCTCGATGGGGCCGGGCGACAAAATGTCGCCCGAC | −72.1 | 2 |
| | | GCCCGGGCGGCCTCATCGAGCGAGCGAGCGCGC | | |
| 273 | | GCGCGCTCGCTCGCTCGATGGGGCCAAAAGACCAAAGGTCTTTTGAC | −65.9 | 3 |
| | | GCCCGGGCGGCCTCATCGAGCGAGCGAGCGCGC | | |
| 274 | | GCGCGCTCGCTCGCTCGATGGGGCCAAAAAAAAAAATTTTTTTTGAC | −59 | 2 |
| | | GCCCGGGCGGCCTCATCGAGCGAGCGAGCGCGC | | |
| 275 | | GCGCGCTCGCTCGCTCGATGGGGCCGGGCAGAAAAATTCTGCCCGAC | −70.5 | 2 |
| | | GCCCGGGCGGCCTCATCGAGCGAGCGAGCGCGC | | |
| 276 | | GCGCGCTCGCTCGCTCGAGAGAACCAGGCGACCAAAGGTCGCCTGAC | −70.9 | 1 |
| | | GCCCGGGCGGTTCTCTCGAGCGAGCGAGCGCGC | | |
| 277 | | GCGCGCTCGCTCGCTCGAGAGAACCGGGCGGCCAAAGGCCGCCCGAC | −74.5 | 1 |
| | | GCCCGGGCGGTTCTCTCGAGCGAGCGAGCGCGC | | |
| 278 | | GCGCGCTCGCTCGCTCGAGAGAACCGGGCGACAAAATGTCGCCCGAC | −70.7 | 1 |
| | | GCCCGGGCGGTTCTCTCGAGCGAGCGAGCGCGC | | |
| 279 | | GCGCGCTCGCTCGCTCGAGAGAACCAAAAGACCAAAGGTCTTTTGAC | −64.5 | 2 |
| | | GCCCGGGCGGTTCTCTCGAGCGAGCGAGCGCGC | | |
| 280 | | GCGCGCTCGCTCGCTCGAGAGAACCAAAAAAAAAAATTTTTTTTGAC | −57.6 | 1 |
| | | GCCCGGGCGGTTCTCTCGAGCGAGCGAGCGCGC | | |
| 281 | | GCGCGCTCGCTCGCTCGAGAGAACCGGGCAGAAAAATTCTGCCCGAC | −69.1 | 1 |
| | | GCCCGGGCGGTTCTCTCGAGCGAGCGAGCGCGC | | |
| 282 | | GCGCGCTCGCTCGCTCGCGGGGGCCAGGCGACCAAAGGTCGCCTGAC | −78.8 | 2 |
| | | GCCCGGGCGGCCCCCGCGAGCGAGCGAGCGCGC | | |
| 283 | | GCGCGCTCGCTCGCTCGCGGGGGCCGGGCGGCCAAAGGCCGCCCGAC | −82.4 | 3 |
| | | GCCCGGGCGGCCCCCGCGAGCGAGCGAGCGCGC | | |
| 284 | | GCGCGCTCGCTCGCTCGCGGGGGCCGGGCGACAAAATGTCGCCCGAC | −78.6 | 2 |
| | | GCCCGGGCGGCCCCCGCGAGCGAGCGAGCGCGC | | |
| 285 | | GCGCGCTCGCTCGCTCGCGGGGGCCAAAAGACCAAAGGTCTTTTGAC | −72.4 | 3 |
| | | GCCCGGGCGGCCCCCGCGAGCGAGCGAGCGCGC | | |
| 286 | | GCGCGCTCGCTCGCTCGCGGGGGCCAAAAAAAAAAATTTTTTTTGAC | −65.5 | 1 |
| | | GCCCGGGCGGCCCCCGCGAGCGAGCGAGCGCGC | | |
| 287 | | GCGCGCTCGCTCGCTCGCGGGGGCCGGGCAGAAAAATTCTGCCCGAC | −77 | 2 |
| | | GCCCGGGCGGCCCCCGCGAGCGAGCGAGCGCGC | | |
| 288 | | GCGCGCTCGCTCGCTCAATAAAACCAGGCGACCAAAGGTCGCCTGAC | −64.3 | 1 |
| | | GCCCGGGCGGTTTTATTGAGCGAGCGAGCGCGC | | |
| 289 | | GCGCGCTCGCTCGCTCAATAAAACCGGGCGGCCAAAGGCCGCCCGAC | −67.9 | 1 |
| | | GCCCGGGCGGTTTTATTGAGCGAGCGAGCGCGC | | |
| 290 | | GCGCGCTCGCTCGCTCAATAAAACCGGGCGACAAAATGTCGCCCGAC | −64.1 | 1 |
| | | GCCCGGGCGGTTTTATTGAGCGAGCGAGCGCGC | | |
| 291 | | GCGCGCTCGCTCGCTCAATAAAACCAAAAGACCAAAGGTCTTTTGAC | −57.9 | 2 |
| | | GCCCGGGCGGTTTTATTGAGCGAGCGAGCGCGC | | |
| 292 | | GCGCGCTCGCTCGCTCAATAAAACCAAAAAAAAAAATTTTTTTTGAC | −51 | 1 |
| | | GCCCGGGCGGTTTTATTGAGCGAGCGAGCGCGC | | |
| 293 | | GCGCGCTCGCTCGCTCAATAAAACCGGGCAGAAAAATTCTGCCCGAC | −62.5 | 1 |
| | | GCCCGGGCGGTTTTATTGAGCGAGCGAGCGCGC | | |

TABLE 5
ITR Sequences with Modifications in Truncated Arm and Spacer

| SEQ | Modified | | | No. |
|---|---|---|---|---|
| ID | Region | Sequence | ΔG | Strut. |
| 294 | Truncated | GCGCGCTCGCTCGCTCACTAAGGCCGGGCGACCAAAGGTCGCCCGA | −71.4 | 1 |
| | Arm & Spacer | CACCCGGGTGGCCTTAGTGAGCGAGCGAGCGCGC | | |
| 295 | | GCGCGCTCGCTCGCTCACTAAGGCCGGGCGACCAAAGGTCGCCCGA | −72.9 | 1 |
| | | CGCCATGGCGGCCTTAGTGAGCGAGCGAGCGCGC | | |
| 296 | | GCGCGCTCGCTCGCTCACTAAGGCCGGGCGACCAAAGGTCGCCCGA | −71 | 1 |
| | | CGACATGTCGGCCTTAGTGAGCGAGCGAGCGCGC | | |
| 297 | | GCGCGCTCGCTCGCTCACTAAGGCCGGGCGACCAAAGGTCGCCCGA | −71.4 | 1 |
| | | CGAACGTTCGGCCTTAGTGAGCGAGCGAGCGCGC | | |
| 298 | | GCGCGCTCGCTCGCTCACTAAGGCCGGGCGACCAAAGGTCGCCCGA | −71.8 | 1 |
| | | AGCAATTGCTGCCTTAGTGAGCGAGCGAGCGCGC | | |
| 299 | | GCGCGCTCGCTCGCTCGATGGGGCCGGGCGACCAAAGGTCGCCCGA | −72.3 | 2 |
| | | CACCCGGGTGGCCTCATCGAGCGAGCGAGCGCGC | | |
| 300 | | GCGCGCTCGCTCGCTCGATGGGGCCGGGCGACCAAAGGTCGCCCGA | −73.8 | 1 |
| | | CGCCATGGCGGCCTCATCGAGCGAGCGAGCGCGC | | |
| 301 | | GCGCGCTCGCTCGCTCGATGGGGCCGGGCGACCAAAGGTCGCCCGA | −71.9 | 1 |
| | | CGACATGTCGGCCTCATCGAGCGAGCGAGCGCGC | | |
| 302 | | GCGCGCTCGCTCGCTCGATGGGGCCGGGCGACCAAAGGTCGCCCGA | −72.3 | 1 |
| | | CGAACGTTCGGCCTCATCGAGCGAGCGAGCGCGC | | |
| 303 | | GCGCGCTCGCTCGCTCGATGGGGCCGGGCGACCAAAGGTCGCCCGA | −72.7 | 1 |
| | | AGCAATTGCTGCCTCATCGAGCGAGCGAGCGCGC | | |
| 304 | | GCGCGCTCGCTCGCTCGAGAGAACCGGGCGACCAAAGGTCGCCCGA | −70.9 | 1 |
| | | CACCCGGGTGGTTCTCTCGAGCGAGCGAGCGCGC | | |
| 305 | | GCGCGCTCGCTCGCTCGAGAGAACCGGGCGACCAAAGGTCGCCCGA | −72.4 | 1 |
| | | CGCCATGGCGGTTCTCTCGAGCGAGCGAGCGCGC | | |
| 306 | | GCGCGCTCGCTCGCTCGAGAGAACCGGGCGACCAAAGGTCGCCCGA | −70.5 | 1 |
| | | CGACATGTCGGTTCTCTCGAGCGAGCGAGCGCGC | | |
| 307 | | GCGCGCTCGCTCGCTCGAGAGAACCGGGCGACCAAAGGTCGCCCGA | −70.9 | 1 |
| | | CGAACGTTCGGTTCTCTCGAGCGAGCGAGCGCGC | | |
| 308 | | GCGCGCTCGCTCGCTCGAGAGAACCGGGCGACCAAAGGTCGCCCGA | −71.3 | 1 |
| | | AGCAATTGCTGTTCTCTCGAGCGAGCGAGCGCGC | | |
| 309 | | GCGCGCTCGCTCGCTCGCGGGGGCCGGGCGACCAAAGGTCGCCCGA | −78.8 | 1 |
| | | CACCCGGGTGGCCCCCGCGAGCGAGCGAGCGCGC | | |
| 310 | | GCGCGCTCGCTCGCTCGCGGGGGCCGGGCGACCAAAGGTCGCCCGA | −80.3 | 1 |
| | | CGCCATGGCGGCCCCCGCGAGCGAGCGAGCGCGC | | |
| 311 | | GCGCGCTCGCTCGCTCGCGGGGGCCGGGCGACCAAAGGTCGCCCGA | −78.4 | 1 |
| | | CGACATGTCGGCCCCCGCGAGCGAGCGAGCGCGC | | |
| 312 | | GCGCGCTCGCTCGCTCGCGGGGGCCGGGCGACCAAAGGTCGCCCGA | −78.8 | 1 |
| | | CGAACGTTCGGCCCCCGCGAGCGAGCGAGCGCGC | | |
| 313 | | GCGCGCTCGCTCGCTCGCGGGGGCCGGGCGACCAAAGGTCGCCCGA | −79.2 | 1 |
| | | AGCAATTGCTGCCCCCGCGAGCGAGCGAGCGCGC | | |
| 314 | | GCGCGCTCGCTCGCTCAATAAAACCGGGCGACCAAAGGTCGCCCGA | −64.3 | 1 |
| | | CACCCGGGTGGTTTTATTGAGCGAGCGAGCGCGC | | |
| 315 | | GCGCGCTCGCTCGCTCAATAAAACCGGGCGACCAAAGGTCGCCCGA | −65.8 | 1 |
| | | CGCCATGGCGGTTTTATTGAGCGAGCGAGCGCGC | | |
| 316 | | GCGCGCTCGCTCGCTCAATAAAACCGGGCGACCAAAGGTCGCCCGA | −63.9 | 1 |
| | | CGACATGTCGGTTTTATTGAGCGAGCGAGCGCGC | | |
| 317 | | GCGCGCTCGCTCGCTCAATAAAACCGGGCGACCAAAGGTCGCCCGA | −64.3 | 1 |
| | | CGAACGTTCGGTTTTATTGAGCGAGCGAGCGCGC | | |
| 318 | | GCGCGCTCGCTCGCTCAATAAAACCGGGCGACCAAAGGTCGCCCGA | −64.7 | 1 |
| | | AGCAATTGCTGTTTTATTGAGCGAGCGAGCGCGC | | |

TABLE 6
ITR Sequences with Modifications in Loop Arm, Truncated Arm and Spacer

| SEQ | Modified | | | No. |
|---|---|---|---|---|
| ID | Region | Sequence | ΔG | Strut. |
| 319 | Loop Arm, | GCGCGCTCGCTCGCTCACTAAGGCCAGGCGACCAAAGGTCGCCTGA | −69.9 | 2 |
| | Truncated | CACCCGGGTGGCCTTAGTGAGCGAGCGAGCGCGC | | |
| | Arm & Spacer | | | |
| 320 | | GCGCGCTCGCTCGCTCACTAAGGCCAGGCGACCAAAGGTCGCCTGA | −71.4 | 1 |
| | | CGCCATGGCGGCCTTAGTGAGCGAGCGAGCGCGC | | |
| 321 | | GCGCGCTCGCTCGCTCACTAAGGCCAGGCGACCAAAGGTCGCCTGA | −69.5 | 1 |
| | | CGACATGTCGGCCTTAGTGAGCGAGCGAGCGCGC | | |
| 322 | | GCGCGCTCGCTCGCTCACTAAGGCCAGGCGACCAAAGGTCGCCTGA | −69.9 | 1 |
| | | CGAACGTTCGGCCTTAGTGAGCGAGCGAGCGCGC | | |
| 323 | | GCGCGCTCGCTCGCTCACTAAGGCCAGGCGACCAAAGGTCGCCTGA | −70.3 | 1 |
| | | AGCAATTGCTGCCTTAGTGAGCGAGCGAGCGCGC | | |
| 324 | | GCGCGCTCGCTCGCTCACTAAGGCCGGGCGGCCAAAGGCCGCCCGA | −73.5 | 2 |
| | | CACCCGGGTGGCCTTAGTGAGCGAGCGAGCGCGC | | |
| 325 | | GCGCGCTCGCTCGCTCACTAAGGCCGGGCGGCCAAAGGCCGCCCGA | −75 | 1 |
| | | CGCCATGGCGGCCTTAGTGAGCGAGCGAGCGCGC | | |
| 326 | | GCGCGCTCGCTCGCTCACTAAGGCCGGGCGGCCAAAGGCCGCCCGA | −73.1 | 1 |
| | | CGACATGTCGGCCTTAGTGAGCGAGCGAGCGCGC | | |
| 327 | | GCGCGCTCGCTCGCTCACTAAGGCCGGGCGGCCAAAGGCCGCCCGA | −73.5 | 1 |
| | | CGAACGTTCGGCCTTAGTGAGCGAGCGAGCGCGC | | |
| 328 | | GCGCGCTCGCTCGCTCACTAAGGCCGGGCGGCCAAAGGCCGCCCGA | −73.9 | 1 |
| | | AGCAATTGCTGCCTTAGTGAGCGAGCGAGCGCGC | | |
| 329 | | GCGCGCTCGCTCGCTCACTAAGGCCGGGCGACAAAATGTCGCCCGA | −69.7 | 1 |
| | | CACCCGGGTGGCCTTAGTGAGCGAGCGAGCGCGC | | |
| 330 | | GCGCGCTCGCTCGCTCACTAAGGCCGGGCGACAAAATGTCGCCCGA | −71.2 | 1 |
| | | CGCCATGGCGGCCTTAGTGAGCGAGCGAGCGCGC | | |
| 331 | | GCGCGCTCGCTCGCTCACTAAGGCCGGGCGACAAAATGTCGCCCGA | −69.3 | 2 |
| | | CGACATGTCGGCCTTAGTGAGCGAGCGAGCGCGC | | |
| 332 | | GCGCGCTCGCTCGCTCACTAAGGCCGGGCGACAAAATGTCGCCCGA | −69.7 | 2 |
| | | CGAACGTTCGGCCTTAGTGAGCGAGCGAGCGCGC | | |
| 333 | | GCGCGCTCGCTCGCTCACTAAGGCCGGGCGACAAAATGTCGCCCGA | −70.1 | 1 |
| | | AGCAATTGCTGCCTTAGTGAGCGAGCGAGCGCGC | | |
| 334 | | GCGCGCTCGCTCGCTCACTAAGGCCAAAAGACCAAAGGTCTTTTGAC | −63.5 | 2 |
| | | ACCCGGGTGGCCTTAGTGAGCGAGCGAGCGCGC | | |
| 335 | | GCGCGCTCGCTCGCTCACTAAGGCCAAAAGACCAAAGGTCTTTTGAC | −65 | 2 |
| | | GCCATGGCGGCCTTAGTGAGCGAGCGAGCGCGC | | |
| 336 | | GCGCGCTCGCTCGCTCACTAAGGCCAAAAGACCAAAGGTCTTTTGAC | −63.1 | 2 |
| | | GACATGTCGGCCTTAGTGAGCGAGCGAGCGCGC | | |
| 337 | | GCGCGCTCGCTCGCTCACTAAGGCCAAAAGACCAAAGGTCTTTTGAC | −63.5 | 2 |
| | | GAACGTTCGGCCTTAGTGAGCGAGCGAGCGCGC | | |
| 338 | | GCGCGCTCGCTCGCTCACTAAGGCCAAAAGACCAAAGGTCTTTTGA | −63.9 | 1 |
| | | AGCAATTGCTGCCTTAGTGAGCGAGCGAGCGCGC | | |
| 339 | | GCGCGCTCGCTCGCTCACTAAGGCCAAAAAAAAAAATTTTTTTTGAC | −57.3 | 2 |
| | | ACCCGGGTGGCCTTAGTGAGCGAGCGAGCGCGC | | |
| 340 | | GCGCGCTCGCTCGCTCACTAAGGCCAAAAAAAAAAATTTTTTTTGAC | −58.1 | 1 |
| | | GCCATGGCGGCCTTAGTGAGCGAGCGAGCGCGC | | |
| 341 | | GCGCGCTCGCTCGCTCACTAAGGCCAAAAAAAAAAATTTTTTTTGAC | −57.5 | 1 |
| | | GACATGTCGGCCTTAGTGAGCGAGCGAGCGCGC | | |
| 342 | | GCGCGCTCGCTCGCTCACTAAGGCCAAAAAAAAAAATTTTTTTTGAC | −56.6 | 2 |
| | | GAACGTTCGGCCTTAGTGAGCGAGCGAGCGCGC | | |
| 343 | | GCGCGCTCGCTCGCTCACTAAGGCCAAAAAAAAAAATTTTTTTTGAA | −57 | 2 |
| | | GCAATTGCTGCCTTAGTGAGCGAGCGAGCGCGC | | |
| 344 | | GCGCGCTCGCTCGCTCACTAAGGCCGGGCAGAAAAATTCTGCCCGA | −68.1 | 1 |
| | | CACCCGGGTGGCCTTAGTGAGCGAGCGAGCGCGC | | |
| 345 | | GCGCGCTCGCTCGCTCACTAAGGCCGGGCAGAAAAATTCTGCCCGA | −69.6 | 1 |
| | | CGCCATGGCGGCCTTAGTGAGCGAGCGAGCGCGC | | |
| 346 | | GCGCGCTCGCTCGCTCACTAAGGCCGGGCAGAAAAATTCTGCCCGA | −67.7 | 1 |
| | | CGACATGTCGGCCTTAGTGAGCGAGCGAGCGCGC | | |
| 347 | | GCGCGCTCGCTCGCTCACTAAGGCCGGGCAGAAAAATTCTGCCCGA | −68.1 | 1 |
| | | CGAACGTTCGGCCTTAGTGAGCGAGCGAGCGCGC | | |
| 348 | | GCGCGCTCGCTCGCTCACTAAGGCCGGGCAGAAAAATTCTGCCCGA | −68.5 | 1 |
| | | AGCAATTGCTGCCTTAGTGAGCGAGCGAGCGCGC | | |
| 349 | | GCGCGCTCGCTCGCTCGATGGGGCCAGGCGACCAAAGGTCGCCTGA | −70.8 | 3 |
| | | CACCCGGGTGGCCTCATCGAGCGAGCGAGCGCGC | | |
| 350 | | GCGCGCTCGCTCGCTCGATGGGGCCAGGCGACCAAAGGTCGCCTGA | −72.3 | 1 |
| | | CGCCATGGCGGCCTCATCGAGCGAGCGAGCGCGC | | |
| 351 | | GCGCGCTCGCTCGCTCGATGGGGCCAGGCGACCAAAGGTCGCCTGA | −70.4 | 1 |
| | | CGACATGTCGGCCTCATCGAGCGAGCGAGCGCGC | | |
| 352 | | GCGCGCTCGCTCGCTCGATGGGGCCAGGCGACCAAAGGTCGCCTGA | −70.8 | 1 |
| | | CGAACGTTCGGCCTCATCGAGCGAGCGAGCGCGC | | |
| 353 | | GCGCGCTCGCTCGCTCGATGGGGCCAGGCGACCAAAGGTCGCCTGA | −71.2 | 1 |
| | | AGCAATTGCTGCCTCATCGAGCGAGCGAGCGCGC | | |
| 354 | | GCGCGCTCGCTCGCTCGATGGGGCCGGGCGGCCAAAGGCCGCCCG | −74.4 | 3 |
| | | ACACCCGGGTGGCCTCATCGAGCGAGCGAGCGCGC | | |
| 355 | | GCGCGCTCGCTCGCTCGATGGGGCCGGGCGGCCAAAGGCCGCCCG | −75.9 | 1 |
| | | ACGCCATGGCGGCCTCATCGAGCGAGCGAGCGCGC | | |
| 356 | | GCGCGCTCGCTCGCTCGATGGGGCCGGGCGGCCAAAGGCCGCCCG | −74 | 1 |
| | | ACGACATGTCGGCCTCATCGAGCGAGCGAGCGCGC | | |
| 357 | | GCGCGCTCGCTCGCTCGATGGGGCCGGGCGGCCAAAGGCCGCCCG | −74.4 | 1 |
| | | ACGAACGTTCGGCCTCATCGAGCGAGCGAGCGCGC | | |
| 358 | | GCGCGCTCGCTCGCTCGATGGGGCCGGGCGGCCAAAGGCCGCCCG | −74.8 | 1 |
| | | AAGCAATTGCTGCCTCATCGAGCGAGCGAGCGCGC | | |
| 359 | | GCGCGCTCGCTCGCTCGATGGGGCCGGGCGACAAAATGTCGCCCGA | −70.6 | 2 |
| | | CACCCGGGTGGCCTCATCGAGCGAGCGAGCGCGC | | |
| 360 | | GCGCGCTCGCTCGCTCGATGGGGCCGGGCGACAAAATGTCGCCCGA | −72.1 | 1 |
| | | CGCCATGGCGGCCTCATCGAGCGAGCGAGCGCGC | | |
| 361 | | GCGCGCTCGCTCGCTCGATGGGGCCGGGCGACAAAATGTCGCCCGA | −70.2 | 2 |
| | | CGACATGTCGGCCTCATCGAGCGAGCGAGCGCGC | | |
| 362 | | GCGCGCTCGCTCGCTCGATGGGGCCGGGCGACAAAATGTCGCCCGA | −70.6 | 2 |
| | | CGAACGTTCGGCCTCATCGAGCGAGCGAGCGCGC | | |
| 363 | | GCGCGCTCGCTCGCTCGATGGGGCCGGGCGACAAAATGTCGCCCGA | −71 | 1 |
| | | AGCAATTGCTGCCTCATCGAGCGAGCGAGCGCGC | | |
| 364 | | GCGCGCTCGCTCGCTCGATGGGGCCAAAAGACCAAAGGTCTTTTGA | −64.4 | 3 |
| | | CACCCGGGTGGCCTCATCGAGCGAGCGAGCGCGC | | |
| 365 | | GCGCGCTCGCTCGCTCGATGGGGCCAAAAGACCAAAGGTCTTTTGA | −65.9 | 2 |
| | | CGCCATGGCGGCCTCATCGAGCGAGCGAGCGCGC | | |
| 366 | | GCGCGCTCGCTCGCTCGATGGGGCCAAAAGACCAAAGGTCTTTTGA | −64 | 2 |
| | | CGACATGTCGGCCTCATCGAGCGAGCGAGCGCGC | | |
| 367 | | GCGCGCTCGCTCGCTCGATGGGGCCAAAAGACCAAAGGTCTTTTGA | −64.4 | 2 |
| | | CGAACGTTCGGCCTCATCGAGCGAGCGAGCGCGC | | |
| 368 | | GCGCGCTCGCTCGCTCGATGGGGCCAAAAGACCAAAGGTCTTTTGA | −64.8 | 1 |
| | | AGCAATTGCTGCCTCATCGAGCGAGCGAGCGCGC | | |
| 369 | | GCGCGCTCGCTCGCTCGATGGGGCCAAAAAAAAAAATTTTTTTTGA | −58.2 | 2* |
| | | CACCCGGGTGGCCTCATCGAGCGAGCGAGCGCGC | | |
| 370 | | GCGCGCTCGCTCGCTCGATGGGGCCAAAAAAAAAAATTTTTTTTGA | −59 | 1 |
| | | CGCCATGGCGGCCTCATCGAGCGAGCGAGCGCGC | | |
| 371 | | GCGCGCTCGCTCGCTCGATGGGGCCAAAAAAAAAAATTTTTTTTGA | −58.4 | 1 |
| | | CGACATGTCGGCCTCATCGAGCGAGCGAGCGCGC | | |
| 372 | | GCGCGCTCGCTCGCTCGATGGGGCCAAAAAAAAAAATTTTTTTTGA | −57.5 | 2 |
| | | CGAACGTTCGGCCTCATCGAGCGAGCGAGCGCGC | | |
| 373 | | GCGCGCTCGCTCGCTCGATGGGGCCAAAAAAAAAAATTTTTTTTGA | −57.9 | 2 |
| | | AGCAATTGCTGCCTCATCGAGCGAGCGAGCGCGC | | |
| 374 | | GCGCGCTCGCTCGCTCGATGGGGCCGGGCAGAAAAATTCTGCCCGA | −69 | 2 |
| | | CACCCGGGTGGCCTCATCGAGCGAGCGAGCGCGC | | |
| 375 | | GCGCGCTCGCTCGCTCGATGGGGCCGGGCAGAAAAATTCTGCCCGA | −70.5 | 1 |
| | | CGCCATGGCGGCCTCATCGAGCGAGCGAGCGCGC | | |
| 376 | | GCGCGCTCGCTCGCTCGATGGGGCCGGGCAGAAAAATTCTGCCCGA | −68.6 | 1 |
| | | CGACATGTCGGCCTCATCGAGCGAGCGAGCGCGC | | |
| 377 | | GCGCGCTCGCTCGCTCGATGGGGCCGGGCAGAAAAATTCTGCCCGA | −69 | 1 |
| | | CGAACGTTCGGCCTCATCGAGCGAGCGAGCGCGC | | |
| 378 | | GCGCGCTCGCTCGCTCGATGGGGCCGGGCAGAAAAATTCTGCCCGA | −69.4 | 1 |
| | | AGCAATTGCTGCCTCATCGAGCGAGCGAGCGCGC | | |
| 379 | | GCGCGCTCGCTCGCTCGAGAGAACCAGGCGACCAAAGGTCGCCTGA | −69.4 | 2 |
| | | CACCCGGGTGGTTCTCTCGAGCGAGCGAGCGCGC | | |
| 380 | | GCGCGCTCGCTCGCTCGAGAGAACCAGGCGACCAAAGGTCGCCTGA | −70.9 | 1 |
| | | CGCCATGGCGGTTCTCTCGAGCGAGCGAGCGCGC | | |
| 381 | | GCGCGCTCGCTCGCTCGAGAGAACCAGGCGACCAAAGGTCGCCTGA | −69 | 1 |
| | | CGACATGTCGGTTCTCTCGAGCGAGCGAGCGCGC | | |
| 382 | | GCGCGCTCGCTCGCTCGAGAGAACCAGGCGACCAAAGGTCGCCTGA | −69.4 | 1 |
| | | CGAACGTTCGGTTCTCTCGAGCGAGCGAGCGCGC | | |
| 383 | | GCGCGCTCGCTCGCTCGAGAGAACCAGGCGACCAAAGGTCGCCTGA | −69.8 | 1 |
| | | AGCAATTGCTGTTCTCTCGAGCGAGCGAGCGCGC | | |
| 384 | | GCGCGCTCGCTCGCTCGAGAGAACCGGGCGGCCAAAGGCCGCCCG | −73 | 1 |
| | | ACACCCGGGTGGTTCTCTCGAGCGAGCGAGCGCGC | | |
| 385 | | GCGCGCTCGCTCGCTCGAGAGAACCGGGCGGCCAAAGGCCGCCCG | −74.5 | 1 |
| | | ACGCCATGGCGGTTCTCTCGAGCGAGCGAGCGCGC | | |
| 386 | | GCGCGCTCGCTCGCTCGAGAGAACCGGGCGGCCAAAGGCCGCCCG | −72.6 | 1 |
| | | ACGACATGTCGGTTCTCTCGAGCGAGCGAGCGCGC | | |
| 387 | | GCGCGCTCGCTCGCTCGAGAGAACCGGGCGGCCAAAGGCCGCCCG | −73 | 1 |
| | | ACGAACGTTCGGTTCTCTCGAGCGAGCGAGCGCGC | | |
| 388 | | GCGCGCTCGCTCGCTCGAGAGAACCGGGCGGCCAAAGGCCGCCCG | −73.4 | 1 |
| | | AAGCAATTGCTGTTCTCTCGAGCGAGCGAGCGCGC | | |
| 389 | | GCGCGCTCGCTCGCTCGAGAGAACCGGGCGACAAAATGTCGCCCGA | −69.2 | 1 |
| | | CACCCGGGTGGTTCTCTCGAGCGAGCGAGCGCGC | | |
| 390 | | GCGCGCTCGCTCGCTCGAGAGAACCGGGCGACAAAATGTCGCCCGA | −70.7 | 1 |
| | | CGCCATGGCGGTTCTCTCGAGCGAGCGAGCGCGC | | |
| 391 | | GCGCGCTCGCTCGCTCGAGAGAACCGGGCGACAAAATGTCGCCCGA | −69.8 | 2 |
| | | CGACATGTCGGTTCTCTCGAGCGAGCGAGCGCGC | | |
| 392 | | GCGCGCTCGCTCGCTCGAGAGAACCGGGCGACAAAATGTCGCCCGA | −69.2 | 2 |
| | | CGAACGTTCGGTTCTCTCGAGCGAGCGAGCGCGC | | |
| 393 | | GCGCGCTCGCTCGCTCGAGAGAACCGGGCGACAAAATGTCGCCCGA | −69.6 | 1 |
| | | AGCAATTGCTGTTCTCTCGAGCGAGCGAGCGCGC | | |
| 394 | | GCGCGCTCGCTCGCTCGAGAGAACCAAAAGACCAAAGGTCTTTTGA | −63 | 2 |
| | | CACCCGGGTGGTTCTCTCGAGCGAGCGAGCGCGC | | |
| 395 | | GCGCGCTCGCTCGCTCGAGAGAACCAAAAGACCAAAGGTCTTTTGA | −64.5 | 2 |
| | | CGCCATGGCGGTTCTCTCGAGCGAGCGAGCGCGC | | |
| 396 | | GCGCGCTCGCTCGCTCGAGAGAACCAAAAGACCAAAGGTCTTTTGA | −62.6 | 2 |
| | | CGACATGTCGGTTCTCTCGAGCGAGCGAGCGCGC | | |
| 397 | | GCGCGCTCGCTCGCTCGAGAGAACCAAAAGACCAAAGGTCTTTTGA | −63 | 2 |
| | | CGAACGTTCGGTTCTCTCGAGCGAGCGAGCGCGC | | |
| 398 | | GCGCGCTCGCTCGCTCGAGAGAACCAAAAGACCAAAGGTCTTTTGA | −63.4 | 1 |
| | | AGCAATTGCTGTTCTCTCGAGCGAGCGAGCGCGC | | |
| 399 | | GCGCGCTCGCTCGCTCGAGAGAACCAAAAAAAAAAATTTTTTTTGA | −56.8 | 2 |
| | | CACCCGGGTGGTTCTCTCGAGCGAGCGAGCGCGC | | |
| 400 | | GCGCGCTCGCTCGCTCGAGAGAACCAAAAAAAAAAATTTTTTTTGA | −57.6 | 1 |
| | | CGCCATGGCGGTTCTCTCGAGCGAGCGAGCGCGC | | |
| 401 | | GCGCGCTCGCTCGCTCGAGAGAACCAAAAAAAAAAATTTTTTTTGA | −57 | 1 |
| | | CGACATGTCGGTTCTCTCGAGCGAGCGAGCGCGC | | |
| 402 | | GCGCGCTCGCTCGCTCGAGAGAACCAAAAAAAAAAATTTTTTTTGA | −56.1 | 2 |
| | | CGAACGTTCGGTTCTCTCGAGCGAGCGAGCGCGC | | |
| 403 | | GCGCGCTCGCTCGCTCGAGAGAACCAAAAAAAAAAATTTTTTTTGA | −56.5 | 2 |
| | | AGCAATTGCTGTTCTCTCGAGCGAGCGAGCGCGC | | |
| 404 | | GCGCGCTCGCTCGCTCGAGAGAACCGGGCAGAAAAATTCTGCCCGA | −67.6 | 1 |
| | | CACCCGGGTGGTTCTCTCGAGCGAGCGAGCGCGC | | |
| 405 | | GCGCGCTCGCTCGCTCGAGAGAACCGGGCAGAAAAATTCTGCCCGA | −69.1 | 1 |
| | | CGCCATGGCGGTTCTCTCGAGCGAGCGAGCGCGC | | |
| 406 | | GCGCGCTCGCTCGCTCGAGAGAACCGGGCAGAAAAATTCTGCCCGA | −67.2 | 1 |
| | | CGACATGTCGGTTCTCTCGAGCGAGCGAGCGCGC | | |
| 407 | | GCGCGCTCGCTCGCTCGAGAGAACCGGGCAGAAAAATTCTGCCCGA | −67.6 | 1 |
| | | CGAACGTTCGGTTCTCTCGAGCGAGCGAGCGCGC | | |
| 408 | | GCGCGCTCGCTCGCTCGAGAGAACCGGGCAGAAAAATTCTGCCCGA | −68 | 1 |
| | | AGCAATTGCTGTTCTCTCGAGCGAGCGAGCGCGC | | |
| 409 | | GCGCGCTCGCTCGCTCGCGGGGGCCAGGCGACCAAAGGTCGCCTGA | −77.3 | 2 |
| | | CACCCGGGTGGCCCCCGCGAGCGAGCGAGCGCGC | | |
| 410 | | GCGCGCTCGCTCGCTCGCGGGGGCCAGGCGACCAAAGGTCGCCTGA | −78.8 | 1 |
| | | CGCCATGGCGGCCCCCGCGAGCGAGCGAGCGCGC | | |
| 411 | | GCGCGCTCGCTCGCTCGCGGGGGCCAGGCGACCAAAGGTCGCCTGA | −76.9 | 1 |
| | | CGACATGTCGGCCCCCGCGAGCGAGCGAGCGCGC | | |
| 412 | | GCGCGCTCGCTCGCTCGCGGGGGCCAGGCGACCAAAGGTCGCCTGA | −77.3 | 1 |
| | | CGAACGTTCGGCCCCCGCGAGCGAGCGAGCGCGC | | |
| 413 | | GCGCGCTCGCTCGCTCGCGGGGGCCAGGCGACCAAAGGTCGCCTGA | −77.7 | 1 |
| | | AGCAATTGCTGCCCCCGCGAGCGAGCGAGCGCGC | | |
| 414 | | GCGCGCTCGCTCGCTCGCGGGGGCCGGGCGGCCAAAGGCCGCCCG | −80.9 | 2 |
| | | ACACCCGGGTGGCCCCCGCGAGCGAGCGAGCGCGC | | |
| 415 | | GCGCGCTCGCTCGCTCGCGGGGGCCGGGCGGCCAAAGGCCGCCCG | −82.4 | 1 |
| | | ACGCCATGGCGGCCCCCGCGAGCGAGCGAGCGCGC | | |
| 416 | | GCGCGCTCGCTCGCTCGCGGGGGCCGGGCGGCCAAAGGCCGCCCG | −80.5 | 1 |
| | | ACGACATGTCGGCCCCCGCGAGCGAGCGAGCGCGC | | |
| 417 | | GCGCGCTCGCTCGCTCGCGGGGGCCGGGCGGCCAAAGGCCGCCCG | −80.9 | 1 |
| | | ACGAACGTTCGGCCCCCGCGAGCGAGCGAGCGCGC | | |
| 418 | | GCGCGCTCGCTCGCTCGCGGGGGCCGGGCGGCCAAAGGCCGCCCG | −81.3 | 1 |
| | | AAGCAATTGCTGCCCCCGCGAGCGAGCGAGCGCGC | | |
| 419 | | GCGCGCTCGCTCGCTCGCGGGGGCCGGGCGACAAAATGTCGCCCGA | −77.1 | 1 |
| | | CACCCGGGTGGCCCCCGCGAGCGAGCGAGCGCGC | | |
| 420 | | GCGCGCTCGCTCGCTCGCGGGGGCCGGGCGACAAAATGTCGCCCGA | −78.6 | 1 |
| | | CGCCATGGCGGCCCCCGCGAGCGAGCGAGCGCGC | | |
| 421 | | GCGCGCTCGCTCGCTCGCGGGGGCCGGGCGACAAAATGTCGCCCGA | −76.7 | 2 |
| | | CGACATGTCGGCCCCCGCGAGCGAGCGAGCGCGC | | |
| 422 | | GCGCGCTCGCTCGCTCGCGGGGGCCGGGCGACAAAATGTCGCCCGA | −77.1 | 2 |
| | | CGAACGTTCGGCCCCCGCGAGCGAGCGAGCGCGC | | |
| 423 | | GCGCGCTCGCTCGCTCGCGGGGGCCGGGCGACAAAATGTCGCCCGA | −77.5 | 1 |
| | | AGCAATTGCTGCCCCCGCGAGCGAGCGAGCGCGC | | |
| 424 | | GCGCGCTCGCTCGCTCGCGGGGGCCAAAAGACCAAAGGTCTTTTGA | −70.9 | 3 |
| | | CACCCGGGTGGCCCCCGCGAGCGAGCGAGCGCGC | | |
| 425 | | GCGCGCTCGCTCGCTCGCGGGGGCCAAAAGACCAAAGGTCTTTTGA | −72.4 | 2 |
| | | CGCCATGGCGGCCCCCGCGAGCGAGCGAGCGCGC | | |
| 426 | | GCGCGCTCGCTCGCTCGCGGGGGCCAAAAGACCAAAGGTCTTTTGA | −70.5 | 2 |
| | | CGACATGTCGGCCCCCGCGAGCGAGCGAGCGCGC | | |
| 427 | | GCGCGCTCGCTCGCTCGCGGGGGCCAAAAGACCAAAGGTCTTTTGA | −70.9 | 2 |
| | | CGAACGTTCGGCCCCCGCGAGCGAGCGAGCGCGC | | |
| 428 | | GCGCGCTCGCTCGCTCGCGGGGGCCAAAAGACCAAAGGTCTTTTGA | −71.3 | 1 |
| | | AGCAATTGCTGCCCCCGCGAGCGAGCGAGCGCGC | | |
| 429 | | GCGCGCTCGCTCGCTCGCGGGGGCCAAAAAAAAAAATTTTTTTTGA | −64.7 | 2 |
| | | CACCCGGGTGGCCCCCGCGAGCGAGCGAGCGCGC | | |
| 430 | | GCGCGCTCGCTCGCTCGCGGGGGCCAAAAAAAAAAATTTTTTTTGA | −65.5 | 1 |
| | | CGCCATGGCGGCCCCCGCGAGCGAGCGAGCGCGC | | |
| 431 | | GCGCGCTCGCTCGCTCGCGGGGGCCAAAAAAAAAAATTTTTTTTGA | −64.9 | 1 |
| | | CGACATGTCGGCCCCCGCGAGCGAGCGAGCGCGC | | |
| 432 | | GCGCGCTCGCTCGCTCGCGGGGGCCAAAAAAAAAAATTTTTTTTGA | −64 | 2 |
| | | CGAACGTTCGGCCCCCGCGAGCGAGCGAGCGCGC | | |
| 433 | | GCGCGCTCGCTCGCTCGCGGGGGCCAAAAAAAAAAATTTTTTTTGA | −64.4 | 2 |
| | | AGCAATTGCTGCCCCCGCGAGCGAGCGAGCGCGC | | |
| 434 | | GCGCGCTCGCTCGCTCGCGGGGGCCGGGCAGAAAAATTCTGCCCGA | −75.5 | 1 |
| | | CACCCGGGTGGCCCCCGCGAGCGAGCGAGCGCGC | | |
| 435 | | GCGCGCTCGCTCGCTCGCGGGGGCCGGGCAGAAAAATTCTGCCCGA | −77 | 1 |
| | | CGCCATGGCGGCCCCCGCGAGCGAGCGAGCGCGC | | |
| 436 | | GCGCGCTCGCTCGCTCGCGGGGGCCGGGCAGAAAAATTCTGCCCGA | −75.1 | 1 |
| | | CGACATGTCGGCCCCCGCGAGCGAGCGAGCGCGC | | |
| 437 | | GCGCGCTCGCTCGCTCGCGGGGGCCGGGCAGAAAAATTCTGCCCGA | −75.5 | 1 |
| | | CGAACGTTCGGCCCCCGCGAGCGAGCGAGCGCGC | | |
| 438 | | GCGCGCTCGCTCGCTCGCGGGGGCCGGGCAGAAAAATTCTGCCCGA | −75.9 | 1 |
| | | AGCAATTGCTGCCCCCGCGAGCGAGCGAGCGCGC | | |
| 439 | | GCGCGCTCGCTCGCTCAATAAAACCAGGCGACCAAAGGTCGCCTGA | −62.8 | 2 |
| | | CACCCGGGTGGTTTTATTGAGCGAGCGAGCGCGC | | |
| 440 | | GCGCGCTCGCTCGCTCAATAAAACCAGGCGACCAAAGGTCGCCTGA | −64.3 | 1 |
| | | CGCCATGGCGGTTTTATTGAGCGAGCGAGCGCGC | | |
| 441 | | GCGCGCTCGCTCGCTCAATAAAACCAGGCGACCAAAGGTCGCCTGA | −62.4 | 1 |
| | | CGACATGTCGGTTTTATTGAGCGAGCGAGCGCGC | | |
| 442 | | GCGCGCTCGCTCGCTCAATAAAACCAGGCGACCAAAGGTCGCCTGA | −62.8 | 1 |
| | | CGAACGTTCGGTTTTATTGAGCGAGCGAGCGCGC | | |
| 443 | | GCGCGCTCGCTCGCTCAATAAAACCAGGCGACCAAAGGTCGCCTGA | −63.2 | 1 |
| | | AGCAATTGCTGTTTTATTGAGCGAGCGAGCGCGC | | |
| 444 | | GCGCGCTCGCTCGCTCAATAAAACCGGGCGGCCAAAGGCCGCCCGA | −66.4 | 1 |
| | | CACCCGGGTGGTTTTATTGAGCGAGCGAGCGCGC | | |
| 445 | | GCGCGCTCGCTCGCTCAATAAAACCGGGCGGCCAAAGGCCGCCCGA | −67.9 | 1 |
| | | CGCCATGGCGGTTTTATTGAGCGAGCGAGCGCGC | | |
| 446 | | GCGCGCTCGCTCGCTCAATAAAACCGGGCGGCCAAAGGCCGCCCGA | −66 | 1 |
| | | CGACATGTCGGTTTTATTGAGCGAGCGAGCGCGC | | |
| 447 | | GCGCGCTCGCTCGCTCAATAAAACCGGGCGGCCAAAGGCCGCCCGA | −66.4 | 1 |
| | | CGAACGTTCGGTTTTATTGAGCGAGCGAGCGCGC | | |
| 448 | | GCGCGCTCGCTCGCTCAATAAAACCGGGCGGCCAAAGGCCGCCCGA | −66.8 | 1 |
| | | AGCAATTGCTGTTTTATTGAGCGAGCGAGCGCGC | | |
| 449 | | GCGCGCTCGCTCGCTCAATAAAACCGGGCGACAAAATGTCGCCCGA | −62.6 | 1 |
| | | CACCCGGGTGGTTTTATTGAGCGAGCGAGCGCGC | | |
| 450 | | GCGCGCTCGCTCGCTCAATAAAACCGGGCGACAAAATGTCGCCCGA | −64.1 | 1 |
| | | CGCCATGGCGGTTTTATTGAGCGAGCGAGCGCGC | | |
| 451 | | GCGCGCTCGCTCGCTCAATAAAACCGGGCGACAAAATGTCGCCCGA | −62.2 | 2 |
| | | CGACATGTCGGTTTTATTGAGCGAGCGAGCGCGC | | |
| 452 | | GCGCGCTCGCTCGCTCAATAAAACCGGGCGACAAAATGTCGCCCGA | −62.6 | 2 |
| | | CGAACGTTCGGTTTTATTGAGCGAGCGAGCGCGC | | |
| 453 | | GCGCGCTCGCTCGCTCAATAAAACCGGGCGACAAAATGTCGCCCGA | −63 | 1 |
| | | AGCAATTGCTGTTTTATTGAGCGAGCGAGCGCGC | | |
| 454 | | GCGCGCTCGCTCGCTCAATAAAACCAAAAGACCAAAGGTCTTTTGA | −56.4 | 2 |
| | | CACCCGGGTGGTTTTATTGAGCGAGCGAGCGCGC | | |
| 455 | | GCGCGCTCGCTCGCTCAATAAAACCAAAAGACCAAAGGTCTTTTGA | −57.9 | 2 |
| | | CGCCATGGCGGTTTTATTGAGCGAGCGAGCGCGC | | |
| 456 | | GCGCGCTCGCTCGCTCAATAAAACCAAAAGACCAAAGGTCTTTTGA | −56 | 2 |
| | | CGACATGTCGGTTTTATTGAGCGAGCGAGCGCGC | | |
| 457 | | GCGCGCTCGCTCGCTCAATAAAACCAAAAGACCAAAGGTCTTTTGA | −56.4 | 2 |
| | | CGAACGTTCGGTTTTATTGAGCGAGCGAGCGCGC | | |
| 458 | | GCGCGCTCGCTCGCTCAATAAAACCAAAAGACCAAAGGTCTTTTGA | −56.8 | 1 |
| | | AGCAATTGCTGTTTTATTGAGCGAGCGAGCGCGC | | |
| 459 | | GCGCGCTCGCTCGCTCAATAAAACCAAAAAAAAAAATTTTTTTTGAC | −50.2 | 2 |
| | | ACCCGGGTGGTTTTATTGAGCGAGCGAGCGCGC | | |
| 460 | | GCGCGCTCGCTCGCTCAATAAAACCAAAAAAAAAAATTTTTTTTGAC | −51 | 1 |
| | | GCCATGGCGGTTTTATTGAGCGAGCGAGCGCGC | | |
| 461 | | GCGCGCTCGCTCGCTCAATAAAACCAAAAAAAAAAATTTTTTTTGAC | −50.4 | 1 |
| | | GACATGTCGGTTTTATTGAGCGAGCGAGCGCGC | | |
| 462 | | GCGCGCTCGCTCGCTCAATAAAACCAAAAAAAAAAATTTTTTTTGAC | −49.5 | 2 |
| | | GAACGTTCGGTTTTATTGAGCGAGCGAGCGCGC | | |
| 463 | | GCGCGCTCGCTCGCTCAATAAAACCAAAAAAAAAAATTTTTTTTGAA | −49.9 | 1 |
| | | GCAATTGCTGTTTTATTGAGCGAGCGAGCGCGC | | |
| 464 | | GCGCGCTCGCTCGCTCAATAAAACCGGGCAGAAAAATTCTGCCCGA | −61 | 1 |
| | | CACCCGGGTGGTTTTATTGAGCGAGCGAGCGCGC | | |
| 465 | | GCGCGCTCGCTCGCTCAATAAAACCGGGCAGAAAAATTCTGCCCGA | −62.5 | 1 |
| | | CGCCATGGCGGTTTTATTGAGCGAGCGAGCGCGC | | |
| 466 | | GCGCGCTCGCTCGCTCAATAAAACCGGGCAGAAAAATTCTGCCCGA | −60.6 | 1 |
| | | CGACATGTCGGTTTTATTGAGCGAGCGAGCGCGC | | |
| 467 | | GCGCGCTCGCTCGCTCAATAAAACCGGGCAGAAAAATTCTGCCCGA | −61 | 1 |
| | | CGAACGTTCGGTTTTATTGAGCGAGCGAGCGCGC | | |
| 468 | | GCGCGCTCGCTCGCTCAATAAAACCGGGCAGAAAAATTCTGCCCGA | −61.4 | 1 |
| | | AGCAATTGCTGTTTTATTGAGCGAGCGAGCGCGC | | |

As disclosed herein, the modified ITR can be generated to include deletion, insertion, or substitution of one or more nucleotides from the wild-type ITR derived from AAV genome. The modified ITR can be generated by genetic modification during propagation in a plasmid in Escherichia coli or as a baculovirus genome in Spodoptera frugiperda cells, or other biological methods, for example in vitro using polymerase chain reaction, or chemical synthesis.

In some embodiments, the modified ITR include deletion, insertion, or substitution of one or more nucleotides from the wild-type ITR of AAV2 (Left) (SEQ ID NO: 51) or the wild-type ITR of AAV2 (Right) (SEQ ID NO: 1). Specifically, one or more nucleotides are deleted, inserted, or substituted from B-C′ or C-C′ of the T-shaped stem-loop structure. Furthermore, the modified ITR includes no modification in the Rep-binding elements (RBE) and the terminal resolution site (trs) of wild-type ITR of AAV2, although the RBE′(TTT) may be or may not be present depending on the whether the template has undergone one round of replication thereby converting the AAA triplet to the complimentary RBE′-TTT.

Three types of modified ITRs are exemplified—(1) a modified ITR having a lowest energy structure comprising a single arm and a single unpaired loop (“single-arm/single-unpaired-loop structure”); (2) a modified ITR having a lowest energy structure with a single hairpin (“single-hairpin structure”); and (3) a modified ITR having a lowest energy structure with two arms, one of which is truncated (“truncated structure”).

Modified ITR with a Single-Arm/Single-Unpaired-Loop Structure

The wild-type ITR can be modified to form a secondary structure comprising a single arm and a single unpaired loop (i.e., “single-arm/single-unpaired-loop structure”). Gibbs free energy (ΔG) of unfolding of the structure can range between −85 kcal/mol and −70 kcal/mol. Exemplary structures of the modified ITRs are provided.

Modified ITRs predicted to form the single-arm/single-unpaired-loop structure can include deletion, insertion, or substitution of one or more nucleotides from the wild-type ITR in the sequences forming B and B′ arm and/or C and C′ arm. Modified ITR can be generated by genetic modification or biological and/or chemical synthesis.

For example, ITR-2, Left and Right provided in FIGS. 9A-9B (SEQ ID NOS:101 and 102), are generated to have deletion of two nucleotides from C-C′ arm and deletion of 16 nucleotides from B-B′ arm in the wild-type ITR of AAV2. Three nucleotides remaining in the B-B′ arm of the modified ITR do not make a complementary pairing. Thus, ITR-2 Left and Right have the lowest energy structure with a single C-C′ arm and a single unpaired loop. Gibbs free energy of unfolding the structure is predicted to be about −72.6 kcal/mol.

ITR-3 Left and Right provided in FIGS. 10A and 10B (SEQ ID NOS: 103 and 104), are generated to include 19 nucleotide deletions in C-C′ arm from the wild-type ITR of AAV2. Three nucleotides remaining in the B-B′ arm of the modified ITR do not make a complementary pairing. Thus, ITR-3 Left and Right have the lowest energy structure with a single B-B′ arm and a single unpaired loop. Gibbs free energy of unfolding the structure is predicted to be about −74.8 kcal/mol.

ITR-4 Left and Right provided in FIGS. 11A and 11B (SEQ ID NOS: 105 and 106), are generated to include 19 nucleotide deletions in B-B′ arm from the wild-type ITR of AAV2. Three nucleotides remaining in the B-B′ arm of modified ITR do not make a complementary pairing. Thus, ITR-4 Left and Right have the lowest energy structure with a single C-C′ arm and a single unpaired loop. Gibbs free energy of unfolding the structure is predicted to be about −76.9 kcal/mol.

ITR-10 Left and Right provided in FIGS. 12A and 12B (SEQ ID NOS: 107 and 108), are generated to include 8 nucleotide deletions in B-B′ arm from the wild-type ITR of AAV2. Nucleotides remaining in the B-B′ and C-C′ arms make new complementary bonds between B and C′ motives (ITR-10 Left) or between C and B′ motives (ITR-10 Right). Thus, ITR-10 Left and Right have the lowest energy structure with a single B-C′ or C-B′ arm and a single unpaired loop. Gibbs free energy of unfolding the structure is predicted to be about −83.7 kcal/mol.

ITR-17 Left and Right provided in FIGS. 13A and 13B (SEQ ID NOS: 109 and 110), are generated to include 14 nucleotide deletions in C-C′ arm from the wild-type ITR of AAV2. Eight nucleotides remaining in the C-C′ arm do not make complementary bonds. As a result, ITR-17 Left and Right have the lowest energy structure with a single B-B′ arm and a single unpaired loop. Gibbs free energy of unfolding the structure is predicted to be about −73.3 kcal/mol.

Sequences of wild-type ITR Left or Right (top) and various modified ITRs Left or Right (bottom) predicted to form the single-arm/single-unpaired-loop structure are aligned and provided below in Table 7.

TABLE 7
Table 7: Alignment of wt-ITR and modified ITRs (ITR-2, ITR-3, ITR-4, ITR-10
and ITR-17) with a single-arm/single-unpaired-loop structure.

| | Sequence alignment of wild-type ITRs; WT-L ITR (SEQ ID | |
|---|---|---|
| | NO: 540) or WT-R ITR (SEQ ID NO: 17)(top sequence) v. | |
| Modified ITR | modified ITR sequences (SEQ ID NOs: 101, 102, 103, 104, | ΔG |
| (SEQ ID NO) | 105, 106, 107, 108, 109, 110)(bottom sequences)) | (kcal/mol) |
| Left | 10 20 30 40 50 60 | −72.6 |
| ITR-2 | GCGCGCTCGCTCGCTCACTGAGGCCGCCCGGGCAAAGCCCGGGCGTCGGGCGACCTTTGG | |
| (SEQ: 101) | :::::::::::::::::::::::::::::::: :: ::::::::: ::: | |
| | GCGCGCTCGCTCGCTCACTGAGGCCGCCCGGGAAA--CCCGGGCGT---GCG-------- | |
| | 10 20 30 40 | |
| | 70 80 90 | |
| | TCGCCCGGCCTCAGTGAGCGAGCGAGCGCGC | |
| | ::::::::::::::::::::::: | |
| | --------CCTCAGTGAGCGAGCGAGCGCGC | |
| | 50 60 70 | |
| Right | 10 20 30 40 50 | −72.6 |
| ITR-2 | GCGCGCTCGCTCGCTCACTGAGGCCGGGCGACCAAAGGTCGCCCGACG--CCCGGGCGGC | |
| (SEQ: 102) | :::::::::::::::::::::::: : :::::: :::::::::: | |
| | GCGCGCTCGCTCGCTCACTGAGGC------------GCACGCCCGGGTTTCCCGGGCGGC | |
| | 10 20 30 40 | |
| | 60 70 80 | |
| | CTCAGTGAGCGAGCGAGCGCGC | |
| | :::::::::::::::::::::: | |
| | CTCAGTGAGCGAGCGAGCGCGC | |
| | 50 60 70 | |
| Left | 10 20 30 40 50 60 | −74.8 |
| ITR-3 | GCGCGCTCGCTCGCTCACTGAGGCCGCCCGGGCAAAGCCCGGGCGTCGGGCGACCTTTGG | |
| (SEQ: 103) | :::::::::::::::::::::::::: ::::::::::::::: | |
| | GCGCGCTCGCTCGCTCACTGAGGCCG-------------------TCGGGCGACCTTTGG | |
| | 10 20 30 40 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 50 60 70 | |
| Right | 10 20 30 40 50 60 | |
| ITR-3 | GCGCGCTCGCTCGCTCACTGAGGCCGGGCGACCAAAGGTCGCCCGACGCCCGGGCGGCCT | −74.8 |
| (SEQ: 104) | :::::::::::::::::::::::::::::::::::::::::::::::: :::: | |
| | GCGCGCTCGCTCGCTCACTGAGGCCGGGCGACCAAAGGTCGCCCGACG--------GCCT | |
| | 10 20 30 40 50 | |
| | 70 80 | |
| | CAGTGAGCGAGCGAGCGCGC | |
| | :::::::::::::::::::: | |
| | CAGTGAGCGAGCGAGCGCGC | |
| | 60 70 | |
| Left | 10 20 30 40 50 60 | −76.9 |
| ITR-4 | GCGCGCTCGCTCGCTCACTGAGGCCGCCCGGGCAAAGCCCGGGCGTCGGGCGACCTTTGG | |
| (SEQ: 105) | ::::::::::::::::::::::::::::::::::::::::::::::::: | |
| | GCGCGCTCGCTCGCTCACTGAGGCCGCCCGGGCAAAGCCCGGGCGTCGG----------- | |
| | 10 20 30 40 | |
| | 70 80 90 | |
| | TCGCCCGGCCTCAGTGAGCGAGCGAGCGCGC | |
| | ::::::::::::::::::::::: | |
| | --------CCTCAGTGAGCGAGCGAGCGCGC | |
| | 50 60 70 | |
| Right | 10 20 30 40 50 60 | −76.9 |
| ITR-4 | GCGCGCTCGCTCGCTCACTGAGGCCGGGCGACCAAAGGTCGCCCGACGCCCGGGCGGCCT | |
| (SEQ: 106) | :::::::::::::::::::::::::: : : :: : ::::::::::::: | |
| | GCGCGCTCGCTCGCTCACTGAGGCCG--------ACGCCCGGGCTTTGCCCGGGCGGCCT | |
| | 10 20 30 40 50 | |
| | 70 80 | |
| | CAGTGAGCGAGCGAGCGCGC | |
| | :::::::::::::::::::: | |
| | CAGTGAGCGAGCGAGCGCGC | |
| | 60 70 | |
| Left | 10 20 30 40 50 60 | −83.7 |
| ITR-10 | GCGCGCTCGCTCGCTCACTGAGGCCGCCCGGGCAAAGCCCGGGCGTCGGGCGACCTTTGG | |
| (SEQ: 107) | ::::::::::::::::::::::::::::::::::::::::::::::::::: ::: | |
| | GCGCGCTCGCTCGCTCACTGAGGCCGCCCGGGCAAAGCCCGGGCGTCGGGC----TTT-- | |
| | 10 20 30 40 50 | |
| | 70 80 90 | |
| | TCGCCCGGCCTCAGTGAGCGAGCGAGCGCGC | |
| | ::::::::::::::::::::::::::::: | |
| | --GCCCGGCCTCAGTGAGCGAGCGAGCGCGC | |
| | 60 70 80 | |
| Right | 10 20 30 40 50 | −83.7 |
| ITR-10 | GCGCGCTCGCTCGCTCACTGAGGCCGGGCGACCAAAG---GTCGCCCGAC----GCCCGG | |
| (SEQ: 108) | ::::::::::::::::::::::::::::: :::: : :::::: :::::: | |
| | GCGCGCTCGCTCGCTCACTGAGGCCGGGC----AAAGCCCGACGCCCGGGCTTTGCCCGG | |
| | 10 20 30 40 50 | |
| | 60 70 80 | |
| | GCGGCCTCAGTGAGCGAGCGAGCGCGC | |
| | ::::::::::::::::::::::::::: | |
| | GCGGCCTCAGTGAGCGAGCGAGCGCGC | |
| | 60 70 80 | |
| Left | 10 20 30 40 50 60 | −73.3 |
| ITR-17 | GCGCGCTCGCTCGCTCACTGAGGCCGCCCGGGCAAAGCCCGGGCGTCGGGCGACCTTTGG | |
| (SEQ: 109) | :::::::::::::::::::::::::: ::: ::::::::::::::::: | |
| | GCGCGCTCGCTCGCTCACTGAGGCCG-------AAA-------CGTCGGGCGACCTTTGG | |
| | 10 20 30 40 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 50 60 70 | |
| Right | 10 20 30 40 50 60 | −73.3 |
| ITR-17 | GCGCGCTCGCTCGCTCACTGAGGCCGGGCGACCAAAGGTCGCCCGACGCCCGGGCGGCCT | |
| (SEQ: 110) | :::::::::::::::::::::::::::::::::::::::::::::::: :::::: | |
| | GCGCGCTCGCTCGCTCACTGAGGCCGGGCGACCAAAGGTCGCCCGACGTTT---CGGCCT | |
| | 10 20 30 40 50 | |
| | 70 80 | |
| | CAGTGAGCGAGCGAGCGCGC | |
| | :::::::::::::::::::: | |
| | CAGTGAGCGAGCGAGCGCGC | |
| | 60 70 | |

Modified ITR with a Single-Hairpin Structure

The wild-type ITR can be modified to have the lowest energy structure comprising a single-hairpin structure. Gibbs free energy (ΔG) of unfolding of the structure can range between −70 kcal/mol and −40 kcal/mol. Exemplary structures of the modified ITRs are provided in FIGS. 14A and 14B.

Modified ITRs predicted to form the single hairpin structure can include deletion, insertion, or substitution of one or more nucleotides from the wild-type ITR in the sequences forming B and B′ arm and/or C and C′ arm. Modified ITR can be generated by genetic modification or biological and/or chemical synthesis.

For example, ITR-6 Left and Right provided in FIGS. 14A and 14B (SEQ ID NOS: 111 and 112), include 40 nucleotide deletions in B-B′ and C-C′ arms from the wild-type ITR of AAV2. Nucleotides remaining in the modified ITR are predicted to form a single hairpin structure. Gibbs free energy of unfolding the structure is about −54.4 kcal/mol.

Sequences of wild-type ITR and ITR-6 (both left and right) are aligned and provide below in Table 8.

TABLE 8
Table 8: Alignment of wt-ITR and modified ITR-6 with a single-hairpin structure.

| | Sequence alignment of wild-type ITRs; WT-L ITR (SEQ ID | |
|---|---|---|
| | NO: 540) or WT-R ITR (SEQ ID NO: 17)(top sequence)) v. | |
| Modified ITR | modified ITR-6 (SEQ ID NO: 111; ITR-6, left)(SEQ ID NO: 112, | ΔG |
| (SEQ ID NO) | ITR-6 right)(bottom sequence) | (kcal/mol) |
| Left | 10 20 30 40 50 60 | −54.4 |
| ITR-6 | GCGCGCTCGCTCGCTCACTGAGGCCGCCCGGGCAAAGCCCGGGCGTCGGGCGACCTTTGG | |
| (SEQ: 111) | :::::::::::::::::::::::: :::::: | |
| | GCGCGCTCGCTCGCTCACTGAGGC---------AAAGCC--------------------- | |
| | 10 20 30 | |
| | 70 80 90 | |
| | TCGCCCGGCCTCAGTGAGCGAGCGAGCGCGC | |
| | ::::::::::::::::::::: | |
| | ----------TCAGTGAGCGAGCGAGCGCGC | |
| | 40 50 | |
| Right | 80 70 60 50 40 30 | −54.4 |
| ITR-6 | , GCGCGCTCGCTCGCTCACTGAGGCCGCCCGGGCGTCGGGCGACCTTTGGTCGCCCGGCCT | |
| (SEQ: 112) | :::::::::::::::::::::::: :::: ::: | |
| | , GCGCGCTCGCTCGCTCACTGAGGCC-------------------TTTG---------CCT | |
| | 10 20 | |
| | 20 10 | |
| | , CAGTGAGCGAGCGAGCGCGC (SEQ ID NO: 543) | |
| | :::::::::::::::::::: | |
| | , CAGTGAGCGAGCGAGCGCGC (SEQ ID NO: 544) | |
| | 40 50 | |

Modified ITR with a Truncated Structure

The wild-type ITR can be modified to have the lowest energy structure comprising two arms, one of which is truncated. Their Gibbs free energy (ΔG) of unfolding ranges between −90 and −70 kcal/mol. Thus, their Gibbs free energies of unfolding are lower than the wild-type ITR of AAV2.

The modified ITRs can include deletion, insertion, or substitution of one or more nucleotides from the wild-type ITR in the sequences forming B and B′ arm and/or C and C′ arm. In some embodiments, a modified ITR can, for example, comprise removal of all of a particular loop, e.g., A-A′ loop, B-B′ loop or C-C′ loop, or alternatively, the removal of 1, 2, 3, 4, 5, 6, 7, 8, 9 or more base pairs forming the stem of the loop so long as the final loop at the end of the stem is still present. Modified ITR can be generated by genetic modification or biological and/or chemical synthesis.

Exemplary structures of the modified ITRs with a truncated structure are provided in FIGS. 15A-15B.

Sequences of various modified ITRs predicted to form a truncated structure are aligned with a sequence of wild-type ITR and provided below in Table 9.

TABLE 9
Table 9: Alignment of wt-ITR and modified ITRs (ITR-5, ITR-7, ITR-8, ITR-9,
ITR-11, ITR-12, ITR-13, ITR-14, ITR-1 and ITR-16) with a truncated structure.

| | Sequence alignment: wild-type ITRs; WT-L ITR (SEQ ID | |
|---|---|---|
| Modified ITR | NO: 540) or WT-R ITR (SEQ ID NO: 17)(top sequence) v. | ΔG |
| (SEQ ID NO) | modified ITRs) (SEQ ID NOs: 115-134)(bottom sequences) | (kcal/mol) |
| Left | 10 20 30 40 50 60 | −73.4 |
| ITR-5 | GCGCGCTCGCTCGCTCACTGAGGCCGCCCGGGCAAAGCCCGGGCGTCGGGCGACCTTTGG | |
| (SEQ: 115) | :::::::::::::::::::::::: :::::::::::::::::::::::: | |
| | GCGCGCTCGCTCGCTCACTGAGGC------------GCCCGGGCGTCGGGCGACCTTTGG | |
| | 10 20 30 40 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC (SEQ ID NO: 545) | |
| | 50 60 70 | |
| Right | 10 20 30 40 50 60 | −73.4 |
| ITR-5 | GCGCGCTCGCTCGCTCACTGAGGCCGGGCGACCAAAGGTCGCCCGACGCCCGGGCGGCCT | |
| (SEQ: 116) | :::::::::::::::::::::::::::::::::::::::::::::::::::::::: ::: | |
| | GCGCGCTCGCTCGCTCACTGAGGCCGGGCGACCAAAGGTCGCCCGACGCCCGGGCG-CCT | |
| | 10 20 30 40 50 | |
| | 70 80 | |
| | CAGTGAGCGAGCGAGCGCGC | |
| | :::::::::::::::::::: | |
| | CAGTGAGCGAGCGAGCGCGC | |
| | 60 70 | |
| Left | 10 20 30 40 50 60 | −89.6 |
| ITR-7 | GCGCGCTCGCTCGCTCACTGAGGCCGCCCGGGCAAAGCCCGGGCGTCGGGCGACCTTTGG | |
| (SEQ: 117) | :::::::::::::::::::::::::::::::::::::::::::::::::::::: :: : | |
| | GCGCGCTCGCTCGCTCACTGAGGCCGCCCGGGCAAAGCCCGGGCGTCGGGCGAC--TTTG | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −89.6 |
| ITR-7 | GCGCGCTCGCTCGCTCACTGAGGCCGGGCGACCAAAGGTCGCCCGACGCCCGGGC----- | |
| (SEQ: 118) | :::::::::::::::::::::::::::::::: :: ::::::::::::::::::: | |
| | GCGCGCTCGCTCGCTCACTGAGGCCGGGCGACAAA--GTCGCCCGACGCCCGGGCTTTGC | |
| | 10 20 30 40 50 | |
| | 60 70 80 | |
| | ------GGCCTCAGTGAGCGAGCGAGCGCGC | |
| | ::::::::::::::::::::::::: | |
| | CCGGGCGGCCTCAGTGAGCGAGCGAGCGCGC | |
| | 60 70 80 | |
| Left | 10 20 30 40 50 60 | −86.9 |
| ITR-8 | GCGCGCTCGCTCGCTCACTGAGGCCGCCCGGGCAAAGCCCGGGCGTCGGGCGACCTTTGG | |
| (SEQ: 119) | ::::::::::::::::::::::::::::::::::::::::::::::::::::: ::: | |
| | GCGCGCTCGCTCGCTCACTGAGGCCGCCCGGGCAAAGCCCGGGCGTCGGGCGA--TTT-- | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −86.9 |
| ITR-8 | GCGCGCTCGCTCGCTCACTGAGGCCGGGCGACCAAAGGTCGCCCGACGCCCGGGC----- | |
| (SEQ: 120) | ::::::::::::::::::::::::::::::: ::: ::::::::::::::::: | |
| | GCGCGCTCGCTCGCTCACTGAGGCCGGGCGA--AAA--TCGCCCGACGCCCGGGCTTTGC | |
| | 10 20 30 40 50 | |
| | 60 70 80 | |
| | ------GGCCTCAGTGAGCGAGCGAGCGCGC | |
| | ::::::::::::::::::::::::: | |
| | CCGGGCGGCCTCAGTGAGCGAGCGAGCGCGC | |
| | 60 70 80 | |
| Left | 10 20 30 40 50 60 | −85.0 |
| ITR-9 | GCGCGCTCGCTCGCTCACTGAGGCCGCCCGGGCAAAGCCCGGGCGTCGGGCGACCTTTGG | |
| (SEQ: 121) | :::::::::::::::::::::::::::::::::::::::::::::::::::: :: | |
| | GCGCGCTCGCTCGCTCACTGAGGCCGCCCGGGCAAAGCCCGGGCGTCGGGCG----TT-- | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −85.0 |
| ITR-9 | GCGCGCTCGCTCGCTCACTGAGGCCGGGCGACCAAAGGTCGCCCGACGCCCGGGC----- | |
| (SEQ: 122) | ::::::::::::::::::::::::::::::: :: :::::::::::::::: | |
| | GCGCGCTCGCTCGCTCACTGAGGCCGGGCGA--AA----CGCCCGACGCCCGGGCTTTGC | |
| | 10 20 30 40 50 | |
| | 60 70 80 | |
| | ------GGCCTCAGTGAGCGAGCGAGCGCGC | |
| | CCGGGCGGCCTCAGTGAGCGAGCGAGCGCGC | |
| | 60 70 80 | |
| Left | 10 20 30 40 50 60 | −89.5 |
| ITR-11 | GCGCGCTCGCTCGCTCACTGAGGCCGCCCGGGCAAAGCCCGGGCGTCGGGCGACCTTTGG | |
| (SEQ: 123) | :::::::::::::::::::::::::::::::: :: :::::::::::::::::::::::: | |
| | GCGCGCTCGCTCGCTCACTGAGGCCGCCCGGGAAA--CCCGGGCGTCGGGCGACCTTTGG | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −89.5 |
| ITR-11 | GCGCGCTCGCTCGCTCACTGAGGCCGGGCGACCAAAGGTCGCCCGACGCCCGGG------ | |
| (SEQ: 124) | :::::::::::::::::::::::::::::::::::::::::::::::::::::: | |
| | GCGCGCTCGCTCGCTCACTGAGGCCGGGCGACCAAAGGTCGCCCGACGCCCGGGTTTCCC | |
| | 70 80 90 100 110 120 | |
| | 60 70 80 | |
| | ---CGGCCTCAGTGAGCGAGCGAGCGCGC | |
| | :::::::::::::::::::::::::: | |
| | GGGCGGCCTCAGTGAGCGAGCGAGCGCGC | |
| | 130 140 150 | |
| Left | 10 20 30 40 50 60 | −86.2 |
| ITR-12 | GCGCGCTCGCTCGCTCACTGAGGCCGCCCGGGCAAAGCCCGGGCGTCGGGCGACCTTTGG | |
| (SEQ: 125) | ::::::::::::::::::::::::::::::: ::: :::::::::::::::::::::: | |
| | GCGCGCTCGCTCGCTCACTGAGGCCGCCCGG--AAA-CCGGGCGTCGGGCGACCTTTGG | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −86.2 |
| ITR-12 | GCGCGCTCGCTCGCTCACTGAGGCCGGGCGACCAAAGGTCGCCCGACGCCCGG------- | |
| (SEQ: 126) | ::::::::::::::::::::::::::::::::::::::::::::::::::::: | |
| | GCGCGCTCGCTCGCTCACTGAGGCCGGGCGACCAAAGGTCGCCCGACGCCCGGTTTCCGG | |
| | 10 20 30 40 50 60 | |
| | 60 70 80 | |
| | GCGGCCTCAGTGAGCGAGCGAGCGCGC | |
| | ::::::::::::::::::::::::::: | |
| | GCGGCCTCAGTGAGCGAGCGAGCGCGC | |
| | 70 80 | |
| Left | 10 20 30 40 50 60 | −82.9 |
| ITR-13 | GCGCGCTCGCTCGCTCACTGAGGCCGCCCGGGCAAAGCCCGGGCGTCGGGCGACCTTTGG | |
| (SEQ: 127) | :::::::::::::::::::::::::::::: ::: ::::::::::::::::::::: | |
| | GCGCGCTCGCTCGCTCACTGAGGCCGCCCG---AAA---CGGGCGTCGGGCGACCTTTGG | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −82.9 |
| ITR-13 | GCGCGCTCGCTCGCTCACTGAGGCCGGGCGACCAAAGGTCGCCCGACGCCCG-----GGC | |
| (SEQ: 128) | :::::::::::::::::::::::::::::::::::::::::::::::::::: ::: | |
| | GCGCGCTCGCTCGCTCACTGAGGCCGGGCGACCAAAGGTCGCCCGACGCCCGTTTCGGGC | |
| | 10 20 30 40 50 60 | |
| | 60 70 80 | |
| | GGCCTCAGTGAGCGAGCGAGCGCGC | |
| | ::::::::::::::::::::::::: | |
| | GGCCTCAGTGAGCGAGCGAGCGCGC | |
| | 70 80 | |
| Left | 10 20 30 40 50 60 | −80.5 |
| ITR-14 | GCGCGCTCGCTCGCTCACTGAGGCCGCCCGGGCAAAGCCCGGGCGTCGGGCGACCTTTGG | |
| (SEQ: 129) | ::::::::::::::::::::::::::::: :::: ::::::::::::::::::: | |
| | GCGCGCTCGCTCGCTCACTGAGGCCGCCC----AAAG----GGCGTCGGGCGACCTTTGG | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −80.5 |
| ITR-14 | GCGCGCTCGCTCGCTCACTGAGGCCGGGCGACCAAAGGTCGCCCGACGCCC---GGGCGG | |
| (SEQ: 130) | ::::::::::::::::::::::::::::::::::::::::::::::::::: :::::: | |
| | GCGCGCTCGCTCGCTCACTGAGGCCGGGCGACCAAAGGTCGCCCGACGCCCTTTGGGCGG | |
| | 10 20 30 40 50 60 | |
| | 60 70 80 | |
| | CCTCAGTGAGCGAGCGAGCGCGC | |
| | ::::::::::::::::::::::: | |
| | CCTCAGTGAGCGAGCGAGCGCGC | |
| | 70 80 | |
| Left | 10 20 30 40 50 60 | −77.2 |
| ITR-15 | GCGCGCTCGCTCGCTCACTGAGGCCGCCCGGGCAAAGCCCGGGCGTCGGGCGACCTTTGG | |
| (SEQ: 131) | :::::::::::::::::::::::::::: :::: :::::::::::::::::: | |
| | GCGCGCTCGCTCGCTCACTGAGGCCGCC-----AAAG-----GCGTCGGGCGACCTTTGG | |
| | 10 20 30 40 50 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 60 70 80 | |
| Right | 10 20 30 40 50 | −77.2 |
| ITR-15 | GCGCGCTCGCTCGCTCACTGAGGCCGGGCGACCAAAGGTCGCCCGACGCCCG-GGCGGCC | |
| (SEQ: 132) | :::::::::::::::::::::::::::::::::::::::::::::::::: ::::::: | |
| | GCGCGCTCGCTCGCTCACTGAGGCCGGGCGACCAAAGGTCGCCCGACGCCTTTGGCGGCC | |
| | 10 20 30 40 50 60 | |
| | 60 70 80 | |
| | TCAGTGAGCGAGCGAGCGCGC | |
| | ::::::::::::::::::::: | |
| | TCAGTGAGCGAGCGAGCGCGC | |
| | 70 80 | |
| Left | 10 20 30 40 50 60 | −73.9 |
| ITR-16 | GCGCGCTCGCTCGCTCACTGAGGCCGCCCGGGCAAAGCCCGGGCGTCGGGCGACCTTTGG | |
| (SEQ: 133) | ::::::::::::::::::::::::::: ::::: :::::::::::::::: | |
| | GCGCGCTCGCTCGCTCACTGAGGCCGC------AAAGC------GTCGGGCGACCTTTGG | |
| | 10 20 30 40 | |
| | 70 80 90 | |
| | TCGCCCGGCCTCAGTGAGCGAGCGAGCGCGC | |
| | ::::::::::::::::::::::::::::::: | |
| | TCGCCCGGCCTCAGTGAGCGAGCGAGCGCGC | |
| | 50 60 70 | |
| Right | 10 20 30 40 50 60 | −73.9 |
| ITR-16 | GCGCGCTCGCTCGCTCACTGAGGCCGGGCGACCAAAGGTCGCCCGACGCCCGGGCGGCCT | |
| (SEQ: 134) | ::::::::::::::::::::::::::::::::::::::::::::::::: : :::::: | |
| | GCGCGCTCGCTCGCTCACTGAGGCCGGGCGACCAAAGGTCGCCCGACGCTTTG-CGGCCT | |
| | 10 20 30 40 50 | |
| | 70 80 | |
| | CAGTGAGCGAGCGAGCGCGC | |
| | :::::::::::::::::::: | |
| | CAGTGAGCGAGCGAGCGCGC | |
| | 60 70 | |

Additional exemplary modified ITRs in each of the above classes for use herein are provided in Tables 10A and 10B. The predicted secondary structure of the Right modified ITRs in Table 10A are shown in FIG. 26A, and the predicted secondary structure of the Left modified ITRs in Table 10B are shown in FIG. 26B.

Table 10A and Table 10B show exemplary right and left modified ITRs.

TABLE 10A
TABLE 10A: Exemplary modified right ITRs. These exemplary
modified right ITRs can comprise the RBE of GCGCGCTCGCTCGCTC-
3′ (SEQ ID NO: 531), spacer of ACTGAGGC (SEQ ID NO: 532), the
spacer complement GCCTCAGT (SEQ ID NO: 535) and RBE′ (i.e.,
complement to RBE) of GAGCGAGCGAGCGCGC (SEQ ID NO: 536).
Exemplary Right modified ITRs

| ITR | | SEQ ID |
|---|---|---|
| Construct | Sequence | NO: |
| ITR-18 | AGGAACCCCTAGTGATGGAGTTGGCCACTCCCTCTCTGCGCGCTCG | 469 |
| Right | CTCGCTCACTGAGGCGCACGCCCGGGTTTCCCGGGCGGCCTCAGTG | |
| | AGCGAGCGAGCGCGCAGCTGCCTGCAGG | |
| ITR-19 | AGGAACCCCTAGTGATGGAGTTGGCCACTCCCTCTCTGCGCGCTCG | 470 |
| Right | CTCGCTCACTGAGGCCGACGCCCGGGCTTTGCCCGGGCGGCCTCAG | |
| | TGAGCGAGCGAGCGCGCAGCTGCCTGCAGG | |
| ITR-20 | AGGAACCCCTAGTGATGGAGTTGGCCACTCCCTCTCTGCGCGCTCG | 471 |
| Right | CTCGCTCACTGAGGCCGGGCGACCAAAGGTCGCCCGACGCCCGGGC | |
| | GCCTCAGTGAGCGAGCGAGCGCGCAGCTGCCTGCAGG | |
| ITR-21 | AGGAACCCCTAGTGATGGAGTTGGCCACTCCCTCTCTGCGCGCTCG | 472 |
| Right | CTCGCTCACTGAGGCTTTGCCTCAGTGAGCGAGCGAGCGCGCAGCT | |
| | GCCTGCAGG | |
| ITR-22 | AGGAACCCCTAGTGATGGAGTTGGCCACTCCCTCTCTGCGCGCTCG | 473 |
| Right | CTCGCTCACTGAGGCCGGGCGACAAAGTCGCCCGACGCCCGGGCTT | |
| | TGCCCGGGCGGCCTCAGTGAGCGAGCGAGCGCGCAGCTGCCTGCAG | |
| | G | |
| ITR-23 | AGGAACCCCTAGTGATGGAGTTGGCCACTCCCTCTCTGCGCGCTCG | 474 |
| Right | CTCGCTCACTGAGGCCGGGCGAAAATCGCCCGACGCCCGGGCTTTG | |
| | CCCGGGCGGCCTCAGTGAGCGAGCGAGCGCGCAGCTGCCTGCAGG | |
| ITR-24 | AGGAACCCCTAGTGATGGAGTTGGCCACTCCCTCTCTGCGCGCTCG | 475 |
| Right | CTCGCTCACTGAGGCCGGGCGAAACGCCCGACGCCCGGGCTTTGCC | |
| | CGGGCGGCCTCAGTGAGCGAGCGAGCGCGCAGCTGCCTGCAGG | |
| ITR-25 | AGGAACCCCTAGTGATGGAGTTGGCCACTCCCTCTCTGCGCGCTCG | 476 |
| Right | CTCGCTCACTGAGGCCGGGCAAAGCCCGACGCCCGGGCTTTGCCCG | |
| | GGCGGCCTCAGTGAGCGAGCGAGCGCGCAGCTGCCTGCAGG | |
| ITR-26 | AGGAACCCCTAGTGATGGAGTTGGCCACTCCCTCTCTGCGCGCTCG | 477 |
| Right | CTCGCTCACTGAGGCCGGGCGACCAAAGGTCGCCCGACGCCCGGGT | |
| | TTCCCGGGCGGCCTCAGTGAGCGAGCGAGCGCGCAGCTGCCTGCAG | |
| | G | |
| ITR-27 | AGGAACCCCTAGTGATGGAGTTGGCCACTCCCTCTCTGCGCGCTCG | 478 |
| Right | CTCGCTCACTGAGGCCGGGCGACCAAAGGTCGCCCGACGCCCGGTT | |
| | TCCGGGCGGCCTCAGTGAGCGAGCGAGCGCGCAGCTGCCTGCAGG | |
| ITR-28 | AGGAACCCCTAGTGATGGAGTTGGCCACTCCCTCTCTGCGCGCTCG | 479 |
| Right | CTCGCTCACTGAGGCCGGGCGACCAAAGGTCGCCCGACGCCCGTTT | |
| | CGGGCGGCCTCAGTGAGCGAGCGAGCGCGCAGCTGCCTGCAGG | |
| ITR-29 | AGGAACCCCTAGTGATGGAGTTGGCCACTCCCTCTCTGCGCGCTCG | 480 |
| Right | CTCGCTCACTGAGGCCGGGCGACCAAAGGTCGCCCGACGCCCTTTG | |
| | GGCGGCCTCAGTGAGCGAGCGAGCGCGCAGCTGCCTGCAGG | |
| ITR-30 | AGGAACCCCTAGTGATGGAGTTGGCCACTCCCTCTCTGCGCGCTCG | 481 |
| Right | CTCGCTCACTGAGGCCGGGCGACCAAAGGTCGCCCGACGCCTTTGG | |
| | CGGCCTCAGTGAGCGAGCGAGCGCGCAGCTGCCTGCAGG | |
| ITR-31 | AGGAACCCCTAGTGATGGAGTTGGCCACTCCCTCTCTGCGCGCTCG | 482 |
| Right | CTCGCTCACTGAGGCCGGGCGACCAAAGGTCGCCCGACGCTTTGCG | |
| | GCCTCAGTGAGCGAGCGAGCGCGCAGCTGCCTGCAGG | |
| ITR-32 | AGGAACCCCTAGTGATGGAGTTGGCCACTCCCTCTCTGCGCGCTCG | 483 |
| Right | CTCGCTCACTGAGGCCGGGCGACCAAAGGTCGCCCGACGTTTCGGC | |
| | CTCAGTGAGCGAGCGAGCGCGCAGCTGCCTGCAGG | |
| ITR-49 | AGGAACCCCTAGTGATGGAGTTGGCCACTCCCTCTCTGCGCGCTCG | 99 |
| Right | CTCGCTCACTGAGGCCGGGCGACCAAAGGTCGCCCGACGGCCTCAG | |
| | TGAGCGAGCGAGCGCGCAGCTGCCTGCAGG | |
| ITR-50 | AGGAACCCCTAGTGATGGAGTTGGCCACTCCCTCTCTGCGCGCTCG | 100 |
| right | CTCGCTCACTGAGGCCGGGCGACCAAAGGTCGCCCGACGCCCGGGC | |
| | GGCCTCAGTGAGCGAGCGAGCGCGCAGCTGCCTGCAGG | |

TABLE 10B
TABLE 10B: Exemplary modified left ITRs.
These exemplary modified left ITRs can
comprise the RBE of GCGCGCTCGCTCGCTC-3′
(SEQ ID NO: 531), spacer of ACTGAGGC
(SEQ ID NO: 532), the spacer complement
GCCTCAGT (SEQ ID NO: 535) and RBE
complement (RBE′) of GAGCGAGCGAGCGCGC
(SEQ ID NO: 536).
Exemplary modified left ITRs

| | | |
|---|---|---|
| ITR-33 | CCTGCAGGCAGCTGCGCGCTCGCTCGCTCACT | 484 |
| Left | GAGGCCGCCCGGGAAACCCGGGCGTGCGCCTC | |
| | AGTGAGCGAGCGAGCGCGCAGAGAGGGAGTGG | |
| | CCAACTCCATCACTAGGGGTTCCT | |
| ITR-34 | CCTGCAGGCAGCTGCGCGCTCGCTCGCTCACT | 485 |
| Left | GAGGCCGTCGGGCGACCTTTGGTCGCCCGGCC | |
| | TCAGTGAGCGAGCGAGCGCGCAGAGAGGGAGT | |
| | GGCCAACTCCATCACTAGGGGTTCCT | |
| ITR-35 | CCTGCAGGCAGCTGCGCGCTCGCTCGCTCACT | 486 |
| Left | GAGGCCGCCCGGGCAAAGCCCGGGCGTCGGCC | |
| | TCAGTGAGCGAGCGAGCGCGCAGAGAGGGAGT | |
| | GGCCAACTCCATCACTAGGGGTTCCT | |
| ITR-36 | CCTGCAGGCAGCTGCGCGCTCGCTCGCTCACT | 487 |
| Left | GAGGCGCCCGGGCGTCGGGCGACCTTTGGTCG | |
| | CCCGGCCTCAGTGAGCGAGCGAGCGCGCAGAG | |
| | AGGGAGTGGCCAACTCCATCACTAGGGGTTCC | |
| | T | |
| ITR-37 | CCTGCAGGCAGCTGCGCGCTCGCTCGCTCACT | 488 |
| Left | GAGGCAAAGCCTCAGTGAGCGAGCGAGCGCGC | |
| | AGAGAGGGAGTGGCCAACTCCATCACTAGGGG | |
| | TTCCT | |
| ITR-38 | CCTGCAGGCAGCTGCGCGCTCGCTCGCTCACT | 489 |
| Left | GAGGCCGCCCGGGCAAAGCCCGGGCGTCGGGC | |
| | GACTTTGTCGCCCGGCCTCAGTGAGCGAGCGA | |
| | GCGCGCAGAGAGGGAGTGGCCAACTCCATCAC | |
| | TAGGGGTTCCT | |
| ITR-39 | CCTGCAGGCAGCTGCGCGCTCGCTCGCTCACT | 490 |
| Left | GAGGCCGCCCGGGCAAAGCCCGGGCGTCGGGC | |
| | GATTTTCGCCCGGCCTCAGTGAGCGAGCGAGC | |
| | GCGCAGAGAGGGAGTGGCCAACTCCATCACTA | |
| | GGGGTTCCT | |
| ITR-40 | CCTGCAGGCAGCTGCGCGCTCGCTCGCTCACT | 491 |
| Left | GAGGCCGCCCGGGCAAAGCCCGGGCGTCGGGC | |
| | GTTTCGCCCGGCCTCAGTGAGCGAGCGAGCGC | |
| | GCAGAGAGGGAGTGGCCAACTCCATCACTAGG | |
| | GGTTCCT | |
| ITR-41 | CCTGCAGGCAGCTGCGCGCTCGCTCGCTCACT | 492 |
| Left | GAGGCCGCCCGGGCAAAGCCCGGGCGTCGGGC | |
| | TTTGCCCGGCCTCAGTGAGCGAGCGAGCGCGC | |
| | AGAGAGGGAGTGGCCAACTCCATCACTAGGGG | |
| | TTCCT | |
| ITR-42 | CCTGCAGGCAGCTGCGCGCTCGCTCGCTCACT | 493 |
| Left | GAGGCCGCCCGGGAAACCCGGGCGTCGGGCGA | |
| | CCTTTGGTCGCCCGGCCTCAGTGAGCGAGCGA | |
| | GCGCGCAGAGAGGGAGTGGCCAACTCCATCAC | |
| | TAGGGGTTCCT | |
| ITR-43 | CCTGCAGGCAGCTGCGCGCTCGCTCGCTCACT | 494 |
| Left | GAGGCCGCCCGGAAACCGGGCGTCGGGCGACC | |
| | TTTGGTCGCCCGGCCTCAGTGAGCGAGCGAGC | |
| | GCGCAGAGAGGGAGTGGCCAACTCCATCACTA | |
| | GGGGTTCCT | |
| ITR-44 | CCTGCAGGCAGCTGCGCGCTCGCTCGCTCACT | 495 |
| Left | GAGGCCGCCCGAAACGGGCGTCGGGCGACCTT | |
| | TGGTCGCCCGGCCTCAGTGAGCGAGCGAGCGC | |
| | GCAGAGAGGGAGTGGCCAACTCCATCACTAG | |
| | GGGTTCCT | |
| ITR-45 | CCTGCAGGCAGCTGCGCGCTCGCTCGCTCACT | 496 |
| Left | GAGGCCGCCCAAAGGGCGTCGGGCGACCTTTG | |
| | GTCGCCCGGCCTCAGTGAGCGAGCGAGCGCGC | |
| | AGAGAGGGAGTGGCCAACTCCATCACTAGGGG | |
| | TTCCT | |
| ITR-46 | CCTGCAGGCAGCTGCGCGCTCGCTCGCTCACT | 497 |
| Left | GAGGCCGCCAAAGGCGTCGGGCGACCTTTGGT | |
| | CGCCCGGCCTCAGTGAGCGAGCGAGCGCGCAG | |
| | AGAGGGAGTGGCCAACTCCATCACTAGGGGTT | |
| | CCT | |
| ITR-47 | CCTGCAGGCAGCTGCGCGCTCGCTCGCTCACT | 498 |
| Left | GAGGCCGCAAAGCGTCGGGCGACCTTTGGTCG | |
| | CCCGGCCTCAGTGAGCGAGCGAGCGCGCAGAG | |
| | AGGGAGTGGCCAACTCCATCACTAGGGGTTCC | |
| | T | |
| ITR-48 | CCTGCAGGCAGCTGCGCGCTCGCTCGCTCACT | 499 |
| Left | GAGGCCGAAACGTCGGGCGACCTTTGGTCGCC | |
| | CGGCCTCAGTGAGCGAGCGAGCGCGCAGAGAG | |
| | GGAGTGGCCAACTCCATCACTAGGGGTTCCT | |

In embodiments of the present invention, the ceDNA vector disclosed herein does not have a modified ITRs having the nucleotide sequence selected from any of the group of SEQ ID Nos: 550, 551, 552, 553, 553, 554, 555, 556, 557.

To the extent the ceDNA vector has a modified ITR that, has one of the modifications in the B, B′, C or C′ region as described in SEQ ID NO: 550-557 as defined in any one or more of the claims of this application, or within any invention to be defined in amended claims that may in the future be filed in this application or in any patent derived therefrom, and to the extent that the laws of any relevant country or countries to which that or those claims apply, we hereby reserve the right to disclaim the said disclosure from the claims of the present application or any patent derived therefrom to the extent necessary to prevent invalidation of the present application or any patent derived therefrom.

For example, and without limitation, we reserve the right to disclaim any one of the following subject-matters from any claim of the present application, now or as amended in the future, or any patent derived therefrom:

A. a modified ITR selected from any of the group consisting of: SEQ ID NOS: 2, 52, 63 64, 113, 114, 550, 551; 552, 553, 553, 554, 555, 556, 557 used in a ceDNA vector without a regulatory switch

B. the above-specified modified ITRs in A., in a ceDNA vector without a regulatory sequence and where the heterologous nucleic acid encodes ABCA4, USA2A varl, VEGFR, CEP290, BDD Factor VIII (FVIII), Factor VIII, vWF His, vWF, lecitchin cholesterol acetyl transferase, PAH, G6PC, or CFTR

Without limitation, we state that the above reservation of a right of disclaimer applies at least to claims 1-57 of this application and all the paragraphs, including, but not limited to paragraphs set forth in [0027] and [00397].

### IV. Regulatory Elements

The ceDNA vectors can be produced from expression constructs that further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, regulatory switches as described herein, to regulate the expression of the transgene, or a kill switch, which can kill a cell comprising the ceDNA vector.

The ceDNA vectors can be produced from expression constructs that further comprise a specific combination of cis-regulatory elements such as WHP posttranscriptional regulatory element (WPRE) (e.g., SEQ ID NO: 8) and BGH polyA (SEQ ID NO: 9). Suitable expression cassettes for use in expression constructs are not limited by the packaging constraint imposed by the viral capsid. Expression cassettes of the present invention include a promoter, which can influence overall expression levels as well as cell-specificity. For transgene expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In preferred embodiments, an expression cassette can contain a synthetic regulatory element, such as a CAG promoter (SEQ ID NO: 3). The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-1-antitrypsin (AAT) promoter (SEQ ID NO: 4 or SEQ ID NO: 74), a liver specific (LP1) promoter (SEQ ID NO: 5 or SEQ ID NO: 16), or a Human elongation factor-1 alpha (EF1a) promoter (e.g., SEQ ID NO: 6 or SEQ ID NO: 15). In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer, e.g., SEQ ID NO: 22). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 18) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 19), a CAG promoter, a human alpha 1-antitypsin (HAAT) promoter (e.g., SEQ ID NO: 21), and the like. In embodiments, these promoters are altered at their downstream intron containing end to include one or more nuclease cleavage sites. In embodiments, the DNA containing the nuclease cleavage site(s) is foreign to the promoter DNA.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the vector may comprise a promoter that is operably linked to the nucleic acid sequence encoding a therapeutic protein. The promoter operably linked to the therapeutic protein coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. The promoter may also be a tissue specific promoter, such as a liver specific promoter, such as human alpha 1-antitypsin (HAAT), natural or synthetic. In one embodiment, delivery to the liver can be achieved using endogenous ApoE specific targeting of the composition comprising a ceDNA vector to hepatocytes via the low density lipoprotein (LDL) receptor present on the surface of the hepatocyte.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e g, enhancers, (e.g. SEQ ID NO: 22 and SEQ ID NO: 23).

Non-limiting examples of suitable promoters for use in accordance with the present invention include the CAG promoter of, for example (SEQ ID NO: 3), the HAAT promoter (SEQ ID NO: 21), the human EF1-α promoter (SEQ ID NO: 6) or a fragment of the EF1a promoter (SEQ ID NO: 15), 1E2 promoter (e.g., SEQ ID NO: 20) and the rat EF1-α promoter (SEQ ID NO: 24).

Polyadenylation Sequences:

A sequence encoding a polyadenylation sequence can be included in the ceDNA vector to stabilize the mRNA expressed from the ceDNA vector, and to aid in nuclear export and translation. In one embodiment, the ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include a poly-adenylation sequence known in the art or a variation thereof, such as a naturally occurring sequence isolated from bovine BGHpA (e.g., SEQ ID NO: 74) or a virus SV40 pA (e.g., SEQ ID NO: 10), or a synthetic sequence (e.g., SEQ ID NO: 27). Some expression cassettes can also include SV40 late polyA signal upstream enhancer (USE) sequence. In some embdiments, the, USE can be used in combination with SV40 pA or heterologous poly-A signal.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 8) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 25 and SEQ ID NO: 26.

### V. Regulatory Switches

A molecular regulatory switch is one which generates a measurable change in state in response to a signal. Such regulatory switches can be usefully combined with the ceDNA vectors described herein to control the output of the ceDNA vector. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA in a controllable and regulatable fashion. In some embodiments, the switch can include a “kill switch” that can instruct the cell comprising the ceDNA vector to undergo cell programmed death once the switch is activated.

A. Binary Regulatory Switches

In some embodiments, the ceDNA vector comprises a regulatory switch that can serve to controllably modulate expression of the transgene. In such an embodiment, the expression cassette located between the ITRs of the ceDNA vector may additionally comprise a regulatory region, e.g., a promoter, cis-element, repressor, enhancer etc., that is operatively linked to the gene of interest, where the regulatory region is regulated by one or more cofactors or exogenous agents. Accordingly, in one embodiment, only when the one or more cofactor(s) or exogenous agents are present in the cell will transcription and expression of the gene of interest from the ceDNA vector occur. In another embodiment, one or more cofactor(s) or exogenous agents may be used to de-repress the transcription and expression of the gene of interest.

Any nucleic acid regulatory regions known by a person of ordinary skill in the art can be employed in a ceDNA vector designed to include a regulatory switch. By way of example only, regulatory regions can be modulated by small molecule switches or inducible or repressible promoters. Nonlimiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter. Classic tetracycline-based or other antibiotic-based switches are encompassed for use, including those disclosed in (Fussenegger et al., Nature Biotechnol. 18: 1203-1208 (2000)).

B. Small molecule Regulatory Switches

A variety of art-known small-molecule based regulatory switches are known in the art and can be combined with the ceDNA vectors disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}. Nat Methods. 2013, 10(11):1085-8.

Other small molecule based regulatory switches known by an ordinarily skilled artisan are also envisioned for use to control transgene expression of the ceDNA and include, but are not limited to, those disclosed in Buskirk et al., Cell; Chem and Biol., 2005; 12(2); 151-161; an abscisic acid sensitive ON-switch; such as that disclosed in Liang, F.-S., et al., (2011) Science Signaling, 4(164); exogenous L-arginine sensitive ON-switches such as those disclosed in Hartenbach, et al. Nucleic Acids Research, 35(20), 2007, synthetic bile-acid sensitive ON-switches such as those disclosed in Rossger et al., Metab Eng. 2014, 21: 81-90; biotin sensitive ON-switches such as those disclosed in Weber et al., Metab. Eng. 2009 March; 11(2): 117-124; dual input food additive benzoate/vanillin sensitive regulatory switches such as those disclosed in Xie et al., Nucleic Acids Research, 2014; 42(14); e116; 4-hydroxytamoxifen sensitive switches such as those disclosed in Giuseppe et al., Molecular Therapy, 6(5), 653-663; and flavinoid (phloretin) sensitive regulatory switches such as those disclosed in Gitzinger et al., Proc. Natl. Acad. Sci. USA. 2009 Jun. 30; 106(26): 10638-10643.

In some embodiments, the regulatory switch to control the transgene or expressed by the ceDNA vector is a pro-drug activation switch, such as that disclosed in U.S. Pat. Nos. 8,771,679, and 6,339,070.

Exemplary regulatory switches for use in the ceDNA vectors include, but are not limited to those in Table 11.

C. “Passcode” Regulatory Switches

In some embodiments the regulatory switch can be a “passcode switch” or “passcode circuit”. Passcode switches allow fine tuning of the control of the expression of the transgene from the ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. As an exemplary example only, if the transgene is insulin, Condition A occurs if the subject has diabetes, Condition B is if the sugar level in the blood is high and Condition C is the level of endogenous insulin not being expressed at required amounts. Once the sugar level declines or the desired level of insulin is reached, the transgene (e.g. insulin), turns off again until the 3 conditions occur, turning it back on. In another exemplary example, if the transgene is EPO, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene (e.g., EPO) turns off again until 3 conditions occur, turning it back on.

Passcode regulatory switches are useful to fine tune the expression of the transgene from the ceDNA vector. For example, the passcode regulatory switch can be modular in that it comprises multiple switches, e.g., a tissue specific, inducible promoter that is turned on only in the presence of a certain level of a metabolite. In such an embodiment, for transgene expression from the ceDNA vector to occur, the inducible agent must be present (condition A), in the desired cell type (condition B) and the metabolite is at, or above or below a certain threshold (Condition C). In alternative embodiments, the passcode regulatory switch can be designed such that the transgene expression is on when conditions A and B are present, but will turn off when condition C is present. Such an embodiment is useful when Condition C occurs as a direct result of the expressed transgene—that is Condition C serves as a positive feedback to loop to turn off transgene expression from the ceDNA vector when the transgene has had a sufficient amount of the desired therapeutic effect.

In some embodiments, a passcode regulatory switch encompassed for use in the ceDNA vector is disclosed in WO2017/059245, which describes a switch referred to as a “Passcode switch” or a “Passcode circuit” or “Passcode kill switch” which is a synthetic biological circuit that uses hybrid transcription factors (TFs) to construct complex environmental requirements for cell survival. The Passcode regulatory switches described in WO2017/059245 are particularly useful for use in the ceDNA vectors, as they are modular and customizable, both in terms of the environmental conditions that control circuit activation and in the output modules that control cell fate. In addition, the Passcode circuit has particular utility to be used in ceDNA vectors, since without the appropriate “passcode” molecules it will allow transgene expression only in the presence of the required predetermined conditions. If something goes wrong with a cell or no further transgene expression is desired for any reason, then the related kill switch (i.e. deadman switch) can be triggered.

In some embodiments, a passcode regulatory switch or “Passcode circuit” encompassed for use in the ceDNA vector comprises hybrid transcription factors (TFs) to expand the range and complexity of environmental signals used to define biocontainment conditions. As opposed to the deadman switch which triggers cell death on in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

In one aspect, a “passcode” system that restricts cell growth to the presence of a predetermined set of at least two selected agents, includes one or more nucleic acid constructs encoding expression modules comprising: i) a toxin expression module that encodes a toxin that is toxic to a host cell, wherein sequence encoding the toxin is operably linked to a promoter P1 that is repressed by the binding of a first hybrid repressor protein hRP1; ii) a first hybrid repressor protein expression module that encodes the first hybrid repressor protein hRP1, wherein expression of hRP1 is controlled by an AND gate formed by two hybrid transcription factors hTF1 and hTF2, the binding or activity of which is responsive to agents A1 and A2, respectively, such that both agents A1 and A2 are required for expression of hRP1, wherein in the absence of either A1 or A2, hRP1 expression is insufficient to repress toxin promoter module P1 and toxin production, such that the host cell is killed. In this system, hybrid factors hTF1, hTF2 and hRP1 each comprise an environmental sensing module from one transcription factor and a DNA recognition module from a different transcription factor that renders the binding of the respective passcode regulatory switch sensitive to the presence of an environmental agent, A1, or A2, that is different from that which the respective subunits would typically bind in nature.

Accordingly, a ceDNA vector can comprise a ‘Passcode regulatory circuit” that requires the presence and/or absence of specific molecules to activate the output module. In some embodiments, where genes that encode for cellular toxins are placed in the output module, this passcode regulatory circuit can not only be used to regulate transgene expression, but also can be used to create a kill switch mechanism in which the circuit kills the cell if the cell behaves in an undesired fashion (e.g., it leaves the specific environment defined by the sensor domains, or differentiates into a different cell type). In one nonlimiting example, the modularity of the hybrid transcription factors, the circuit architecture, and the output module allows the circuit to be reconfigured to sense other environmental signals, to react to the environmental signals in other ways, and to control other functions in the cell in addition to induced cell death, as is understood in the art.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches in Table 11.

D. Nucleic Acid-Based Regulatory Switches to Control Transgene Expression

In some embodiments, the regulatory switch to control the transgene expressed by the ceDNA is based on a nucleic-acid based control mechanism. Exemplary nucleic acid control mechanisms are known in the art and are envisioned for use. For example, such mechanisms include riboswiches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the ceDNA vector can comprise a regulatory switch that encodes a RNAi molecule that is complementary to the transgene expressed by the ceDNA vector. When such RNAi is expressed even if the transgene is expressed by the ceDNA vector, it will be silenced by the complementary RNAi molecule, and when the RNAi is not expressed when the transgene is expressed by the ceDNA vector the transgene is not silenced by the RNAi. Such an example of a RNAi molecule controlling gene expression, or as a regulatory switch is disclosed in US2017/0183664. In some embodiments, the regulatory switch comprises a repressor that blocks expression of the transgene from the ceDNA vector. In some embodiments, the on/off switch is a Small transcription activating RNA (STAR)-based switch, for example, such as the one disclosed in Chappell J. et al., Nat Chem Biol. 2015 March; 11(3):214-20; and Chappell et al., Microbiol Spectr. 2018 May; 6(3. In some embodiments, the regulatory switch is a toehold switch, such as that disclosed in US2009/0191546, US2016/0076083, WO2017/087530, US2017/0204477, WO2017/075486 and in Green et al, Cell, 2014; 159(4); 925-939.

In some embodiments, the regulatory switch is a tissue-specific self-inactivating regulatory switch, for example as disclosed in US2002/0022018, whereby the regulatory switch deliberately switches transgene expression off at a site where transgene expression might otherwise be disadvantageous. In some embodiments, the regulatory switch is a recombinase reversible gene expression system, for example as disclosed in US2014/0127162 and U.S. Pat. No. 8,324,436.

In some embodiments, the regulatory switch to control the transgene or gene of interest expressed by the ceDNA vector is a hybrid of a nucleic acid-based control mechanism and a small molecule regulator system. Such systems are well known to persons of ordinary skill in the art and are envisioned for use herein. Examples of such regulatory switches include, but are not limited to, an LTRi system or “Lac-Tet-RNAi” system, e.g., as disclosed in US2010/0175141 and in Deans T. et al., Cell., 2007, 130(2); 363-372, WO2008/051854 and U.S. Pat. No. 9,388,425.

In some embodiments, the regulatory switch to control the transgene or gene of interest expressed by the ceDNA vector involves circular permutation, as disclosed in U.S. Pat. No. 8,338,138. In such an embodiment, the molecular switch is multistable, i.e., able to switch between at least two states, or alternatively, bistable, i.e., a state is either “ON” or “OFF,” for example, able to emit light or not, able to bind or not, able to catalyze or not, able to transfer electrons or not, and so forth. In another aspect, the molecular switch uses a fusion molecule, therefore the switch is able to switch between more than two states. For example, in response to a particular threshold state exhibited by an insertion sequence or acceptor sequence, the respective other sequence of the fusion may exhibit a range of states (e.g., a range of binding activity, a range of enzyme catalysis, etc.). Thus, rather than switching from “ON” or “OFF,” the fusion molecule can exhibit a graded response to a stimulus.

In some embodiments, a nucleic acid based regulatory switch can be selected from any or a combination of the switches in Table 11.

E. Post-Transcriptional and Post-Translational Regulatory Switches.

In some embodiments, the regulatory switch to control the transgene or gene of interest expressed by the ceDNA vector is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Zhong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

In some embodiments, the regulatory switch to control the transgene or gene of interest expressed by the ceDNA vector is a post-translational modification system. In alternative embodiments, the gene of interest or protein is expressed as pro-protein or pre-proprotein, or has a signal response element (SRE) or a destabilizing domain (DD) attached to the expressed protein, thereby preventing correct protein folding and/or activity until post-translation modification has occurred. In the case of a destabilizing domain (DD) or SRE, the de-stabilization domain is post-translationally cleaved in the presence of an exogenous agent or small molecule. One of ordinary skill in the art can utilize such control methods as disclosed in U.S. Pat. No. 8,173,792 and PCT application WO2017180587. Other post-transcriptional control switches envisioned for use in the ceDNA vector for controlling functional transgene activity are disclosed in Rakhit et al., Chem Biol. 2014; 21(9):1238-52 and Navarro et al., ACS Chem Biol. 2016; 19; 11(8): 2101-2104A.

In some embodiments, a regulatory switch to control the transgene or gene of interest expressed by the ceDNA vector is a post-translational modification system that incorporates ligand sensitive inteins into the transgene coding sequence, such that the transgene or expressed protein is inhibited prior to splicing. For example, this has been demonstrated using both 4-hydroxytamoxifen and thyroid hormone (see, e.g., U.S. Pat. Nos. 7,541,450, 9,200,045; 7,192,739, Buskirk, et al, Proc Natl Acad Sci USA. 2004 Jul. 20; 101(29): 10505-10510; ACS Synth Biol. 2016 Dec. 16; 5(12): 1475-1484; and 2005 February; 14(2): 523-532. In some embodiments, a post-transcriptional based regulatory switch can be selected from any or a combination of the switches in Table 11.

F. Other Exemplary Regulatory Switches

Any known regulatory switch can be used in the ceDNA vector to control the gene expression of the transgene expressed by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the ceDNA vector is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, S368, as well as FROG, TOAD and NRSE elements and conditionally inducable silence elements, including hypoxia response elements (HREs), inflammatory response elements (IREs) and shear-stress activated elements (SSAEs), e.g., as disclosed in U.S. Pat. No. 9,394,526. Such an embodiment is useful for turning on expression of the transgene from the ceDNA vector after ischemia or in ischemic tissues, and/or tumors.

In some embodiments, a regulatory switch envisioned for use in the ceDNA vector is an optogenetic (e.g., light controlled) regulatory switch, e.g., such as one of the switches reviewed in Polesskaya et al., BMC Neurosci. 2018; 19(Suppl 1): 12, and are also envisioned for use herein. In such embodiments, a ceDNA vector can comprise genetic elements are light sensitive and can regulate transgene expression in response to visible wavelengths (e.g. blue, near IR). ceDNA vectors comprising optogenetic regulatory switches are useful when expressing the transgene in locations of the body that can receive such light sources, e.g., the skin, eye, muscle etc., and can also be used when ceDNA vectors are expressing transgenes in internal organs and tissues, where the light signal can be provided by a suitable means (e.g., implantable device as disclosed herein). Such optogenetic regulatory switches include use of the light responsive elements, or light-inducible transcriptional effector (LITE) (e.g., disclosed in 2014/0287938), a Light-On system (e.g., disclosed in Wang et al., Nat Methods. 2012 Feb. 12; 9(3):266-9; which has reported to enable in vivo control of expression of an insulin transgene, the Cry2/CIB1 system (e.g., disclosed on Kennedy et al., Nature Methods; 7, 973-975 (2010); and the FKF1/GIGANTEA system (e.g., disclosed in Yazawa et al., Nat Biotechnol. 2009 October; 27(10):941-5).

G. Kill Switches

Other embodiments of the invention relate to a ceDNA vector comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the ceDNA vectors of the invention would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector herein can restrict cell survival of a cell comprising a ceDNA vector to an environment defined by specific input signals. Such kill switches serve as a biological biocontainment function should it be desirable to remove the ceDNA vector from a subject or to ensure that it will not express the encoded transgene. Accordingly, kill switches are synthetic biological circuits in the ceDNA vector that couple environmental signals with conditional survival of the cell comprising the ceDNA vector. In some embodiments different ceDNA vectors can be designed to have different kill switches. This permits one to be able to control which transgene expressing cells are killed if cocktails of ceDNA vectors are used.

In some embodiments, a ceDNA vector can comprise a kill switch which is a modular biological containment circuit. In some embodiments, a kill switch encompassed for use in the ceDNA vector is disclosed in WO2017/059245, which describes a switch referred to as a “Deadman kill switch” that comprises a mutually inhibitory arrangement of at least two repressible sequences, such that an environmental signal represses the activity of a second molecule in the construct (e.g., a small molecule-binding transcription factor is used to produce a ‘survival’ state due to repression of toxin production). In cells comprising a ceDNA vector comprising a deadman kill switch, upon loss of the environmental signal, the circuit switches permanently to the ‘death’ state, where the toxin is now derepressed, resulting in toxin production which kills the cell. In another embodiment, a synthetic biological circuit referred to as a “Passcode circuit” or “Passcode kill switch” that uses hybrid transcription factors (TFs) to construct complex environmental requirements for cell survival, is provided. The Deadman and Passcode kill switches described in WO2017/059245 are particularly useful for use in ceDNA vectors, as they are modular and customizable, both in terms of the environmental conditions that control circuit activation and in the output modules that control cell fate. With the proper choice of toxins, including, but not limited to an endonuclease, e.g., a EcoRI, Passcode circuits present in the ceDNA vector can be used to not only kill the host cell comprising the ceDNA vector, but also to degrade its genome and accompanying plasmids.

Other kill switches known to a person of ordinary skill in the art are encompassed for use in the ceDNA vector as disclosed herein, e.g., as disclosed in US2010/0175141; US2013/0009799; US2011/0172826; US2013/0109568, as well as kill switches disclosed in Jusiak et al, Reviews in Cell Biology and molecular Medicine; 2014; 1-56; Kobayashi et al., PNAS, 2004; 101; 8419-9; Marchisio et al., Int. Journal of Biochem and Cell Biol., 2011; 43; 310-319; and in Reinshagen et al., Science Translational Medicine, 2018, 11.

Accordingly, in some embodiments, the ceDNA vector can comprise a kill switch nucleic acid construct, which comprises the nucleic acid encoding an effector toxin or reporter protein, where the expression of the effector toxin (e.g., a death protein) or reporter protein is controlled by a predetermined condition. For example, a predetermined condition can be the presence of an environmental agent, such as, e.g., an exogenous agent, without which the cell will default to expression of the effector toxin (e.g., a death protein) and be killed. In alternative embodiments, a predetermined condition is the presence of two or more environmental agents, e.g., the cell will only survive when two or more necessary exogenous agents are supplied, and without either of which, the cell comprising the ceDNA vector is killed.

In some embodiments, the ceDNA vector is modified to incorporate a kill-switch to destroy the cells comprising the ceDNA vector to effectively terminate the in vivo expression of the transgene being expressed by the ceDNA vector (e.g., therapeutic gene, protein or peptide etc). Specifically, the ceDNA vector is further genetically engineered to express a switch-protein that is not functional in mammalian cells under normal physiological conditions. Only upon administration of a drug or environmental condition that specifically targets this switch-protein, the cells expressing the switch-protein will be destroyed thereby terminating the expression of the therapeutic protein or peptide. For instance, it was reported that cells expressing HSV-thymidine kinase can be killed upon administration of drugs, such as ganciclovir and cytosine deaminase. See, for example, Dey and Evans, Suicide Gene Therapy by Herpes Simplex Virus-1 Thymidine Kinase (HSV-TK), in Targets in Gene Therapy, edited by You (2011); and Beltinger et al., Proc. Natl. Acad. Sci. USA 96(15):8699-8704 (1999). In some embodiments the ceDNA vector can comprise a siRNA kill switch referred to as DISE (Death Induced by Survival gene Elimination) (Murmann et al., Oncotarget. 2017; 8:84643-84658. Induction of DISE in ovarian cancer cells in vivo).

In some aspects, a deadman kill switch is a biological circuit or system rendering a cellular response sensitive to a predetermined condition, such as the lack of an agent in the cell growth environment, e.g., an exogenous agent. Such a circuit or system can comprise a nucleic acid construct comprising expression modules that form a deadman regulatory circuit sensitive to the predetermined condition, the construct comprising expression modules that form a regulatory circuit, the construct including:

i) a first repressor protein expression module, wherein the first repressor protein binds a first repressor protein nucleic acid binding element and represses transcription from a coding sequence comprising the first repressor protein binding element, and wherein repression activity of the first repressor protein is sensitive to inhibition by a first exogenous agent, the presence or absence of the first exogenous agent establishing a predetermined condition;

ii) a second repressor protein expression module, wherein the second repressor protein binds a second repressor protein nucleic acid binding element and represses transcription from a coding sequence comprising the second repressor protein binding element, wherein the second repressor protein is different from the first repressor protein; and

iii) an effector expression module, comprising a nucleic acid sequence encoding an effector protein, operably linked to a genetic element comprising a binding element for the second repressor protein, such that expression of the second repressor protein causes repression of effector expression from the effector expression module, wherein the second expression module comprises a first repressor protein nucleic acid binding element that permits repression of transcription of the second repressor protein when the element is bound by the first repressor protein, the respective modules forming a regulatory circuit such that in the absence of the first exogenous agent, the first repressor protein is produced from the first repressor protein expression module and represses transcription from the second repressor protein expression module, such that repression of effector expression by the second repressor protein is relieved, resulting in expression of the effector protein, but in the presence of the first exogenous agent, the activity of the first repressor protein is inhibited, permitting expression of the second repressor protein, which maintains expression of effector protein expression in the “off” state, such that the first exogenous agent is required by the circuit to maintain effector protein expression in the “off” state, and removal or absence of the first exogenous agent defaults to expression of the effector protein.

In some embodiments, the effector is a toxin or a protein that induces a cell death program. Any protein that is toxic to the host cell can be used. In some embodiments the toxin only kills those cells in which it is expressed. In other embodiments, the toxin kills other cells of the same host organism. Any of a large number of products that will lead to cell death can be employed in a deadman kill switch. Agents that inhibit DNA replication, protein translation or other processes or, e.g., that degrade the host cell's nucleic acid, are of particular usefulness. To identify an efficient mechanism to kill the host cells upon circuit activation, several toxin genes were tested that directly damage the host cell's DNA or RNA. The endonuclease ecoRI²¹, the DNA gyrase inhibitor ccdB²² and the ribonuclease-type toxin mazF²³ were tested because they are well-characterized, are native to E. coli, and provide a range of killing mechanisms. To increase the robustness of the circuit and provide an independent method of circuit-dependent cell death, the system can be further adapted to express, e.g., a targeted protease or nuclease that further interferes with the repressor that maintains the death gene in the “off” state. Upon loss or withdrawal of the survival signal, death gene repression is even more efficiently removed by, e.g., active degradation of the repressor protein or its message. As non-limiting examples, mf-Lon protease was used to not only degrade Lad but also target essential proteins for degradation. The mf-Lon degradation tag pdt#1 can be attached to the 3′ end of five essential genes whose protein products are particularly sensitive to mf-Lon degradation²⁰, and cell viability was measured following removal of ATc. Among the tested essential gene targets, the peptidoglycan biosynthesis gene murC provided the strongest and fastest cell death phenotype (survival ratio <1×10⁻⁴ within 6 hours).

As used herein, the term “predetermined input” refers to an agent or condition that influences the activity of a transcription factor polypeptide in a known manner. Generally, such agents can bind to and/or change the conformation of the transcription factor polypeptide to thereby modify the activity of the transcription factor polypeptide. Examples of predetermined inputs include, but are not limited to, environmental input agents that are not required for the survival of a given host organism (i.e., in the absence of a synthetic biological circuit as described herein). Conditions that can provide a predetermined input include, for example temperature, e.g., where the activity of one or more factors is temperature-sensitive, the presence or absence of light, including light of a given spectrum of wavelengths, and the concentration of a gas, salt, metal or mineral. Environmental input agents include, for example, a small molecule, biological agents such as pheromones, hormones, growth factors, metabolites, nutrients, and the like and analogs thereof; concentrations of chemicals, environmental byproducts, metal ions, and other such molecules or agents; light levels; temperature; mechanical stress or pressue; or electrical signals, such as currents and voltages.

In some embodiments, reporters are used to quantify the strength or activity of the signal received by the modules or programmable synthetic biological circuits of the invention. In some embodiments, reporters can be fused in-frame to other protein coding sequences to identify where a protein is located in a cell or organism. Luciferases can be used as effector proteins for various embodiments described herein, for example, measuring low levels of gene expression, because cells tend to have little to no background luminescence in the absence of a luciferase. In other embodiments, enzymes that produce colored substrates can be quantified using spectrophotometers or other instruments that can take absorbance measurements including plate readers. Like luciferases, enzymes like β-galactosidase can be used for measuring low levels of gene expression because they tend to amplify low signals. In some embodiments, an effector protein can be an enzyme that can degrade or otherwise destroy a given toxin. In some embodiments, an effector protein can be an odorant enzyme that converts a substrate to an odorant product. In some embodiments, an effector protein can be an enzyme that phosphorylates or dephosphorylates either small molecules or other proteins, or an enzyme that methylates or demethylates other proteins or DNA.

In some embodiments, an effector protein can be a receptor, ligand, or lytic protein. Receptors tend to have three domains: an extracellular domain for binding ligands such as proteins, peptides or small molecules, a transmembrane domain, and an intracellular or cytoplasmic domain which frequently can participate in some sort of signal transduction event such as phosphorylation. In some embodiments, transporter, channel, or pump gene sequences are used as effector proteins. Non-limiting examples and sequences of effector proteins for use with the kill switches as described herein can be found at the Registry of Standard Biological Parts on the world wide web at parts.igem.org.

As used herein, a “modulator protein” is a protein that modulates the expression from a target nucleic acid sequence. Modulator proteins include, for example, transcription factors, including transcriptional activators and repressors, among others, and proteins that bind to or modify a transcription factor and influence its activity. In some embodiments, a modulator protein includes, for example, a protease that degrades a protein factor involved in the regulation of expression from a target nucleic acid sequence. Preferred modulator proteins include modular proteins in which, for example, DNA-binding and input agent-binding or responsive elements or domains are separable and transferrable, such that, for example, the fusion of the DNA binding domain of a first modulator protein to the input agent-responsive domain of a second results in a new protein that binds the DNA sequence recognized by the first protein, yet is sensitive to the input agent to which the second protein normally responds. Accordingly, as used herein, the term “modulator polypeptide,” and the more specific “repressor polypeptide” include, in addition to the specified polypeptides, e.g., “a Lad (repressor) polypeptide,” variants, or derivatives of such polypeptides that responds to a different or variant input agent. Thus, for a Lad polypeptide, included are Lad mutants or variants that bind to agents other than lactose or IPTG. A wide range of such agents are known in the art.

TABLE 11
Exemplary regulatory switches

| | | ON | OFF | | | |
|---|---|---|---|---|---|---|
| no. | name | switch^{b} | switch^{c} | origin | effector^{d} | references^{e} |
| Transcriptional Switches | | | | | | |
| 1 | ABA | yes | no | Arabidopsis | abscisic acid | [19] |
| | | | | thaliana, yeast | | |
| 2 | AIR | yes | no | Aspergillus | acetaldehyde | [20] |
| | | | | nidulans | | |
| 3 | ART | yes | no | Chlamydia | l-arginine | [21] |
| | | | | pneumoniae | | |
| 4 | BEARON, | yes | yes | Campylobacter | bile acid | [22] |
| | BEAROFF | | | jejuni | | |
| 5 | BirA-tTA | no | yes | Escherichia coli | biotin (vitamin H) | [23] |
| 6 | BIT | yes | no | Escherichia coli | biotin (vitamin H) | [24] |
| 7 | Cry2-CIB1 | yes | no | Arabidopsis | blue light | [25] |
| | | | | thaliana, yeast | | |
| 8 | CTA, CTS | yes | yes | Comamonas | food additives | [26] |
| | | | | testosteroni, | (benzoate, vanillate) | |
| | | | | Homo sapiens | | |
| 9 | cTA, rcTA | yes | yes | Pseudomonas | cumate | [27] |
| | | | | putida | | |
| 10 | Ecdysone | yes | no | Homo sapiens, | Ecdysone | [28] |
| | | | | Drosophila | | |
| | | | | melanogaster | | |
| 11 | EcR:RXR | yes | no | Homo sapiens, | ecdysone | [29] |
| | | | | Locusta | | |
| | | | | migratoria | | |
| 12 | electro- | yes | no | Aspergillus | electricity, | [30] |
| | genetic | | | nidulans | acetaldehyde | |
| 13 | ER-p65-ZF | yes | no | Homo sapiens, | 4,4′- | [31] |
| | | | | yeast | dyhydroxybenzil | |
| 14 | E.REX | yes | yes | Escherichia coli | erythromycin | [32] |
| 15 | EthR | no | yes | Mycobacterium | 2-phenylethyl- | [33] |
| | | | | tuberculosis | butyrate | |
| 16 | GAL4-ER | yes | yes | yeast, Homo | oestrogen, 4- | [34] |
| | | | | sapiens | hydroxytamoxifen | |
| 17 | GAL4-hPR | yes | yes | yeast, Homo | mifepristone | [35, 36] |
| | | | | sapiens | | |
| 18 | GAL4- | yes | yes | yeast, Homo | rapamycin and | [37] |
| | Raps | | | sapiens | rapamycin | |
| | | | | | derivatives | |
| 19 | GAL4-TR | yes | no | yeast, Homo | thyroid hormone | [38] |
| | | | | sapiens | | |
| 20 | GyrB | yes | yes | Escherichia coli | coumermycin, | [39] |
| | | | | | novobiocin | |
| 21 | HEA-3 | yes | no | Homo sapiens | 4-hydroxytamoxifen | [40] |
| 22 | Intramer | no | yes | synthetic | theophylline | [41] |
| | | | | SELEX-derived | | |
| | | | | aptamers | | |
| 23 | LacI | yes | no | Escherichia coli | IPTG | [42-46] |
| 24 | LAD | yes | no | Arabidopsis | blue light | [47] |
| | | | | thaliana, yeast | | |
| 25 | LightOn | yes | no | Neurospora | blue light | [48] |
| | | | | crassa, yeast | | |
| 26 | NICE | yes | yes | Arthrobacter | 6-hydroxynicotine | [49] |
| | | | | nicotinovorans | | |
| 27 | PPAR* | yes | no | Homo sapiens | rosiglitazone | [50] |
| 28 | PEACE | no | yes | Pseudomonas | flavonoids (e.g. | [51] |
| | | | | putida | phloretin) | |
| 29 | PIT | yes | yes | Streptomyces | pristinamycin I, | [12] |
| | | | | coelicolor | virginiamycin | |
| 30 | REDOX | no | yes | Streptomyces | NADH | [52] |
| | | | | coelicolor | | |
| 31 | QuoRex | yes | yes | Streptomyces | butyrolactones (e.g. | [53] |
| | | | | coelicolor, | SCB1) | |
| | | | | Streptomyces | | |
| | | | | pristinaespiralis | | |
| 32 | ST-TA | yes | yes | Streptomyces | γ-butyrolactone, | [54] |
| | | | | coelicolor, | tetracycline | |
| | | | | Escherichia | | |
| | | | | coli, Herpes | | |
| | | | | simplex | | |
| 33 | TIGR | no | yes | Streptomyces | temperature | [55] |
| | | | | albus | | |
| 34 | TraR | yes | no | Agrobacterium | N-(3-oxo- | [56] |
| | | | | tumefaciens | octanoyl)homoserine | |
| | | | | | lactone | |
| 35 | TET-OFF, | yes | yes | Escherichia | tetracycline, | [11,57] |
| | TET-ON | | | coli, Herpes | doxycycline | |
| | | | | simplex | | |
| 36 | TRT | yes | no | Chlamydia | l-tryptophan | [58] |
| | | | | trachomatis | | |
| 37 | UREX | yes | no | Deinococcus | uric acid | [59] |
| | | | | radiodurans | | |
| 38 | VAC | yes | yes | Caulobacter | vanillic acid | [60] |
| | | | | crescentus | | |
| 39 | ZF-ER, ZF- | yes | yes | Mus musculus, | 4- | [61] |
| | RXR/EcR | | | Homo sapiens, | hydroxytamoxifen, | |
| | | | | Drosophila | ponasterone-A | |
| | | | | melanogaster | | |
| 40 | ZF-Raps | yes | no | Homo sapiens | rapamycin | [62] |
| 41 | ZF | yes | no | Mus musculus, | 4- | [63] |
| | switches | | | Homo sapiens, | hydroxytamoxifen, | |
| | | | | Drosophila | mifepristone | |
| | | | | melanogaster | | |
| 42 | ZF(TF)s | yes | no | Xenopus laevis, | ethyl-4- | [64] |
| | | | | Homo sapiens | hydroxybenzoate, | |
| | | | | | propyl-4- | |
| | | | | | hydroxybenzoate | |

post-transcriptional switches

| | | | | | | |
|---|---|---|---|---|---|---|
| 1 | aptamer | yes | no | synthetic | theophylline | [65] |
| | RNAi | | | SELEX-derived | | |
| | | | | aptamer | | |
| 2 | aptamer | no | yes | synthetic | theophylline | [66] |
| | RNAi | | | SELEX-derived | | |
| | | | | aptamer | | |
| 3 | aptamer | yes | no | synthetic | theophylline, | [67] |
| | RNAi | | | SELEX-derived | tetracycline, | |
| | miRNA | | | aptamer | hypoxanthine | |
| 4 | aptamer | yes | yes | Homo sapiens, | MS2, p65, p50, b- | [68] |
| | Splicing | | | MS2 | catenin | |
| | | | | bacteriophage | | |
| 5 | aptazyme | no | yes | synthetic | theophylline | [69] |
| | | | | SELEX-derived | | |
| | | | | aptamer, | | |
| | | | | Schistosoma | | |
| | | | | mansoni | | |
| 6 | replicon | yes | no | Sindbis virus | temperature | [70] |
| | CytTS | | | | | |
| 7 | TET-OFF- | yes | yes | Escherichia | doxycycline | [71] |
| | shRNA, | | | coli, Herpes | | |
| | TET-ON- | | | simplex, Homo | | |
| | shRNA | | | sapiens | | |
| 8 | theo | no | yes | synthetic | theophylline | [72] |
| | aptamer | | | SELEX-derived | | |
| | | | | aptamer | | |
| 9 | 3′ UTR | yes | no | synthetic | theophylline, | [73] |
| | aptazyme | | | SELEX-derived | tetracycline | |
| | | | | aptamers, | | |
| | | | | tobacco | | |
| | | | | ringspot virus | | |
| 10 | 5′ UTR | no | yes | synthetic | theophylline | [74] |
| | aptazyme | | | SELEX-derived | | |
| | | | | aptamer, | | |
| | | | | Schistosoma | | |
| | | | | mansoni | | |

translational switches

| | | | | | | |
|---|---|---|---|---|---|---|
| 1 | Hoechst | no | yes | synthetic RNA | Hoechst dyes | [75] |
| | aptamer | | | sequence | | |
| 2 | H23 | no | yes | Archaeoglobus | L7Ae, L7KK | [76] |
| | aptamer | | | fulgidus | | |
| 3 | L7Ae | yes | yes | Archaeoglobus | L7Ae | [77] |
| | aptamer | | | fulgidus | | |
| 4 | MS2 | no | yes | MS2 | MS2 | [78] |
| | aptamer | | | bacteriophage | | |

post-translational switches

| | | | | | | |
|---|---|---|---|---|---|---|
| 1 | AID | no | yes | Arabidopsis | auxins (e.g. IAA) | [79] |
| | | | | thaliana, Oryza | | |
| | | | | sativa, | | |
| | | | | Gossypium | | |
| | | | | hirsutum | | |
| 2 | ER DD | no | yes | Homo sapiens | CMP8, 4- | [80] |
| | | | | | hydroxytamoxifen | |
| 3 | FM | yes | no | Homo sapiens | AP21998 | [81] |
| 4 | HaloTag | no | yes | Rhodococcus | HyT13 | [82, 83] |
| | | | | sp. RHA1 | | |
| 5 | HDV- | no | yes | hepatitis delta | theophylline, | [84] |
| | aptazyme | | | vims | guanine | |
| 6 | PROTAC | no | yes | Homo sapiens | proteolysis targeting | [85] |
| | | | | | chimeric molecules | |
| | | | | | (PROTACS) | |
| 7 | shield DD | yes | no | Homo sapiens | shields (e.g. Shld1) | [86] |
| 8 | shield LID | no | yes | Homo sapiens | shields (e.g. Shld1) | [87] |
| 9 | TMP DD | yes | no | Escherichia coli | trimethoprim (TMP) | [88] |

^{b}ON switchability by an effector; other than removing the effector which confers the OFF state.
^{c}OFF switchability by an effector; other than removing the effector which confers the ON state.
^{d}A ligand or other physical stimuli (e.g. temperature, electromagnetic radiation, electricity) which stabilizes the switch either in its ON or OFF state.
^{e}refers to the reference number cited in Kis et al., J R Soc Interface. 12: 20141000 (2015), where both the article and the references cited therein are hereby incorporated by reference herein.

### VI. Detailed Method of Production of a ceDNA Vector

A. Production in General

As described herein, the ceDNA vector can be obtained by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the invention provides for use of host cell lines that have stably integrated the DNA vector polynucleotide expression template (ceDNA template) into their own genome in production of the non-viral DNA vector, e.g. as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors described herein are insect cells, and baculovirus is used to deliver both the polynucleotide that encodes Rep protein and the non-viral DNA vector polynucleotide expression construct template for ceDNA, e.g., as described in FIGS. 4A-4C and Example 1. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

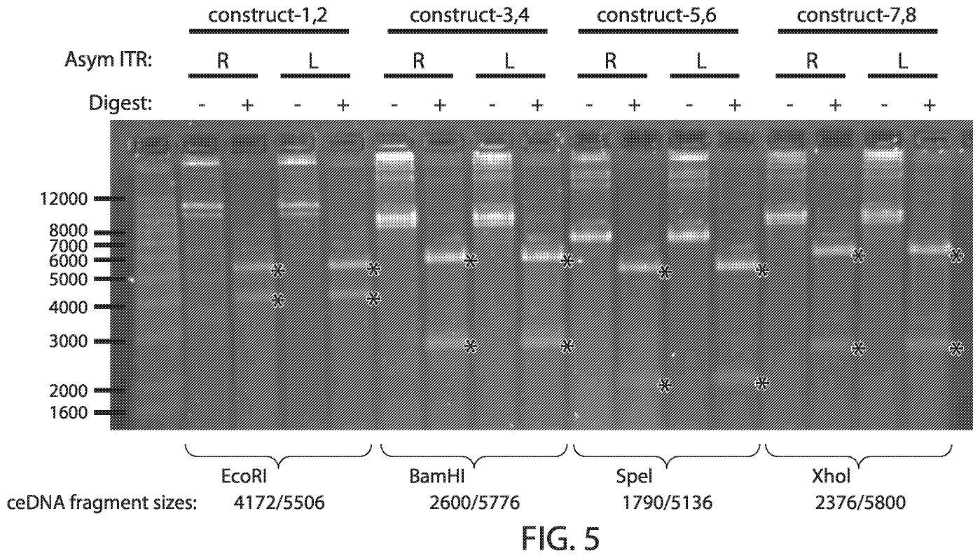

The presence of the ceDNA vector can be confirmed by digesting the vector DNA isolated from the cells with a restriction enzyme having a single recognition site on the DNA vector and analyzing both digested and undigested DNA material using gel eletrophoresis to confirm the presence of characteristic bands of linear and continuous DNA as compared to linear and non-continuous DNA. FIGS. 4C and 4E illustrate one embodiment for identifying the presence of the closed ended ceDNA vectors produced by the processes herein. For example, FIG. 5 is a gel confirming the production of ceDNA from multiple plasmid constructs using one embodiment for producing these vectors as described in the Examples.

B. ceDNA Plasmid

A ceDNA-plasmid is a plasmid used for later production of a ceDNA vector. In some embodiments, a ceDNA-plasmid can be constructed using known techniques to provide at least the following as operatively linked components in the direction of transcription: (1) a 5′ ITR sequence; (2) an expression cassette containing a cis-regulatory element, for example, a promoter, inducible promoter, regulatory switch, enhancers and the like; and (3) a 3′ ITR sequence, where the 3′ ITR sequence is asymmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) wild-type AAV ITR, wherein said ceDNA-plasmid is devoid of AAV capsid protein coding sequences, and wherein the 5′ and 3′ ITRs are asymmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are different and do not have the same modifications.

In a further embodiment, the ceDNA-plasmid sytem is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present invention can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at www web address: oesys.springer.de/viruses/database/mkchapter.asp?virID=42.04.) (note—references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An Exemplary ceDNA (e.g., rAAVO) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors are also provided herein, notably a method with a sufficiently high yield to provide sufficient vector for in vivo experiments.

In some embodiments, a method for the production of a ceDNA vector comprises the steps of: (1) introducing the nucleic acid construct comprising an expression cassette and two asymmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of capsid-free AAV vector can be in the form of a cfAAV-plasmid, or Bacmid or Baculovirus generated with the cfAAV-plasmid as described below. The nucleic acid construct can be introduced into a host cell by transfection, viral transduction, stable integration, or other methods known in the art.

D. Cell Lines:

Host cell lines used in the production of a ceDNA vector can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the ceDBA-plasmid for high yield ceDNA vector production.

ceDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

E. Isolating and Purifying ceDNA Vectors:

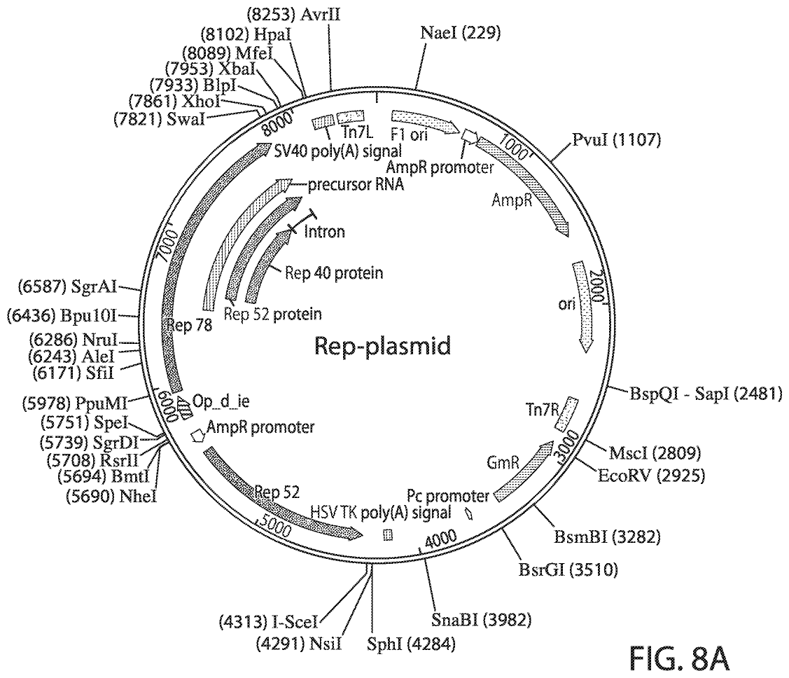

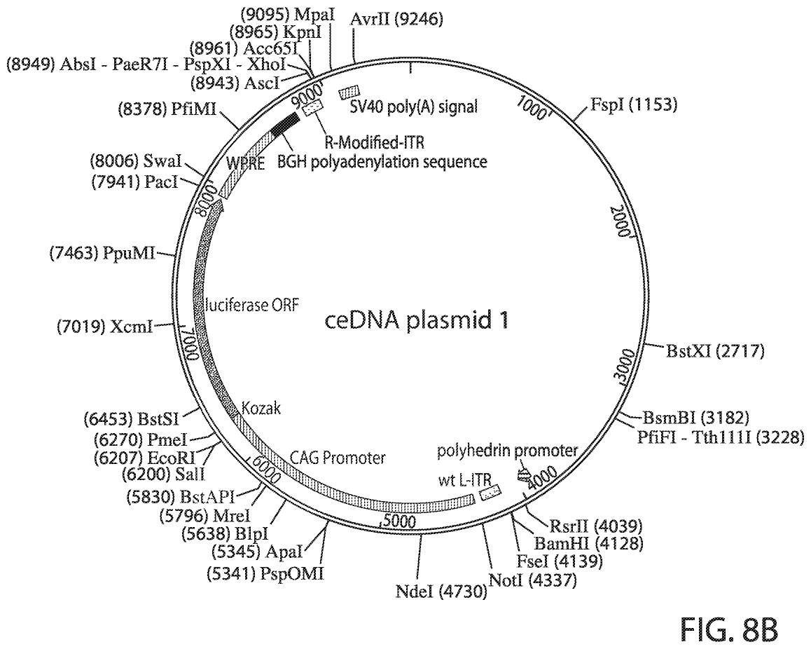

Examples of the process for obtaining and isolating ceDNA vectors are described in FIGS. 4A-4E and the specific examples below. ceDNA-vectors disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids shown in FIG. 8A (useful for Rep BIICs production), FIG. 8B (plasmid used to obtain a ceDNA vector).

In one aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA-vector, which is an exemplary ceDNA vector, are described herein. Expression constructs used for generating a ceDNA vectors of the present invention can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors can be generated from the cells stably transected with a construct comprising a sequence encoding the AAV Rep protein (Rep78/52) delivered in Rep-plasmids, Rep-bacmids, or Rep-baculovirus. ceDNA-Baculovirus can be transiently transfected to the cells, be replicated by Rep protein and produce ceDNA vectors.

The bacmid (e.g., ceDNA-bacmid) can be transfected into a permissive insect cells such as Sf9, Sf21, Tni (Trichoplusia ni) cell, High Five cell, and generate ceDNA-baculovirus, which is a recombinant baculovirus including the sequences comprising the asymmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one nonlimiting example, the process can be performed by loading the supernatant on an ion exchange column (e.g. SARTOBIND Q®) which retains nucleic acids, and then eluting (e.g. with a 1.2 M NaCl solution) and performing a further chromatographic purification on a gel filtration column (e.g. 6 fast flow GE). The capsid-free AAV vector is then recovered by, e.g., precipitation.

In some embodiments, ceDNA vectors can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the invention herein relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

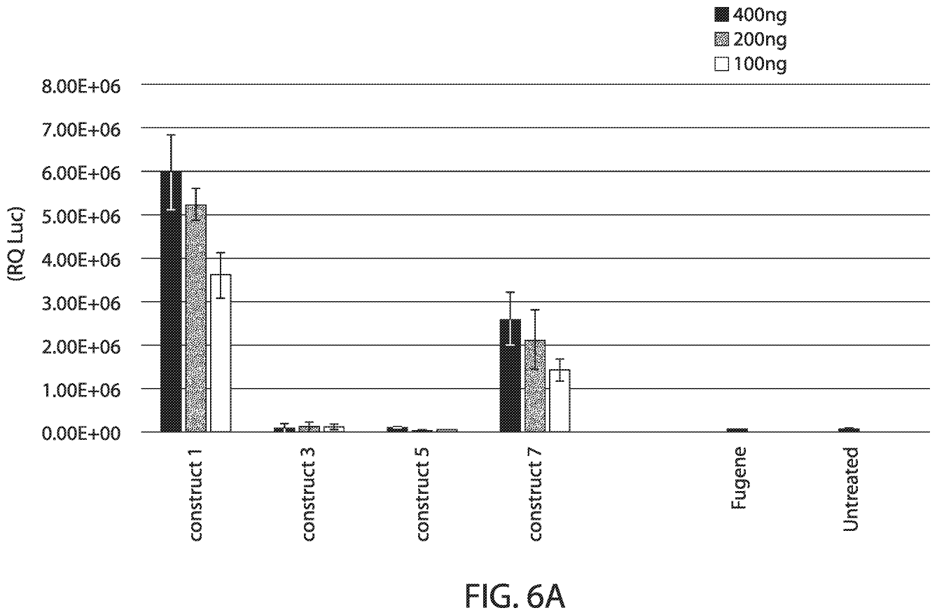

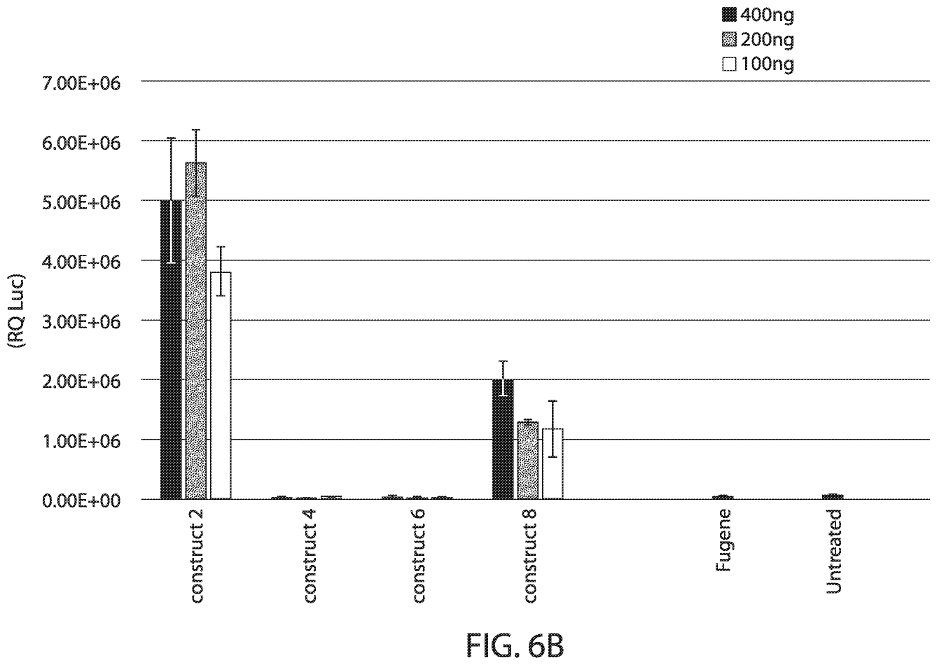

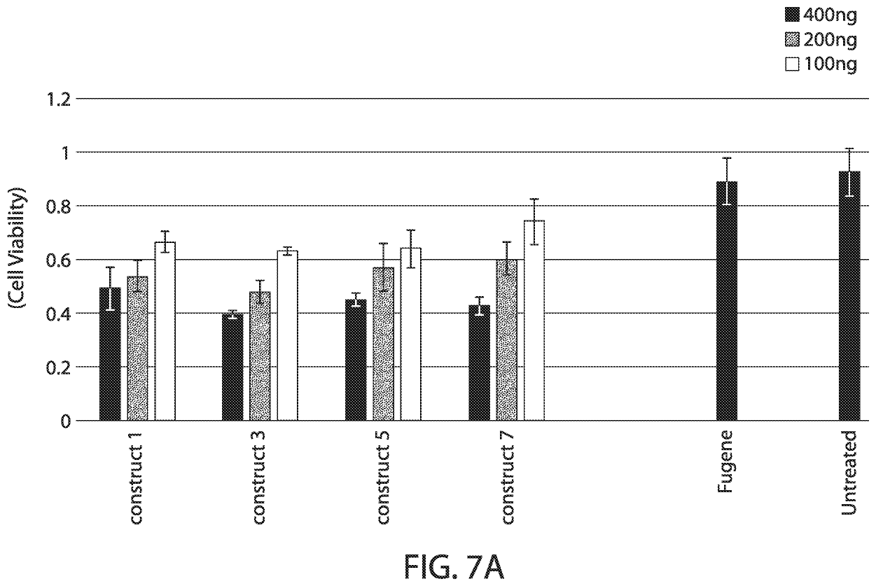

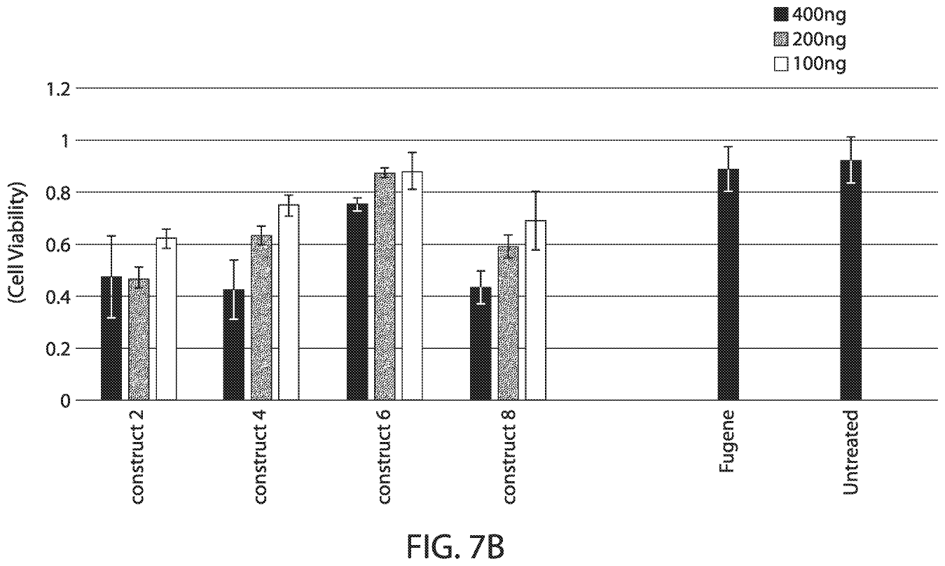

FIG. 5 shows a gel confirming the production of ceDNA from multiple ceDNA-plasmid constructs using the method described in the Examples. The ceDNA is confirmed by a characteristic band pattern in the gel, as discussed with respect to FIG. 4D in the Examples. Other characteristics of the ceDNA production process and intermediates are summarized in FIGS. 6A and 6B, and FIGS. 7A and 7B, as described in the Examples.

### VII. Pharmaceutical Compositions

In another aspect, pharmaceutical compositions are provided. The pharmaceutical composition comprises a ceDNA vector as disclosed herein and a pharmaceutically acceptable carrier or diluent.

The DNA-vectors disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier. For example, the ceDNA vectors described herein can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

Pharmaceutically active compositions comprising a ceDNA vector can be formulated to deliver a transgene in the nucleic acid to the cells of a recipient, resulting in the therapeutic expression of the transgene therein. The composition can also include a pharmaceutically acceptable carrier.

A ceDNA vector as disclosed herein can be incorporated into a pharmaceutical composition suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

Various techniques and methods are known in the art for delivering nucleic acids to cells. For example, nucleic acids, such as ceDNA can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA, can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

Methods of non-viral delivery of nucleic acids in vivo or ex vivo include electroporation, lipofection (see, U.S. Pat. Nos. 5,049,386; 4,946,787 and commercially available reagents such as Transfectam™ and Lipofectin™), microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, and agent-enhanced uptake of DNA. Sonoporation using, e.g., the Sonitron 2000 system (Rich-Mar) can also be used for delivery of nucleic acids.

ceDNA vectors as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector as disclosed herein can be delivered into hematopoietic stem cells, for example, by the methods as decribed, for example, in U.S. Pat. No. 5,928,638.

The ceDNA vectors in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is wither unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In other aspects, the disclosure provides for a liposome formulation comprising phospholipids, lecithin, phosphatidylcholine and phosphatidylethanolamine.

Delivery reagents such as liposomes, nanocapsules, microparticles, microspheres, lipid particles, vesicles, and the like, can be used for the introduction of the compositions of the present disclosure into suitable host cells. In particular, the nucleic acids can be formulated for delivery either encapsulated in a lipid particle, a liposome, a vesicle, a nanosphere, a nanoparticle, a gold particle, or the like. Such formulations can be preferred for the introduction of pharmaceutically acceptable formulations of the nucleic acids disclosed herein.

Various delivery methods known in the art or modification thereof can be used to deliver ceDNA vectors in vitro or in vivo. For example, in some embodiments, ceDNA vectors are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells.

In some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

In some embodiments, electroporation is used to deliver ceDNA vectors. Electroporation causes temporary destabilization of the cell membrane target cell tissue by insertion of a pair of electrodes into the tissue so that DNA molecules in the surrounding media of the destabilized membrane would be able to penetrate into cytoplasm and nucleoplasm of the cell. Electroporation has been used in vivo for many types of tissues, such as skin, lung, and muscle.

In some cases, a ceDNA vector is delivered by hydrodynamic injection, which is a simple and highly efficient method for direct intracellular delivery of any water-soluble compounds and particles into internal organs and skeletal muscle in an entire limb.

In some cases, ceDNA vectors are delivered by ultrasound by making nanoscopic pores in membrane to facilitate intracellular delivery of DNA particles into cells of internal organs or tumors, so the size and concentration of plasmid DNA have great role in efficiency of the system. In some cases, ceDNA vectors are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

A. Exosomes:

In some embodiments, a ceDNA vector as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

B. Microparticle/Nanoparticles:

In some embodiments, a ceDNA vector as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector disclosed herein. For example, various delivery methods using lipid nanoparticles are described in U.S. Pat. Nos. 9,404,127, 9,006,417 and 9,518,272.

In some embodiments, a ceDNA vector disclosed herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

C. Liposomes

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 ANG., containing an aqueous solution in the core.

In some embodiments, a liposome comprises cationic lipids. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In some embodiments, cationic lipids include saturated and unsaturated alkyl and alicyclic ethers and esters of amines, amides, or derivatives thereof. In some embodiments, cationic lipids comprise straight-chain, branched alkyl, alkenyl groups, or any combination of the foregoing. In some embodiments, cationic lipids contain from 1 to about 25 carbon atoms (e.g., 1, 2, 3, 4, 5, 6, 7, 8, 9, 10, 11, 12, 13, 14, 15, 16, 17, 18, 19, 20, 21, 22, 23, 24, or 25 carbon atoms. In some embodiments, cationic lipids contain more than 25 carbon atoms. In some embodiments, straight chain or branched alkyl or alkene groups have six or more carbon atoms. A cationic lipid can also comprise, in some embodiments, one or more alicyclic groups. Non-limiting examples of alicyclic groups include cholesterol and other steroid groups. In some embodiments, cationic lipids are prepared with a one or more counterions. Examples of counterions (anions) include but are not limited to Cl⁻, Br⁻, I⁻, F⁻, acetate, trifluoroacetate, sulfate, nitrite, and nitrate.

Non-limiting examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N-(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., CELiD) can also be complexed with, e.g., poly (L-lysine) or avidin and lipids can, or can not, be included in this mixture, e.g., steryl-poly (L-lysine).

In some embodiments, a ceDNA vector as disclosed herein is delivered using a cationic lipid described in U.S. Pat. No. 8,158,601, or a polyamine compound or lipid as described in U.S. Pat. No. 8,034,376.

D. Conjugates

In some embodiments, a ceDNA vector as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a ceDNA vector as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a ceDNA vector as disclosed herein is conjugated to a carbohydrate, for example as described in U.S. Pat. No. 8,450,467.

E. Nanocapsule

Alternatively, nanocapsule formulations of a ceDNA vector as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### VIII. Methods of Delivering ceDNA Vectors

In some embodiments, a ceDNA vector can be delivered to a target cell in vitro or in vivo by various suitable methods. ceDNA vectors alone can be applied or injected. CeDNA vectors can be delivered to a cell without the help of a transfection reagent or other physical means. Alternatively, ceDNA vectors can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

In contrast, transductions with capsid-free AAV vectors disclosed herein can efficiently target cell and tissue-types that are difficult to transduce with conventional AAV virions using various delivery reagent.

In another embodiment, a ceDNA vector is administered to the CNS (e.g., to the brain or to the eye). The ceDNA vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may also be administered to different regions of the eye such as the retina, cornea and/or optic nerve. The ceDNA vector may be delivered into the cerebrospinal fluid (e.g., by lumbar puncture). The ceDNA vector may further be administered intravascularly to the CNS in situations in which the blood-brain barrier has been perturbed (e.g., brain tumor or cerebral infarct).

In some embodiments, the ceDNA vector can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and pen-ocular (e.g., sub-Tenon's region) delivery as well as intramuscular delivery with retrograde delivery to motor neurons.

In some embodiments, the ceDNA vector is administered in a liquid formulation by direct injection (e.g., stereotactic injection) to the desired region or compartment in the CNS. In other embodiments, the ceDNA vector can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898). In yet additional embodiments, the ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vector can be delivered to muscle tissue from which it can migrate into neurons.

### VIII. Additional Uses of the ceDNA Vectors

The compositions and ceDNA vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment, prevention, or amelioration of disease states or disorders in a mammalian subject. The transgene can be transferred (e.g., expressed in) to a subject in a sufficient amount to treat a disease associated with reduced expression, lack of expression or dysfunction of the gene. In some embodiments the transgene can be transferred to (e.g., expressed in) a subject in a sufficient amount to treat a disease associated with increased expression, activity of the gene product, or inappropriate upregulation of a gene that the transgene suppresses or otherwise causes the expression of which to be reduced.

### IX. Methods of Use

The ceDNA vector of the invention can also be used in a method for the delivery of a nucleotide sequence of interest to a target cell. The method may in particular be a method for delivering a therapeutic gene of interest to a cell of a subject in need thereof. The invention allows for the in vivo expression of a polypeptide, protein, or oligonucleotide encoded by a therapeutic exogenous DNA sequence in cells in a subject such that therapeutic levels of the polypeptide, protein, or oligonucleotide are expressed. These results are seen with both in vivo and in vitro modes of ceDNA vector delivery.

A method for the delivery of a nucleic acid of interest in a cell of a subject can comprise the administration to said subject of a ceDNA vector of the invention comprising said nucleic acid of interest. In addition, the invention provides a method for the delivery of a nucleic acid of interest in a cell of a subject in need thereof, comprising multiple administrations of the ceDNA vector of the invention comprising said nucleic acid of interest. Since the ceDNA vector of the invention does not induce an immune response, such a multiple administration strategy will not be impaired by the host immune system response against the ceDNA vector of the invention, contrary to what is observed with encapsidated vectors.

The ceDNA vector nucleic acid(s) are administered in sufficient amounts to transfect the cells of a desired tissue and to provide sufficient levels of gene transfer and expression without undue adverse effects. Conventional and pharmaceutically acceptable routes of administration include, but are not limited to, intravenous (e.g., in a liposome formulation), direct delivery to the selected organ (e.g., intraportal delivery to the liver), intramuscular, and other parental routes of administration. Routes of administration may be combined, if desired.

CeDNA vector delivery is not limited to one species of ceDNA vector. As such, in another aspect, multiple ceDNA vectors comprising different exogenous DNA sequences can be delivered simultaneously or sequentially to the target cell, tissue, organ, or subject. Therefore, this strategy can allow for the expression of multiple genes. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The invention also provides for a method of treating a disease in a subject comprising introducing into a target cell in need thereof (in particular a muscle cell or tissue) of the subject a therapeutically effective amount of a ceDNA vector, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

### X. Methods of Treatment

The technology described herein also demonstrates methods for making, as well as methods of using the disclosed ceDNA vectors in a variety of ways, including, for example, ex situ, in vitro and in vivo applications, methodologies, diagnostic procedures, and/or gene therapy regimens.

Provided herein is a method of treating a disease or disorder in a subject comprising introducing into a target cell in need thereof (for example, a muscle cell or tissue, or other affected cell type) of the subject a therapeutically effective amount of a ceDNA vector, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

Any transgene, may be delivered by the ceDNA vectors as disclosed herein. Transgenes of interest include nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides.

In certain embodiments, the transgenes to be expressed by the ceDNA vectors described herein will express or encode one or more polypeptides, peptides, ribozymes, peptide nucleic acids, siRNAs, RNAis, antisense oligonucleotides, antisense polynucleotides, antibodies, antigen binding fragments, or any combination thereof.

In particular, the transgene can encode one or more therapeutic agent(s), including, but not limited to, for example, protein(s), polypeptide(s), peptide(s), enzyme(s), antibodies, antigen binding fragments, as well as variants, and/or active fragments thereof, agonists, antagonists, mimetics for use in the treatment, prophylaxis, and/or amelioration of one or more symptoms of a disease, dysfunction, injury, and/or disorder. In one aspect, the disease, dysfunction, trauma, injury and/or disorder is a human disease, dysfunction, trauma, injury, and/or disorder.

As noted herein, the transgene can encode a therapeutic protein or peptide, or therapeutic nucleic acid sequence or therapeutic agent, including but not limited to one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

In some embodiments, a transgene in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

In some embodiments, the ceDNA vector expresses the transgene in a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

Disclosed herein are ceDNA vector compositions and formulations that include one or more of the ceDNA vectors of the present invention together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a ceDNA vector, the method comprising providing to a cell, tissue or organ of a subject in need thereof, an amount of the ceDNA vector as disclosed herein; and for a time effective to enable expression of the transgene from the ceDNA vector thereby providing the subject with a diagnostically- or a therapeutically-effective amount of the protein, peptide, nucleic acid expressed by the ceDNA vector. In a further aspect, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed ceDNA vectors, in an amount and for a time sufficient to diagnose, prevent, treat or ameliorate the one or more symptoms of the disease, disorder, dysfunction, injury, abnormal condition, or trauma in the subject. In a further aspect, the subject is human.

Another aspect is use of the ceDNA vector as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, ceDNA vectors can be used to deliver transgenes to bring a normal gene into affected tissues for replacement therapy, as well, in some embodiments, to create animal models for the disease using antisense mutations. For unbalanced disease states, ceDNA vectors can be used to create a disease state in a model system, which could then be used in efforts to counteract the disease state. Thus the ceDNA vectors and methods disclosed herein permit the treatment of genetic diseases. As used herein, a disease state is treated by partially or wholly remedying the deficiency or imbalance that causes the disease or makes it more severe.

In general, the ceDNA vector as disclosed herein can be used to deliver any transgene to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., omithine transcarbamylase deficiency).

In some embodiments, the ceDNA vector described herein can be used to treat, ameliorate, and/or prevent a disease or disorder caused by mutation in a gene or gene product. Exemplary diseases or disorders that can be treated with a ceDNA vectors include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

As still a further aspect, a ceDNA vector as disclosed herein may be employed to deliver a heterologous nucleotide sequence in situations in which it is desirable to regulate the level of transgene expression (e.g., transgenes encoding hormones or growth factors, as described herein).

Accordingly, in some embodiments, the ceDNA vector described herein can be used to correct an abnormal level and/or function of a gene product (e.g., an absence of, or a defect in, a protein) that results in the disease or disorder. The ceDNA vector can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MLD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In alternative embodiments, the ceDNA vectors as disclosed herein can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In some embodiments, exemplary transgenes encoded by the ceDNA vector include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-α and -β, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is a antigen-binding domain or a immunoglobulin variable domain sequence, as that is defined hereinOther illustrative transgene sequences encode suicide gene products (thymdine kinase, cytosine deaminase, diphtheria toxin, cytochrome P450, deoxycytidine kinase, and tumor necrosis factor), proteins conferring resistance to a drug used in cancer therapy, and tumor suppressor gene products.

In a representative embodiment, the transgene expressed by the ceDNA vector can be used for the treatment of muscular dystrophy in a subject in need thereof, the method comprising: administering a treatment-, amelioration- or prevention-effective amount of ceDNA vector described herein, wherein the ceDNA vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, a-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the ceDNA vector can be administered to skeletal, diaphragm and/or cardiac muscle as described elsewhere herein.

In some embodiments, the ceDNA vector can be used to deliver a transgene to skeletal, cardiac or diaphragm muscle, for production of a polypeptide (e.g., an enzyme) or functional RNA (e.g., RNAi, microRNA, antisense RNA) that normally circulates in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid.alpha.-glucosidase] or Fabry disease [.alpha.-galactosidase A]) or a glycogen storage disorder (such as Pompe disease [lysosomal acid a glucosidase]). Other suitable proteins for treating, ameliorating, and/or preventing metabolic disorders are described above.

In other embodiments, the ceDNA vector as disclosed herein can be used to deliver a transgene in a method of treating, ameliorating, and/or preventing a metabolic disorder in a subject in need thereof. Illustrative metabolic disorders and transgenes encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art).

Another aspect of the invention relates to a method of treating, ameliorating, and/or preventing congenital heart failure or PAD in a subject in need thereof, the method comprising administering a ceDNA vector as described herein to a mammalian subject, wherein the ceDNA vector comprises a transgene encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, .beta.2-adrenergic receptor kinase (BARK), PI3 kinase, calsarcan, a .beta.-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active βARKct, Pim-1, PGC-1α, SOD-1, SOD-2, EC-SOD, kallikrein, HIF, thymosin-β4, mir-1, mir-133, mir-206 and/or mir-208.

The ceDNA vectors as disclosed herein can be administered to the lungs of a subject by any suitable means, optionally by administering an aerosol suspension of respirable particles comprising the ceDNA vectors, which the subject inhales. The respirable particles can be liquid or solid. Aerosols of liquid particles comprising the ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the ceDNA vectors may likewise be produced with any solid particulate medicament aerosol generator, by techniques known in the pharmaceutical art.

In some embodiments, the ceDNA vectors can be administered to tissues of the CNS (e.g., brain, eye). In particular embodiments, the ceDNA vectors as disclosed herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Ocular disorders that may be treated, ameliorated, or prevented with the ceDNA vectors of the invention include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, the ceDNA vector as disclosed herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly. Additional ocular diseases that may be treated, ameliorated, or prevented with the ceDNA vectors of the invention include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by the ceDNA vectors of the invention. One or more anti-inflammatory factors can be expressed by intraocular (e.g., vitreous or anterior chamber) administration of the ceDNA vector as disclosed herein. In other embodiments, ocular diseases or disorders characterized by retinal degeneration (e.g., retinitis pigmentosa) can be treated, ameliorated, or prevented by the ceDNA vectors of the invention. intraocular (e.g., vitreal administration) of the ceDNA vector as disclosed herein encoding one or more neurotrophic factors can be used to treat such retinal degeneration-based diseases. In some embodiments, diseases or disorders that involve both angiogenesis and retinal degeneration (e.g., age-related macular degeneration) can be treated with the ceDNA vectors of the invention. Age-related macular degeneration can be treated by administering the ceDNA vector as disclosed herein encoding one or more neurotrophic factors intraocularly (e.g., vitreous) and/or one or more anti-angiogenic factors intraocularly or periocularly (e.g., in the sub-Tenon's region). Glaucoma is characterized by increased ocular pressure and loss of retinal ganglion cells. Treatments for glaucoma include administration of one or more neuroprotective agents that protect cells from excitotoxic damage using the ceDNA vector as disclosed herein. Accordingly, such agents include N-methyl-D-aspartate (NMDA) antagonists, cytokines, and neurotrophic factors, can be delivered intraocularly, optionally intravitreally using the ceDNA vector as disclosed herein.

In other embodiments, the ceDNA vector as disclosed herein may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the ceDNA vector as disclosed herein can also be used to treat epilepsy, which is marked by multiple seizures over time. In one representative embodiment, somatostatin (or an active fragment thereof) is administered to the brain using the ceDNA vector as disclosed herein to treat a pituitary tumor. According to this embodiment, the ceDNA vector as disclosed herein encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art. In particular embodiments, the ceDNA vector can encode a transgene that comprises a secretory signal as described in U.S. Pat. No. 7,071,172.

Another aspect of the invention relates to the use of a ceDNA vector as described herein to produce antisense RNA, RNAi or other functional RNA (e.g., a ribozyme) for systemic delivery to a subject in vivo. Accordingly, in some embodiments, the ceDNA vector can comprise a transgene that encodes an antisense nucleic acid, a ribozyme (e.g., as described in U.S. Pat. No. 5,877,022), RNAs that affect spliceosome-mediated trans-splicing (see, Puttaraju et al., (1999) Nature Biotech. 17:246; U.S. Pat. Nos. 6,013,487; 6,083,702), interfering RNAs (RNAi) that mediate gene silencing (see, Sharp et al., (2000) Science 287:2431) or other non-translated RNAs, such as “guide” RNAs (Gorman et al., (1998) Proc. Nat. Acad. Sci. USA 95:4929; U.S. Pat. No. 5,869,248 to Yuan et al.), and the like.

In some embodiments, the ceDNA vector can further also comprise a transgene that encodes a reporter polypeptide (e.g., an enzyme such as Green Fluorescent Protein, or alkaline phosphatase). In some embodiments, a transgene that encodes a reporter protein useful for experimental or diagnostic purposes, is selected from any of: 0-lactamase, (3-galactosidase (LacZ), alkaline phosphatase, thymidine kinase, green fluorescent protein (GFP), chloramphenicol acetyltransferase (CAT), luciferase, and others well known in the art. In some aspects, ceDNA vectors comprising a transgene encoding a reporter polypeptide may be used for diagnostic purposes or as markers of the ceDNA vector's activity in the subject to which they are administered.

In some embodiments, the ceDNA vector can comprise a transgene or a heterologous nucleotide sequence that shares homology with, and recombines with a locus on the host chromosome. This approach may be utilized to correct a genetic defect in the host cell.

In some embodiments, the ceDNA vector can comprise a transgene that can be used to express an immunogenic polypeptide in a subject, e.g., for vaccination. The transgene may encode any immunogen of interest known in the art including, but not limited to, immunogens from human immunodeficiency virus, influenza virus, gag proteins, tumor antigens, cancer antigens, bacterial antigens, viral antigens, and the like.

### XI. Administration

In particular embodiments, more than one administration (e.g., two, three, four or more administrations) may be employed to achieve the desired level of gene expression over a period of various intervals, e.g., daily, weekly, monthly, yearly, etc.

Exemplary modes of administration of the ceDNA vector disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ceDNA vector can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the ceDNA vector can also be to a tumor (e.g., in or near a tumor or a lymph node). The most suitable route in any given case will depend on the nature and severity of the condition being treated, ameliorated, and/or prevented and on the nature of the particular ceDNA vector that is being used. Additionally, ceDNA permits one to administer more than one transgene in a single vector, or multiple ceDNA vectors (e.g. a ceDNA cocktail).

Administration of the ceDNA vector disclosed herein to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In embodiments, the ceDNA vector as disclosed herein can be administered without employing “hydrodynamic” techniques.

Administration of the ceDNA vector as disclosed herein to cardiac muscle includes administration to the left atrium, right atrium, left ventricle, right ventricle and/or septum. The ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, a ceDNA vector according to the present invention is administered to skeletal muscle, diaphragm muscle and/or cardiac muscle (e.g., to treat, ameliorate and/or prevent muscular dystrophy or heart disease (e.g., PAD or congestive heart failure).

A. Ex Vivo Treatment

In some embodiments, cells are removed from a subject, a ceDNA vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, the ceDNA vector can encode a transgene (sometimes called a heterologous nucleotide sequence) that is any polypeptide that is desirably produced in a cell in vitro, ex vivo, or in vivo. For example, in contrast to the use of the ceDNA vectors in a method of treatment as discussed herein, in some embodiments the ceDNA vectors may be introduced into cultured cells and the expressed gene product isolated therefrom, e.g., for the production of antigens or vaccines.

The ceDNA vectors can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

One aspect of the technology described herein relates to a method of delivering a transgene to a cell. Typically, for in vitro methods, the ceDNA vector may be introduced into the cell using the methods as disclosed herein, as well as other methods known in the art. ceDNA vectors disclosed herein are preferably administered to the cell in a biologically-effective amount. If the ceDNA vector is administered to a cell in vivo (e.g., to a subject), a biologically-effective amount of the ceDNA vector is an amount that is sufficient to result in transduction and expression of the transgene in a target cell.

B. Dose Ranges

In vivo and/or in vitro assays can optionally be employed to help identify optimal dosage ranges for use. The precise dose to be employed in the formulation will also depend on the route of administration, and the seriousness of the condition, and should be decided according to the judgment of the person of oridinary skill in the art and each subject's circumstances. Effective doses can be extrapolated from dose-response curves derived from in vitro or animal model test systems.

A ceDNA vector is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vector required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver the ceDNA vector, then a therapeutically effective dose can be determined experimentally, but is expected to deliver from 1 μg to about 100 g of vector.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a ceDNA vector to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

Treatment can involve administration of a single dose or multiple doses. In some embodiments, more than one dose can be administered to a subject; in fact multiple doses can be administered as needed, because the ceDNA vector elicits does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a ceDNA vector as described by the disclosure (i.e., the absence of capsid components) allows the ceDNA vector to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a ceDNA vector is delivered to a subject more than 10 times.

In some embodiments, a dose of a ceDNA vector is administered to a subject no more than once per calendar day (e.g., a 24-hour period). In some embodiments, a dose of a ceDNA vector is administered to a subject no more than once per 2, 3, 4, 5, 6, or 7 calendar days. In some embodiments, a dose of a ceDNA vector is administered to a subject no more than once per calendar week (e.g., 7 calendar days). In some embodiments, a dose of a ceDNA vector is administered to a subject no more than bi-weekly (e.g., once in a two calendar week period). In some embodiments, a dose of a ceDNA vector is administered to a subject no more than once per calendar month (e.g., once in 30 calendar days). In some embodiments, a dose of a ceDNA vector is administered to a subject no more than once per six calendar months. In some embodiments, a dose of a ceDNA vector is administered to a subject no more than once per calendar year (e.g., 365 days or 366 days in a leap year).

C. Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### XII. Various Applications

The compositions and ceDNA vectors provided herein can be used to deliver a transgene for various purposes as described above. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease.

In some embodiments, the transgene encodes one or more peptides, polypeptides, or proteins, which are useful for the treatment, amelioration, or prevention of disease states in a mammalian subject. The transgene can be transferred (e.g., expressed in) to a patient in a sufficient amount to treat a disease associated with reduced expression, lack of expression or dysfunction of the gene.

In some embodiments, the ceDNA vectors are envisioned for use in diagnostic and screening methods, whereby a transgene is transiently or stably expressed in a cell culture system, or alternatively, a transgenic animal model.

Another aspect of the technology described herein provides a method of transducing a population of mammalian cells. In an overall and general sense, the method includes at least the step of introducing into one or more cells of the population, a composition that comprises an effective amount of one or more of the ceDNA disclosed herein.

Additionally, the present invention provides compositions, as well as therapeutic and/or diagnostic kits that include one or more of the disclosed ceDNA vectors or ceDNA compositions, formulated with one or more additional ingredients, or prepared with one or more instructions for their use.

A cell to be administered the ceDNA vector as disclosed herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

In some embodiments, the present application may be defined in any of the following paragraphs:

1A. A ceDNA vector, comprising:

an expression cassette comprising a cis-regulatory element, wherein the cis-regulatory element is selected from the group consisting of a posttranscriptional regulatory element and a BGH poly-A signal;

a wild-type ITR on the upstream (5′-end) of the expression cassette, wherein the wild-type ITR comprises a polynucleotide of SEQ ID NO: 51; and

a modified ITR on the downstream (3′-end) of the expression cassette, wherein the modified ITR comprises a polynucleotide of SEQ ID NO:2,

wherein said DNA vector has is devoid of a prokaryote-specific methylation, and is not encapsidated in an AAV capsid protein.

2A. The DNA vector of paragraph 1A, wherein the DNA vector has a linear and continuous structure.
3A. The DNA vector of any of paragraphs 1A-2A, wherein the posttranscriptional regulatory element comprises a WHP posttranscriptional regulatory element (WPRE).
4A. The DNA vector of any of paragraphs 1A-3A, wherein the expression cassette further comprises a cloning site.
5A. The DNA vector of any of paragraphs 1A-4A, wherein the expression cassette comprises a promoter selected from the group consisting of CAG promoter, AAT promoter, LP1 promoter, and EF1a promoter.
6A. The DNA vector of paragraph 1A, wherein the expression cassette comprises polynucleotides of SEQ ID NO: 3, SEQ ID NO: 7, SEQ ID NO: 8 and SEQ ID NO: 9.
7A. The DNA vector of any of paragraphs 1A-6A, wherein the expression cassette further comprises a cloning site and an exogenous sequence inserted into the cloning site.
8A. The DNA vector of paragraph 7A, wherein the exogenous sequence comprises at least 2000 nucleotides.
9A. The DNA vector of paragraph 7A, wherein the exogenous sequence encodes a protein.
10A. The DNA vector of paragraph 7A, wherein the exogenous sequence encodes a reporter protein.
11A. A cell comprising the DNA vector of any of paragraphs 1A-10A.
12A. The cell of paragraph 11A, further comprising a replication protein selected from the group consisting of: AAV Rep 78, AAV Rep 68, AAV Rep52, and AAV Rep 40.
13A. The cell of paragraph 12A, wherein said replication protein is encoded by a helper virus.
14A. The cell of any of paragraphs 11A-13A, wherein the cell is devoid of a gene encoding an AAV capsid protein.
15A. A pharmacologically active ingredient comprising the DNA vector of any of paragraphs 1A-10A, and optionally, an excipient.
16A. A method of delivering an exogenous sequence to a cell, comprising the step of: introducing said DNA vector of any of paragraphs 1A-10A to said cell.
17A. The method of paragraph 16A, wherein said step of introducing the DNA vector comprises hydrodynamic injection.
18A. A method of preparing a DNA vector comprising the steps of:

introducing into a cell a nucleic acid construct or a virus comprising:

an expression cassette comprising a cis-regulatory element, wherein the cis-regulatory element is selected from the group consisting of a posttranscriptional regulatory element, and a BGH poly-A signal;

a wild-type ITR on the upstream (5′-end) of the expression cassette, wherein the wild-type ITR comprises a polynucleotide of SEQ ID NO: 51; and

a modified ITR on the downstream (3′-end) of the expression cassette, wherein the modified ITR comprises a polynucleotide of SEQ ID NO:2,

wherein said cell is devoid of an AAV capsid protein; and

collecting said DNA vector produced from said nucleic acid construct or said virus,

wherein said DNA vector is devoid of a prokaryote-specific methylation.

19A. The method of paragraph 18A, wherein the DNA vector has a linear and continuous structure.
20A. The method of any of paragraphs 18A-19A, wherein the posttranscriptional regulatory element comprises a WHP posttranscriptional regulatory element (WPRE).
21A. The method of any of paragraphs 18A-20A, wherein the expression cassette further comprises a promoter selected from the group consisting of CAG promoter, AAT promoter, LP1 promoter, and EF1a promoter.
22A. The method of paragraph 18, wherein said expression cassette comprises polynucleotides of SEQ ID NO: 3, SEQ ID NO: 7, SEQ ID NO: 8 and SEQ ID NO: 9.
23A. The method of any of paragraphs 18A-22A, wherein said expression cassette further comprises an exogenous sequence.
24A. The method of paragraph 23A, wherein the exogenous sequence comprises at least 2000 nucleotides.
25A. The method of paragraph 23A, wherein the exogenous sequence encodes a protein.
26A. The method of paragraph 23A, wherein the exogenous sequence encodes a reporter protein.
27A. The method of any of paragraphs 18A-26A, wherein said cell is an insect cell.
28A. A DNA vector generated by the method of any of paragraphs 18-27.
29A. A cell for producing a DNA vector comprising:

a first polynucleotide comprising:

an expression cassette comprising a cis-regulatory element, wherein the cis-regulatory element is selected from the group consisting of a posttranscriptional regulatory element, and a BGH poly-A signal;

a wild-type ITR on the upstream (5′-end) of the expression cassette, wherein the wild-type ITR comprises a polynucleotide of SEQ ID NO: 51; and

a modified ITR on the downstream (3′-end) of the expression cassette, wherein the modified ITR comprises a polynucleotide of SEQ ID NO:2; and

a second polynucleotide encoding a replication protein selected from the group consisting of AAV78, AAV52, AAV Rep68, and AAV Rep 40, wherein said cell is devoid of a gene encoding an AAV capsid protein.

30A. The cell of paragraph 29A, wherein the posttranscriptional regulatory element comprises a WHP posttranscriptional regulatory element (WPRE).
31A. The cell of any of paragraphs 29A-30A, wherein said cell is an insect cell. 32A. A DNA vector produced from the cell of any of paragraphs 29A-31A, by replication of said first polynucleotide.
33A. A polynucleotide for generating a DNA vector comprising:

an expression cassette comprising a cis-regulatory element, wherein the cis-regulatory element is selected from the group consisting of a posttranscriptional regulatory element, and a BGH poly-A signal;

a wild-type ITR on the upstream (5′-end) of the expression cassette, wherein the wild-type ITR comprises a polynucleotide of SEQ ID NO: 51; and

a modified ITR on the downstream (3′-end) of the expression cassette, wherein the modified ITR comprises a polynucleotide of SEQ ID NO:2.

34A. The polynucleotide of paragraph 33A, wherein the posttranscriptional regulatory element comprises a WHP posttranscriptional regulatory element (WPRE).
35A. The polynucleotide of any of paragraphs 33A-34A, wherein the polynucleotide is in a plasmid, in a bacmid, or in a baculovirus.
36A. The polynucleotide of any of paragraphs 33A-35A, further comprising an exogenous sequence.
37A. The polynucleotide of paragraph 36A, wherein the exogenous sequence comprises at least 2000 nucleotides.
38A. The polynucleotide of paragraph 36A, wherein the exogenous sequence encodes a protein.
39A. The polynucleotide of paragraph 36A, wherein the exogenous sequence encodes a reporter
40A. A DNA vector produced by replication of the polynucleotide of any of paragraphs 33A-39A by a replication protein selected from the group consisting of AAV78, AAV52, AAV Rep68, and AAV Rep 40, wherein the DNA vector has a linear and continuous structure, is devoid of a prokaryote-specific methylation and is not encapsidated in an AAV capsid protein.
41A. A DNA vector, comprising:

an expression cassette;

a modified ITR on the upstream (5′-end) of the expression cassette, wherein the modified ITR comprises a polynucleotide of SEQ ID NO: 52; and

a wild-type ITR on the downstream (3′-end) of the expression cassette, wherein the wild-type ITR comprises a polynucleotide of SEQ ID NO:1,

wherein the DNA vector is devoid of a prokaryote-specific methylation, and is not encapsidated in an AAV capsid protein.

42A. The DNA vector of paragraph 41A, wherein the expression cassette comprises a cis-regulatory element, wherein the cis-regulatory element is selected from the group consisting of a posttranscriptional response element and a poly-A signal.
43A. The DNA vector of paragraph 42A, wherein the posttranscriptional response element comprises a WHP posttranscriptional response element (WPRE).
44A. The DNA vector of any of paragraphs 42A-43A, wherein the poly-A signal comprises a BGH poly-A signal.
45A. The DNA vector of any of paragraphs 41A-44A, wherein the expression cassette comprises a promoter selected from the group consisting of CAG promoter, AAT promoter, LP1 promoter, and EF1a promoter.
46A. The DNA vector of paragraph 41A, wherein the expression cassette comprises polynucleotides of SEQ ID NO: 3, SEQ ID NO: 7, SEQ ID NO: 8 and SEQ ID NO: 9.
47A. The DNA vector of any of paragraphs 41A-46A, wherein the expression cassette comprises an exogenous sequence.
48A. The DNA vector of paragraph 47A, wherein the exogenous sequence comprises at least 2000 nucleotides.
49A. The DNA vector of paragraph 47A, wherein the exogenous sequence encodes a protein.
50A. The DNA vector of paragraph 47A, wherein the exogenous sequence encodes a reporter protein.
51A. A cell comprising the DNA vector of any of paragraphs 41A-50A.
52A. The cell of paragraph 52A, further comprising a replication protein selected from the group consisting of: AAV Rep 78, AAV Rep 68, AAV Rep52, and AAV Rep 40.
53A. The cell of paragraph 51A, wherein said replication protein is encoded by a helper virus.
54A. The cell of any of paragraphs 51-53, wherein the cell is devoid of a gene encoding an AAV capsid protein.
55A. A pharmacologically active ingredient comprising: the DNA vector of any of paragraphs 1A-10A; and optionally, an excipient.
56A. A method of delivering an exogenous sequence to a cell, comprising the step of: introducing said non-encapsidated DNA vector of any of paragraphs 1A-10A to said cell.
57A. The method of paragraph 16A, wherein said step of introducing the DNA vector comprises hydrodynamic injection.
58A. A method of preparing a DNA vector comprising the steps of:

introducing into a cell a nucleic acid construct or a virus comprising:

an expression cassette;

a modified ITR on the upstream (5′-end) of the expression cassette, wherein the modified ITR comprises a polynucleotide of SEQ ID NO: 52; and

a wild-type ITR on the downstream (3′-end) of the expression cassette, wherein the wild-type ITR comprises a polynucleotide of SEQ ID NO:1,

wherein said cell is devoid of an AAV capsid protein; and

collecting said DNA vector produced from said nucleic acid construct or said virus,

wherein said DNA vector is devoid of a prokaryote-specific methylation, and is not encapsidated in an AAV capsid protein.

59A. The method of paragraph 18A, wherein the expression cassette comprises a cis-regulatory element, wherein the cis-regulatory element is selected from the group consisting of a posttranscriptional response element and a poly-A signal.
60A. The method of paragraph 59A, wherein the posttranscriptional response element comprises a WHP posttranscriptional response element (WPRE).
61A. The method of any of paragraphs 59A-60A, wherein the poly-A signal comprises a BGH poly-A signal.
62A. The method of any of paragraphs 18A-61A, wherein the expression cassette comprises a promoter selected from the group consisting of CAG promoter, AAT promoter, LP1 promoter, and EF1a promoter.
63A. The method of paragraph 18A, wherein said expression cassette comprises polynucleotides of SEQ ID NO: 3, SEQ ID NO: 7, SEQ ID NO: 8 and SEQ ID NO: 9.
64A. The method of any of paragraphs 18A-63A, wherein the expression cassette further comprises an exogenous sequence.
65A. The method of paragraph 23A, wherein the exogenous sequence comprises at least 2000 nucleotides.
66A. The method of paragraph 23A, wherein the exogenous sequence encodes a protein.
67A. The method of paragraph 23A, wherein the exogenous sequence encodes a reporter protein.
68A. The method of any of paragraphs 18A-26A, wherein said cell is an insect cell.
69A. A DNA vector generated by the method of any of paragraphs 18A-27A.
70A. The DNA vector of paragraph 69A, wherein the DNA vector has a linear and continuous structure.
71A. A cell for producing a DNA vector comprising:

a first polynucleotide comprising:

an expression cassette;

a modified ITR on the upstream (5′-end) of the expression cassette, wherein the modified ITR comprises a polynucleotide of SEQ ID NO: 52; and

a wild-type ITR on the downstream (3′-end) of the expression cassette, wherein the wild-type ITR comprises a polynucleotide of SEQ ID NO:1; and

a second polynucleotide encoding a replication protein selected from the group consisting of AAV78, AAV52, AAV Rep68, and AAV Rep 40, wherein said cell is devoid of a gene encoding an AAV capsid protein. 72A. The cell of paragraph 71A, wherein said cell is an insect cell.

73A. A DNA vector produced from the cell of any of paragraphs 29A-50A, by replication of said first polynucleotide.
74A. A polynucleotide for generating a DNA vector comprising:

an expression cassette;

a modified ITR on the upstream (5′-end) of the expression cassette, wherein the modified ITR comprises a polynucleotide of SEQ ID NO: 52; and

a wild-type ITR on the downstream (3′-end) of the expression cassette, wherein the wild-type ITR comprises a polynucleotide of SEQ ID NO:1.

75A. The polynucleotide of paragraph 74A, wherein the polynucleotide is in a plasmid, in a bacmid, or in a baculovirus.
76A. The polynucleotide of any of paragraphs 74A-75A, further comprising an exogenous sequence.
77A. The polynucleotide of paragraph 74A, wherein the exogenous sequence comprises at least 2000 nucleotides.
78A. The polynucleotide of paragraph 74A, wherein the exogenous sequence encodes a protein.
79A. The polynucleotide of paragraph 74A, wherein the exogenous sequence encodes a reporter
80A. A DNA vector produced by replication of the polynucleotide of any of paragraphs 33-39 by a replication protein selected from the group consisting of AAV78, AAV52, AAV Rep68, and AAV Rep 40, wherein the DNA vector is devoid of a prokaryote-specific methylation and is not encapsidated in an AAV capsid protein.
81A. The DNA vector of paragraph 80A, wherein the DNA vector is produced in an insect cell.
82A. A ceDNA vector obtained from a plasmid comprising a mutated AAV ITR sequence in any of Tables 2-6 or Tables 7-10A or 10B.
In some embodiments, the present application may be defined in any of the following paragraphs:
1B. A DNA vector obtained by a process comprising:

(a) transfecting a first population of insect cells with a first recombinant bacmid, obtained by transposing a first DNA plasmid construct into a baculovirus expression vector, the first DNA plasmid construct proceeding sequentially in the following order in a single direction: a first replicative protein site (RPS-1), a promoter operatively linked to a ORF reporter polynucleotide sequence, a post-translational and termination signal, and a second replicative protein site (RPS-2), wherein RPS-1 and RPS-2 are independently intramolecularly duplexed and covalently joined, and RPS-1 has one or more polynucleotide base pair deletions, substitutions or truncations relative to RPS-2, and then harvesting therefrom a first population of baculo-injected insect cells (BIICs-1);

(b) transfecting a second population of insect cells with a second recombinant bacmid, obtained by transposing a second DNA plasmid construct into a second baculovirus expression vector, the second DNA plasmid construct encoding a protein that binds to at least one of RPS-1 and RPS-2, and then harvesting therefrom a second population of baculo-injected insect cells (BIICs-2);

(c) combining the BIICs-1 and the BIICs-2 with a third population of insect cells in relative amounts providing substantially equal multiplicity of infection (MOD, and then incubating the third population of insect cells, BIICs-1 and BIICs-2 under cell growth conditions where the protein encoded by the second DNA plasmid construct reacts with the RPS-1 and/or RPS-2 sites to induce replication of a DNA vector encoded by the first DNA plasmid, until observation of an insect cell diameter of about 15-20 micrometers and a cell viability of about 50-80%, and then harvesting DNA from the insect cell pellets by centrifugation;

(d) extracting the DNA from the insect cell pellets to obtain DNA produced from the insect cell pellets;

(e) identifying DNA obtained from the insect cell pellets on a native gel in both a primary band and a secondary band at a relative distance to one another that is indicative of the second band representing material that is approximately twice the weight of the primary band;

(f) confirming the DNA vector in the DNA obtained from the insect cell pellets on a denatured gel by a shift in both a primary and a secondary band upward toward the lower weight area of the gel after restriction endonuclease digestion of the DNA; and

(g) isolating the remaining DNA vector obtained from the insect cell pellets to obtain the high stability DNA vector.

2B. The DNA vector of paragraph 1B, wherein the RPS-1 and RPS-2 are different and are independently selected from the DNA polynucleotide sequence pairs of SEQ ID NOs:
SEQ ID NO: 1 and SEQ ID NO: 52; or SEQ ID NO: 2 and SEQ ID NO: 51; or SEQ ID NO: 101 and SEQ ID NO: 102; or SEQ ID NO: 103 and SEQ ID NO: 104; or SEQ ID NO: 105 and SEQ ID NO: 106; or SEQ ID NO: 107 and SEQ ID NO: 108; or SEQ ID NO: 109 and SEQ ID NO: 110; or SEQ ID NO: 111 and SEQ ID NO: 112; or SEQ ID NO: 113 or SEQ ID NO: 114; and SEQ ID NO: 115 or SEQ ID NO: 116.
In some embodiments, the present application may be defined in any of the following paragraphs:
1C. A capsid free AAV (cfAAV) vector, comprising:
an expression cassette comprising a cis-regulatory element, a promoter and an exogenous sequence; and
two self-complementary sequences flanking said expression cassette,
wherein the cfAAV vector is not associated with a capsid protein and is devoid of prokaryote-specific methylation.
2C. The cfAAV vector of paragraph 1C, wherein the cis-regulatory elements is selected from the group consisting of a riboswitch, an insulator, a mir-regulatable element, and a post-transcriptional regulatory element.
3C. The cfAAV vector of any of paragraphs 1C-2C, wherein the DNA vector is CELiD.
4C. The cfAAV vector of any of paragraphs 1C-3C, wherein the two self-complementary sequences are ITR sequences of AAV2.
5C. The cfAAV vector of any of paragraphs 1C-4C, wherein the exogenous sequence comprises an internal ribosome entry site (IRES) and 2A element.
6C. The cfAAV vector of paragraph 5C, wherein the expression cassette comprises a sequence encoding more than one proteins.
7C. The cfAAV vector of any of paragraphs 1C-6C, wherein the expression cassette comprises more than 4000 nucleotides, 5000 nucleotides, 10,000 nucleotides or 20,000 nucleotides.
8C. A pharmacological composition comprising the cfAAV vector of any of paragraphs 1C-7C.
9C. An expression construct, comprising:
an expression cassette comprising a cis-regulatory element, a promoter and an exogenous sequence; and two inverted terminal repeat (ITR) sequences flanking said expression cassette,
wherein the expression construct is devoid of an open reading frame encoding a capsid protein.
10C. The expression construct of paragraph 9C, wherein the cis-regulatory elements are selected from the group consisting of a riboswitch, an insulator, a mir-regulatable element, and a post-transcriptional regulatory element.
11C. The expression cassette of any of paragraphs 9C-10C, wherein the exogenous sequence comprises an internal ribosome entry site (IRES) and 2A element.
12C. The expression cassette of paragraph 11, wherein the expression cassette comprises a sequence encoding more than one proteins.
13C. The expression construct of any of paragraphs 9C-12C, wherein the expression construct is in a plasmid, a Bacmid, or a baculovirus.
14C. A method of generating a cfAAV vector, comprising:
introducing the expression construct of any of paragraphs 9C-13C into a cell; and
collecting the cfAAV vector generated by replication of the expression construct.
15C. The method of paragraph 14C, further comprising the step of replicating the expression construct multiple times before introducing the expression construct into the cell.
16C. The method of paragraph 15C, wherein the expression construct is in a plasmid and the step of replicating is done in an E. coli.
17C. The method of paragraph 16C, further comprising the step of transferring the expression construct from the plasmid to a Bacmid before introducing the expression construct into the cell.
18C. The method of paragraph 17C, further comprising the step of transferring the expression construct from the Bacmid to a baculovirus before introducing the expression construct into the cell.
19C. The method of any of paragraphs 14C-18C, further comprising the step of introducing a Rep protein to the cell.
20C. The method of any of paragraphs 14C-19C, wherein the cell is an insect cell.
21C. The cfAAV vector produced by the method of any of paragraphs 14C-20C.
22C. The cfAAV vector of paragraph 21C, wherein the DNA vector is CELiD.
In some embodiments, the present application may be defined in any of the following paragraphs:
1D. A capsid-free, non-viral DNA vector obtained from a vector polynucleotide wherein the vector polynucleotide encodes a heterologous nucleic acid operatively positioned between a first and a second AAV2 inverted terminal repeat DNA polynucleotide sequence (ITRs), with at least one of the ITRs having at least one polynucleotide deletion, insertion, or substitution with respect to the corresponding AAV2 wild type ITR of SEQ ID NO:1 or SEQ ID NO:51 to induce replication of the DNA vector in an insect cell in the presence of Rep protein, the DNA vector being obtainable from a process comprising the steps of:
a. incubating a population of insect cells harboring the vector polynucleotide, which is devoid of viral capsid coding sequences, in the presence of Rep protein, under conditions effective and for a time sufficient to induce production of the capsid-free, non-viral DNA within the insect cells, wherein the insect cells do not comprise viral capsid coding sequences; and
b. harvesting and isolating the capsid-free, non-viral DNA from the insect cells;
wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.
   2D. The capsid-free, non-viral DNA vector of paragraph 1, wherein the mutated ITR is selected from the group consisting of: SEQ ID NO:2 and SEQ ID NO:52.
   3D. The capsid-free, non-viral DNA vector of paragraph 1, wherein the vector polynucleotide comprises a pair of ITRs selected from the group consisting of: SEQ ID NO:1 and SEQ ID NO:52; and SEQ ID NO:2 and SEQ ID NO:51.
   4D. A capsid-free, non-viral DNA vector obtained from a vector polynucleotide, wherein the vector polynucleotide encodes a heterologous nucleic acid operatively positioned between two different inverted terminal repeat sequences (ITRs), at least one of the ITRs is a functional ITR comprising a functional terminal resolution site and a Rep binding site, and one of the ITRs comprising a deletion, insertion, or substitution, relative to the functional ITR; the presence of Rep protein inducing replication of the vector polynucleotide and production of the DNA vector in an insect cell, the DNA vector being obtainable from a process comprising the steps of:
   a. incubating a population of insect cells harboring the vector polynucleotide, which is devoid of viral capsid coding sequences in the presence of Rep protein under conditions effective and for time sufficient to induce production of the capsid-free, non-viral DNA vector within the insect cells, wherein the insect cells do not comprise production capsid-free, non-viral DNA within the insect cells; and
   b. harvesting and isolating the capsid-free, non-viral DNA from the insect cells;
   wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.
   In some embodiments, the present application may be defined in any of the following paragraphs:
   1E. A vector comprising (i) a non-viral capsid-free DNA vector with covalently-closed ends (ceDNA vector), wherein the ceDNA vector comprises a heterologous nucleic acid sequence encoding the transgene operably positioned between two different AAV inverted terminal repeat sequences (ITRs), one of the ITRs comprising a functional AAV terminal resolution site and a Rep binding site, one of the ITRs comprising a deletion, insertion, or substitution relative to the other ITR (modified ITR), wherein the vector is not in a viral capsid.
   2E. The ceDNA vector of paragraph 1, wherein the ceDNA vector when digested with a restriction enzyme having a single recognition site on the ceDNA vector has the presence of characteristic bands of linear and continuous DNA as compared to linear and non-continuous DNA controls when analyzed on a non-denaturing gel.
   3E. The ceDNA vector of paragraph 1E, wherein the modified ITR is from a different serotype.
   4E. The ceDNA vector of paragraph 3E, wherein the modified ITR is not a wild type ITR.
   5E. The ceDNA vector of paragraph 1E, wherein the deletion, insertion, or substitution is in at least one of the ITR regions selected from the group consisting of A, A′, B, B′, C, C′, and D.
   6E. The ceDNA vector of paragraph 5E, wherein the deletion, insertion, or substation results in the deletion of one of the loops formed by the A, A′, B, B′ C, or C′ regions.
   7E. The ceDNA vector of paragraph 1E, wherein the modification corresponds to the alteration in an AAV2 modified ITR selected from the group consisting of SEQ ID NOS: 101-498 and 499 or 545-547.
   8E. The ceDNA vector of paragraph 1E, wherein the modification corresponds to the alteration in an AAV2 modified ITR selected from the group consisting of SEQ ID NOS. 2, 52, 63, and 64.
   9E. The ceDNA vector of paragraphs 1E-8E, wherein the vector is in a nanocarrier.
   10E. The ceDNA vector of paragraph 9E, wherein the nanocarrier comprises a lipid nanoparticle (LNP).
   11E. A method comprising:
   producing a non-viral capsid-free DNA with covalently-closed ends (ceDNA) by using a vector polynucleotide, wherein the vector polynucleotide encodes a heterologous nucleic acid operatively positioned between two different inverted terminal repeat sequences (ITRs), wherein at least one of the ITRs comprising a functional terminal resolution site and a Rep binding site, and one of the ITRs comprising a deletion, insertion, or substitution, relative to the other ITR; the presence of Rep protein inducing replication of the vector polynucleotide and production of the DNA vector in an insect cell, the DNA vector being obtainable from a process comprising the steps of:
   a. incubating a population of insect cells harboring the vector polynucleotide, which is devoid of viral capsid coding sequences in the presence of Rep protein under conditions effective and for time sufficient to induce production of the capsid-free, non-viral DNA vector within the insect cells, wherein the insect cells do not comprise production capsid-free, non-viral DNA within the insect cells; and
   b. harvesting and isolating the capsid-free, non-viral DNA from the insect cells;

wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.

12E. A capsid-free, non-viral DNA vector obtained from a vector polynucleotide wherein the vector encodes a heterologous gene operatively positioned between a first and a second AAV2 inverted terminal repeat DNA polynucleotide sequence (ITRs), with at least one of the ITRs having at least one polynucleotide deletion, insertion, or substitution with respect to the corresponding AAV2 wild type ITR of SEQ ID NO:1 or SEQ ID NO:51 to induce replication of the DNA vector in an insect cell in the presence of Rep protein, the DNA vector being obtainable from a process comprising the steps of:
(a) incubating a population of insect cells harboring the vector polynucleotide, which is devoid of viral capsid coding sequences, in the presence of Rep protein, under conditions effective and for a time sufficient to induce production of the capsid-free, non-viral DNA within the insect cells, wherein the insect cells do not comprise viral capsid coding sequences; and
(b) harvesting and isolating the capsid-free, non-viral DNA from the insect cells;

wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.

13E. The capsid-free, non-viral DNA vector of paragraph 12E, wherein the modified ITR is selected from the group consisting of: SEQ ID NO:2 and SEQ ID NO:52.
14E. The capsid-free, non-viral DNA vector of paragraph 12E, wherein the vector polynucleotide comprises a pair of ITRs selected from the group consisting of: SEQ ID NO:1 and SEQ ID NO:52; SEQ ID NO:2 and SEQ ID NO:51.
15E. A capsid-free, non-viral DNA vector obtained from a vector polynucleotide, wherein the vector polynucleotide encodes a heterologous gene operatively positioned between two different inverted terminal repeat sequences (ITRs), at least one of the ITRs comprising a functional terminal resolution site and a Rep binding site, and one of the ITRs comprising a deletion, insertion, or substitution, the presence of Rep protein inducing replication of the vector polynucleotide and production of the DNA vector in an insect cell, the DNA vector being obtainable from a process comprising the steps of:
(a) incubating a population of insect cells harboring the vector polynucleotide, which is devoid of viral capsid coding sequences in the presence of Rep protein under conditions effective and for time sufficient to induce production of the capsid-free, non-viral DNA vector within the insect cells, wherein the insect cells do not comprise production capsid-free, non-viral DNA within the insect cells; and
(b) harvesting and isolating the capsid-free, non-viral DNA from the insect cells;
   wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.
   17E. A capsid-free, non-viral DNA vector of paragraph 15E, wherein one ITR is a wild-type AAV ITR.
   18E. A method for treating disease in a subject, the method comprising:
   co-administering to a subject in need thereof, a composition comprising (i) a non-viral capsid-free DNA vector with covalently-closed ends (ceDNA vector), wherein the ceDNA vector comprises a heterologous nucleic acid sequence encoding a transgene operably positioned between two different AAV inverted terminal repeat sequences (ITRs), one of the ITRs comprising a functional AAV terminal resolution site and a Rep binding site, one of the ITRs comprising a deletion, insertion, or substitution relative to the other ITR.
   19E. The method of paragraph 17E, wherein the ceDNA vector is administered in combination with a pharmaceutically acceptable carrier.
   20E. A method for delivering a therapeutic to a subject, the method comprising:
   administering to a subject, a composition comprising the ceDNA vector of paragraphs 1E-10E and 12E-16E.

### EXAMPLES

The following examples are provided by way of illustration not limitation.

### Example 1: Constructing ceDNA Vectors

Production of the ceDNA vectors using a polynucleotide construct template is described. For example, a polynucleotide construct template used for generating the ceDNA vectors of the present invention can be a ceDNA-plasmid, a ceDNA-Bacmid, and/or a ceDNA-baculovirus. Without being limited to theory, in a permissive host cell, in the presence of e.g., Rep, the polynucleotide construct template having two ITRs and an expression construct, where at least one of the ITRs is modified, replicates to produce ceDNA vectors. ceDNA vector production undergoes two steps: first, excision (“rescue”) of template from the template backbone (e.g. ceDNA-plasmid, ceDNA-bacmid, ceDNA-bacliovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left ITR and a right mutated ITR with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIGS. 1A and 1B) were also introduced between each component to facilitate the introduction of new genetic components into the specific sites in the construct. R3 (Pmel) GTTTAAAC (SEQ ID NO: 7) and R4 (Pad) TTAATTAA (SEQ ID NO: 542) enzyme sites are engineered into the cloning site to introduce an open reading frame of a transgene. These sequences were cloned into a pFastBac HT B plasmid obtained from ThermoFisher Scientific.

In brief, a series of ceDNA vectors were obtained from the ceDNA-plasmid constructs shown in Table 12, using the process shown in FIGS. 4A-4C. Table 12 indicates the number of the corresponding polynucleotide sequence for each component, including sequences active as replication protein site (RPS) (e.g. Rep binding site) on either end of a promoter operatively linked to a transgene. The numbers in Table 12 refer to SEQ ID NOs in this document, corresponding to the sequences of each component.

TABLE 12
Exemplary ceDNA constructs.

| Plasmid | ITR-L | Promoter | Transgene | ITR-R |
|---|---|---|---|---|
| Constuct-1 | 51 | 3 | Luciferase | 2 |
| Constuct-2 | 52 | 3 | Luciferase | 1 |
| Constuct-3 | 51 | 4 w/SV40 intr | Luciferase | 2 |
| Constuct-4 | 52 | 4 w/SV40 intr | Luciferase | 1 |
| Constuct-5 | 51 | 5 w/SV40 intr | Luciferase | 2 |
| Constuct-6 | 52 | 5 w/SV40 intr | Luciferase | 1 |
| Constuct-7 | 51 | 6 | Luciferase | 2 |
| Constuct-8 | 52 | 6 | Luciferase | 1 |

In some embodiments, a construct to make ceDNA vectors comprises a promoter which is a regulatory switch as described herein, e.g., an inducible promoter. Other constructs were used to make ceDNA vectors, e.g., constructs 10, constructs 11, constructs 12 and construct 13 (see, e.g., Table 14A) which comprise a MND or HLCR promoter operatively linked to a luciferase transgene.

Production of ceDNA-Bacmids:

With reference to FIG. 4A, DH10Bac competent cells (MAX EFFICIENCY® DH10Bac™ Competent Cells, Thermo Fisher) were transformed with either test or control plasmids following a protocol according to the manufacturers instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (080dlacZAM15 marker provides a-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of −4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test contructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

With reference to FIG. 4A, a “Rep-plasmid” according to FIG. 8A was produced in a pFASTBAC™-Dual expression vector (ThermoFisher) comprising both the Rep78 (SEQ ID NO: 13) or Rep68 (SEQ ID NO: 12) and Rep52 (SEQ ID NO: 14) or Rep40 (SEQ ID NO: 11).

The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (D80dlacZAM15 marker provides a-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of −70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm. Yields of various ceDNA vectors determined based on UV absorbance are provided below in Table 13.

TABLE 13
Yield of ceDNA vectors from exemplary constructs.

| | | Culture Parameters | | Estimated |
|---|---|---|---|---|
| | Culture | (Diameter in | Yield | Yield |
| Construct | Volume | micrometers) | (mg/L) | (pg/cell) |
| construct-1 | 2 × 1L | Total: 6.02 × 10e6 | 15.8 | 5.23 |
| | | Viability: 53.3% | | |
| | | Diameter: 18.4 | | |

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIG. 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

FIG. 5 provides an exemplary picture of a denaturing gel with ceDNA vectors as follows: construct-1, construct-2, construct-3, construct-4, construct-5, construct-6, construct-7 and construct-8 (all described in Table 12 above), with (+) or without (−) digestion by the endonuclease. Each ceDNA vector from constructs-1 to construct-8 produced two bands (*) after the endonuclease reaction. Their two band sizes determined based on the size marker are provided on the bottom of the picture. The band sizes confirm that each of the ceDNA vectors produced from plasmids comprising construct-1 to construct-8 has a continuous structure.

As used herein, the phrase “Assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately 1/3× and 2/3× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8 1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and nonlimiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 2: Viral DNA Production in ceDNA Cells

ceDNA vectors were also generated from constructs 11, 12, 13 and 14 shown in Table 14A. ceDNA-plasmids comprising constructs 11-14 were generated by molecular cloning methods well known in the art. The plasmids in Table 14A were constructed with the WPRE comprising SEQ ID NO: 8 followed by BGHpA comprising SEQ ID NO: 9 in the 3′ untranslated region between the transgene and the right side ITR.

TABLE 14A

| Plasmid | ITR-L | Promoter | Transgene | ITR-R |
|---|---|---|---|---|
| Construct | (SEQ ID | (SEQ ID | Luciferase | (SEQ ID |
| 11 | NO: 63) | NO: 70) | (SEQ ID | NO: 1) |
| | | | NO: 71) | |
| Construct | (SEQ ID | (SEQ ID | Luciferase | (SEQ ID |
| 12 | NO: 51) | NO: 70) | (SEQ ID | NO: 64) |
| | | | NO: 71) | |
| Construct | (SEQ ID | (SEQ ID | Luciferase | (SEQ ID |
| 13 | NO: 63) | NO: 74) | (SEQ ID | NO: 1) |
| | | | NO: 71) | |
| Construct | (SEQ ID | (SEQ ID | Luciferase | (SEQ ID |
| 14 | NO: 51) | NO: 74) | (SEQ ID | NO: 64) |
| | | | NO: 71) | |

The Backbone vector for constructs for constructs 11-14 is as follows: (i) asymlTR-MNDluciferase-wPRE-BGH-polyA-ITR in pFB-HTb (construct 11), (ii) ITR-MND-luciferase-wPRE-BGH-polyA-asymlTR in pFB-HTb (contract 12), (iii) asymlTR-HLCR-AAT-luc-wPRE(O)-BGH-polyA-ITR in pFB-HTb (construct 13); and ITR-HLCR-AAT-luc-wPRE(O)-BGH-polyA-asymlTR in pFB-HTb (construct 14), each construct having at least one asymmetric ITR with respect to each other. These constructs also comprise one or more of the following sequences: wPREO (SEQ ID NO:72) and BGH-PolyA sequence (SEQ ID NO:73), or sequences at least 85%, or at least 90% or at least 95% sequence indentity thereto.

Next, ceDNA vector production was performed according to the procedure in FIG. 4A-4C, for example, (a) Generation of recombinant ceDNA-Bacmid DNA and Transfection of insect cell with recombinant ceDNA-Bacmid DNA; (b) generation of P1 stock (low titer), P2 stock (high titer), and determination of virus titer by Quantitative-PCR, to obtain a deliverable of 5 ml, >1E+7 plaque forming or infectious units “pfu” per ml BV Stock, BV Stock COA. ceDNA vector isolation was performed by co-infection of 50 ml insect cells with BV stock for the following pairs of infections: Rep-bacmid as disclosed herein and at least one of the following constructs: construct 11, construct 12, construct 13 and construct 14. ceDNA vector isolation was performed using QIAGEN Plasmid Midi Kit to obtain purified DNA material for further analysis. Table 14B and Table 14C show the yield (as detected by OD dection) of ceDNA vector produced from contracts 11-14.

TABLE 14B
Yield (as detected by OD dection) of exemplary
ceDNA vectors produced from contructs 11-14.

| | | | total DNA [ug] | |
|---|---|---|---|---|
| | DNA | | amount from | |
| | Concentration | | 50 ml infection | Yield total |
| | OD260 and | | (ceDNA | DNA [mg] |
| Construct | Standard | 260/280 | production | per 1 liter |
| No | Coefficient 50 | ratio | volume) | (estimate) |
| Contruct 11 | 342.7 ng/μl | 1.79 | 8.57 | 0.171 |
| Contruct 12 | 197.5 ng/μl | 1.9 | 4.54 | 0.090 |
| Contruct 13 | 145 ng/μl | 1.9 | 3.62 | 0.072 |
| Contruct 14 | 443.1 ng/μl | 1.79 | 11.08 | 0.221 |

TABLE 14C
Yield (as detected by OD dection) of
ceDNA vector produced from contracts

| | | | | DNA Conc. | Yield | Yield total |
|---|---|---|---|---|---|---|
| | | | | OD260 and | ug/0.2 g | DNA [mg] |
| Con- | | | | Standard | cell | per 1 liter |
| struct # | A₂₃₀ | 260/230 | 260/280 | Coefficient 50 | pellet | (estimate) |
| 14 | 0.038 | 2.789 | 1.860 | 265 ng/ul | 53.0 | 2.6 |
| 12 | 0.017 | 6.176 | 1.842 | 263 ng/ul | 52.6 | 2.6 |

Table 14C shows the amount of DNA material obtained (as detected by OD detection) using the constructs 12 and 14 from Table 14C. The yield of total DNA material was acceptable, compared to typical yields of about 3 mg/L of DNA material from the process in Example 1 (Table 13) above.

### Example 3: ceDNA Vectors Express Luciferase Transgene In Vitro

Constructs were generated by introducing an open reading frame encoding the Luciferase reporter gene into the cloning site of ceDNA-plasmid constructs: construct-1, construct-3, construct-5, and construct-7. The ceDNA-plasmids (see above in Table 12) including the Luciferase coding sequence are named plasmid construct 1-Luc, c plasmid construct-3-Luc, plasmid construct-5-Luc, and plasmid construct 7-Luc, respectively.

HEK293 cells were cultured and transfected with 100 ng, 200 ng, or 400 ng of plasmid constructs 1, 3, 5 and 7, using FUGENE® (Promega Corp.) as a transfection agent. Expression of Luciferase from each of the plasmids was determined based on Luciferase activity in each cell culture and the results are provided in FIG. 6A. Luciferase activity was not detected from the untreated control cells (“Untreated”) or cells treated with Fugene alone (“Fugene”), confirming that the Luciferase activity resulted from gene expression from the plasmids. As illustrated in FIG. 6A and FIG. 6B, robust expression of Luciferase was detected from constructs 1 and 7. The expression from construct-7 expressed Luciferase with a dose-dependent increase of Luciferase activity being detected.

Growth and viability of cells transfected with each of the plasmids were also determined and presented in FIG. 7A and FIG. 7B. Cell growth and viability of transfected cells were not significantly different between different groups of cells treated with different constructs.

Accordingly, Luciferase activity measured in each group and normalized based on cell growth and viability was not different from Luciferase activity without the normalization. ceDNA-plasmid with construct 1-Luc showed the most robust expression of Luciferase with or without normalization.

Thus, the data presented in FIGS. 6A, 6B, 7A and 7B demonstrate that construct 1, comprising from 5′ to 3′-WT-ITR (SEQ ID NO: 51), CAG promoter (SEQ ID NO:3), R3/R4 cloning site (SEQ ID NO:7), WPRE (SEQ ID NO: 8), BGHpA (SEQ ID NO:9) and a modified ITR (SEQ ID NO:2), is effective in producing a ceDNA vector that can express a protein of a transgene within the ceDNA vector.

### Example 4: In Vivo Protein Expression of Luciferase Transgene from ceDNA Vectors

In vivo protein expression of a transgene from ceDNA vectors produced from the constructs 1-8 described above is assessed in mice. The ceDNA vector obtained from ceDNA-plasmid construct 1 (as described in Table 12) was tested and demonstrated sustained and durable luciferase transgene expression in a mouse model following hydrodynamic injection of the ceDNA construct without a liposome, redose (at day 28) and durability (up to Day 42) of exogenous firefly luciferase ceDNA. In different experiments, the luciferase expression of selected ceDNA vectors is assessed in vivo, where the ceDNA vectors comprise the luciferase transgene and at least one modified ITR selected from any shown in Tables 10A-10B, or an ITR comprising at least one sequences shown in FIGS. 26A-26B

In Vivo Luciferase Expression:

5-7 week male CD-1 IGS mice (Charles River Laboratories) are administered 0.35 mg/kg of ceDNA vector expressing luciferase in 1.2 mL volume via i.v. hydrodynamic administration to the tail vein on Day 0. . Luciferase expression is assessed by IVIS imaging on Day 3, 4, 7, 14, 21, 28, 31, 35, and 42. Briefly, mice are injected intraperitoneally with 150 mg/kg of luciferin substrate and then whole body luminescence was assessed via IVIS® imaging.

IVIS imaging is performed on Day 3, Day 4, Day 7, Day 14, Day 21, Day 28, Day 31, Day 35, and Day 42, and collected organs are imaged ex vivo following sacrifice on Day 42.

During the course of the study, animals are weighed and monitored daily for general health and well-being. At sacrifice, blood is collected from each animal by terminal cardiac stick, and split into two portions and processed to 1) plasma and 2) serum, with plasma snap-frozen and serum used for liver enzyme panel and subsequently snap frozen. Additionally, livers, spleens, kidneys, and inguinal lymph nodes (LNs) are collected and imaged ex vivo by IVIS.

Luciferase expression is assessed in livers by MAXDISCOVERY® Luciferase ELISA assay (BIOO Scientific/PerkinElmer), qPCR for Luciferase of liver samples, histopathology of liver samples and/or a serum liver enzyme panel (VetScanVS2; Abaxis Preventative Care Profile Plus).

### Example 5: ITR Walk Mutant Screening

Further analyses of the relationship of ITR structure to ceDNA formation were performed. A series of mutants were constructed to query the impact of specific structural changes on ceDNA formation and ability to express the ceDNA-encoded transgene. Mutant construction, assay of ceDNA formation, and assessment of ceDNA transgene expression in human cell culture are described in further detail below.

A. Mutant ITR Construction

A library of 31 plasmids with unique asymmetric AAV type II ITR mutant cassettes was designed in silico and subsequently evaluated in Sf9 insect cells and human embryonic kidney cells (HEK293). Each ITR cassette contained either a luciferase (LUC) or green fluorescent protein (GFP) reporter gene driven by a p10 promoter sequence for expression in insect cells, and a CAG promoter sequence for expression in mammalian cells. Mutations to the ITR sequence were created on either the right or left ITR region. The library contained 15 right-sided (RS) and 16 left-sided (LS) mutants, disclosed in Table 10A and 10B and FIGS. 26A and 26B herein.

Sf9 suspension cultures were maintained in Sf900 III media (Gibco) in vented 200 mL tissue culture flasks. Cultures were passaged every 48 hours and cell counts and growth metrics were measured prior to each passage using a ViCell Counter (Beckman Coulter). Cultures were maintained under shaking conditions (1″ orbit, 130 rpm) at 27° C. Adherent cultures of HEK293 cells were maintained in GlutiMax DMEM (Dulbecco's Modified Eagle Medium, Gibco) with 1% fetal bovine serum and 0.1% PenStrep in 250 mL culture flasks at 37° C. with 5% CO₂. Cultures were trypsinized and passaged every 96 hours. A 1:10 dilution of a 90-100% confluent flask was used to seed each passage.

ceDNA vectors were generated and constructed as described in Example 1 above. In brief, referring to FIG. 4B, Sf9 cells transduced with plasmid constructs were allowed to grow adherently for 24 hours under stationary conditions at 27° C. After 24 hours, transfected Sf9 cells were infected with Rep vector via baculovirus infected insect cells (BIICs). BIICs had been previously assayed to characterize infectivity and were used at a final dilution of 1:2000. BIICs diluted 1:100 in Sf900 insect cell media were added to each previously transfected cell well. Non-Rep vector BIICs were added to a subset of wells as a negative control. Plates were mixed by gentle rocking on a plate rocker for 2 minutes. Cells were then grown for an additional 48 hours at 27° C. under stationary conditions. All experimental constructs and controls were assayed in triplicate.

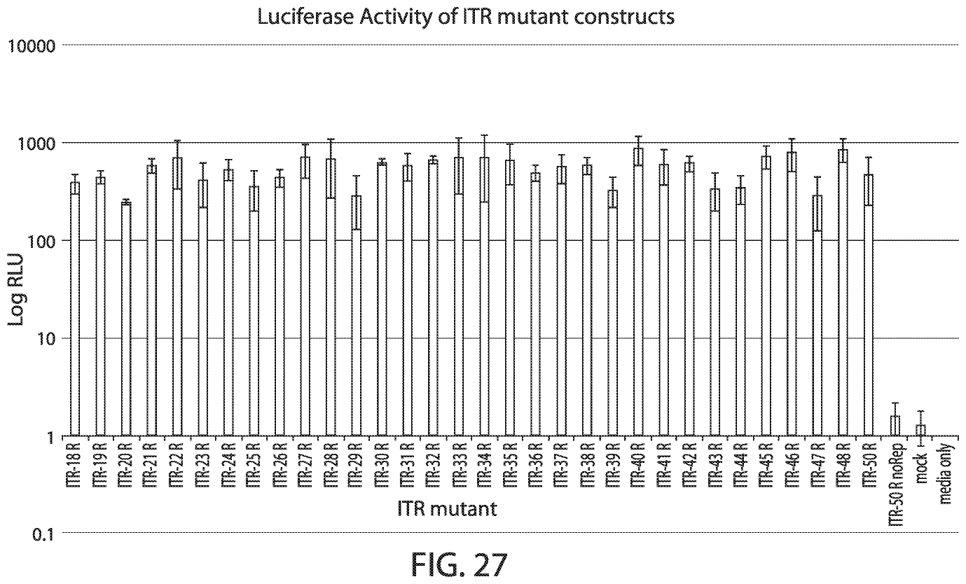

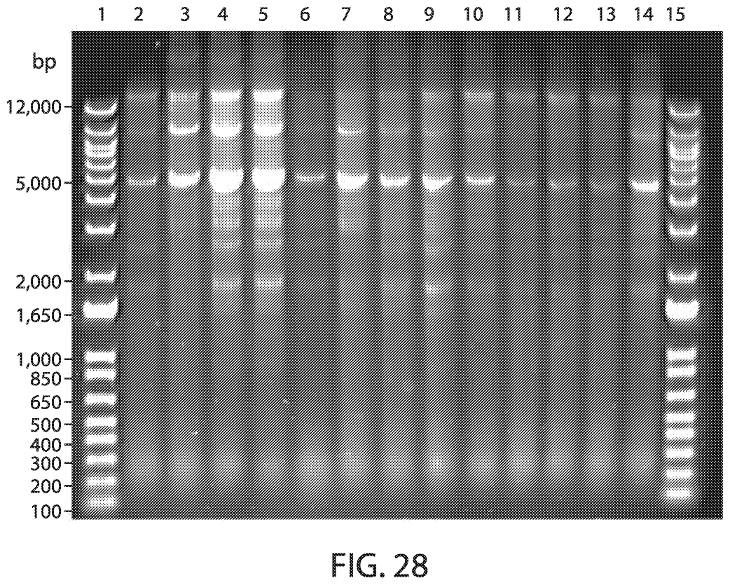

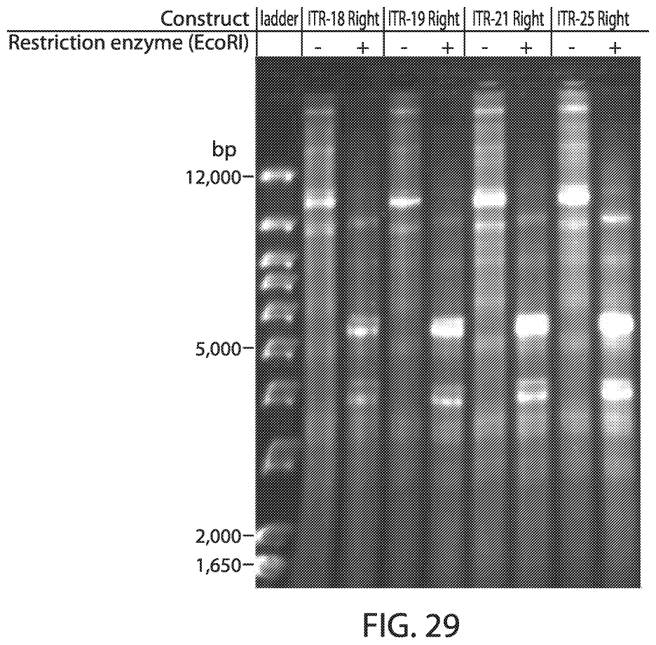

After 48 hours the 96-well plate was removed to from the incubator, briefly equilibrated to room temperature, and assayed for luciferase expression (OneGlo Luciferase Assay (Promega Corporation)). Total luminescence was measured using a SpectraMax M Series microplate reader. Replicates were averaged. The results are shown in FIG. 27. As expected, the three negative controls (media only, mock transfection lacking donor DNA, and sample that was processed in the absence of Rep-containing baculovirus cells) showed no significant luciferase expression. Robust luciferase expression was observed in each of the mutant samples, indicating that for each sample the ceDNA-encoded transgene was successfully transfected and expressed irrespective of the mutation.

B. Assay of ceDNA Formation

To ensure that the ceDNA generated in the preceding study was of the expected close-ended structure, experiments were performed to produce sufficient amounts of each ceDNA which could subsequently be tested for proper structure. Briefly, Sf9 suspension cultures were transfected with DNA belonging to a single ITR mutant plasmid from the library. Cultures were seeded at 1.25×10⁶ cells/mL in Erlenmeyer culture flasks with limited gas exchange. DNA:lipid transfection complexes were prepared using fuGene transfection reagent according to the manufacturer's instructions. Complex mixes were prepared and incubated in the same manner as previously described for the luciferase plate assay, with increased volumes proportionate to the number of cells being transfected. As with the reporter gene assay, a ratio of 4.5:1 (volume reagent/mass DNA) was used. Mock (transfection reagents only) and untreated growth controls were prepared in parallel with experimental cultures. Following the addition of transfection reagents, cultures were allowed to recover for 10-15 minutes at room temperature with gentle swirling before being transferred to a 27° C. shaking incubator. After 24 hours of incubation under shaking conditions, cell counts and growth metrics for all flasks (experimental and control) were measured using a ViCell counter (Beckman Coulter). All flasks (except growth control) were infected with Rep-vector-containing BIICs at a final dilution of 1:5,000. A positive control using the established BIIC dual infection procedure for ceDNA production was also prepared. The dual infection culture was seeded with the number of cells equal to the average viable cell count of all experimental cultures. Dual infection control was infected with Rep and reporter gene BIICs at a final dilution of 1:5,000 for each construct, respectively. After infection, cultures were placed back in the incubator under previously described shaking conditions. Cell counts, growth and viability metrics were measured daily for all flasks for 3 days post infection. T=0 timepoint measurements were taken after newly infected cultures had been allowed to recover for ˜2 hours under shaking incubation conditions. After 3 days cells were harvested by centrifugation for 15 minutes. Supernatant was discarded, mass of pellets was recorded, and pellets were frozen −80° C. until DNA extraction.

Putative crude ceDNA was extracted from all flasks (experimental and control) using the Qiagen Plasmid Plus Midi Purification kit (Qiagen) according to manufacturers “high yield” protocol. Eluates were quantified using optical density measurements obtained from a NanoDrop OneC (ThermoFisher). The resulting ceDNA extracts were stored at 4° C.

The foregoing ceDNA extracts were run on a native agarose (1% agarose, 1×TAE buffer) gel prepared with 1:10,000 dilution of SYBR Safe Gel Stain (ThermoFisher Scientific), alongside the Tracklt 1 kb Plus DNA ladder. The gel was subsequently visualized using a Gbox Mini Imager under UV/blue lighting. As previously described, two primary bands are expected in ceDNA samples run on native gels: a 5,500 bp band representing a monomeric species and a ˜11,000 bp band corresponding to a dimeric species. All mutant samples were tested and displayed the expected monomer and dimer bands on native agarose gels. The results for a representative sample of the mutants are shown in FIG. 28. Putative crude ITR-mutant ceDNA and control extracts from small scale production were further assayed using a coupled restriction digest and denaturing agarose gel to confirm a double stranded DNA structure diagnostic of ceDNA. Each mutant ceDNA is expected to have a single EcoRI restriction site, and so, if properly formed, to produce two characteristic fragments upon EcoRI digestion. High-fidelity restriction endonuclease EcoRI (New England Biolabs) was used to digest putative ceDNA extract according to manufacturer's instructions. Extracts from mock and growth controls were not assayed because spectrophotometric quantification using NanoDrop (ThermoFisher) as well as native agarose gel analysis had revealed there to be no detectible ceDNA/plasmid like product in the eluates. Digested material was purified using Qiagen PCR Clean-up Kit (Qiagen) according to manufacturers instructions with the exception that purified digested material was eluted in nuclease free water instead of Qiagen Elution Buffer. An alkaline agarose gel (0.8% alkaline agarose) was equilibrated in Equilibration Buffer (1 mM EDTA, 200 mM NaOH) overnight at 4° C. 10× Denaturing Solution (50 mM NaOH, 1 mM EDTA) was added to the samples of the purified ceDNA digests and corresponding un-digested ceDNA (1 ug total) and samples were heated at 65° C. for 10 minutes. 10× loading dye (Bromophenol blue, 50% glycerol) was added to each denatured sample and mixed. The TrackIt 1 kb Plus DNA ladder (ThermoFisher Scientific) was also loaded on the gel as a reference. The gel was run for ˜18 hrs at 4° C. and constant voltage (25 V), followed by rinsing with de-ionized H₂O and neutralization in 1×TAE (Tris-acetate, EDTA) buffer, pH 7.6, for 20 minutes with gentle agitation. The gel was then transferred to 1×TAE/1×SYBR Gold solution for ˜1 hour under gentle agitation. The gel was then visualized using a Gbox Mini Imager (Syngene) under UV/blue lighting. Uncut denatured samples were expected to migrate at ˜11,000 bp and the EcoRI treated samples were expected to have two bands, one at ˜4,000 bp and one at ˜6,000 bp.

All mutant samples had similar results in this experiment. Two significant bands were visible in each sample lane in the EcoRI-treated samples, migrating on the denaturing gel at the expected sizes, in sharp contrast to the undigested mutant samples, which migrated at the expected 11,000 bp size. FIG. 27 shows the results for a representative sample of mutants, where two bands above background are seen for each digested mutant sample, in comparison to the single band visible in the undigested mutant samples. Thus, the mutant samples seemed to correctly form ceDNA.

C. Functional Expression in Human Cell Culture

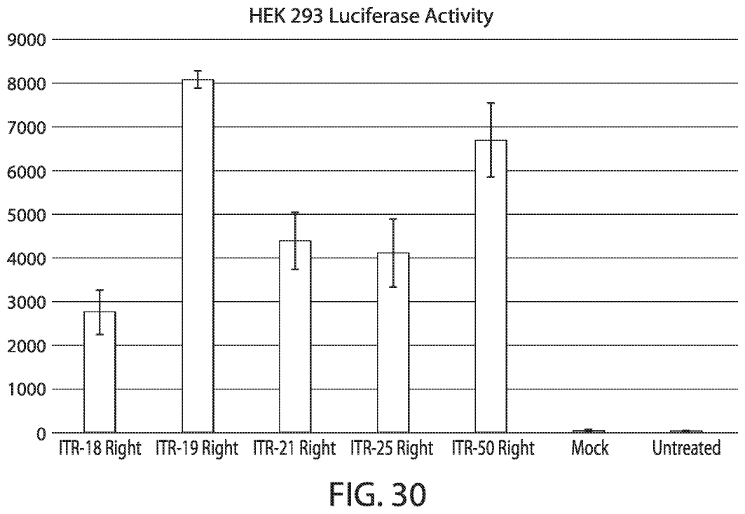

To assess the functionality of mutant ITR ceDNA produced by the small-scale production process, HEK293 cells were transfected with some representative mutant ceDNA samples. Actively dividing HEK293 cells were plated in 96-well microtiter plates at 3×10⁶ cells per well (80% confluency) and incubated for 24 hours at previously described conditions for adherent HEK293 cultures. After 24 hours, 200 ng total of crude small-scale ceDNA was transfected using Lipofectamine (Invitrogen, TheromoFisher Scientific). Transfection complexes were prepared according to manufacturers instructions and a total volume of 10 uL transfection complex was used to transfect previously plated HEK293 cells. All experimental constructs and controls were assayed in triplicate. Transfected cells were incubated at previously described conditions for 72 hours. After 72 hours the 96-well plate was removed to from the incubator and allowed to briefly equilibrate to room temperature. The OneGlo Luciferase Assay was performed. After 10 minutes on the orbital shaker, total luminescence was measured using a SpectraMax M Series microplate reader. Replicates were averaged. The results are shown in FIG. 30. Each of the tested mutant samples expressed luciferase in human cell culture, indicating that ceDNA was correctly formed and expressed for each sample in the context of human cells.

### REFERENCES

All references listed and disclosed in the specification and Examples, including patents, patent applications, International patent applications and publications are incorporated herein in their entirety by reference.

1. A non-viral capsid-free DNA vector with covalently-closed ends (ceDNA vector), wherein the ceDNA vector comprises at least one heterologous nucleotide sequence operably positioned between asymmetric inverted terminal repeat sequences (asymmetric ITRs), wherein at least one of the asymmetric ITRs comprises a functional terminal resolution site and a Rep binding site.

2. The ceDNA vector of claim 1, wherein the ceDNA vector when digested with a restriction enzyme having a single recognition site on the ceDNA vector and analyzed by both native and denaturing gel electrophoresis displays characteristic bands of linear and continuous DNA as compared to linear and non-continuous DNA controls.

3. The ceDNA vector of claim 1 or 2, wherein one or more of the asymmetric ITR sequences are from a virus selected from a parvovirus, a dependovirus, and an adeno-associated virus (AAV).

4. The ceDNA vector of claim 3, wherein the asymmetric ITRs are from different viral serotypes.

5. The ceDNA vector of claim 4, wherein the one or more asymmetric ITRs are from an AAV serotype selected from AAV1, AAV2, AAV3, AAV4, AAV5, AAV6, AAV7, AAV8, AAV9, AAV10, AAV11, and AAV12.

6. The ceDNA vector of any one of claims 1-3, wherein one or more of the asymmetric ITR sequences are synthetic.

7. The ceDNA vector of any one of claims 1-3 and 6, wherein one or more of the ITRs is not a wild type ITR.

8. The ceDNA vector of any one of claims 1-7, wherein one or more both of the asymmetric ITRs is modified by a deletion, insertion, and/or substitution in at least one of the ITR regions selected from A, A′, B, B′, C, C′, D, and D′.

9. The ceDNA vector of claim 8, wherein the deletion, insertion, and/or substitution results in the deletion of all or part of a stem-loop structure normally formed by the A, A′, B, B′ C, or C′ regions.

10. The ceDNA vector of claim 8 or claim 9, wherein one or both of the asymmetric ITRs is modified by a deletion, insertion, and/or substitution that results in the deletion of all or part of a stem-loop structure normally formed by the B and B′ regions.

11. The ceDNA vector of any one of claims 8-10, wherein one or both of the asymmetric ITRs is modified by a deletion, insertion, and/or substitution that results in the deletion of all or part of a stem-loop structure normally formed by the C and C′ regions.

12. The ceDNA vector of claim 10 or claim 11, wherein one or both of the asymmetric ITRs is modified by a deletion, insertion, and/or substitution that results in the deletion of part of a stem-loop structure normally formed by the B and B′ regions and/or part of a stem-loop structure normally formed by the C and C′ regions.

13. The ceDNA vector of any one of claims 1-12, wherein one or both of the asymmetric ITRs comprises a single stem-loop structure in the region that normally comprises a first stem-loop structure formed by the B and B′ regions and a second stem-loop structure formed by the C and C′ regions.

14. The ceDNA vector of claim 13, wherein one or both of the asymmetric ITRs comprises a single stem and two loops in the region that normally comprises a first stem-loop structure formed by the B and B′ regions and a second stem-loop structure formed by the C and C′ regions.

15. The ceDNA vector of claim 13 or claim 14, wherein one or both of the asymmetric ITRs comprises a single stem and a single loop in the region that normally comprises a first stem-loop structure formed by the B and B′ regions and a second stem-loop structure formed by the C and C′ regions.

16. The ceDNA vector of any one of claims 1-15, wherein at least one asymmetric ITR is a modified AAV2 ITR comprising a nucleotide sequence selected from: the ITRs in FIG. 26A or 26B, SEQ ID NOS: 101-499 or 545-547, an ITR having at least 95% sequence identity to an ITR in FIG. 26A or 26B, and an ITR having at least 95% sequence identity to SEQ ID NOS: 101-499 and 545-547.

17. The ceDNA vector of any one of claims 1-16, wherein at least one asymmetric ITR is a modified AAV2 ITR comprising a nucleotide sequence of SEQ ID NOS. 2, 52, 63, or 64, or a nucleotide sequence having at least 95% sequence identity to SEQ ID NOS. 2, 52, 63, or 64.

18. The ceDNA vector of any one of claims 1-16, wherein the 5′ ITR is a wild type AAV ITR and the 3′ ITR comprises a sequence selected from SEQ ID NO: 2, 64, 102, 104, 106, 108, 110, 112, 114, 116, 118, 120, 122, 124, 126, 128, 130, 132, 134, 469-483 and 546, and ITR sequences shown in FIG. 26A, and sequences having at least 95% sequence identity to any of the foregoing sequences.

19. The ceDNA vector of any one of claims 1-16, wherein the 3′ ITR is a wild type AAV ITR and the 5′ ITR comprises a sequence selected from SEQ ID NO: 52, 63, 101, 103, 105, 107, 109, 111, 113, 115, 117, 119, 121, 123, 125, 127, 129, 131, 133, 484-499, 545 and 547, and ITR sequences shown in FIG. 26B, and sequences having at least 95% sequence identity to any of the foregoing sequences.

20. The ceDNA vector of any one of claims 1-16, wherein the 5′ ITR comprises a sequence selected from SEQ ID NO: 52, 63, 101, 103, 105, 107, 109, 111, 113, 115, 117, 119, 121, 123, 125, 127, 129, 131, 133, 484-499, 545 and 547, and ITR sequences shown in FIG. 26B, and sequences having at least 95% sequence identity to any of the foregoing sequences; and the 3′ ITR comprises a sequence selected from SEQ ID NO: 2, 64, 102, 104, 106, 108, 110, 112, 114, 116, 118, 120, 122, 124, 126, 128, 130, 132, 134, 469-483 and 546, and ITR sequences shown in FIG. 26A, and sequences having at least 95% sequence identity to any of the foregoing sequences.

21. The ceDNA vector of claim 1, comprising at least two asymmetric ITRs selected from:
a. SEQ ID NO: 1 and SEQ ID NO:52; and
b. SEQ ID NO: 2 and SEQ ID NO: 51.

22. The ceDNA vector of claim 1, comprising a pair of asymmetric ITRs selected from:
a. SEQ ID NO:1 and SEQ ID NO:52; and
b. SEQ ID NO:2 and SEQ ID NO:51.

23. The ceDNA vector of any one of claims 1-20, wherein one or both asymmetric ITRs comprises a sequence other than SEQ ID NO: 2, 52, 63 64, 113, 114, and 557.

24. The ceDNA vector of any one of claims 1-24, wherein all or part of the heterologous nucleotide sequence is under the control of at least one regulatory switch.

25. The ceDNA vector of claim 24, wherein at least one regulatory switch is selected from the regulatory switches in Table 11.

26. The ceDNA vector of any one of claims 1-25, wherein the vector is in a nanocarrier.

27. The ceDNA vector of claim 26, wherein the nanocarrier comprises a lipid nanoparticle (LNP).

28. A non-viral capsid-free DNA vector with covalently-closed ends (ceDNA vector) of any one of claims 1-25, the ceDNA vector being obtained from a process comprising the steps of:
a. incubating a population of insect cells harboring a ceDNA expression construct in the presence of at least one Rep protein, wherein the ceDNA expression construct encodes the ceDNA vector, under conditions effective and for a time sufficient to induce production of the ceDNA vector within the insect cells; and
b. isolating the ceDNA vector from the insect cells.

29. The ceDNA vector of claim 28, wherein the ceDNA expression construct is selected from a ceDNA plasmid, a ceDNA bacmid, and a ceDNA baculovirus.

30. The ceDNA vector of claim 28 or claim 29, wherein the insect cell expresses at least one Rep protein.

31. The ceDNA vector of claim 30, wherein at least one Rep protein is from a virus selected from a parvovirus, a dependovirus, and an adeno-associated virus (AAV).

32. The ceDNA vector of claim 31, wherein at least one Rep protein is from an AAV serotype selected from AAV1, AAV2, AAV3, AAV4, AAV5, AAV6, AAV7, AAV8, AAV9, AAV10, AAV11, and AAV12.

33. A ceDNA expression construct that encodes the ceDNA vector of any one of claims 1-25.

34. The ceDNA expression construct of claim 33, which is a ceDNA plasmid, ceDNA bacmid, or ceDNA baculovirus.

35. A host cell comprising the ceDNA expression construct of claim 33 or claim 34.

36. The host cell of claim 35, which expresses at least one Rep protein.

37. The host cell of claim 36, wherein at least one Rep protein is from a virus selected from a parvovirus, a dependovirus, and an adeno-associated virus (AAV).

38. The host cell of claim 37, wherein at least one Rep protein is from an AAV serotype selected from AAV1, AAV2, AAV3, AAV4, AAV5, AAV6, AAV7, AAV8, AAV9, AAV10, AAV11, and AAV12.

39. The host cell of any one of claims 35 to 38, which is an insect cell.

40. The host cell of claim 39, which is an Sf9 cell.

41. A method of producing a ceDNA vector, comprising:
a. incubating the host cell of any one of claims 35-40 under conditions effective and for time sufficient to induce production of the ceDNA vector; and
b. isolating the ceDNA from the host cells.

42. A method for treating, preventing, ameliorating, monitoring, or diagnosing a disease or disorder in a subject, the method comprising: administering to a subject in need thereof, a composition comprising the ceDNA vector of any one of claims 1-25, wherein the at least one heterologous nucleotide sequence is selected to treat, prevent, ameliorate, diagnose, or monitor the disease or disorder.

43. The method of claim 42, wherein the at least one heterologous nucleotide sequence, when transcribed or translated corrects for an abnormal amount of an endogenous protein in the subject.

44. The method of claim 42, wherein the at least one heterologous nucleotide sequence, when transcribed or translated corrects for an abnormal function or activity of an endogenous protein or pathway in the subject.

45. The method of any one of claims 42-44, wherein the at least one heterologous nucleotide sequence encodes or comprises a nucleotide molecule selected from an RNAi, an siRNA, an miRNA, an lncRNA, and an antisense oligo- or polynucleotide.

46. The method of any one of claims 42-44, wherein the at least one heterologous nucleotide sequence encodes a protein.

47. The method of claim 42, wherein the at least one heterologous nucleotide sequence encodes a marker protein.

48. The method of any one of claims 42-46, wherein the at least one heterologous nucleotide sequence encodes an agonist or an antagonist of an endogenous protein or pathway associated with the disease or disorder.

49. The method of any one of claims 42-46, wherein the at least one heterologous nucleotide sequence encodes an antibody.

50. The method of claims 42-49, wherein the ceDNA vector is administered in combination with a pharmaceutically acceptable carrier.

51. A method for delivering a therapeutic protein to a subject, the method comprising:
administering to a subject a composition comprising the ceDNA vector of any of claims 1-25, wherein at least one heterologous nucleotide sequence encodes a therapeutic protein.

52. The method of claim 51, wherein the therapeutic protein is a therapeutic antibody.

53. A kit comprising a ceDNA vector of any of claims 1-25, and a nanocarrier, packaged in a container with a packet insert.

54. A kit for producing a ceDNA vector, the kit comprising:
a. an expression construct comprising at least one restriction site for insertion of at least one heterologous nucleotide sequence, or regulatory switch, or both, the at least one restriction site operatively positioned between asymmetric inverted terminal repeat sequences (asymmetric ITRs), wherein at least one of the asymmetric ITRs comprises a functional terminal resolution site and a Rep binding site.

55. The kit of claim 54, which is suitable for producing the ceDNA vector of any one of claims 1-25.

56. The kit of claim 54 or claim 55, further comprising a population of insect cells which is devoid of viral capsid coding sequences, that in the presence of Rep protein can induce production of the ceDNA vector.

57. The kit of any one of claims 54-56, further comprising a vector comprising a polynucleotide sequence that encodes at least one Rep protein, wherein the vector is suitable for expressing the at least one Rep protein in an insect cell.
